(19) United States
(12) Patent Application Publication
(10) Pub. No.: US 2010/0190824 A1
(43) Pub. Date: Jul. 29, 2010

(54) Novel Substituted Piperidones as HSP Inducers

(76) Inventors: Prabhat Kumar, Gujarat (IN); Anookh Mohanan, Gujarat (IN); Navnath Argade, Gujarat (IN); Chakradhar Hadole, Gujarat (IN); Appaji Mandhare, Gujarat (IN); Ramesh Gupta, Gujarat (IN); Shailesh Deshpande, Gujarat (IN); Prashant Jamadarkhana, Gujarat (IN); Poonam Joshi, Gujarat (IN)

Correspondence Address: JACOBSON HOLMAN PLLC, 400 SEVENTH STREET N.W., SUITE 600, WASHINGTON, DC, 20004, US

(21) Appl. No.: 12/452,389

(22) PCT Filed: Jun. 24, 2008
(86) PCT No.: PCT/IN2008/000400

§ 371 Date: Mar. 29, 2010

(30) Foreign Application Priority Data
Jun. 29, 2007 (IN) 947/KOL/2007

(51) Int. Cl. A61K 31/4545; C07D 211/68; A61P 25/28; A61P 25/08; A61P 9/10

(52) U.S. Cl. 514/318; 546/193

(57) ABSTRACT

The present invention relates to novel compounds of formula (I) or (II), their pharmaceutically acceptable salts and their hydrates, solvates, stereoisomers, conformers, tautomers, polymorphs and prodrugs and also pharmaceutically acceptable compositions containing them Wherein R₁, R₂, R₃, R₄, R₅, R₆ and R₇ are as defined in the specification. The compounds of the present invention are HSP inducers and by virtue of this effect, useful for the treatment of various diseases accompanying pathological stress. The present invention also relates to a process for the preparation of the said novel compounds. The invention also relates to the use of the above-mentioned compounds for the preparation of medicament for use as pharmaceuticals.

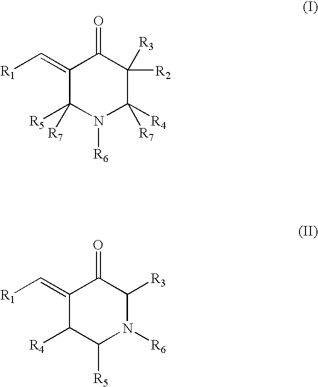

### FIELD OF THE INVENTION

The present invention relates to novel substituted piperidones, their pharmaceutically acceptable salts and their hydrates, solvates, stereoisomers, conformers, tautomers, polymorphs and prodrugs and also pharmaceutically acceptable compositions containing them. The compounds of the present invention are HSP inducers and by virtue of this effect, useful for the treatment of various diseases accompanying pathological stress selected from ischemic stroke, myocardial infarction, inflammatory disorders, diseases of viral origin, tumourous diseases, brain haemorrhage, endothelial dysfunctions, diabetic complications, hepatotoxicity, acute renal failure, glaucoma, sepsis, gastric mucosal damage, allograft rejection, neurodegenerative diseases, epilepsy, post-traumatic neuronal damage and aging-related skin degeneration. The present invention also relates to a process for the preparation of the said novel compounds. The invention also relates to the use of the above-mentioned compounds for the preparation of medicament for use as pharmaceuticals.

### BACKGROUND OF THE INVENTION

Heat shock proteins (HSPs) have been well documented to play a cytoprotective role in almost all living cells under various pathological stresses through a mechanism known as thermotolerance or cross tolerance. Heat shock proteins function as molecular chaperones or proteases that, under physiological conditions, have a number of intracellular functions. Chaperones are involved in the assembly and folding of misfolded or denatured oligomeric proteins, whereas proteases mediate the degradation of damaged proteins.

Heat shock proteins are categorized into several families that are named on the basis of their approximate molecular mass (e.g. the 70 kDa HSP-70, ubiquitin, HSP-10, HSP-27, HSP-32, HSP-60, HSP-90 etc). HSP-70 is the most abundant HSP found in normal cells. HSP-70, and its inducible form, called HSP-72, is found in all living cells. Following heat shock, its synthesis increases to a point to where it becomes the most abundant single protein in the cell.

Although some proteins refold spontaneously, in vitro, when diluted at low concentrations from denaturants, larger, multidomain proteins often have a propensity to misfold and aggregate. Consequently, the challenge within the densely packed cellular environment is to ensure that non-native intermediates are efficiently captured, maintained in intermediate folded states, and subsequently either refolded or degraded. Molecular chaperones such as HSP-90, HSP-70 and HSP-60 accomplish this by capturing non-native intermediates and, together with co-chaperones and ATP.

The HSP-70 chaperones, for example, recognize stretches of hydrophobic residues in polypeptide chains that are transiently exposed in early folding intermediates and typically confined to the hydrophobic core in the native state. The consequence of chaperone interactions, therefore, is to shift the equilibrium of protein folding and refolding reactions toward productive on-pathway events and to minimize the appearance of non-productive intermediates that have a propensity to aggregate as misfolded species.

Over the past years, a number of studies have shown that the major heat-inducible protein, HSP-72, is critical for protection of cells and tissues from heat shock and other stresses. HSP-72 functions as molecular chaperone in refolding and degradation of damaged proteins. This has led to the common assumption that chaperoning activities of HSP-72 determine its role in ability of a cell to protect itself against stresses. Upon exposure to stresses that lead to a massive protein damage and necrotic death, the anti-aggregating and protein refolding activities of HSP-72 may indeed become critical for cell protection. On the other hand, upon exposure to stresses that lead to apoptosis, the protective function of HSP-72 could be fully accounted for by its distinct role in cell signaling. Under these conditions, protein damage on its own is not sufficient for cell death because suppression of the apoptotic signaling pathway restores cell viability.

The term heat shock protein is somewhat of a misnomer, as they are not induced solely by heat shock. Indeed, in addition to being constitutively expressed (making up 5-10% of the total protein content under normal growth conditions), these proteins can be markedly induced (up to 15% of the total cellular protein content) by a range of stimuli including various pathological stresses.

Pathological stresses inducing heat shock protein expression include a wide variety of conditions associated with many diseases. The synthesis of heat shock proteins in cells exposed to such stresses indicates the first line of defense of the cell against the pathological stresses.

### Stroke

One such pathological condition wherein protective role of HSP-70 has been implicated is cerebral ischemic injury (stroke). Cerebral ischaemia causes severe depletion of blood supply to the brain tissues, as a result of which the cells gradually proceed to death due to lack of oxygen. In such a situation, there is increased expression of heat shock protein in the brain tissue. Transient ischemia induces HSPs in the brain and the ability of neuronal population to survive an ischemic trauma is correlated with increased expression of HSP-70. HSP-70 mRNA was induced in neurons at the periphery of ischemia. It is proposed that the peripheral zone of ischemia, penumbra can be rescued by pharmacological agents. It was in this zone that HSP-70 protein was found to be localized primarily in neurons. [Dienel G. A. et al., J. Cereb. Blood Flow Metab., 1986, Vol. 6, pp. 505-510; Kihouchi H. et al., Brain Research, 1993, Vol. 619, pp. 334-338]. The direct assessment of the protective role of HSP-70 is shown by using transgenic mice overexpressing the rat HSP (HSP-70tg mice). In contrast to wild-type littermates, high levels of HSP messenger RNA and protein were detected in brains of HSP-70tg mice under normal conditions, immunohistochemical analysis revealed primarily neuronal expression of HSP-70. Heterozygous HSP-70tg mice and their wild type littermates were subjected to permanent focal cerebral ischemia by intraluminal blockade of middle cerebral artery. Cerebral infarction after 6 hours of ischemia, as evaluated by nissl staining, was significantly less in HSP-70tg mice compared with wild type littermate mice. The HSP-70tg mice were still protected against cerebral infarction 24 hours after permanent focal ischemia. The data suggest that HSP-70 can markedly protect the brain against ischemic damage. [Rajdev S., Hara K, et al., Ann. Neurol., 2000 June, Vol. 47 (6), pp. 782-791] The 72-kD inducible heat shock protein (HSP-72) plays a very important role in attenuating cerebral ischemic injury. Striatal neuronal survival was significantly improved when HSP-72 vectors was delivered after ischemia onset into each striatum. [Hoehn B. et al., J. Cereb. Blood Flow Metab., 2001 November, Vol. 21(11), pp. 1303-1309].

Experiments have proved that neurological deficits induced by ischemia were found to be reduced on treatment with HSP-inducers like lithium. These neuroprotective effects were associated with an up-regulation of cytoprotective heat shock protein-70 in the ischemic hemisphere [Ren M. et al., Proc. Natl. Acad. Sci. USA., 2003 May 13; Vol. 100(10), pp. 6210-6215]. Thus induction of HSP-70 would confer a protective effect in cerebral ischaemic injury (stroke).

### Myocardial Infarction

Another pathological condition analogous to cerebral ischaemia is myocardial infarction, in which case, severe ischemia even for relatively short periods of time, lead to extensive death of cardiomyocytes. Induction of HSP-70 has been shown to confer protection against subsequent ischemia as is evident by a direct correlation to post-ischemic myocardial preservation, reduction in infarct size and improved metabolic and functional recovery. Overexpression of inducible HSP-70 in adult cardiomyocytes were associated with a 34% decrease in lactate dehydrogenase in response to ischemic injury. [Hutter M. M. et al., Circulation, 1994, Vol. 89, pp. 355-360; Liu X. et al., Circulation, 1992, Vol. 86, pp. 11358-11363; Martin J. L., Circulation, 1997, Vol. 96, pp. 4343-4348].

Experiments have shown that oral pretreatment of rats with an HSP inducer Bimoclomol elevated myocardial HSP-70 and reduced infarct size in a rat model of ischemia [Lubbers N. L. et al., Eur. J. Pharmacol., 2002 Jan. 18, Vol. 435(1), pp. 79-83]. There was a significant correlation between HSP-70 induction and infarct size reduction after oral administration of Bimoclomol. Further, Bimoclomol also improved cell survival in rat neonatal cardiomyocytes by increasing the levels of HSP-70 [Polakowski J. S. et al., Eur. J. Pharmacol., 2002 Jan. 18, Vol. 435 (1), pp. 73-77].

In further experiments, transgenic mice were engineered to express high levels of the rat-inducible HSP-70 [Marber M. S. et al., J. Clin. Invest., 1995 April, Vol. 95, pp. 1446-1456]. It was observed that there was a significant reduction in infarct size by about 40% after 20 minutes of global ischemia in the heart of the transgenic mice, and contractile function doubled during reperfusion period compared to wild type.

Moreover, evidence indicate that myocardial stress protein HSP-70 is directly protective, is provided by the observation that transfected myocyte lines overexpressing HSP-70 have enhanced resistance to hypoxic stress [Mestril R. et al., J. Clin. Invest., 1994 February, Vol. 93, pp. 759-767].

Further investigations into the role of HSP-70 overexpression through gene therapy on mitochondrial function and ventricular recovery has shown that, HSP-70 upregulation protects mitochondrial function after ischemia-reperfusion injury and was associated with reproved preservation of myocardial function.

Post ischemic mitochondrial respiratory control indices linked to NAD and FAD were better preserved and recovery of mechanical function was greater in HSP transfected than control hearts. [Jayakumar J. et al., Circulation, 2001 Sep. 18, Vol. 104 (12 Suppl 1), pp. 1303-1307]. Thus, the foregoing evidence indicates that induction of HSP-70 would be useful for treating myocardial infarction.

### Inflammatory Disorders

Yet another example of pathological stress on tissues and organs causing HSP-70 induction is provided by inflammatory diseases.

Inflammation is caused by activation of phagocytic cells like leucocytes, primarily by monocytes-macrophages, which generate high levels of reactive oxygen species (ROS) as well as cytokines. Both ROS and cytokines upregulate the expression of heat shock proteins (HSP), while HSPs in turn protect cells and tissues from the deleterious effects of inflammation. In an in vivo model for adult respiratory distress syndrome, an acute pulmonary inflammatory condition which caused HSP induction, HSP completely prevented mortality. [Jacquier-Salin M. R. et al., Experientia, 1994 Nov. 30, Vol. 50 (11-12), pp. 1031-1038].

HSP exert multiple protective effects in inflammation, including self/non-self discrimination, enhancement of immune responses, immune protection, thermotolerance and protection against the cytotoxicity of inflammatory mediators [Polla B. S. et al., EXS., 1996, Vol. 77, pp. 375-91].

Heat shock proteins (HSPs) have been repeatedly implicated in the control of the progression of rheumatoid arthritis. An up-regulation of HSP-70 expression in synovial tissue is consistently observed in patients with rheumatoid arthritis. Recent investigations have shown that, pro-inflammatory cytokines induced activation of HSF 1-DNA binding and HSP-70 expression in cultivated synovial fibroblast-like cells [Georg Schett et. al., J. Clin. Invest., 1998 July, Vol. 102 (2), pp. 302-311]. Since HSP-70 is critically involved in protein folding and may prevent apoptotic cell death, facilitating synovial growth and pannus formation, their elevated levels would play a crucial role in controlling the progression of the disease state.

Anti-inflammatory agents such as NSAIDS activate HSF-1 DNA binding and glucocortcoids at high dose activate HSF-1 as well as induce HSP expression [Georg Schett et. al., J. Clin. Invest., 1998 July, Vol. 102 (2), pp. 302-311].

HSP-70 has a role in controlling inflammation. The induction of HSP-70 before the onset of inflammation can reduce organ damage [Hayashi Y. et al, Circulation, 2002 Nov. 12, Vol. 106(20), pp. 2601-2607]. Preoperative administration of HSP-70 inducers seem to be useful in attenuating cardiopulmonary bypass (CPB)-induced inflammatory response.

Investigations into the anti-inflammatory property of 2-cyclopentene-1-one demonstrated that the heat shock factor 1(HSF1) activation, subsequent induction of HSP-72 expression occurs in inflamed tissue and this effect is associated with the remission of the inflammatory reaction. [Ianaro A. et al., Mol. Pharmacol., 2003 July, Vol. 64(1), pp. 85-93]. The anti-inflammatory properties of 2-cyclopenten-1-one were associated with HSF-1 induced HSP-72 expression in vivo.

The HSP co-inducer BRX-220 has been examined for effects on the Cholecystokinin-octapeptide (CCK)-induced acute pancreatitis in rats [Rakonczay Z. Jr. et al., Free Radic. Biol. Med., 2002 Jun. 15, Vol. 32 (12), pp. 1283-1292]. The pancreatic levels of HSP-60 and HSP-72 were significantly increased in the animals treated with BRX-220. Further, pancreatic total protein content, amylase and trypsinogen activities were higher with increased glutathione peroxidase activity. A decrease in plasma trypsinogen activation peptide concentration, pancreatic lipid peroxidation, protein oxidation, and the activity of Cu/Zn-Superoxide dismutase were also observed. Thd protective action of BRX-220 on pancreatitis was ascribed directly to its HSP-70 inducing action.

Whole body hyperthermia in rats leading to induction of HSP-70 has been shown to protect against subsequent caerulein-induced acute pancreatitis. More specifically the degradation and disorganization of the actin cytoskeleton, an important early component of pancreatitis was prevented [Tashiro M. et al., Digestion, 2002, Vol. 65 (2), pp. 118-126], hence, reducing damage in pancreatitis secondary to inflammation. Thus induction of HSP-70 would be beneficial in treating inflammatory disorders.

### Hepatotoxicity

Another example of a pathological stress wherein protective role of HSP-70 has been implicated is hepatotoxicity. Overproduction of heat shock protein 70 (HSP-70) in the liver protects hepatocytes under various pathologic conditions. Studies aimed at examining the effects of HSP-70 inducers, on acute hepatic failure after 95% hepatectomy have shown significantly suppressed release of aspartate or alanine aminotransferase and elevation of the serum interleukin-6 level [Oda H. et al, J. Gastrointest. Surg., 2002 May-June, Vol. 6(3), pp. 464-472].

The effect of HSP Inducer gadolinium chloride was studied in relation to its effect on metallothionein and heat shock protein expression in an in-vivo model of liver necrosis induced by thioacetamide [Andrés D. et al., Biochem. Pharmacol., 2003 Sep. 15, Vol. 66 (6), pp. 917-926]. Gadolinium significantly reduced serum myeloperoxidase activity and serum concentration of TNF-alpha and IL-6, increased by thioacetamide. The extent of necrosis, the degree of oxidative stress and lipoperoxidation and microsomal FAD monoxygenase activity were significantly diminished. These beneficial effects are attributed to enhanced expression of HSP-70 following Gadolinium administration.

Thus induction of HSP-70 would exert a protective effect in case of hepatotoxicity.

### Sepsis

Yet another pathological condition wherein induction of HSP-70 has been found to be beneficial is sepsis. Sepsis is a severe illness caused by overwhelming infection of the bloodstream by toxin-producing bacteria. Induction of HSPs by heat shock treatment significantly decreased the mortality rate of late sepsis. The involvement of HSPs during the progression of sepsis could add to a first line of host defense against invasive pathogens.

Expression of HSP-72 and their protective role has been studied using a rat model of cecal ligation and puncture [Yang R. C. et al., Kaohsiung J. Med. Sci., 1998 November, Vol. 14 (11), pp. 664-672]. Induction of HSP-70 expression by Geranylgeranyl acetone has shown to protect against cecal ligation and perforation induced diaphragmatic dysfunction. It showed a time dependant induction of HSP-70 in the diaphragm, which attenuated septic diaphragm impairment. [Masuda Y. et al., Crit. Care Med., 2003 November, Vol. 31(11), pp. 2585-2591]. GGA has found to induce HSP-70 expression in the diaphragm, which was attributed to be the underlying mechanism for the protective action of GGA

Further experiments indicate that induction of HSP-70 by the administration of sodium arsenite conferred significant protection against cecal ligation and perforation-induced mortality [Ribeiro S. P. et al., Crit. Care Med., 1994 June, Vol. 22(6), pp. 922-929]. In-vivo Sodium arsenite injection in the absence of an increase in body temperature, induced expression of HSP-72 in the lungs and protected against experimental sepsis. Protection conferred resulting in reduced mortality correlated directly with the expression of heat shock protein 72 in the lungs at 18 and 24 hours after perforation.

It was observed that induction of heat shock proteins by thermal stress reduced organ injury and death in a rat model of intra-abdominal sepsis and sepsis-induced acute lung injury [Villar J. et al., Crit. Care Med., 1994 June, Vol. 22 (6), pp. 914-921].

Acute respiratory distress syndrome (ARDS) provokes three pathologic processes: unchecked inflammation, interstitial/alveolar protein accumulation and destruction of pulmonary epithelial cells. Heat shock protein HSP-70 can limit all three responses, only if expressed adequately. Restoring expression of HSP-70 using adenovirus-mediated gene therapy has shown to be beneficial [Yoram G. W. et al., J. Clin. Invest. 2002, Vol. 110, pp. 801-806]. HSP-70 administration significantly attenuated interstitial and alveolar edema along with protein exudation and dramatically decreased neutrophil accumulation. Approximately 2-fold higher expression of HSP-70 conferred 68% survival at 48 hours as opposed to only 25% in untreated animals. Modulation of HSP-70 production reduced the pathological changes and improved outcome in experimental acute respiratory distress syndrome. Thus, inducers of HSP-70 would confer protective effect in sepsis.

### Viral Diseases

Another pathological condition in which induction of HSP-70 occurs is in case of viral diseases. Heat shock proteins (HSPs) and molecular chaperones have been known for several years to protect cells against virus infection [Lindquist S. et al., Annu. Rev. Genet., 1988, Vol. 22, pp. 631-637]. It has been demonstrated that induction of HSP-70 is associated with inhibition of infectious virus production and viral protein synthesis in monkey kidney epithelial cells infected with vesicular stomatitis virus (VSV) [Antonio R. et al., J. of Biol. Chem., 1996 Issue of December 13, Vol. 271 (50), pp. 32196-32196]. The pathogenic activity of Viral protein R (Vpr) of human immunodeficiency virus type 1 (HIV-1) is related in part to its capacity to induce cell cycle G2 arrest and apoptosis of target T cells. Overexpression of HSP-70 reduced the Vpr-dependent G2 arrest and apoptosis and also reduced replication of the Vpr-positive, but not Vpr-deficient, HIV-1. [Iordanskiy S. et al., J. Virol., 2004 September, Vol. 78 (18), pp. 9697-9704]. Induction of HSP-70 by prostaglandin A1 (PGA1) caused the suppression of influenza virus production. [Hirayama E., Yakugaku Zasshi, 2004 July, Vol. 124 (7), pp. 437-442].

The antiviral activity of Cyclopentenone prostaglandins is mediated by induction of HSP-70. It has been shown that increased synthesis of HSP-70 exerts potent antiviral activity in several DNA and RNA virus models—vesicular stomatitis virus, sindbis virus, sendai virus, polio virus etc. [Santoro M. G., Experientia, 1994 Nov. 30, Vol. 50 (11-12), pp. 1039-1047; Amici C. et al., J. Gen. Virol., 1991 August, Vol. 72, pp. 1877-1885; Amici C. et al., J. Virol., 1994 November, Vol. 68(11), pp. 6890-6899; Conti C. et al., Antimicrob. Agents Chemother., 1996 February, Vol. 40(2), pp. 367-372; Conti C. et al., Antimicrob. Agents Chemother., 1999 April, Vol. 43 (4), pp. 822-829]. Therefore, induction of HSP-70 would exert antiviral effect.

### Allograft Rejection

Allograft (transplant of an organ or tissue from one individual to another of the same species with a different genotype) rejection is a pathological condition causing induction of HSP-70. HSP-70 induction has a protective effect, which preserves organ function after transplantation. Kidneys can be preserved only for a limited time without jeopardizing graft function and survival. Induction of heat shock proteins (HSPs) has been found to improve the outcome following isotransplantation after an extended period of cold storage. Heat precondition induced the expression of HSP-70 and the grafts were protected against structural ischemia-reperfusion injuries when assessed histologically. [Wagner M. et al., Kidney Int., 2003 April, Vol. 63 (4), pp. 1564-1573]. There was inhibition of apoptosis and activation of caspase-3 was found to be inhibited.

Geranylgeranyl acetone, a non-toxic heat shock protein inducer has been studied in a rat orthotopic liver transplantation model to study the beneficial effects in warm ischemia-reperfusion injury [Fudaba Y. et al., Transplantation, 2001 Jul. 27, Vol. 72(2), pp. 184-189]. GGA administration accumulated mRNA for both HSP-72 and HSP 90 in the livers even before warm ischemia and facilitated the syntheses of HSP-72 and HSP 90 after warm ischemia. Further, GGA pretreatment also significantly reduced the serum levels of tumor necrosis factor-alpha after reperfusion. The findings indicate that both the enhanced induction of HSPs and the downstream events would be involved in the beneficial effects of GGA on ischemia-reperfusion injury. Besides, compared to donors treated with vehicle were all recipients died of primary non-function, when donors were treated with Geranylgeranyl acetone (GGA) the 7-day survival of the recipients was closed to 90%.

Investigations revealed an inverse relationship between HSP expression and rejection with the possibility that elevated levels of HSP in the myocardium results in low rejection of heart transplants. [Baba H. A. et al., Transplantation, 1998 Mar. 27, Vol. 65 (6), pp. 799-804]. Significant improvement of post-ischemic recovery of mechanical function in HSP-70 gene transfected hearts compared to controls were observed following a protocol mimicking conditions of preservation for heart transplantation. These results confirmed the findings observed previously in cell culture models and extended then to show the role of HSP-70 in protecting against ischemia-reperfusion injury in a whole-heart model, which parallels more closely the clinical situation. [Jayakumar J. et al., Circulation, 2000, Vol. 102 [ suppl III], pp. III-302 to 111-306].

The heat shock response also exerts a protective effect on skin flap ischemia. Heat shock protein (HSP) expression is augmented in-vivo with the administration of high dose aspirin before heat treatment [Ghavami A. et al., Ann. Plast. Surg., 2002 January, Vol. 48(1), pp. 60-67]. Immunohistochemistry confirmed HSP expression, and skin flap survival was improved significantly. Thus, HSP-70 induction would be beneficial in preserving organ function after transplantation.

### Tumorous Diseases

Induction of HSP-70 has also been shown to be advantageous in treating neoplasms. Enhanced expression of HSP-70 has been found to help in causing tumor regression in various animal models. Heat shock proteins (HSPs) are involved in the development of resistance (thermotolerance) to subsequent hyperthermic stresses as well as enhancement of the clinical response of certain chemotherapeutic agents in cancers such as the prostate. Colony formation assays revealed sensitizing effect of hyperthermia when simultaneously combined with each chemotherapeutic agent, resulting in a potentiated localized cytotoxicity [Roigas J. et al., Prostate, 1998 Feb. 15, Vol. 34 (3), pp. 195-202]. Synchronous application of chemotherapeutic agents and hyperthermia has been shown to have synergistic cytotoxic effect on Dunning rat adenocarcinoma of the prostate. Furthermore it is demonstrated that the induction of HSPs in thermotolerant cells, as measured by HSP-70 induction, results in a modulation of the chemotherapeutic-mediated cytotoxicity.

Direct induction of heat shock proteins are recognized to contribute significantly in cancer immunity. Anti-tumor immunity is induced by hyperthermia and further enhanced by administration of recombinant HSP-70 protein into the tumor in-situ. [Ito A. et al., Cancer Immunol. Immunother., 2004 January, Vol 53(1), pp. 26-32]. The induction of hyperthermia using a 500 KHz alternating magnetic field combined with magnetite cationic liposomes, which have a positive charge and generate heat in an alternating magnetic field along with administration of recombinant HSP-70 protein into the subcutaneous murine melanoma inhibited tumor growth over a 30-day period and complete regression of tumors was observed in 20% of mice. It was also found that systemic anti-tumor immunity was induced in cured mice. In another study carried out to determine whether anti-tumor immunity induced by hyperthermia is enhanced by HSP-70 gene transfer [Ito A. et al., Cancer Gene Ther., 2003 December, Vol. 10(12), pp. 918-925] showed that the combined treatment strongly arrested tumor growth over a 30-day period and complete regression of tumors was observed in 30% mice. Thus, induction of HSP-70 would be useful for the treatment of tumorous diseases.

### Gastric Mucosal Damage

Gastric mucosal damage caused by insults derived from ingested foods and Helicobacter pylori infection constitute another pathological condition causing induction of HSP-70. Gastric surface mucous cells are the first line of defense against such insults. Primary cultures of gastric surface mucous cells from guinea-pig fundic glands exhibited a typical heat shock response after exposure to elevated temperature or metabolic insults, such as ethanol and hydrogen peroxide, and they were able to acquire resistance to these stressors. HSP-70 mRNA protein has been induced in rat gastric mucosa following stress and the extent of induction inversely correlated with the severity of mucosal lesions suggesting protective role of HSP-70 in gastric mucosal defense. [Rokutan K., J. Gastroenterol. Hepatol., 2000 March, Vol. 15 Suppl, pp. D12-9].

### Brain Haemorrhage

Another pathological condition causing induction of HSP-70 is in case of brain haemorrhage. Studies with Bimoclomol showed an ability to reduce the pathological increase in the permeability of blood brain barrier during cerebrovascular injury, particularly if the vascular insult is evoked by sub-arachnoidal autologous blood [Erdo F. et al., Brain Research Bulletin, 1998, Vol. 45(2), pp. 163-166]. Bimoclomol strongly reduced the size of cerebral tissue stained with Evans blue leakage by 39%. Bimoclomol confers beneficial influences in experimental sub-arachnoid haemorrhage through its co-inducer effect on HSP-72 expression.

### Endothelial Dysfunctions

Various endothelial dysfunctions constitute pathological conditions which results in induction of HSP-70 in the body cells. The effect of a co-inducer of heat shock proteins, Bimoclomol treatment on endothelial function and expression of 72 Kd heat shock protein was investigated in spontaneously hypertensive rats [Jednakovits A. et. al., Life Sci., 2000 Aug. 25, Vol. 67(14), pp. 1791-1797]. Significant age-dependant decline in relaxation to acetylcholine and vascular HSP-72 mRNA levels were observed in SHR animals. These changes were found to be prevented by application of Bimoclomol suggesting the relationship between preservation of endothelial function with sustained levels of HSP-72.

### Diabetic Complications

Complications arising in diabetic patients such as neuropathy, retinopathy, nephropathy and delayed wound healing constitute pathological conditions wherein protective role of HSP-70 has been implicated.

### (a) Diabetic Neuropathy

Endoneurial microangiopathy causing nerve infarctions is considered to be involved in the pathogenesis of diabetic neuropathy [Malik R. A. et al., Diabetic Neuropathy: New Concepts and Insights, 1995, pp 131-135]. Experimental evidence is suggestive of a protective effect of HSP-72 induction on diabetic neuropathy [Biro K. et. al., Brain Research Bulletin, 1997, Vol. 44(3), pp. 259-263]. Treatment with Bimoclomol, by virtue of its HSP-70 inducing property significantly reduced nerve conduction slowing, motor by 38% and sensory by 42%, which show a dose dependant response. It also retarded the typical elevated ischemic resistance due to streptozotocin-induced neuropathy by 71%. These effects were observed at doses known to induce transcription of HSP-72 in other tissues like heart and kidney in response to ischemia.

### (b) Diabetic Retinopathy

Diabetic retinopathy is associated with the breakdown of the blood-retinal barrier (BRB) and results in macular edema, the leading cause of visual loss in diabetes. The HSP co-inducer Bimoclomol (BRLP-42) has shown efficacy in diabetes-induced retinopathy [Hegedius S. et al., Diabetologia, 1994, Vol. 37, p. 138]. The protection reflected in lower degree of edema in and beneath the photoreceptor zone, almost normal arrangement of retinal pigment epithelial microvilli and a more compact and even retinal capillary basement membrane. [Biro K. et al, Neuro Report, 1998 Jun. 22, Vol. 9(9), pp. 2029-2033]. Improvements are attributed to the cytoprotective effect of Bimoclomol on retinal glia and/or neurons against diabetes related ischemic cell damages. Further, overexpression of HSP-70 has shown protective effect on retinal photic injuries [Kim J. H. et al., Korean J. Opthalmol. 2003 June, Vol. 17(1), pp. 7-13].

### (c) Chronic Wound Healing

HSPs are involved in regulation of cell proliferation. Impaired expression of HSP-70 has been associated with delayed wound healing in diabetic animals [McMurtry A. L. et al., J. Surg. Res., 1999, Vol. 86, pp. 36-41]. Faster and stronger healing is achieved by activation of HSP-70 in a wound by laser [Capon A. et al., Lasers Surg. Med., 2001, Vol. 28, pp. 168-175].

Thus, induction of HSP-70 would be beneficial in treating various diabetic complications.

### Neuro-Degenerative Diseases

Neurodegenerative diseases such as Alzheimer's disease, Amyotrophic lateral sclerosis and Parkinson's disease constitute a set of pathological conditions wherein HSP-70 has been implicated to exert a protective affect and delay the progression of these diseases.

(a) Alzheimer's disease is a neurodegenerative disorder characterized by beta-amyloid and tau protein aggregates (neurofibrillary tangles) Increased levels of HSP (8-10 fold increase) in various cellular models have shown to promote tau solubility and tau binding to microtubules, reduce insoluble tau and cause reduced tau phosphorylation. Hence upregulation of HSP will suppress formation of neurofibrillary tangles. [Dou F. et al., Proc. Natl. Acad. Sci. USA, 2003 Jan. 21, Vol. 100 (2), pp. 721-726]. Studies have shown that virally mediated HSP-70 overexpression rescued neurons from the toxic effects of intracellular beta-amyloid accumulation. [Magrané J. et al., J. Neurosci., 2004 Feb. 18, Vol. 24 (7), pp. 1700-1706].
(b) Amyotrophic lateral sclerosis (ALS) is a fatal neurodegenerative condition in which motor-neurons of the spinal cord and motor cortex die, resulting in progressive paralysis. Etiology of ALS involves mutation in the gene encoding Cu/Zn superoxide dismutase-1 (SOD1). Treatment with arimoclomol, an inducer of heat shock proteins (HSPs), significantly delays disease progression in transgenic mice overexpressing human mutant SOD1 that shows a phenotype and pathology that is very similar to that seen in human ALS patients. [Kieran D. et al., Nat. Med., 2004 April, Vol 10 (4), pp. 402-405; Susanna C. B. et al., Nat. Med., 2004, Vol. 10, pp. 345-347].
(c) Parkinson's disease is a common neurodegenerative disease characterized by the loss of dopaminergic neurons in the substantia nigra pars compacta and the accumulation of the misfolded protein alpha-synuclein into aggregates called Lewy bodies and Lewy neuritis, which are very cytotoxic. Mitochondrial dysfunction, oxidative stress, protein misfolding, aggregation, and failure in the proteasomal degradation of specific neuronal proteins have been implicated in pathogenesis of Parkinson disease (PD). Upregulation of HSP-70 by HSP-70 gene transfer to dopamine neurons by a recombinant adeno-associated virus significantly protects the mouse dopaminergic system against MPTP-induced dopamine neuron loss and the associated decline in striatal dopamine levels. [Dong Z. et al., Mol. Ther., 2005 January, Vol. 11(1), pp. 80-88]. Recent experimental evidences show that deprenyl and other propargylamines which are used clinically in treating Parkinson's disease increase neuronal survivability by increasing synthesis of HSP-70 and other anti-apoptotic proteins. [Tatton W. et al., J. Neural. Transm., 2003 May, Vol. 110(5), pp. 509-515]. Introducing HSP-70 in alpha-synuclein transgenic mice by breeding with HSP-70 overexpressing mice led to significant reduction in misfolded and aggregated alpha-synuclein in the progeny. [Klucken J. et al., J. Biol. Chem., 2004 Jun. 11, Vol. 279 (24), pp. 5497-5502]. Recent evidences show that Geldanamycin protects neurons against alpha-synuclein toxicity by enhancing the HSP-70 mediated chaperonic activity. [Auluck P. K. et al., J. Biol. Chem., 2005 Jan. 28, Vol. 280 (4), pp. 2873-2878].

Thus, HSP-70 inducers would be useful in the treatment and delaying the progression of the above neurodegenerative disease conditions.

### Epilepsy

One of the pathological condition wherein protective role of HSP-70 has been implicated is seizures (epilepsy). Studies have shown that hsp70 mRNA and protein are upregulated in response to kainic acid induced seizures in many areas of the limbic system and cortex in rat brain (Hashimoto K, Minabe Y.; Brain Res. 1998; 212-23; Akbar et al.; J. Brain Res Mol Brain Res. 2001; 93(2):148-63) Kainic acid induced seizures in rats represent an established animal model for human temporal lobe epilepsy, the most common form of adult human epilepsy. HSP70 expression in the hippocampus positively correlates with the severity of KA induced limbic seizure (Zhang et al.; Eur J Neurosci. 1997; 9(4):760-9). Hsp72 over expression (gene therapy) in rats improved survival of hippocampal neurons (Yenari et al.; Ann Neurol. 1998; 44(4):584-91). Kainic acid shows a dose dependent severity of seizure which positively correlates with hsp70 induction.

### Post-Traumatic Neuronal Damage

Pathological stress associated with post-traumatic neuronal damage cause induction of HSP-70 in the neuronal tissues. The expression of HSP-70 following traumatic injury to the neuronal tissue has been speculated to be part of a cellular response, which is involved in the repair of damaged proteins [Dutcher S. A et al., J. Neurotrauma, 1998, Vol. 15 (6), pp. 411-420]. BRX-220, an inducer of HSP-70 has been examined for its effect on the survival of injured motoneurones following rat pup sciatic nerve crush [Kalmar B. et al., Exp. Neurol., 2002 July, Vol. 176 (1), pp. 87-97]. It has been found that significantly more number of neurons survived with BRX-220 treatment and there was no further loss of motoneurones. 14 days after injury, 39% of motoneurones survived in BRX220 treated group compared to 21% in vehicle group. Moreover in BRX 220 treated group no further loss of motoneurones occurred, at 10 weeks 42% of motoneurons survived compared to 15% in untreated group. There were also more functional motor units in the hind limb muscles of the treated group compared to that of the control. These observations were correlated to elevated levels of HSP-70 and this compound protects motoneurones from axotomy-induced cell death through a HSP-70 mediated mechanism. Therefore, induction of HSP-70 would be beneficial in post-traumatic neuronal damage.

### Acute Renal Failure

Another pathological condition causing induction of HSP-70 is acute renal failure. Acute renal failure is the sudden loss of the ability of the kidneys to excrete wastes, concentrate urine and conserve the electrolytes. Induction of heat shock proteins (HSPs) plays a protective role in ischaemic acute renal failure. Administration of Sodium arsenite or Uranyl acetate in cisplatin-induced acute renal failure resulted in significant increase in HSP-72 expression. Both Sodium arsenite and Uranyl acetate attenuated the cisplatin-induced increase in serum creatinine and tubular damage scores [Zhou H. et al., Pflugers Arch., 2003 April, Vol. 446 (1), pp. 116-124]. Findings suggest that HSP-72 attenuates CDDP-induced nephrotoxicity. The protective effects of HSP-72 are associated with an increased Bcl-2/Bax ratio and reduced apoptosis.

### Glaucoma

Still another pathological condition which causes induction of HSP-70 is glaucoma. Glaucoma is characterized by rising intraocular pressure and subsequent damage to the optic nerve with selective loss of retinal ganglion cells (RGCs). It has been postulated that apoptosis, a highly regulated process of cell death, is the final common pathway for RGC death in glaucoma. Studies suggest that the induced expression of HSP-72 enhances RGC survival in harmful conditions and ameliorates glaucomatous damage in a rat model [Ishii Y. et al., Invest. Opthalmol. Vis. Sci., 2003 May, Vol. 44(5), pp. 1982-1992]. The study revealed that HSP-72 expression was increased in retinal ganglion cells after administration of HSP inducer geranylgeranyl acetone. The treatment further reduced the loss of retinal ganglion cells, reduced optic nerve damage and decreased the number of TUNEL positive cells in retinal ganglion cell layer.

### Aging Related Skin Degeneration

There is an attenuation of induction of HSP-70 in human keratocytes with aging [Verbeke P. et al., Cell Biol. Int, 2001, Vol. 25 (9), pp. 845-857]. Furthermore, human skin cells have been shown to maintain several characteristics of young cells until late in life, when exposed to repetitive mild heat shocks [Rattan S. I. et al., Biochem. Mol. Biol. Int., 1998, Vol. 45(4), pp. 753-759].

Over expression of heat shock protein gene is sufficient to protect against otherwise lethal exposures to heat, ischemia, cytotoxic drugs, and toxins. The above examples illustrate the ability of HSP-70 to protect cells against various pathological stresses contributing towards different diseases.

U.S. Pat. No. 5,348,945 describes methods for enhancing the survivality of cells and tissues and thereby combating various disease conditions by administering an exogenous HSP-70.

A number of compounds have been reported to be useful for increasing levels of HSPs thereby treating a range of disorders.

U.S. Pat. No. 6,096,711 discloses methods for inducing HSP-72 production in an aged cell by contacting the aged cell with a proteasome inhibitor, and treating stress-induced pathologies associated with apoptosis and inflammation in aged individuals.

U.S. Pat. No. 6,174,875 discloses methods for inducing HSP-70 and treating neurological injuries resulting from cardiac arrest and stroke by inhibiting cell death induced by oxidative stress, with benzoquinoid ansamycins.

U.S. Pat. No. 6,653,326 describes methods for increasing expression of molecular chaperones, including HSP-70 using hydroxylamine derivatives, and thereby treating stress related diseases like stroke, cerebrovascular ischaemia, coronarial diseaseas, allergic diseases, immune diseases, autoimmune diseases, diseases of viral or bacterial origin, tumourous, skin and/or mucous diseases, epithelial disease of renal tubules, atherosclerosis, pulmonary hypertonia and traumatic head injury.

In view of the advantages associated with increased expression of HSP-70 in cells, a method, which increases such expression or increases activity of HSP-70 would be highly advantageous for prevention and treatment of various diseases. Small molecules that either enhances the expression or function of heat shock proteins could have promise in chronic or acute treatment of certain human diseases.

Compounds of the present invention have been categorically shown to induce HSP-70. Therefore, these compounds would be beneficial in the prevention and treatment of conditions where HSP induction has been shown to protect in various diseased states, for example in stroke, myocardial infarction, inflammatory diseases, diseases of viral origin, tumourous diseases, brain haemorrhage, endothelial dysfunctions, diabetic complications, hepatotoxicity, acute renal failure, glaucoma, sepsis, gastric mucosal damage, allograft rejection, neurodegenerative diseases, epilepsy, post-traumatic neuronal damage and aging-related skin degeneration.

Reference may be made to U.S. Pat. No. 4,177,271, which describes hydroxy- and oxo-substituted alpha-benzylidenecycloalkanones having pharmacological activity on the central nervous system such as antidepressant. The phenyl ring is essentially a disubstituted ring where in the substitution is selected from methoxy or methylenedioxy group.

U.S. Pat. No. 6,288,235 describes the 2,4-dioxopiperidine compounds as useful intermediates which can be used for synthesizing libraries on solid supports.

WO 01/40188 US2004009914, US2005069551, US20060089378 describes the compounds which structurally differs from the compounds of the present invention.

WO06087194 relates to 4-piperidone compounds useful as a dye composition comprising an oxonol type methine direct dye for the process of dyeing keratin fibres.

None of the prior art as mentioned above teaches or suggest use of the compounds as HSP inducers.

### SUMMARY OF THE INVENTION

One embodiment of the present invention provides a compound of formula (I),

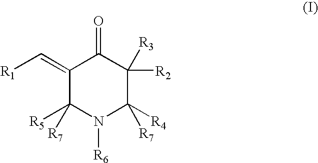

their pharmaceutically acceptable salts and their hydrates, solvates, stereoisomers, conformers, tautomers, polymorphs and prodrugs thereof.

In an another embodiment of the present invention, there is provided a compound of formula (II),

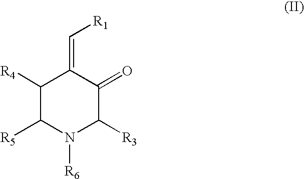

their pharmaceutically acceptable salts and their hydrates, solvates, stereoisomers, conformers, tautomers, polymorphs and prodrugs thereof,
wherein, R₁ is selected from unsubstituted or substituted:
a. Five to twelve membered monocyclic or bicyclic aryl,
b. Five to twelve membered monocyclic or bicyclic heteroaryl wherein, it contains one or more heteroatoms selected from nitrogen, oxygen and sulphur, or
c. Four to twelve membered monocyclic or bicyclic heterocyclyl wherein, it contains one or more heteroatoms selected from nitrogen, oxygen and sulphur.

Examples of such aryl, heteroaryl and heterocyclyl systems are phenyl, naphthyl, heptalenyl, benzocycloheptalenyl, cyclobutadienyl, cyclobutenyl, pyridinyl, pyrazinyl, pyridazinyl, pyrimidinyl, quinolinyl, isoquinolinyl, quinazolinyl, quinoxalinyl, cinnolinyl, phthalazinyl, pyrazolyl, pyrrolyl, triazolyl, tetrazolyl, thienyl, oxazolyl, isoxazolyl, thiazolyl, isothiazolyl, imidazolyl, oxadiazolyl, thiadiazolyl, piperazinyl, morpholinyl, thiomorpholinyl, thiomorpholin 1,1-dioxide, piperidinyl, pyrrolidinyl, imidazolidinyl, pyrazolidinyl, thiazolidinyl, hexahydropyridazinyl, hexahydropyrimidinyl, hexahydropyrazinyl, azepanyl, diazepanyl, thiazepanyl, azepinyl, benzopyrazolyl, indolinyl, indolyl, phthalanyl, benzothiophenyl, benzofuryl, benzopyrrolyl, benzimidazolyl, benzoxazolyl, benzoisoxazolyl, benzothiazolyl, benzoisothiazolyl, benzotriazolyl, benzothiadiazolyl and benzoxadiazolyl;

Said aryl, heteroaryl, heterocyclyl when substituted, it is substituted by one to four substituents of R₈, preferably one to three substituents of R₈, more preferably one to two substituents of R₈, wherein R₈ is independently selected from the group consisting of:

halogen, —OH, —SH, —C₁₋₈alkyl, nitro, amino, cyano, —N(R₉)C(O)(C₁₋₈alkyl), —N(R₉)C(O)(aryl), —N(R₉)C(O)(heteroaryl), —N(R₉)C(O)(heterocyclyl), —N(R₉)SO₂(C₁₋₉alkyl), —N(R₉)SO₂(aryl), —N(R₉)SO₂(heteroaryl), —N(R₉)SO₂(heterocyclyl), —N(R₉)SO₂CF₃, —COON, —C(O)N(R₉)(R₉), —C(O)N(R₉)(aryl), —C(O)N(R₉)(heteroaryl), —C(O)N(R₉)(heterocyclyl), —SO₂N(R₉)(R₉), —SO₂N(R₉)(aryl), —SO₂N(R₉)(heteroaryl), —SO₂N(R₉)(heterocyclyl), —C(O)O—(C₁₋₈alkyl), —C(O)O-aryl, —C(O)O-heteroaryl, —C(O)O-heterocyclyl, —N(R₉)C(O)O—(C₁₋₈alkyl), —N(R₉)C(O)O-aryl, —N(R₉)C(O)O-heteroaryl, —N(R₉)C(O)O-heterocyclyl, —CF₃, —C(O)CF₃, —SO₂CF₃, —(C₁₋₈alkyl)ₘ-O(C₁₋₈alkyl), —(C₁₋₈alkyl)ₘ-O(aryl), —(C₁₋₈alkyl)ₘ-O(heteroaryl), —(C₁₋₈alkyl)ₘ-O(heterocyclyl), —(C₁₋₈alkyl)ₘ-N(R₉)(C₁₋₈alkyl), —(C₁₋₈alkyl)ₘ-N(R₉)(aryl), —(C₁₋₈alkyl)ₘ-N(R₉)(heteroaryl), —(C₁₋₈alkyl)ₘ-N(R₉)(heterocyclyl), —(C₁₋₈alkyl)ₘ, —C(O)(C₁₋₈alkyl), —(C₁₋₈alkyl)ₘ-C(O)(aryl), —(C₁₋₈alkyl)ₘ-C(O)(heteroaryl), —(C₁₋₈alkyl)ₘ-C(O)(heterocyclyl), —C(O)(C₁₋₈alkyl)-aryl, —C(O)(C₁₋₈alkyl)-heteroaryl, —C(O)(C₁₋₈alkyl)-heterocyclyl, —(C₁₋₈alkyl)ₘ-S(O)(C₁₋₈alkyl), —(C₁₋₈alkyl)ₘ-S(O)(aryl), —(C₁₋₈alkyl)ₘ-S(O)(heteroaryl), —(C₁₋₈alkyl)ₘ-S(O)(heterocyclyl), —(C₁₋₈alkyl)ₘ, —S(O)₂(C₁₋₈alkyl), —(C₁₋₈alkyl)ₘ-S(O)₂O—(C₁₋₈alkyl), —(C₁₋₈alkyl)ₘ-SO₂(aryl), —(C₁₋₈alkyl)ₘ-SO₂(heteroaryl), (C₁₋₈alkyl)ₘ-SO₂(heterocyclyl), —N(R₉)(SO₂-aryl), —N(R₉)(SO₂-heteroaryl), —N(R₉)(SO₂-heterocyclyl), —N(R₉)C(O)N(R₉)(R₉), —N(R₉)C(O)N(R₉)(aryl), —N(R₉)C(O)N(R₉)(heteroaryl), —N(R₉)C(O)N(R₉)(heterocyclyl), —N(R₉)C(O)C(O)N(R₉)(R₉), —N(R₉)C(O)C(O)N(R₉)(aryl), —NR₉C(O)C(O)N(R₉) (heteroaryl), —N(R₉)C(O)C(O)N(R₉)(heterocyclyl), —N(R₉)C(S)N(R₉)(R₉), —N(R₉)C(S)N(R₉)(aryl), —N(R₉)C(S)N(R₉)(heteroaryl), —N(R₉)C(S)N(R₉) (heterocyclyl), —N(R₉)SO₂N(R₉)(R₉), —N(R₉)SO₂N(R₉)(aryl), —N(R₉)SO₂N(R₉) (heteroaryl), —N(R₉)SO₂N(R₉)(heterocyclyl), —SO₂OH, —NHC(NH)NH₂, —N(R₉)(aryl), —N(R₉)(heteroaryl), —N(R₉)(heterocyclyl), —(C₁₋₈alkyl)ₘ-aryl, —(C₁₋₈alkyl)ₘ-heteroaryl, —(C₁₋₈alkyl)ₘ-heterocyclyl-oxo, and -thioxo;

R₉ is selected from hydrogen or (C₁₋₈alkyl);

wherein, aryl present as a substituent in R₈ is five to seven membered monocyclic ring and heteroaryl and heterocyclyl present as a substituent in R₈ is three to seven membered monocyclic ring system which contains one or more heteroatoms selected from nitrogen, oxygen and sulphur; wherein the aryl, heteroaryl and heterocyclyl are unsubstituted or substituted with one to three substituents independently selected from the group consisting of:
oxo, thioxo, halogen, —OH, —SH, —(C₁₋₈alkyl), —O(C₁₋₈alkyl), nitro, amino, mono(C₁₋₈alkyl)amino, di(C₁₋₈alkyl)amino, —COOH, —CONH₂, —CF₃, —C(O)CF₃, —SO₂CF₃,
—SO₂(C₁₋₈alkyl), and —SO₂NH₂;
wherein, the above said C₁₋₈alkyl is straight, branched or cyclic and may contain one double bond and is substituted with one to two substituents independently selected from the group consisting of:
—OH, —SH, oxo, thioxo, amino, mono(C₁₋₃alkyl)amino, di(C₁₋₃alkyl)amino, —S(C₁₋₃alkyl), and —C₁₋₃ alkoxy;
wherein C₁₋₃alkoxy is straight or branched, may contain one or two double or triple bonds; C₁₋₃alkyl is straight or branched;
R₉ is selected from hydrogen or (C₁-C₈)alkyl;
m is zero or one;
with the proviso that when R₁ is selected from unsubstituted or substituted
a) cyclohexane,
b) cyclohexene or
c) six membered monocyclic heteroaryl or heterocyclyl having one to two heteroatoms selected from nitrogen, oxygen or sulphur, then R₈ as substituent on R₁ is not selected from hydroxyl and oxo group.

R₂ is selected from the group consisting of:

hydrogen, halogen, —C₁₋₃alkyl, —OH, —SH, —O(C₁₋₃alkyl), amino, mono(C₁₋₃alkyl)amino, di(C₁₋₃alkyl)amino, —C(O)CF₃, —C(O)CH₃, —SO₂CF₃, —CF₃₋₈alkyl), —SO₂(C₁₋₈alkyl), and —SO₂NH₂;
wherein, the above said C₁₋₈alkyl is straight, branched or cyclic and may contain one or two double or triple bonds and is substituted with one to two substituents independently selected from the group consisting of:
—OH, —SH, oxo, thioxo, amino, mono(C₁₋₃ alkyl)amino, di(C₁₋₃alkyl)amino, —S(C₁₋₃alkyl), and —C₁₋₃ alkoxy;

Wherein, C₁₋₃alkoxy is straight or branched, may contain one double bond; C₁₋₃alkyl is straight or branched.

R₃ is selected from the group consisting of:

halogen, nitro, amino, —OH, —SH, —N(R₉)C(O)(C₁₋₈alkyl), —N(R₉)C(O)(aryl), —N(R₉)C(O)(heteroaryl), —N(R₉)C(O)(heterocyclyl), —N(R₉)SO₂(C₁₋₈alkyl), —N(R₉)SO₂(aryl), —N(R₉)SO₂(heteroaryl), —N(R₉)SO₂(heterocyclyl), —(C₁₋₃alkyl)ₘ-aryl, —(C₁₋₃alkyl)ₘ-heteroaryl, —(C₁₋₃alkyl)ₘ-heterocyclyl, —C(O)N(R₉) (R₉), —C(O)N(R₉)(aryl), —C(O)N(R₉) (heteroaryl), —C(O)N(R₉) (heterocyclyl), —SO₂N((R₉) (R₉), —SO₂N(R₉)(aryl), —SO₂N(R₉)(heteroaryl), —SO₂N(R₉)(heterocyclyl), —N(R₉)SO₂CF₃, —C(O)O—(C₁₋₈alkyl), —C(O)O-aryl, —C(O)O-heteroaryl, —C(O)O-heterocyclyl, —N(R₉)C(O)O—(C₁₋₈alkyl), —N(R₉)C(O)O-aryl, —N(R₉)C(O)O-heteroaryl, —N(R₉)C(O)O-heterocyclyl, —CF₃, —C(O)CF₃, —SO₂CF₃, —COOH, —(C₁₋₃alkyl)ₘ-N((R₉)(C₁₋₈alkyl), —(C₁₋₃alkyl), —C(O)(C₁₋₈alkyl), —(C₁₋₃alkyl)ₘ-C(O)(aryl), —(C₁₋₃alkyl)ₘ-C(O)(heteroaryl), —(C₁₋₃alkyl)ₘ-C(O)(heterocyclyl), —C(O)(C₁₋₃alkyl)-aryl, —C(O)(C₁₋₃alkyl)-heteroaryl, —C(O)(C₁₋₃alkyl)-heterocyclyl, —(C₁₋₃alkyl)-C(O)(C₁₋₃alkyl)-aryl, —(C₁₋₃alkyl)-(O)(C₁₋₃alkyl)-heteroaryl, —(C₁₋₃alkyl)-C(O)(C₁₋₃alkyl)-heterocyclyl, —(C₁₋₃alkyl)ₘ-S(O)(C₁₋₈alkyl), —(C₁₋₃alkyl)ₘ-S(O)(aryl), —(C₁₋₃alkyl)ₘ-S(O)(heteroaryl), —(C₁₋₃alkyl)ₘ-S(O)(heterocyclyl), —(C₁₋₃alkyl)ₘ-S(O)₂(C₁₋₈alkyl), —(C₁₋₃alkyl)ₘ-S(O)₂O—(C₁₋₈alkyl), —(C₁₋₃alkyl)ₘ-SO₂(aryl), —(C₁₋₃alkyl)ₘ-SO₂(heteroaryl), —(C₁₋₃alkyl)ₘ-SO₂(heterocyclyl), —S(O)₂—(C₁₋₃alkyl)-aryl, —S(O)₂—(C₁₋₃alkyl)-heteroaryl, —S(O)₂—(C₁₋₃alkyl)-heterocyclyl, —(C₁₋₃alkyl)SO₂—(C₁₋₃alkyl)-aryl, —(C₁₋₃alkyl)SO₂—(C₁₋₃alkyl)-heteroaryl, —(C₁₋₃alkyl)SO₂—(C₁₋₃alkyl)-hetrocyclyl, —N(R₉)SO₂(aryl), —N(R₉)SO₂(heteroaryl), —N(R₉)SO₂(heterocyclyl), —N(R₉)C(O)N((R₉)(R₉), —N(R₉)C(O)N(R₉)(aryl), —N(R₉)C(O)N(R₉)(heteroaryl), —N(R₉)C(O)N(R₉) (heterocyclyl), —N(R₉)C(O)C(O)N((R₉)(R₉), —N(R₉)C(O)C(O)N(R₉)(aryl), N(R₉)C(O)C(O)N(R₉)(heteroaryl), —N(R₉)C(O)C(O)N(R₉)(heterocyclyl), —N(R₉)C(S)N(R₉)(R₉), —N(R₉)C(S)N(R₉)(aryl), —N(R₉)C(S)N(R₉)(heteroaryl), —N(R₉)C(S)N(R₉)(heterocyclyl), —N(R₉)SO₂N(R₉)(R₉), —N(R₉)SO₂N(R₉)(aryl), —N(R₉)SO₂N(R₉)(heteroaryl), —N(R₉)SO₂N(R₉)(heterocyclyl), —SO₂OH, —NHC(═NH)NH₂, —(C₁₋₃alkyl), —O(aryl), —(C₁₋₃alkyl)ₘ-O(heteroaryl), —(C₁₋₃alkyl)ₘ-O(heterocyclyl), —(C₁₋₃alkyl)ₘ-N(R₉)(aryl), —(C₁₋₃alkyl)ₘ-N(R₉)(heteroaryl), —(C₁₋₃alkyl)ₘ-N(R₉)(heterocyclyl), —C(O)C(O)(aryl), —C(O)C(O)(heteroaryl), and —C(O)C(O)(heterocyclyl);
wherein, said aryl present as a substituent in R₃ is five to seven membered monocyclic ring and heteroaryl and heterocyclyl present as a substituent in R₃ are three to seven membered monocyclic ring containing one or more heteroatoms selected from nitrogen, oxygen and sulphur, wherein the said aryl, heteroaryl and heterocyclyl are unsubstituted or substituted with one to three substituents independently selected from the group consisting of:
oxo, thioxo, —OH, —SH, halogen, —C₁₋₈alkyl, —O(C₁₋₈alkyl), nitro, amino, mono(C₁₋₈alkyl)amino, di(C₁₋₈alkyl)amino, —COOH, —CONH₂, —CF₃, —C(O)CF₃, —SO₂CF₃, —S(C₁₋₈alkyl), —N(R₉)SO₂(C₁₋₈alkyl), —SO₂(C₁₋₈alkyl), and —SO₂NH₂;

Wherein, the above said C₁₋₈alkyl is straight, branched or cyclic, may contain one or two double or triple bonds and is with one to two substituents independently selected from the group consisting of:

—OH, —SH, Oxo, thioxo, amino, mono(C₁₋₃alkyl)amino, di(C₁₋₃alkyl)amino, —S(C₁₋₃alkyl), and —C₁₋₃ alkoxy;
wherein C₁₋₃alkoxy is straight or branched, may contain one double bond; C₁₋₃alkyl is straight or branched;
m is zero or one.

R₄ and R₅ is independently selected at each occurrence from hydrogen or R₈ or either R₄ or R₅ together with R₇ is oxo;

with the proviso that when R₄ is oxo, R₃ is not selected from —C(O)(C₁₋₈alkyl), —C(O)O(C₁₋₃alkyl), —C(O)(C₁₋₈alkyl)-aryl, —C(O)aryl, —C(O)thienyl, and —C(O)furyl;

R₆ is selected from the group consisting of:

—(C₁₋₈alkyl), —C(O)N(R₉)(R₉), —C(O)N(R₉)(aryl), —C(O)N(R₉)((C₁₋₈alkyl)-aryl), —C(O)N(R₉)(heteroaryl), —C(O)N(R₉)SO₂(aryl), —C(O)N(R₉)(heterocyclyl), —C(S)N(R₉)(R₉), —C(S)N(R₉)(aryl), —C(S)N(R₉) (heteroaryl), —C(S)N(R₉)(heterocyclyl), —SO₂N(R₉)(R₉), —SO₂N(R₉)(aryl), —SO₂N(R₉)(heteroaryl), —SO₂N(R₉)(heterocyclyl), —C(O)C(O)N(R₉)(R₉), —C(O)C(O)N(R₉)(aryl), —C(O)C(O)N(R₉)(heteroaryl), —C(O)C(O)N(R₉) (heterocyclyl), —C(O)O—(C₁₋₈alkyl), —C(O)O—(C₁₋₈alkyl)ₘ-aryl, —C(O)O—(C₁₋₈alkyl)ₘ-heteroaryl, —C(O)O—(C₁₋₈alkyl)ₘ-heterocyclyl, —CF₃, —C(O)CF₃, —SO₂CF₃, —(C₁₋₈alkyl)O(C₁₋₈alkyl), —(C₁₋₈alkyl)-O(aryl), —(C₁₋₈alkyl)-O(heteroaryl), —(C₁₋₈alkyl)-O(heterocyclyl), —(C₁₋₈alkyl)-N(R₉)(C₁₋₈alkyl), —(C₁₋₈alkyl)-N(R₉)(aryl), —(C₁₋₈alkyl)-N(R₉)(heteroaryl), —(C₁₋₈alkyl)-N(R₉)(heterocyclyl), —(C₁₋₈alkyl)ₘC(O)(C₁₋₈alkyl), —(C₁₋₈alkyl)ₘ-C(O)(aryl), —(C₁₋₈alkyl)ₘ-C(O)(heteroaryl), —(C₁₋₈alkyl)ₘ-C(O)(heterocyclyl), —C(O)—(C₁₋₃alkyl)-aryl, —C(O)—(C₁₋₃alkyl)-heteroaryl, —C(O)—(C₁₋₃alkyl)-heterocyclyl, —(C₁₋₈alkyl)-C(O)(C₁₋₈alkyl)-aryl, —(C₁₋₈alkyl)-C(O)(C₁₋₈alkyl)-heteroaryl, C(O)(C₁₋₈alkyl)-heterocyclyl, —(C₁₋₈alkyl)ₘ-SO₂(C₁₋₈alkyl), —(C₁₋₈alkyl)ₘ-SO₂(aryl), —(C₁₋₈alkyl)ₘ-SO₂(heteroaryl), —(C₁₋₈alkyl)ₘ-SO₂(heterocyclyl), —(C₁₋₈alkyl)-S(O)(C₁₋₈alkyl), —(C₁₋₈alkyl)-S(O)(aryl), —(C₁₋₈alkyl)-S(O)(heteroaryl), —(C₁₋₈alkyl)-S(O)(heterocyclyl), —S(O)₂(C₁₋₈alkyl)-aryl, —S(O)₂(C₁₋₈alkyl)-heteroaryl, —S(O)₂(C₁₋₈alkyl)-heterocyclyl, —(C₁₋₈alkyl)SO₂—(C₁₋₈alkyl)-aryl, —(C₁₋₈alkyl)SO₂—(C₁₋₈alkyl)-heteroaryl, —(C₁₋₈alkyl)SO₂—(C₁₋₈alkyl)-heterocyclyl, —(C₁₋₈alkyl), —S(C₁₋₈alkyl), —(C₁₋₈alkyl)-S(C₁₋₈alkyl)-aryl, —(C₁₋₈alkyl)-S(C₁₋₈alkyl)-heteroaryl, —(C₁₋₈alkyl)-S(C₁₋₈alkyl)-hetrocyclyl, —(C₁₋₈alkyl)-S(aryl), —(C₁₋₈alkyl)-S(heteroaryl), —(C₁₋₈alkyl)-S(heterocyclyl), —(C₁₋₈alkyl)ₘ-aryl, —(C₁₋₈alkyl)ₘ-heteroaryl, —(C₁₋₈alkyl)ₘ-heterocyclyl, —C(O)C(O)(heteroaryl), —C(O)C(O)(heterocyclyl) and —C(O)C(O)(aryl);
wherein aryl present as a substituent in R₆ is five to seven membered monocyclic ring and heteroaryl and heterocyclyl present as a substituent in R₆ are three to seven membered monocyclic ring containing one or more heteroatoms selected from nitrogen, oxygen and sulphur; wherein said aryl, heteroaryl and heterocyclyl are unsubstituted or substituted with one to three groups independently selected from:
oxo, thioxo, halogen, —OH, —SH, —C₁₋₈alkyl, —O(C₁₋₈alkyl), nitro, amino, mono(C₁₋₈alkyl)amino, —CO(C₁₋₈alkyl), di(C₁₋₈alkyl)amino, —COOH, —COO(C₁₋₈alkyl), —CONH₂, —CF₃, —C(O)CF₃, —S(C₁₋₈alkyl), —SO₂(C₁₋₈alkyl), —SO₂CF₃, and —SO₂NH₂;
wherein, the above said C₁₋₈alkyl is straight, branched or cyclic, may contain one or two double or triple bonds and may be substituted with one to two substituents independently selected from:
—OH, —SH, oxo, thioxo, amino, mono(C₁₋₃alkyl)amino, di(C₁₋₃alkyl)amino, —S(C₁₋₃alkyl), —COON, CONH₂, and —C₁₋₃ alkoxy;
wherein, C₁₋₃alkoxy is straight or branched, may contain one double bond; C₁₋₃alkyl is straight or branched; m is independently selected at each occurrence, from zero to one.
with the proviso that:
i) when R₆ is selected from methyl, —CH₂—CH═CH₂ or —CH₂-phenyl and R₂═H or methyl, then R₁ is not selected from:

a. trimethoxyphenyl,

b. benzdioxole or chlorosubstituted benzdioxole or

c. furyl;

ii) when R₆ is selected from methyl and R₂═H, R₃=Phenyl then R₁ is not selected from unsubstituted phenyl;
iii) when R₄, R₅ and R₇ are hydrogen and R₆ is selected from the group consisting of
—(C₁₋₈alkyl), —(C₁₋₈alkyl)-O(C₁₋₈alkyl), —(C₁₋₈alkyl)-O(aryl), —(C₁₋₈alkyl)-O(heteroaryl), —(C₁₋₈alkyl)-O(heterocyclyl), —(C₁₋₈alkyl)-N(R₉)(C₁₋₈alkyl), —(C₁₋₈alkyl)-N(R₉)(aryl), —(C₁₋₈alkyl)-N(R₉)(heteroaryl), —(C₁₋₈alkyl)-N(R₉)(heterocyclyl), —(C₁₋₈alkyl)-C(O)(C₁₋₈alkyl), —(C₁₋₈alkyl)-C(O)(aryl), —(C₁₋₈alkyl)-C(O)(heteroaryl), —(C₁₋₈alkyl)-C(O)(heterocyclyl), —(C₁₋₈alkyl)-C(O)(C₁₋₈alkyl)-aryl, —(C₁₋₈alkyl)-C(O)(C₁₋₈alkyl)-heteroaryl, —(C₁₋₈alkyl)-C(O)(C₁₋₈alkyl)-heterocyclyl, —(C₁₋₈alkyl)ₘ-aryl, —(C₁₋₈alkyl)ₘ-heteroaryl, —(C₁₋₈alkyl)ₘ-heterocyclyl, —C(O)N(R₉)(R₉), —(C₁₋₈alkyl)-SO₂(C₁₋₈alkyl), —(C₁₋₈alkyl)-S(O)(C₁₋₈alkyl), —(C₁₋₈alkyl)-S(O)(aryl), —(C₁₋₈alkyl)-S(O)(heteroaryl), —(C₁₋₈alkyl)-S(O)(heterocyclyl), —(C₁₋₈alkyl)-SO₂(C₁₋₈alkyl)-aryl, —(C₁₋₈alkyl)-SO₂(C₁₋₈alkyl)-heteroaryl, —(C₁₋₈alkyl)-SO₂(C₁₋₈alkyl)-hetrocyclyl, —(C₁₋₅alkyl)-S(C₁₋₈alkyl), —(C₁₋₈alkyl)-S(C₁₋₈alkyl)-aryl, —(C₁₋₈alkyl)-S(C₁₋₈alkyl)-heteroaryl, —(C₁₋₈alkyl)-S(C₁₋₈alkyl)-hetrocyclyl, —(C₁₋₈alkyl)-S(aryl), —(C₁₋₈alkyl)-S(heteroaryl), —(C₁₋₈alkyl)-S(heterocyclyl), —(C₁₋₈alkyl)-SO₂(aryl), —(C₁₋₈alkyl)-SO₂(heteroaryl), —(C₁₋₈alkyl)-SO₂(heterocyclyl), acyl, and —C(O)O—(C₁₋₈alkyl),
then R₃ is not
—CH₂-phenyl, —CH₂-substituted phenyl, —CH₂-pyridyl, —CH₂-substituted pyridyl, —CH₂-pyrimidinyl, —CH₂-substituted pyrimidinyl wherein the substitution on aryl, pyridyl and pyrimidinyl is selected from hydroxyl, alkoxy, halogen and CF₃;

R₇ is selected from the group consisting of:

hydrogen, halogen, —OH, —SH, —C₁₋₈alkyl, —O(C₁₋₈alkyl), nitro, amino, mono(C₁₋₈alkyl)amino, di(C₁₋₃alkyl)amino, —COON, —CONH₂, —CF₃, —C(O)CF₃, —SO₂CF₃, —S(C₁₋₈alkyl), —SO₂(C₁₋₈alkyl), and —SO₂NH₂;

Wherein, the above said C₁₋₈alkyl is straight, branched or cyclic, may containing one or two double or triple bonds and substituted with one to two substituents selected from the group consisting of:

—OH, —SH, oxo, thioxo, amino, mono(C₁₋₃alkyl)amino, di(C₁₋₃alkyl)amino, —S(C₁₋₃alkyl), and —C₁₋₃ alkoxy;
wherein, C₁₋₃alkoxy is straight or branched, may contain one double bond and C₁₋₃alkyl is straight or branched.

In another embodiment, the present invention pertains to pharmaceutically acceptable salts of a compound as above.

Another embodiment of the present invention is a method for preparation of a compound of formula (I) & (II) as herein described in Schemes below.

Another embodiment of the present invention is a pharmaceutical composition comprising a compound of formula (I) or (II), optionally in admixture with a pharmaceutically acceptable adjuvant, diluent or carrier.

Yet another embodiment of the present invention provides a method of treating various disease conditions, accompanying pathological stress are selected from ischemic stroke, myocardial infarction, inflammatory disorders, diseases of viral origin, tumourous diseases, brain haemorrhage, endothelial dysfunctions, diabetic complications, hepatotoxicity, acute renal failure, glaucoma, sepsis, gastric mucosal damage, allograft rejection, neurodegenerative diseases, epilepsy, post-traumatic neuronal damage and aging-related skin degeneration, wherein the underlying mechanism is Heat Shock Protein (HSP) induction in a mammal, including a human being, by administering to a mammal in need thereof a therapeutically effective amount of compounds of present invention.

Yet another embodiment of the instant invention is the use of above compounds in the manufacture of medicaments, useful for treatment of various disease conditions accompanying pathological stress selected from ischemic stroke, myocardial infarction, inflammatory disorders, diseases of viral origin, tumourous diseases, brain haemorrhage, endothelial dysfunctions, diabetic complications, hepatotoxicity, acute renal failure, glaucoma, sepsis, gastric mucosal damage, allograft rejection, neurodegenerative diseases, epilepsy, post-traumatic neuronal damage and aging-related skin degeneration, in a mammal including human being by induction of HSP.

### DETAILED DESCRIPTION OF THE INVENTION

### Definitions

The following definitions apply to the terms as used throughout this specification, unless otherwise limited in specific instances:

The term “compound” employed herein refers to any compound encompassed by the generic formula disclosed herein. The compounds described herein may contain one or more double bonds and therefore, may exist as stereoisomers, such as geometric isomers, E and Z isomers, and may possess asymmetric carbon atoms (chiral centres) such as enantiomers, diastereoisomers. Accordingly, the chemical structures depicted herein encompass all possible stereoisomers of the illustrated compounds including the stereoisomerically pure form (e.g., geometrically or enantiomerically pure) and stereoisomeric mixtures (racemates). The compound described herein, may exist as a conformational isomers such as chair or boat form. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to ²H, 3H, ¹³C, ¹⁴C, ¹⁵N, ¹⁸O, ¹⁷O, etc. Compounds may exist in unsolvated forms as well as solvated forms, including hydrated forms. In general, compounds may be hydrated or solvated. Certain compounds may exist in multiple crystalline or amorphous forms. In general, all physical forms are equivalent for the uses contemplated herein and are intended to be within the scope of the present invention.

The use of the terms “a” and “an” and “the” and similar referents in the context of describing the invention (especially in the context of the appended claims) are to be construed to cover both the singular and the plural, unless otherwise indicated herein or clearly contradicted by context.

Further, it should be understood, when partial structures of the compounds are illustrated, a dash (“-”) indicate the point of attachment of the partial structure to the rest of the molecule.

The nomenclature of the compounds of the present invention as indicated herein is according to MDL ISIS® Draw Version 2.5.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, carbonic acid, phosphoric acid, and the like; or formed with organic acids such as acetic acid, propionic acid, isobutyric acid, hexanoic acid, cyclopentanepropionic acid, oxalic acid, glycolic acid, pyruvic acid, lactic acid, malonic acid, succinic acid, suberic acid, malic acid, maleic acid, fumaric acid, tartaric acid, citric acid, benzoic acid, 3-(4-hydroxybenzoyl)benzoic acid, phthalic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glucuronic acid, galactunoric acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. Also included are salts of amino acids such as arginate and the like (see, for example, Berge, S. M., et al., “Pharmaceutical Salts”, Journal of Pharmaceutical Science, 1977, 66, 1-19).

As used herein, the term “polymorphs” pertains to compounds having the same chemical formula, the same salt type and having the same form of hydrate/solvate but having different crystallographic properties.

As used herein, the term “hydrates” pertains to a compound having a number of water molecules bonded to the molecule.

As used herein, the term “solvates” pertains to a compound having a number of solvent molecules bonded to the molecule.

The present invention also encompasses compounds which are in a prodrug form. Prodrugs of the compounds described herein are those compounds that readily undergo chemical changes under physiological conditions (in vivo) to provide the active compounds of the present invention. Additionaqlly, prodrugs can be converted to the compounds of the present invention by chemical or biochemical methods in an ex vivo environment, for example, transdermal patch reservoir with a suitable enzyme or chemical. Prodrugs are, in some situation, easier to administer than the active drug. They may, for instance, be bioavailable by oral administration whereas the active drug is not. The prodrug may also have improved solubility in pharmacological composition over the active drug. Esters, peptidyl derivatives and the like, of the compounds are the examples of prodrugs of the present invention.

In vivo hydrolysable (or cleavable) ester of a compound of the present invention that contains a carboxy group is, for example, a pharmaceutically acceptable ester which is hydrolysed in the human or animal body to produce the parent acid. Suitable pharmaceutically acceptable esters for carboxy include C₁-C₈ alkoxymethyl esters, for example, methoxymethyl, C₁-C₈ alkanoloxymethyl ester, for example, pivaloyloxymethyl; phthalidyl esters; C₃-C₈ cycloalkoxycarbonyloxy-C₁-C₈ alkyl esters, for example, 1-cyclohexylcarbonyloxyethyl; 1,3-dioxolen-2-onylmethyl esters, for example, 5-methyl-1,3-dioxolen-2-onylmethyl; and C₁-C₈ alkoxycarbonyloxyethyl esters, for example, 1-methoxycarbonyloxymethyl; and may be formed at any carboxy group in the compounds of the present invention.

The term “substituted”, as used herein, means that any one or more hydrogens on the designated atom is replaced with a selection from the indicated group, provided that the designated atom's normal valency is not exceeded, and that the substitution results in a stable compound, for example when a substituent is keto, then two hydrogens on the atom are replaced. All substituents (R₁, R₂ . . . ) and their further substituents described herein may be attached to the main structure at any heteroatom or carbon atom which results in formation of stable compound.

As used herein, the term “oxo” or “thioxo” is intended to mean that the group when bound to a saturated carbon atom may represent C═O or C═S and when bound to unsaturated carbon atom may be represented in the tautomeric enol form.

In the present context the term “aryl” is intended to mean a fully or partially aromatic carbocyclic ring or ring system.

The term “heteroaryl” is intended to mean a fully or partially aromatic carbocyclic ring or ring system where one or more of the carbon atoms have been replaced with heteroatoms, e.g. nitrogen (═N— or —NH—), oxygen and sulphur atoms.

The term “heterocyclyl” is intended to mean a non-aromatic carbocyclic ring or ring system where one or more of the carbon atoms have been replaced with heteroatoms, e.g. nitrogen (═N— or —NH—), oxygen and sulphur atoms.

As used herein, “room temperature” refers to a temperature between 25° C. and 35° C.

As used herein, a “halo” or “halogen” substituent is a monovalent halogen radical chosen from chloro, bromo, iodo and fluoro.

As used herein, the term “mammal” means a human or an animal such as monkeys, primates, dogs, cats, horses, cows, etc.

“Treating” or “treatment” of any disease or disorder refers, in one embodiment, to ameliorating the disease or disorder (i.e. arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter) or both. In yet another embodiment, “treating” or “treatment” refers to delaying the onset of the disease or disorder. As used herein, amelioration of the symptoms of a particular disorder by administration of a particular compound or pharmaceutical composition refers to any lessening, whether permanent or temporary, lasting or transient that can be attributed to or associated with administration of the composition.

The phrase “a therapeutically effective amount” means the amount of a compound that, when administered to a patient for treating a disease, is sufficient to effect such treatment for the disease. The “therapeutically effective amount” will vary depending on the compound, mode of administration, the disease and its severity and the age, weight, etc., of the patient to be treated.

When used, the expressions “comprise” and “comprising” denote “include” and “including” but not limited to. Thus, other ingredients, carriers and additives may be present.

One embodiment of the present invention provides a compound of formula (I),

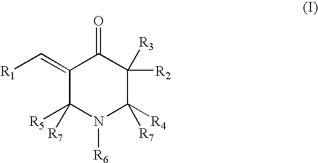

wherein R₁, R₂, R₃, R₄, R₅, R₆ and R₇ are as defined above

In an another embodiment of the present invention, there is provided a compound of formula (II),

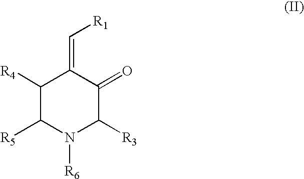

Wherein R₁, R₂, R₃, R₄, R₅ & R₆ are as defined above.

The invention also provides pharmaceutically acceptable salts and their hydrates, solvates, stereoisomers, conformers, tautomers, polymorphs and prodrugs thereof,

One of the preferred embodiment of the present invention is a compound of formula (I) or (II) as mentioned above, wherein R₁ is selected from optionally substituted phenyl, pyridyl, pyrimidinyl, pyrazinyl, pyridazinyl, quinolinyl, quinoxalinyl, quinazolinyl, cinnolinyl, phthalazinyl, pyrazolyl, pyrrolyl, imidazolyl, oxazolyl, isoxazolyl, thienyl and R₂ is selected from hydrogen, methyl, ethyl, isopropyl, —SO₂CH₃ and SO₂NH₂

In one of the embodiment of the present invention is a family of specific compound of particular interest within the above formula I or II consists of compound or their pharmaceutically acceptable salts:

| Compd. | |
|---|---|
| No. | Nomenclature |
| 1 | 1-Benzyl-3,3-dimethyl-5-[1-pyridin-2-yl-methylidene]-piperidin-4- |
| | one |
| 2 | 3,3-Dimethyl-4-oxo-5-[1-pyridin-2-yl-methylidene]-piperidine-1- |
| | carboxylic acid benzyl ester |
| 3 | 3,3-Dimethyl-4-oxo-5-[1-pyridin-2-yl-methylidene]-piperidine-1- |
| | carboxylic acid ethyl ester |
| 4 | 3,3-Dimethyl-4-oxo-5-[1-pyridin-2-yl-methylidene]-piperidine-1- |
| | carboxylic acid phenyl ester |
| 5 | 1-Acetyl-3,3-dimethyl-5-[1-pyridin-2-yl-methylidene]-piperidin-4- |
| | one |
| 6 | 1-Benzyl-3-methyl-5-[1-pyridin-2-yl-methylidene]-piperidin-4-one |
| 7 | 1-Benzyl-3,3-dimethyl-5-[1-[4-(morpholine-4-carbonyl)-phenyl]- |
| | methylidene]-piperidin-4-one |
| 8 | 1-Benzyl-3,3-dimethyl-5-[1-(4-methylsulfanyl-phenyl)- |
| | methylidene]-piperidin-4-one |
| 9 | 1-Benzyl-3,3-dimethyl-5-[1-(4-nitro-phenyl)-methylidene]- |
| | piperidin-4-one |
| 10 | 1-Benzyl-3,3-dimethyl-5-[1-phenyl-methylidene]-piperidin-4-one |
| 11 | 1-Benzyl-3,3-dimethyl-5-[1-(3-methyl-thiophen-2-yl)-methylidene]- |
| | piperidin-4-one |
| 12 | 1-Benzyl-5-[1-(4-methanesulfonyl-piperazin-1-yl)-methylidene]- |
| | 3,3-dimethyl-piperidin-4-one |
| 13 | 2-(4-Methoxy-benzyl)-3,3-dimethyl-4-oxo-5-[1-pyridin-2-yl- |
| | methylidene]-piperidine-1-carboxylic acid ethyl ester |
| 14 | 2-(4-Methoxy-benzyl)-3,3-dimethyl-4-oxo-5-[1-pyridin-2-yl- |
| | methylidene]-piperidine-1-carboxylic acid phenyl ester |
| 15 | 2-(4-Methoxy-benzyl)-3,3-dimethyl-4-oxo-5-[1-pyridin-2-yl- |
| | methylidene]-piperidine-1-carboxylic acid isobutyl ester |
| 16 | 1-(2,2-Dimethyl-propionyl)-2-(4-methoxy-benzyl)-3,3-dimethyl-5- |
| | [1-pyridin-2-yl-methylidene]-piperidin-4-one |
| 17 | 2-(4-Methoxy-benzyl)-3,3-dimethyl-4-oxo-5-[1-pyridin-2-yl- |
| | methylidene]-piperidine-1-carboxylic acid (2,6-dimethyl-phenyl)- |
| | amde |
| 18 | 1-Benzyl-3,3-dimethyl-5-[1-quinolin-2-yl-methylidene]-piperidin-4- |
| | one |
| 19 | 1-Benzyl-3,3-dimethyl-5-[1-(1H-pyrrol-2-yl)-methylidene]- |
| | piperidin-4-one |
| 20 | 1-Benzyl-3,3-dimethyl-5-[1-(6-morpholin-4-yl-pyridin-2-yl)- |
| | methylidene]-piperidin-4-one and its hydrochloride salt |
| 21 | 1-Benzyl-3,3-dimethyl-5-[1-quinoxalin-2-yl-methylidene]-piperidin- |
| | 4-one |
| 22 | 1-Benzyl-3,3-dimethyl-5-[1-thiophen-2-yl-methylidene]-piperidin- |
| | 4-one |
| 23 | 1-Benzyl-3,3-dimethyl-5-[1-(3,4,5,6-tetrahydro-2H- |
| | [1,2′]bipyridinyl-6′-yl)-methylidene]-piperidin-4-one |
| 24 | 1-Benzyl-5-[1-(3-hydroxy-quinoxalin-2-yl)-methylidene]-3,3- |
| | dimethyl-piperidin-4-one |
| 25 | 1-Benzyl-5,5-dimethyl-2-phenyl-3-[1-pyridin-2-yl-methylidene]- |
| | piperidin-4-one |
| 26 | 1-Benzyl-5,5-dimethyl-2-phenyl-3-[1-quinoxalin-2-yl-methylidene]- |
| | piperidin-4-one |
| 27 | 1-Benzyl-5,5-dimethyl-2-phenyl-3-[1-(1H-pyrrol-2-yl)- |
| | methylidene]-piperidin-4-one |
| 28 | 1-Benzyl-5,5-dimethyl-3-[1-(6-morpholin-4-yl-pyridin-2-yl)- |
| | methylidene]-2,3,5,6-tetrahydro-1H-[2,2′] |
| | bipyridinyl-4-one |
| 29 | 1-Benzyl-5,5-dimethyl-3-[1-pyridin-2-yl-methylidene]-2,3,5,6- |
| | tetrahydro-1H-[2,2′]bipyridinyl-4-one |
| 30 | 1-Benzyl-5,5-dimethyl-3-[1-(4-methylsulfanyl-phenyl)- |
| | methylidene]-2-phenyl-piperidin-4-one |
| 31 | 1-Benzyl-5,5-dimethyl-3-[1-(6-morpholin-4-yl-pyridin-2-yl)- |
| | methylidene]-2-phenyl-piperidin-4-one |
| 32 | 1-Benzyl-5,5-dimethyl-3-[1-pyridin-2-yl-methylidene]-2-thiophen- |
| | 2-yl-piperidin-4-one |
| 33 | 1-Benzyl-5,5-dimethyl-3-[1-(6-morpholin-4-yl-pyridin-2-yl)- |
| | methylidene]-2-thiophen-2-yl-piperidin-4-one |
| 34 | 1-Benzyl-5,5-dimethyl-3-[1-(3,4,5,6-tetrahydro-2H- |
| | [1,2′]bipyridinyl-6′-yl)-methylidene]-2,3,5,6-tetrahydro-1H- |
| | [2,2′]bipyridinyl-4-one |
| 35 | 3,3-Dimethyl-4-oxo-5-[1-(3,4,5,6-tetrahydro-2H-[1,2′]bipyridinyl-6′- |
| | yl)-methylidene]-piperidine-1-carboxylic acid phenyl ester |
| 36 | 3,3-Dimethyl-5-[1-(6-morpholin-4-yl-pyridin-2-yl)-methylidene]-4- |
| | oxo-piperidine-1-carboxylic acid phenyl ester |
| 37 | 2-[1-Benzyl-5,5-dimethyl-4-oxo-piperidin-3-ylidenemethyl]-3H- |
| | quinazolin-4-one |
| 38 | 1-Benzyl-3,3-dimethyl-5-[1-pyridin-3-yl-methylidene]-piperidin-4- |
| | one |
| 39 | 5′-[1-Benzyl-5,5-dimethyl-4-oxo-piperidin-3-ylidenemethyl]- |
| | 3,4,5,6-tetrahydro-2H-[1,2′]bipyridinyl-4-carboxylic acid |
| 40 | 1-Benzyl-2-(4-dimethylamino-phenyl)-5,5-dimethyl-3-[1-pyridin-2- |
| | yl-methylidene]-piperidin-4-one |
| 41 | 1-Benzyl-5-[1-[6-(3,5-dimethyl-morpholin-4-yl)-pyridin-3-yl]- |
| | methylidene]-3,3-dimethyl-piperidin-4-one |
| 42 | 1-Benzyl-5,5-dimethyl-2-(4-methylsulfanyl-phenyl)-3-[1-(6- |
| | morpholin-4-yl-pyridin-2-yl)-meth-(E)-ylidene]-piperidin-4-one |
| 43 | 1-Benzyl-5,5-dimethyl-3-[1-(6-morpholin-4-yl-pyridin-2-yl)- |
| | methylidene]-2-(4-trifluoromethyl-phenyl)- |
| | piperidin-4-one |
| 44 | 1-Benzyl-5,5-dimethyl-3-[1-pyridin-2-yl-methylidene]-2-(4- |
| | trifluoromethyl-phenyl)-piperidin-4-one |
| 45 | 1-Benzyl-2-(3,4-dichloro-phenyl)-5,5-dimethyl-3-[1-pyridin-2-yl- |
| | methylidene]-piperidin-4-one |
| 46 | 1-Benzyl-5,5-dimethyl-2-(4-methylsulfanyl-phenyl)-3-[1-pyridin-2- |
| | yl-methylidene]-piperidin-4-one and its hydrochloride salt |
| 47 | 1-(4-Methoxy-benzyl)-5,5-dimethyl-2-phenyl-3-[1-pyridin-2-yl- |
| | methylidene]-piperidin-4-one and its hydrochloride salt |
| 48 | 1-(4-Methoxy-benzyl)-5,5-dimethyl-3-[1-pyridin-2-yl-methylidene]- |
| | 2-thiophen-2-yl-piperidin-4-one |
| 49 | 1-Cyclopropyl-3,3-dimethyl-5-[1-pyridin-2-yl-methylidene]- |
| | piperidin-4-one |
| 50 | 3,3-Dimethyl-5-[1-(6-morpholin-4-yl-pyridin-2-yl)-methylidene]-1- |
| | thiophen-2-ylmethyl-piperidin-4-one |
| 51 | 1-Cyclopropyl-3,3-dimethyl-5-[1-(6-morpholin-4-yl-pyridin-2-yl)- |
| | methylidene]-piperidin-4-one |
| 52 | 2-(4-Methoxy-benzyl)-3,3-dimethyl-4-oxo-5-[1-pyridin-2-yl- |
| | methylidene]-piperidine-1-carboxylic acid methyl ester |
| 53 | 2-(4-Methoxy-benzyl)-3,3-dimethyl-4-oxo-5-[1-pyridin-2-yl- |
| | methylidene]-piperidine-1-carboxylic acid |
| | (4-methylsulfanyl-phenyl)-amide |
| 54 | 2-(4-Methoxy-benzyl)-3,3-dimethyl-4-oxo-5-[1-pyridin-2-yl- |
| | methylidene]-piperidine-1-carboxylic acid (2,6-dimethoxy- |
| | phenyl)-amde |
| 55 | 3,3-Dimethyl-1-(5-methyl-isoxazol-3-yl)-5-[1-(6-morpholin-4-yl- |
| | pyridin-2-yl)-methylidene]-piperidin-4-one |
| 56 | 2-(2-Hydroxy-phenyl)-5,5-dimethyl-1-(5-methyl-isoxazol-3-yl)-3- |
| | [1-(6-morpholin-4-yl-pyridin-2-yl)-methylidene]-piperidin-4-one |
| 57 | 2-(2-Fluoro-phenyl)-5,5-dimethyl-3-[1-(6-morpholin-4-yl-pyridin-2- |
| | yl)-methylidene]-1-thiophen-2-ylmethyl-piperidin-4-one |
| 58 | (2-Fluoro-phenyl)-5,5-dimethyl-3-[1-pyridin-2-yl-methylidene]-1- |
| | thiophen-2-ylmethyl-piperidin-4-one |
| 59 | 2-(4-Methoxy-benzyl)-3,3-dimethyl-4-oxo-5-[1-pyridin-2-yl- |
| | methylidene]-piperidine-1-carboxylic acid cyclohexylamide |
| 60 | 2-(4-Methoxy-benzyl)-3,3-dimethyl-4-oxo-5-[1-pyridin-2-yl- |
| | methylidene]-piperidine-1-carbothioic acid phenylamide |
| 61 | 5,5-Dimethyl-2-(4-methylsulfanyl-phenyl)-3-[1-pyridin-2-yl- |
| | methylidene]-1-thiophen-2-ylmethyl-piperidin-4-one |
| 62 | 1-(4-Methoxy-benzyl)-5,5-dimethyl-3-[1-(6-morpholin-4-yl-pyridin- |
| | 2-yl)-methylidene]-2-phenyl-piperidin-4-one |
| 63 | 1-(4-Methoxy-benzyl)-5,5-dimethyl-3-[1-(6-morpholin-4-yl-pyridin- |
| | 2-yl)-methylidene]-2-(4-trifluoromethyl-phenyl)-piperidin-4-one |
| 64 | 3,3-Dimethyl-1-(5-methyl-isoxazol-3-yl)-5-[1-pyridin-2-yl- |
| | methylidene]-piperidin-4-one |
| 65 | 5,5-Dimethyl-1-(5-methyl-isoxazol-3-yl)-3-[1-(6-morpholin-4-yl- |
| | pyridin-2-yl)-methylidene]-2-phenyl-piperidin-4-one |
| 66 | 2-(4-Methoxy-benzyl)-3,3-dimethyl-4-oxo-5-[1-pyridin-2-yl- |
| | methylidene]-piperidine-1-carboxylic acid benzylamide |
| 67 | 2-(4-Methoxy-benzyl)-3,3-dimethyl-4-oxo-5-[1-pyridin-2-yl- |
| | methylidene]-piperidine-1-carboxylicacid(4-fluoro-phenyl)-amide |
| 68 | 2-(4-Methoxy-benzyl)-3,3-dimethyl-4-oxo-5-[1-pyridin-2-yl- |
| | methylidene]-piperidine-1-carboxylic acid (2,6-diisopropyl- |
| | phenyl)-amide |
| 69 | 3,3-Dimethyl-5-[1-(6-morpholin-4-yl-pyridin-2-yl)-methylidene]-1- |
| | (2-thiophen-2-yl-ethyl)-piperidin-4-one and its hydrochloride salt |
| 70 | 2-(2-Fluoro-phenyl)-5,5-dimethyl-3-[1-pyridin-3-yl-methylidene]-1- |
| | thiophen-2-ylmethyl-piperidin-4-one |
| 71 | 1-Benzyl-5,5-dimethyl-3-[1-pyridin-2-yl-methylidene]-2-(3,4,5- |
| | trimethoxy-phenyl)-piperidin-4-one |
| 72 | 1-(4-Fluoro-benzyl)-3,3-dimethyl-5-[1-pyridin-2-yl-methylidene]- |
| | piperidin-4-one |
| 73 | 1-(4-Fluoro-benzyl)-3,3-dimethyl-5-[1-(6-morpholin-4-yl-pyridin-2- |
| | yl)-methylidene]-piperidin-4-one |
| 74 | 3,3-Dimethyl-5-[1-(6-morpholin-4-yl-pyridin-2-yl)-methylidene]-1- |
| | (4-trifluoromethyl-benzyl)-piperidin-4-one |
| 75 | 4-({2-(4-Methoxy-benzyl)-3,3-dimethyl-4-oxo-5-[1-pyridin-2-yl- |
| | methylidene]-piperidine-1-carbonyl}-amino)-benzoic acid ethyl |
| | ester |
| 76 | 1-(4-Fluoro-benzyl)-5,5-dimethyl-2-phenyl-3-[1-pyridin-2-yl- |
| | methylidene]-piperidin-4-one |
| 77 | 1-(4-Methoxy-benzyl)-5,5-dimethyl-3-[1-pyridin-2-yl-methylidene]- |
| | 2-(4-trifluoromethyl-phenyl)-piperidin-4-one |
| 78 | 2-(2-Fluoro-phenyl)-1-(4-methoxy-benzyl)-5,5-dimethyl-3-[1- |
| | pyridin-2-yl-methylidene]-piperidin-4-one |
| 79 | 3,3-Dimethyl-5-[1-pyridin-2-yl-methylidene]-1-(2-thiophen-2-yl- |
| | ethyl)-piperidin-4-one and its hydrochloride salt |
| 80 | 5,5-Dimethyl-3-[1-(6-morpholin-4-yl-pyridin-2-yl)-methylidene]-2- |
| | phenyl-1-(2-thiophen-2-yl-ethyl)-piperidin-4-one |
| 81 | 1-(4-Fluoro-benzyl)-5,5-dimethyl-3-[1-(6-morpholin-4-yl-pyridin-2- |
| | yl)-methylidene]-2-phenyl-piperidin-4-one |
| 82 | 1-Furan-2-ylmethyl-5,5-dimethyl-2-phenyl-3-[1-pyridin-2-yl- |
| | methylidene]-piperidin-4-one and its hydrochloride salt |
| 83 | 1-(3,4-Difluoro-benzyl)-5,5-dimethyl-2-phenyl-3-[1-pyridin-2-yl- |
| | methylidene]-piperidin-4-one |
| 84 | 5,5-Dimethyl-2-phenyl-3-[1-pyridin-2-yl-methylidene]-1-(2- |
| | thiophen-2-yl-ethyl)-piperidin-4-one |
| 85 | 1,5,5-Trimethyl-2-phenyl-3-[1-pyridin-2-yl-methylidene]-piperidin- |
| | 4-one |
| 86 | 2-(2-Fluoro-phenyl)-1-(4-methoxy-benzyl)-5,5-dimethyl-3-[1-(6- |
| | morpholin-4-yl-pyridin-2-yl)-methylidene]-piperidin-4-one |
| 87 | 1-(4-Fluoro-benzyl)-3,3-dimethyl-5-[1-(4-methylsulfanyl-phenyl)- |
| | methylidene]-piperidin-4-one |
| 88 | 5,5-Dimethyl-1-(5-methyl-isoxazol-3-yl)-2-(4-methylsulfanyl- |
| | phenyl)-3-[1-(6-morpholin-4-yl-pyridin-2-yl)-methylidene]- |
| | piperidin-4-one |
| 89 | 3,3-Dimethyl-1-(5-methyl-isoxazol-3-yl)-5-[1-(4-methylsulfanyl- |
| | phenyl)-methylidene]-piperidin-4-one |
| 90 | 1-Furan-2-ylmethyl-5,5-dimethyl-3-[1-pyridin-2-yl-methylidene]-2- |
| | (3,4,5-trimethoxy-phenyl)-piperidin-4-one |
| 91 | 1-Benzyl-2-(2-fluoro-4-methoxy-phenyl)-5,5-dimethyl-3-[1-pyridin- |
| | 2-yl-methylidene]-piperidin-4-one |
| 92 | 1-Benzyl-2-(2-fluoro-4-methoxy-phenyl)-5,5-dimethyl-3-[1-(6- |
| | morpholin-4-yl-pyridin-2-yl)-methylidene]-piperidin-4-one |
| 93 | 5,5-Dimethyl-3-[1-(6-morpholin-4-yl-pyridin-2-yl)-methylidene]-2- |
| | phenyl-1-(3,4,5-trimethoxy-benzyl)-piperidin-4-one |
| 94 | 5,5-Dimethyl-1-phenethyl-2-phenyl-3-[1-pyridin-2-yl-methylidene]- |
| | piperidin-4-one |
| 95 | 5,5-Dimethyl-3-[1-(6-morpholin-4-yl-pyridin-2-yl)-methylidene]-1- |
| | phenethyl-2-phenyl-piperidin-4-one |
| 96 | 5,5-Dimethyl-1-(5-methyl-isoxazol-3-yl)-3-[1-(6-morpholin-4-yl- |
| | pyridin-2-yl)-methylidene]-2-(4-trifluoromethyl-phenyl)-piperidin-4- |
| | one |
| 97 | 5,5-Dimethyl-1-(5-methyl-isoxazol-3-yl)-3-[1-[6-(4-methyl- |
| | piperazin-1-yl)-pyridin-2-yl]-methylidene]-2-(4-trifluoromethyl- |
| | phenyl)-piperidin-4-one |
| 98 | 5,5-Dimethyl-1-(5-methyl-isoxazol-3-yl)-3-[1-pyridin-2-yl- |
| | methylidene]-2-(4-trifluoromethyl-phenyl)-piperidin-4-one |
| 99 | {5,5-Dimethyl-3-[1-(6-morpholin-4-yl-pyridin-2-yl)-methylidene]-4- |
| | oxo-2-phenyl-piperidin-1-yl}-acetic acid |
| 100 | {5,5-Dimethyl-4-oxo-2-phenyl-3-[1-pyridin-2-yl-methylidene]- |
| | piperidin-1-yl}-acetic acid |
| 101 | {2-(4-Fluoro-phenyl)-5,5-dimethyl-4-oxo-3-[1-pyridin-2-yl- |
| | methylidene]-piperidin-1-yl}-acetic acid |
| 102 | {5,5-Dimethyl-3-[1-[6-(4-methyl-piperazin-1-yl)-pyridin-2-yl]- |
| | methylidene]-4-oxo-2-phenyl-piperidin-1-yl}-acetic acid |
| 103 | 1-Benzyl-3-[1-(6-morpholin-4-yl-pyridin-2-yl)-methylidene]-5- |
| | phenyl-piperidine-2,4-dione |
| 104 | 2-(4-Methanesulfonyl-phenyl)-3,3-dimethyl-5-[1-(6-morpholin-4-yl- |
| | pyridin-2-yl)-methylidene]-4-oxo-piperidine-1-carbothioic acid |
| | phenylamide |
| 105 | 2-(4-Methanesulfonyl-phenyl)-3,3-dimethyl-4-oxo-5-[1-pyridin-2- |
| | yl-methylidene]-piperidine-1-carbothioic acid phenylamide |
| 106 | 2-(4-Methanesulfonyl-phenyl)-3,3-dimethyl-4-oxo-5-[1-pyridin-2- |
| | yl-methylidene]-piperidine-1-carboxylic acid benzylamide |
| 107 | 1-Benzyl-5-phenyl-3-[1-pyridin-2-yl-methylidene]-piperidine-2,4- |
| | dione |
| 108 | 1-Benzyl-3-[1-[6-(4-methyl-piperazin-1-yl)-pyridin-2-yl]- |
| | methylidene]-5-phenyl-piperidine-2,4-dione |
| 109 | 1-(3,4-Dimethoxy-benzyl)-5,5-dimethyl-2-phenyl-3-[1-pyridin-2-yl- |
| | methylidene]-piperidin-4-one |
| 110 | 5,5-Dimethyl-1-(4-methyl-benzyl)-3-[1-[6-(4-methyl-piperazin-1- |
| | yl)-pyridin-2-yl]-methylidene]-2-phenyl-piperidin-4-one |
| 111 | 2-(4-Methanesulfonyl-phenyl)-3,3-dimethyl- |
| | 4-oxo-5-[1-pyridin-2-yl-methylidene]-piperidine-1- |
| | carboxylic acid (4-fluoro-phenyl)-amide |
| 112 | 5,5-Dimethyl-1-(2-morpholin-4-yl-ethyl)-2-phenyl-3-[1-pyridin-2-yl- |
| | methylidene]-piperidin-4-one |
| 113 | 5,5-Dimethyl-1-(2-morpholin-4-yl-ethyl)-3-[1-(6-morpholin-4-yl- |
| | pyridin-2-yl)-methylidene]-2-phenyl-piperidin-4-one |
| 114 | 1-Benzyl-3-(3,4-dimethoxy-phenyl)-4-hydroxy-5-[1-(6-morpholin- |
| | 4-yl-pyridin-2-yl)-methylidene]-5,6-dihydro-1H-pyridin-2-one |
| 115 | 5,5-Dimethyl-1-(2-morpholin-4-yl-ethyl)-3-[1-(6-morpholin-4-yl- |
| | pyridin-2-yl)-methylidene]-2-p-tolyl-piperidin-4-one |
| 116 | 4-Hydroxy-1-(4-methyl-benzyl)-3-[1-(6-morpholin-4-yl-pyridin-2- |
| | yl)-methylidene]-5-phenyl-3,6-dihydro-1H-pyridin-2-one |
| 117 | 2-(4-Dimethylamino-phenyl)-5,5-dimethyl-1-(4-methyl-benzyl)-3- |
| | [1-(6-morpholin-4-yl-pyridin-2-yl)-methylidene]-piperidin-4-one |
| 118 | 2-(4-Dimethylamino-phenyl)-5,5-dimethyl-1-(4-methyl-benzyl)-3- |
| | [1-pyridin-2-yl-meth-ylidene]-piperidin-4-one |
| 119 | 5,5-Dimethyl-2-(4-methylsulfanyl-phenyl)-3-[1-(6-morpholin-4-yl- |
| | pyridin-2-yl)-methylidene]-1-thiophen-2-ylmethyl-piperidin-4-one |
| 120 | 2-(2,5-Dimethoxy-phenyl)-3-[1-(4-methanesulfonyl-phenyl)- |
| | methylidene]-5,5-dimethyl-1-(4-methyl-benzyl)-piperidin-4-one |
| 121 | 2-(2,5-Dimethoxy-phenyl)-5,5-dimethyl-1-(4-methyl-benzyl)-3-[1- |
| | (4-methylsulfanyl-phenyl)-methylidene]-piperidin-4-one |
| 122 | N-(4-{1-Benzyl-4-hydroxy-5-[1-(6-morpholin-4-yl-pyridin-2-yl)- |
| | methylidene]-6-oxo-1,2,5,6-tetrahydro-pyridin-3-yl}-phenyl)- |
| | methanesulfonamide |
| 123 | 1-Benzyl-5-(3,5-dimethyl-phenyl)-3-[1-pyridin-2-yl-methylidene]- |
| | piperidine-2,4-dione |
| 124 | 1-Methanesulfonyl-3-[1-(6-morpholin-4-yl-pyridin-2-yl)- |
| | methylidene]-5-phenyl-piperidine-2,4-dione |
| 125 | 2-(4-Dimethylamino-phenyl)-5,5-dimethyl-1-(4-methyl-benzyl)-3- |
| | [1-quinolin-2-yl-methylidene]-piperidin-4-one |
| 126 | 1-Benzoyl-4-hydroxy-5-phenyl-3-[1-pyridin-2-yl-methylidene]-3,6- |
| | dihydro-1H-pyridin-2-one |
| 127 | 2-(4-Fluoro-phenyl)-5,5-dimethyl-1-(4-methyl-benzyl)-3-[1-(6- |
| | morpholin-4-yl-pyridin-2-yl)-methylidene]-piperidin-4-one |
| 128 | 4-Hydroxy-1-(4-methyl-benzyl)-5-phenyl-3-[1-pyridin-2-yl- |
| | methylidene]-3,6-dihydro-1H-pyridin-2-one |
| 129 | 1-(4-Methyl-benzyl)-3-[1-(4-methylsulfanyl-phenyl)-methylidene]- |
| | 5-phenyl-piperidine-2,4-dione |
| 130 | 1-(3-Methoxy-benzyl)-5-phenyl-3-[1-pyridin-2-yl-methylidene]- |
| | piperidine-2,4-dione |
| 131 | 5,5-Dimethyl-3-[1-(6-morpholin-4-yl-pyridin-2-yl)-methylidene]-2- |
| | phenyl-1-(2-piperidin-1-yl-ethyl)-piperidin-4-one |
| 132 | 2-(4-Fluoro-phenyl)-5,5-dimethyl-3-[1-(6-morpholin-4-yl-pyridin-2- |
| | yl)-methylidene]-1-(2-piperidin-1-yl-ethyl)-piperidin-4-one |
| 133 | 5,5-Dimethyl-2-phenyl-1-(2-piperidin-1-yl-ethyl)-3-[1-pyridin-2-yl- |
| | methylidene]-piperidin-4-one |
| 134 | 2-(4-Fluoro-phenyl)-5,5-dimethyl-1-(2-piperidin-1-yl-ethyl)-3-[1- |
| | pyridin-2-yl-methylidene]-piperidin-4-one |
| 135 | 5,5-Dimethyl-3-[1-(6-morpholin-4-yl-pyridin-2-yl)-methylidene]-1- |
| | (2-piperidin-1-yl-ethyl)-2-p-tolyl-piperidin-4-one |
| 136 | 2-(4-Dimethylamino-phenyl)-5,5-dimethyl-1-(2-piperidin-1-yl- |
| | ethyl)-3-[1-pyridin-2-yl-methylidene]-piperidin-4-one |
| 137 | 5,5-Dimethyl-3-[1-[6-(4-methyl-piperazin-1-yl)-pyridin-2-yl]- |
| | methylidene]-1-(2-piperidin-1-yl-ethyl)-2-p-tolyl-piperidin-4-one |
| 138 | 5,5-Dimethyl-1-(2-morpholin-4-yl-2-oxo-ethyl)-3-[1-(6-morpholin- |
| | 4-yl-pyridin-2-yl)-methylidene]-2-phenyl-piperidin-4-one |
| 139 | 5,5-Dimethyl-1-(2-piperidin-1-yl-ethyl)-3-[1-pyridin-2-yl- |
| | methylidene]-2-p-tolyl-piperidin-4-one |
| 140 | 2-(4-Fluoro-phenyl)-5,5-dimethyl-3-[1-[6-(4-methyl-piperazin-1-yl)- |
| | pyridin-2-yl]-methylidene]-1-(2-piperidin-1-yl-ethyl)-piperidin-4- |
| | one |
| 141 | 3,3-Dimethyl-5-[1-quinolin-2-yl-methylidene]-1-thiophen-2- |
| | ylmethyl-piperidin-4-one |
| 142 | 3,3-Dimethyl-5-[1-[6-(4-methyl-piperazin-1-yl)-pyridin-2-yl]- |
| | methylidene]-1-thiophen-2-ylmethyl-piperidin-4-one |
| 143 | 3,3-Dimethyl-5-[1-pyridin-2-yl-methylidene]-1-thiophen-2- |
| | ylmethyl-piperidin-4-one |
| 144 | 5,5-Dimethyl-3-[1-pyridin-2-yl-methylidene]-1-thiophen-2- |
| | ylmethyl-2-p-tolyl-piperidin-4-one |
| 145 | 5,5-Dimethyl-3-[1-(6-morpholin-4-yl-pyridin-2-yl)-methylidene]-1- |
| | thiophen-2-ylmethyl-2-p-tolyl-piperidin-4-one |
| 146 | 5,5-Dimethyl-3-[1-(6-morpholin-4-yl-pyridin-2-yl)-methylidene]-2- |
| | phenyl-1-thiophen-2-ylmethyl-piperidin-4-one |
| 147 | 5,5-Dimethyl-2-phenyl-3-[1-pyridin-2-yl-methylidene]-1-thiophen- |
| | 2-ylmethyl-piperidin-4-one |
| 148 | 2-(2,5-Dimethoxy-phenyl)-5,5-dimethyl-1-(4-methyl-benzyl)-3-[1- |
| | quinolin-2-yl-methylidene]-piperidin-4-one |
| 149 | 2-(4-Dimethylamino-phenyl)-5,5-dimethyl-3-[1-pyridin-2-yl- |
| | methylidene]-1-(2-thiophen-2-yl-ethyl)-piperidin-4-one |
| 150 | 2-(4-Dimethylamino-phenyl)-5,5-dimethyl-3-[1-(6-morpholin-4-yl- |
| | pyridin-2-yl)-methylidene]-1-(2-thiophen-2-yl-ethyl)-piperidin-4- |
| | one |
| 151 | 1-Benzyl-3-(3,4-dimethoxy-phenyl)-5-[1-pyridin-2-yl-methylidene]- |
| | piperidine-2,4-dione |
| 152 | 3,3-Dimethyl-5-[1-[6-(4-methyl-piperazin-1-yl)-pyridin-2-yl]- |
| | methylidene]-1-(2-thiophen-2-yl-ethyl)-piperidin-4-one |
| 153 | 5,5-Dimethyl-2-(4-methylsulfanyl-phenyl)-3-[1-(6-morpholin-4-yl- |
| | pyridin-2-yl)-methylidene]-1-(2-thiophen-2-yl-ethyl)-piperidin-4- |
| | one |
| 154 | 2-(4-Dimethylamino-phenyl)-5,5-dimethyl-3-[1-pyridin-2-yl- |
| | methylidene]-1-thiophen-2-ylmethyl-piperidin-4-one |
| 155 | 2-(4-Dimethylamino-phenyl)-5,5-dimethyl-3-[1-(6-morpholin-4-yl- |
| | pyridin-2-yl)-methylidene]-1-thiophen-2-ylmethyl-piperidin-4-one |
| 156 | 1-Benzyl-3-(3,4-dimethoxy-phenyl)-5-[1-[6-(4-methyl-piperazin-1- |
| | yl)-pyridin-2-yl]-methylidene]-piperidine-2,4-dione |
| 157 | 5,5-Dimethyl-2-(4-methylsulfanyl-phenyl)-3-[1-pyridin-2-yl- |
| | methylidene]-1-(2-thiophen-2-yl-ethyl)-piperidin-4-one |
| 158 | 5,5-Dimethyl-3-[1-quinolin-2-yl-methylidene]-1-thiophen-2- |
| | ylmethyl-2-p-tolyl-piperidin-4-one |
| 159 | 5,5-Dimethyl-2-(4-methylsulfanyl-phenyl)-3-[1-quinolin-2-yl- |
| | methylidene]-1-thiophen-2-ylmethyl-piperidin-4-one |
| 160 | 2-(4-Dimethylamino-phenyl)-5,5-dimethyl-3-[1-quinolin-2-yl- |
| | methylidene]-1-(2-thiophen-2-yl-ethyl)-piperidin-4-one |
| 161 | 5,5-Dimethyl-2-(4-methylsulfanyl-phenyl)-3-[1-quinolin-2-yl- |
| | methylidene]-1-(2-thiophen-2-yl-ethyl)-piperidin-4-one |
| 162 | 5,5-Dimethyl-3-[1-(6-morpholin-4-yl-pyridin-2-yl)-methylidene]-1- |
| | (2-thiophen-2-yl-ethyl)-2-p-tolyl-piperidin-4-one |
| 163 | 2-(2,5-Dimethoxy-phenyl)-5,5-dimethyl-1-(4-methyl-benzyl)-3-[1- |
| | pyrazin-2-yl-methylidene]-piperidin-4-one |
| 164 | 5,5-Dimethyl-3-[1-pyridin-2-yl-methylidene]-1-(2-thiophen-2-yl- |
| | ethyl)-2-p-tolyl-piperidin-4-one |
| 165 | 1-Benzyl-3-(3,4-dimethyl-phenyl)-5-[1-(6-morpholin-4-yl-pyridin-2- |
| | yl)-methylidene]-piperidine-2,4-dione |
| 166 | 1-Benzyl-5,5-dimethyl-3-[1-(4-methylsulfanyl-phenyl)- |
| | methylidene]-2,3,5,6-tetrahydro-1H-[2,3′]bipyridinyl-4-one |
| 167 | 1-Benzyl-5,5-dimethyl-3-[1-(4-trifluoromethyl-phenyl)- |
| | methylidene]-2,3,5,6-tetrahydro-1H-[2,3′]bipyridinyl-4-one |
| 168 | 1-(2-Fluoro-benzyl)-5,5-dimethyl-2-(4-methylsulfanyl-phenyl)-3-[1- |
| | pyridin-2-yl-methylidene]-piperidin-4-one |
| 169 | 1-(2-Fluoro-benzyl)-5,5-dimethyl-2-phenyl-3-[1-pyridin-2-yl- |
| | methylidene]-piperidin-4-one |
| 170 | 1-(2-Fluoro-benzyl)-5,5-dimethyl-3-[1-(6-morpholin-4-yl-pyridin-2- |
| | yl)-methylidene]-2-phenyl-piperidin-4-one |
| 171 | {5,5-Dimethyl-3-[1-(6-morpholin-4-yl-pyridin-2-yl)-methylidene]-4- |
| | oxo-2-p-tolyl-piperidin-1-yl}-acetic acid |
| 172 | 1-Benzyl-3-[1-(6-morpholin-4-yl-pyridin-2-yl)-methylidene]-5- |
| | phenyl-piperidin-4-one |
| 173 | 1-Benzyl-3-phenyl-5-[1-pyridin-2-yl-methylidene]-piperidin-4-one |
| 174 | 4-Oxo-3-phenyl-5-[1-pyridin-2-yl-methylidene]-piperidine-1- |
| | carboxylic acid (4-chloro-phenyl)-amide |
| 175 | 4-Oxo-3-phenyl-5-[1-pyridin-2-yl-methylidene]-piperidine-1- |
| | carboxylic acid (4-methylsulfanyl-phenyl)-amide |
| 176 | 3,3-Dimethyl-5-[1-(6-morpholin-4-yl-pyridin-2-yl)-methylidene]-4- |
| | oxo-2-phenyl-piperidine-1-carboxylic acid phenylamide |
| 177 | 2-Benzyl-3,3-dimethyl-5-[1-(6-morpholin-4-yl-pyridin-2-yl)- |
| | methylidene]-4-oxo-piperidine-1-carboxylic acid (4-methoxy- |
| | phenyl)-amide |
| 178 | 2-Benzyl-3,3-dimethyl-5-[1-(6-morpholin-4-yl-pyridin-2-yl)- |
| | methylidene]-4-oxo-piperidine-1-carbothioic acid phenylamide |
| 179 | 2-Benzyl-3,3-dimethyl-5-[1-(6-morpholin-4-yl-pyridin-2-yl)- |
| | methylidene]-4-oxo-piperidine-1-carboxylic acid (4-fluoro-phenyl)- |
| | amide |
| 180 | 2-Benzyl-3,3-dimethyl-5-[1-(6-morpholin-4-yl-pyridin-2-yl)- |
| | methylidene]-4-oxo-piperidine-1-carboxylic acid isopropylamide |
| 181 | 2-Benzyl-3,3-dimethyl-5-[1-(6-morpholin-4-yl-pyridin-2-yl)- |
| | methylidene]-4-oxo-piperidine-1-carboxytic acid p-tolylamide |
| 182 | 2-Benzyl-3,3-dimethyl-5-[1-(6-morpholin-4-yl-pyridin-2-yl)- |
| | methylidene]-4-oxo-piperidine-1-carboxylic acid phenylamide |
| 183 | 3,3-Dimethyl-5-[1-(6-morpholin-4-yl-pyridin-2-yl)-methylidene]-4- |
| | oxo-2-phenyl-piperidine-1-carboxylic acid p-tolylamide |
| 184 | 3,3-Dimethyl-5-[1-(6-morpholin-4-yl-pyridin-2-yl)-methylidene]-4- |
| | oxo-2-phenyl-piperidine-1-carboxylic acid (4-methoxy-phenyl)- |
| | amide |
| 185 | 4-Oxo-3-phenyl-5-[1-pyridin-2-yl-methylidene]-piperidine-1- |
| | carboxylic acid (2,4-dimethoxy-phenyl)-amide |
| 186 | 4-Oxo-3-phenyl-5-[1-pyridin-2-yl-methylidene]-piperidine-1- |
| | carboxylic acid phenylamide |
| 187 | 4-Oxo-3-phenyl-5-[1-pyridin-2-yl-methylidene]-piperidine-1- |
| | carboxylic acid p-tolylamide |
| 188 | 3,3-Dimethyl-5-[1-(6-morpholin-4-yl-pyridin-2-yl)-methylidene]-4- |
| | oxo-2-phenyl-piperidine-1-carboxylic acid (4-fluoro-phenyl)-amide |
| 189 | 3-[1-(4-Methylsulfanyl-phenyl)-methylidene]-4-oxo-5-phenyl- |
| | piperidine-1-carboxylic acid phenylamide |
| 190 | 3-[1-(4-Methylsulfanyl-phenyl)-methylidene]-4-oxo-5-phenyl- |
| | piperidine-1-carboxylic acid (4-chloro-phenyl)-amide |
| 191 | 3-[1-(4-Methanesulfonyl-phenyl)-methylidene]-4-oxo-5-phenyl- |
| | piperidine-1-carboxylic acid phenylamide |
| 192 | 1,5,5-Trimethyl-3-[1-(6-morpholin-4-yl-pyridin-2-yl)-methylidene]- |
| | 2-phenyl-piperidin-4-one |
| 193 | 3,3-Dimethyl-2-morpholin-4-ylmethyl-4-oxo-5-[1-pyridin-2-yl- |
| | methylidene]-piperidine-1-carboxylic acid (4-methylsulfanyl- |
| | phenyl)-amide |
| 194 | 3,3-Dimethyl-2-morpholin-4-ylmethyl-4-oxo-5-[1-pyridin-2-yl- |
| | methylidene]-piperidine-1-carboxylic acid (4-methoxy-phenyl)- |
| | amide |
| 195 | 4-({3,3-Dimethyl-2-morpholin-4-ylmethyl-4-oxo-5-[1-pyridin-2-yl- |
| | methylidene]-piperidine-1-carbonyl}-amino)-benzoic acid ethyl |
| | ester |
| 196 | N-{3,3-Dimethyl-5-[1-(6-morpholin-4-yl-pyridin-2-yl)-methylidene]- |
| | 4-oxo-2-phenyl-piperidine-1-carbonyl}-benzenesulfonamide |
| 197 | 1-Methanesulfonyl-3,3-dimethyl-2-morpholin-4-ylmethyl-5-[1- |
| | pyridin-2-yl-methylidene]-piperidin-4-one |
| 198 | 3,3-Dimethyl-2-morpholin-4-ylmethyl-5-[1-pyridin-2-yl- |
| | methylidene]-1-(toluene-4-sulfonyl)-piperidin-4-one |
| 199 | 1-Methanesulfonyl-3,3-dimethyl-2-phenyl-5-[1-pyridin-2-yl- |
| | methylidene]-piperidin-4-one |
| 200 | 1-Methanesulfonyl-3,3-dimethyl-5-[1-(6-morpholin-4-yl-pyridin-2- |
| | yl)-methylidene]-2-phenyl-piperidin-4-one |
| 201 | 3-[1-(6-Morpholin-4-yl-pyridin-2-yl)-methylidene]-5-phenyl-1- |
| | (toluene-4-sulfonyl)-piperidin-4-one |
| 202 | 3-Phenyl-5-[1-pyridin-2-yl-methylidene]-1-(toluene-4-sulfonyl)- |
| | piperidin-4-one |
| 203 | 1-Acetyl-3-[1-(6-morpholin-4-yl-pyridin-3-yl)-methylidene]-5- |
| | phenyl-piperidin-4-one |
| 204 | 1-Acetyl-3-methyl-5-[1-(6-morpholin-4-yl-pyridin-2-yl)- |
| | methylidene]-3-phenyl-piperidin-4-one |
| 205 | 3-[1-(6-Morpholin-4-yl-pyridin-2-yl)-methylidene]-4-oxo-5-phenyl- |
| | piperidine-1-carboxylic acid phenylamide |
| 206 | 1-Methanesulfonyl-3-[1-(6-morpholin-4-yl-pyridin-2-yl)- |
| | methylidene]-5-phenyl-piperidin-4-one |
| 207 | 3-[1-(6-Morpholin-4-yl-pyridin-2-yl)-methylidene]-4-oxo-5-phenyl- |
| | piperidine-1-carboxylic acid p-tolylamide |
| 208 | 3-[1-(6-Morpholin-4-yl-pyridin-2-yl)-methylidene]-4-oxo-5-phenyl- |
| | piperidine-1-carboxylic (2,4-dimethoxy-phenyl)-amide |
| 209 | 4-Oxo-3-phenyl-5-[1-pyridin-2-yl-methylidene]-piperidine-1- |
| | carboxylic acid (4-acetyl-phenyl)-amide |
| 210 | 1-Methanesulfonyl-3-phenyl-5-[1-pyridin-2-yl-methylidene]- |
| | piperidin-4-one |
| 211 | 4-Oxo-3-phenyl-5-[1-pyridin-2-yl-methylidene]-piperidine-1- |
| | carboxylic acid (2,4-dihydroxy-phenyl)-amide |
| 212 | 4-Oxo-3-phenyl-5-[1-pyridin-2-yl-methylidene]-piperidine-1- |
| | carboxylic acid (4-hydroxy-phenyl)-amide |
| 213 | 4-Oxo-3-phenyl-5-[1-pyridin-2-yl-methylidene]-piperidine-1- |
| | carboxylic (4-methanesulfonyl-phenyl)-amide |
| 214 | 1-(2,4-Dihydroxy-benzenesulfonyl)-3-phenyl-5-[1-pyridin-2-yl- |
| | methylidene]-piperidin-4-one |
| 215 | 4-{4-Oxo-3-phenyl-5-[1-pyridin-2-yl-methylidene]-piperidine-1- |
| | carbonyl}-benzenesulfonamide |
| 216 | 3-(4-Hydroxy-phenyl)-4-oxo-5-[1-pyridin-2-yl-methylidene]- |
| | piperidine-1-carboxylic acid phenylamide |
| 217 | 3-(4-Hydroxy-phenyl)-4-oxo-5-[1-pyridin-2-yl-methylidene]- |
| | piperidine-1-carboxylic acid (4-hydroxy-phenyl)-amide |
| 218 | 1-(4-Acetyl-benzoyl)-3-phenyl-5-[1-pyridin-2-yl-methylidene]- |
| | piperidin-4-one |
| 219 | 3-(4-Hydroxy-phenyl)-5-[1-pyridin-2-yl-methylidene]-1-(toluene-4- |
| | sulfonyl)-piperidin-4-one |
| 220 | 3-(4-Hydroxy-phenyl)-1-(4-methyl-benzoyl)-5-[1-pyridin-2-yl- |
| | methylidene]-piperidin-4-one |
| 221 | 1-Benzenesulfonyl-3-phenyl-5-[1-pyridin-2-yl-methylidene]- |
| | piperidin-4-one |
| 222 | 1-Benzoyl-3-(4-hydroxy-phenyl)-5-[1-pyridin-2-yl-methylidene]- |
| | piperidin-4-one |
| 223 | 1-(4-Hydroxy-benzyl)-2-(4-hydroxy-phenyl)-5,5-dimethyl-3-[1- |
| | pyridin-2-yl-methylidene]-piperidin-4-one |
| 224 | 1-(4-Hydroxy-benzyl)-2-(5-hydroxy-2-methoxy-phenyl)-5,5- |
| | dimethyl-3-[1-pyridin-2-yl-methylidene]-piperidin-4-one |
| 225 | 1-Methanesulfonyl-2-phenyl-4-(1-pyridin-2-yl-methylidene]- |
| | piperidin-3-one |
| 226 | 1-Benzenesulfonyl-3-(4-hydroxy-phenyl)- |
| | 5-[1-pyridin-2-yl-methylidene]-piperidin-4-one |
| 227 | 1-Benzyl-2-(4-methanesulfonyl-phenyl)-5,5-dimethyl-3-[1-(6- |
| | morpholin-4-yl-pyridin-2-yl)-methylidene]-piperidin-4-one |
| 228 | 1-Benzyl-5-[1-(4-methanesulfonyl-phenyl)-methylidene]-3,3- |
| | dimethyl-piperidin-4-one |
| 229 | 1-Benzyl-2-(4-methanesulfonyl-phenyl)-5,5-dimethyl-3-[1-pyridin- |
| | 2-yl-methylidene]-piperidin-4-one |
| 230 | 2-(2,5-Dimethoxy-phenyl)-5,5-dimethyl-1-(4-methyl-benzyl)-3-[1- |
| | (6-morpholin-4-yl-pyridin-2-yl)-methylidene]-piperidin-4-one |
| 231 | 5,5-Dimethyl-1-(4-methyl-benzyl)-2-phenyl-3-[1-pyridin-2-yl- |
| | methylidene]-piperidin-4-one |
| 232 | 5,5-Dimethyl-1-(4-methyl-benzyl)-3-[1-(6-morpholin-4-yl-pyridin-2- |
| | yl)-methylidene]-2-phenyl-piperidin-4-one |
| 233 | 2-(2,5-Dimethoxy-phenyl)-5,5-dimethyl-1-(4-methyl-benzyl)-3-[1- |
| | pyridin-2-yl-methylidene]-piperidin-4-one |
| 234 | 2-(2,5-Dimethoxy-phenyl)-5,5-dimethyl-1-(4-methyl-benzyl)-3-[1- |
| | [6-(4-methyl-piperazin-1-yl)-pyridin-2-yl]-methylidene]-piperidin-4- |
| | one |
| 235 | 1-(3,4-Dimethoxy-benzyl)-5,5-dimethyl-3-[1-(6-morpholin-4-yl- |
| | pyridin-2 yl)-methylidene]-2-phenyl-piperidin-4-one |
| 236 | 3-(4-Hydroxy-phenyl)-1-methanesulfonyl-5-[1-pyridin-2-yl- |
| | methylidene]-piperidin-4-one |
| 237 | 1-Benzenesulfonyl-3-(4-hydroxy-phenyl)-5-[1-pyridin-2-yl- |
| | methylidene]-piperidin-4-one |
| 238 | 1-(4-Amino-benzenesulfonyl)-3-phenyl-5-[1-pyridin-2-yl- |
| | methylidene]-piperidin-4-one |
| 239 | 1-(4-Hydroxy-benzoyl)-3-(4-hydroxy-phenyl)-5-[1-pyridin-2-yl- |
| | methylidene]-piperidin-4-one |
| 240 | 1-(3,5-Dihydroxy-benzoyl)-3-phenyl-5-[1-pyridin-2-yl- |
| | methylidene]-piperidin-4-one |
| 241 | 1-(4-Amino-benzenesulfonyl)-3-phenyl-5-[1-pyridin-2-yl- |
| | methylidene]-piperidin-4-one |
| 242 | 4-{4-Oxo-3-phenyl-5-[1-pyridin-2-yl-methylidene]-piperidine-1- |
| | sulfonyl}-benzamide |
| 243 | 4-{3-(4-Hydroxy-phenyl)-4-oxo-5-[1-pyridin-2-yl-methylidene]- |
| | piperidine-1-sulfonyl}-benzamide |
| 244 | 1-(3-Amino-4-hydroxy-benzoyl)-3-phenyl-5-[1-pyridin-2yl- |
| | methylidene]-piperidin-4-one |
| 245 | 1-(3-Amino-4-hydroxy-benzoyl)-3-(4-hydroxy-phenyl)-5-[1-pyridin- |
| | 2-yl-methylidene-piperidin-4-one |
| 246 | 1-(2,4-Dihydroxy-benzenesulfonyl)-3-(4-hydroxy-phenyl)-5-[1- |
| | pyridin-2-yl-methylidene]-piperidin-4-one |
| 247 | 2-{4-Oxo-3-phenyl-5-[1-pyridin-2-yl-methylidene]-piperidin-1-yl}- |
| | acetamide |
| 248 | 2-{3-(4-Hydroxy-phenyl)-4-oxo-5-[1-pyridin-2-yl-methylidene]- |
| | piperidin-1-yl}-acetamide |
| 249 | 4-Oxo-3-phenyl-5-[1-pyridin-2-yl-methylidene]-piperidine-1- |
| | sulfonic acid amide |
| 250 | 3-(4-Hydroxy-phenyl)-4-oxo-5-[1-pyridin-2-yl-methylidene]- |
| | piperidine-1-sulfonic acid amide |
| 251 | 4-Oxo-3-phenyl-5-[1-pyridin-2-yl-methylidene]-piperidine-1- |
| | carboxylic acid (4-amino-phenyl)-amide |
| 252 | 3-(4-Hydroxy-phenyl)-4-oxo-5-[1-pyridin-2-yl-methylidene]-piperidine- |
| | 1-carboxylic acid (4-amino-phenyl)-amide |
| 253 | 1-(4-Amino-benzoyl)-3-(4-hydroxy-phenyl)-5-[1-pyridin-2-yl- |
| | methylidene]-piperidin-4-one |
| 254 | 1-(4-Amino-benzoyl)-3-phenyl-5-[1-pyridin-2-yl-methylidene]- |
| | piperidin-4-one |
| 255 | 4-{4-Oxo-3-phenyl-5-[1-pyridin-2-yl-methylidene]-piperidine-1- |
| | carbonyl}-benzamide |
| 256 | 4-{3-(4-Hydroxy-phenyl)-4-oxo-5-[1-pyridin-2-yl-methylidene]- |
| | piperidine-1-carbonyl}-benzamide |
| 257 | 3-{4-Oxo-3-phenyl-5-[1-pyridin-2-yl-methylidene]-piperidine-1- |
| | sulfonyl}-benzoic acid |
| 258 | 3-{3-(4-Hydroxy-phenyl)-4-oxo-5-[1-pyridin-2-yl-methylidene]- |
| | piperidine-1-sulfonyl}-benzoic acid |
| 259 | 3-{4-Oxo-3-phenyl-5-[1-pyridin-2-yl-methylidene]-piperidine-1- |
| | carbonyl}-benzoic acid |
| 260 | 3-{3-(4-Hydroxy-phenyl)-4-oxo-5-[1-pyridin-2-yl-methylidene]- |
| | piperidine-1-carbonyl}-benzoic acid |
| 261 | 4-{4-Oxo-3-phenyl-5-[1-pyridin-2-yl-methylidene]-piperidine-1- |
| | carbonyl}-benzenesulfonamide |
| 262 | 1-(4-Methanesulfonyl-benzoyl)-3-phenyl-5-[1-pyridin-2-yl- |
| | methylidene]-piperidin-4-one |
| 263 | 4-({4-Oxo-3-phenyl-5-[1-pyridin-2-yl-methylidene]-piperidine-1- |
| | carbonyl}-amino)-benzoic acid |
| 264 | 4-({3-(4-Hydroxy-phenyl)-4-oxo-5-[1-pyridin-2-yl-methylidene]-piperidine- |
| | 1-carbonyl}-amino)-benzoic acid |
| 265 | 1-(4-Hydroxy-benzoyl)-3-(4-hydroxy-phenyl)-5-[1-(4- |
| | methanesulfonyl-phenyl)- |
| | methylidene]-piperidin-4-one |
| 266 | 3-(4-Hydroxy-phenyl)-5-[1-(4-methanesulfonyl-phenyl)- |
| | methylidene]- |
| | 4-oxo-piperidine-1-carboxylic acid (4-hydroxy-phenyl)-amide |
| 267 | 2-(4-Amino-phenyl)-5,5-dimethyl-3-[1-pyridin-2-yl-methylidene]-1- |
| | (2-thiophen-2-yl-ethyl)-piperidin-4-one |
| 268 | 2-(2,4-Dihydroxy-phenyl)-5,5-dimethyl-3-[1-pyridin-2-yl- |
| | methylidene]-1-(2-thiophen-2-yl-ethyl)-piperidin-4-one |
| 269 | 2-(3-Amino-4-hydroxy-phenyl)-5,5-dimethyl-3-[1-pyridin-2-yl- |
| | ethylidene]-1-(2-thiophen-2-yl-ethyl)-piperidin-4-one |
| 270 | 4-[5,5-Dimethyl-4-oxo-3-[1-pyridin-2-yl-methylidene]-1-(2- |
| | thiophen-2-yl-ethyl)-piperidin-2-yl]-benzamide |
| 271 | 1-(3-Hydroxy-benzenesulfonyl)-3,3-dimethyl-2-phenyl-5-[1- |
| | pyridin-2-yl-methylidene]-piperidin-4-one |
| 272 | 1-(2,5-Dihydroxy-benzenesulfonyl)-3,3-dimethyl-2-phenyl-5-[1- |
| | pyridin-2- |
| | yl-methylidene]-piperidin-4-one |
| 273 | 4-{3,3-Dimethyl-4-oxo-2-phenyl-5-[1-pyridin-2-yl-methylidene]- |
| | piperidine-1-carbonyl}-benzenesulfonamide |
| 274 | 2-(4-Amino-phenyl)-1-(4-hydroxy-benzyl)-5,5-dimethyl-3-[1- |
| | pyridin-2-yl- |
| | methylidene]-piperidin-4-one |
| 275 | 4-{1-(4-Hydroxy-benzyl)-5,5-dimethyl-4-oxo-3-[1-pyridin-2-yl- |
| | methylidene]-piperidin-2-yl}-benzamide |
| 276 | 2-(4-Amino-phenyl)-1-(3,4-dihydroxy-benzyl)-5,5-dimethyl-3-[1- |
| | pyridin-2-yl-methylidene]-piperidin-4-one |
| 277 | 4-{1-(3,4-Dihydroxy-benzyl)-5,5-dimethyl-4-oxo-3-[1-pyridin-2-yl- |
| | methylidene]-piperidin-2-yl}-benzamide |
| 278 | 1-(3,4-Dihydroxy-benzyl)-2-(4-hydroxy-phenyl)-5,5-dimethyl-3-[1- |
| | pyridin-2-yl-methylidene]-piperidin-4-one |
| 279 | 4-({4-Oxo-3-phenyl-5-[1-pyridin-2-yl-methylidene]-piperidine-1- |
| | carbonyl}-amino)-benzoic acid |
| 280 | 4-Oxo-3-phenyl-5-[1-pyridin-2-yl-methylidene]-piperidine-1- |
| | carboxylic acid (4-carbamoyl-phenyl)-amide |
| 281 | 1-(4-Hydroxy-benzoyl)-3-(4-hydroxy-phenyl)-5-[1-pyridin-2-yl- |
| | methylidene]-piperidin-4-one |
| 282 | 3-(4-Hydroxy-phenyl)-4-oxo-5-[1-pyridin-2-yl-methylidene]-piperidine- |
| | 1-carboxylic acid (4-hydroxy-phenyl)-amide |
| 283 | 3-Oxo-2-phenyl-4-[1-pyridin-2-yl-methylidene]-piperidine-1- |
| | carboxylic acid amide |
| 284 | 3-Oxo-2-phenyl-4-[1-pyridin-2-yl-methylidene]-piperidine-1- |
| | sulfonic acid amide |
| 285 | 1-(4-Hydroxy-benzenesulfonyl)-2-phenyl-4-[1-pyridin-2-yl- |
| | methylidene]-piperidin-3-one |
| 286 | 1-(4-Hydroxy-benzoyl)-2-phenyl-4-[1-pyridin-2-yl-methylidene]- |
| | piperidin-3-one |
| 287 | 3-Oxo-2-phenyl-4-[1-pyridin-2-yl-methylidene]-piperidine-1- |
| | carboxylic acid (4-hydroxy-phenyl)-amide |
| 288 | 3-Oxo-2-phenyl-4-[1-pyridin-2-yl-methylidene]-piperidine-1- |
| | carboxylic acid (4-sulfamoyl-phenyl)-amide |

In a further embodiment of the invention there is provided pharmaceutically acceptable compositions containing compounds of the present invention, in admixture with a pharmaceutically acceptable adjuvant, diluent or carrier.

### Pharmaceutical Compositions

In another embodiment, the present invention provides pharmaceutical composition comprising a therapeutically effective amount of one or more of a compound of formula (I) or (II). While it is possible to administer therapeutically effective quantity of compounds of formula (I) or (II) either individually or in combination, directly without any formulation, it is common practice to administer the compounds in the form of pharmaceutical dosage forms comprising pharmaceutically acceptable excipient(s) and at least one active ingredient. These dosage forms may be administered by a variety of routes including oral, topical, transdermal, subcutaneous, intramuscular, intravenous, intranasal, pulmonary etc.

Oral compositions may be in the form of solid or liquid dosage form. Solid dosage form may comprise pellets, pouches, sachets or discrete units such as tablets, multi-particulate units, capsules (soft & hard gelatin) etc. Liquid dosage forms may be in the form of elixirs, suspensions, emulsions, solutions, syrups etc. Composition intended for oral use may be prepared according to any method known in the art for the manufacture of the composition and such pharmaceutical compositions may contain in addition to active ingredients, excipients such as diluents, disintegrating agents, binders, solubilizers, lubricants, glidants, surfactants, suspending agents, emulsifiers, chelating agents, stabilizers, flavours, sweeteners, colours etc. Some example of suitable excipients include lactose, cellulose and its derivatives such as microcrystalline cellulose, methylcelulose, hydroxy propyl methyl cellulose, ethylcellylose, dicalcium phosphate, mannitol, starch, gelatin, polyvinyl pyrolidone, various gums like acadia, tragacanth, xanthan, alginates & its derivatives, sorbitol, dextrose, xylitol, magnesium Stearate, talc, colloidal silicon dioxide, mineral oil, glyceryl mono Stearate, glyceryl behenate, sodium starch glycolate, Cross Povidone, crosslinked carboxymethylcellulose, various emulsifiers such as polyethylene glycol, sorbitol fatty acid, esters, polyethylene glycol alkylethers, sugar esters, polyoxyethylene polyoxypropyl block copolymers, polyethoxylated fatty acid monoesters, diesters and mixtures thereof.

Sterile compositions for injection can be formulated according to conventional pharmaceutical practice by dissolving or suspending the active substance in a vehicle such as water for injection, N-Methyl-2-Pyrrolidone, propylene glycol and other glycols, alcohols, a naturally occurring vegetable oil like sesame oil, coconut oil, peanut oil, cottonseed oil or a synthetic fatty vehicle like ethyl oleate or the like. Buffers, anti-oxidants, preservatives, complexing agents like cellulose derivatives, peptides, polypeptides and cyclodextrins and the like can be incorporated as required. The dosage form can have a slow, delayed or controlled release of active ingredients in addition to immediate release dosage forms.

The amount of active ingredient which is required to achieve a therapeutic effect will, of course, vary with the particular compound, the route of administration, the subject under treatment, and the particular disorder or disease being treated. The compounds of the invention may be administered orally or parenteraly at a dose of from 0.001 to 1500 mg/kg per day, preferably from 0.01 to 1500 mg/kg per day, more preferably from 0.1 to 1500 mg/kg per day, most preferably from 0.1 to 500 mg/kg per day. The dose range for adult humans is generally from 5 mg to 35 g per day and preferably 5 mg to 2 g per day. Tablets or other dosage forms of presentation provided in discrete Units may conveniently contain an amount of compound of the invention which is effective at such dosage or as a multiple of the same, for example units containing 5 mg to 500 mg.

Yet another embodiment of the present invention is to provide a process for the preparation of the compounds of the present invention.

The following, reaction schemes give the alternate routes for synthesis of the compounds according to the present invention.

The compounds of formula (I) & (II) of the present invention may be prepared as shown in the schemes below and further described herein after.

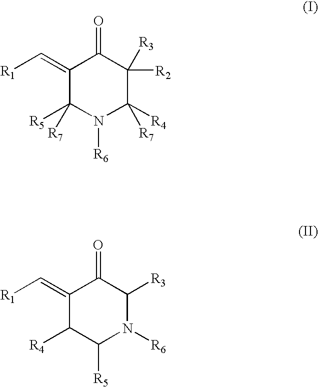

The compounds of formula (I) & (II) may be obtained through the intermediate (III) or (IV), wherein the R₁, R₂, R₃, R₄, R₅, R₆ and R₇ are as defined above.

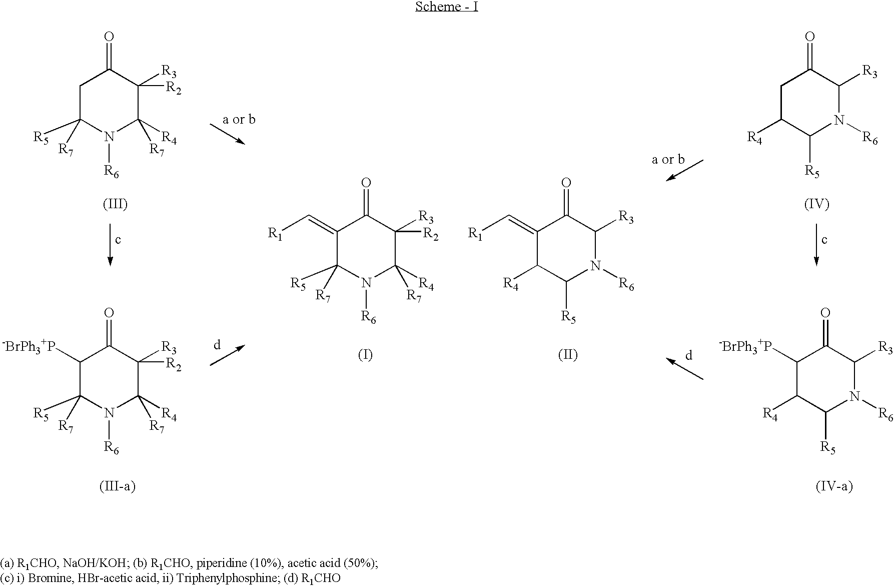

In one of the specific embodiment of the present invention, as shown in scheme-I, the compounds of formula (I) or (II) can be prepared by reacting, an aldehyde of formula, RiCHO wherein, R₁ is as defined above, such as unsubstituted or substituted benzaldehyde, pyridine carboxaldehyde, pyrrole carboxaldehyde, quinoline carboxaldehyde, quinoxaline carboxaldehyde or quinazoline carboxaldehyde, with a substituted piperidone of formula (III) or (IV) respectively, in the presence of a base such as aqueous NaOH or KOH, sodium methoxide, sodium ethoxide, potassium tertiary-butoxide, in the solvent such as methanol, ethanol, n-propanol, isopropanol, n-butanol, iso-butanol, t-butanol or sodium hydride in a solvent like toluene, tetrahydrofuran, dimethylformamide or pyridine and piperidine in toluene and at a temperature, in the range of 0° C. to 110° C., for a period of 2 to 12 hours.

Reference: (Furniss, et al, Vogel's Textbook of Practical Organic Chemistry, Fifth Edition, New York; John Wiley & Sons, Inc, (1989), Page:1033 and Canadian Journal of Chemistry, 1968, 46, 1952-1956) to obtain the compound of formula (I) & (II) respectively, and all other substituents are as defined above.

In an alternate process, the compounds of formula (I) or (II) is prepared by refluxing the solution of aldehyde of formula R₁CHO and a substituted piperidone of formula (III) or (IV) respectively, in ethanol containing 10% piperidine and 50% acetic acid with Soxhlet on 4A° molecular sieves, for a period of 24 to 30 hours.

Alternatively, the compound of formula (III) & (IV) is dissolved in an appropriate solvent such as carbon tetrachloride or methanol, containing HBr-acetic acid and treated with an equimolar quantity of bromine at a temperature of 0° C. to 80° C. for a period of 2 hours. The crude product obtained is treated with triphenyl phosphine in an appropriate solvent such as toluene at a temperature of 60° C. to 110° C. for a period of 30 min to 2 hours. The triphenylphosphine salt (III-a) & (IV-a) so obtained is treated with R₁CHO in a suitable solvent like pyridine at a temperature in the range of 100° C. to 115° C. for a period of 4 to 6 hours to obtain the compound of formula (I) & (II) respectively.

In another specific embodiment, as shown below in scheme-II, the compounds of formula (I) is obtained in following manner:

i) By treating the amine of formula R₆NH₂, such as unsubstituted or substituted benzylamine, thiophene ethylamine, thiophene methylamine, furyl methylamine, morpholine ethylamine, piperidine ethylamine, piperazine ethylamine, cyclopropylamine, cyclopentylamine, 2-amino-5-methyl-isoxazole, with one or two equivalent amount of R₄CHO such as paraformaldehyde, benzaldehyde, in an alcoholic solvent like methanol, ethanol, propanol or butanol at a temperature in the range of 0° C. to 110° C., for a period of 2 to 16 hours.

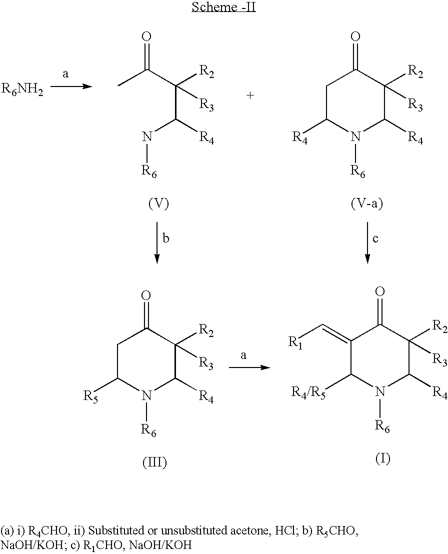

The reaction mixture thus obtained is added dropwise to the refluxing solution (1-2 hours) of substituted or unsubstituted acetone such as 2-methyl-3-butanone, 3-phenyl-butan-2-one, phenyl acetone, in an alcoholic solvent containing 10% to 50% inorganic acid such as hydrochloric acid, sulphuric acid, perchloric acid or organic acid such as acetic acid, propanoic acid, butanoic acid, heptanoic acid and further refluxed for a period of 8-10 hours to obtain the compound of formula (V) or (V-a).

ii) Further, the compound of formula (III) is prepared by dissolving the compound of formula (V), in an appropriate solvent such as ethanol, methanol, propanol, butanol containing base such as sodium hydroxide or potassium hydroxide, sodium methoxide, sodium ethoxide, potassium tertiary-butoxide; sodium hydride in the solvent like toluene, tetrahydrofuran, dimethylformamide or pyridine and piperidine in toluene and treating, with the compound of formula R₅CHO like unsubstituted or substituted benzaldehyde, pyridine carboxaldehyde, thiophene carboxaldehyde, furyl carboxaldehyde, pyrrole carboxaldehyde at a temperature from 0° C. to 110° C., for a period of 2 to 16 hours.
iii) The compound of formula (I) is prepared from compound of formula (III) or (V-a) by the methods as described in Scheme-I.

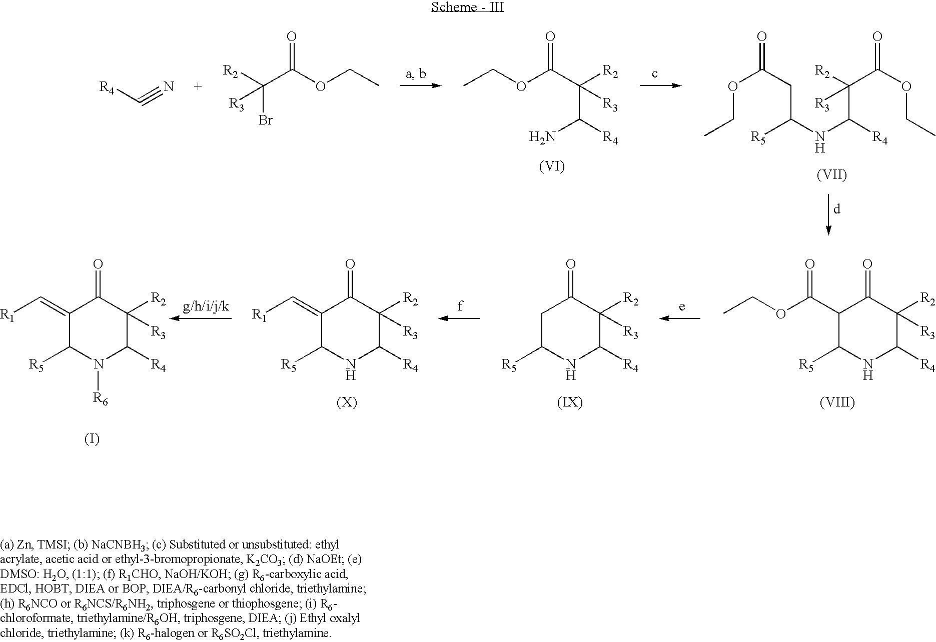

In still another specific embodiment, as shown in scheme-III, the compounds of formula (I) is prepared in following manner:

i) The solution of iodotrimethylsilane is added to the suspension of zinc in a solvent such as dichloromethane, chloroform, carbon tetrachloride, tetrahydrofuran, toluene, and is stirred at a temperature ranging from 0° C. to 110° C., for a period of 1 to 2 hours, further the ethyl bromoisobutyrate is added and stirred for a period of 15 min to 1 hour, followed by addition of the compound of formula R₄CN like unsubstituted or substituted phenylacetonitrile, benzonitrile or morpholin-4-yl acetonitrile, and the stirring is continued at a temperature of 60° C. to 110° C. for a period of 2 to 8 hours. The reaction mixture is cooled, filtered over celite and evaporated under vacuo. The crude product obtain is reduced with sodium borohydride or sodium cyanoborohydride in an appropriate solvents such as alcohols at a temperature from 0° C. to 110° C., for a period of 1 to 6 hours to obtain the compound of formula (VI),
ii) The compound of formula (VII) is prepared by reacting the compound of formula (VI) with substituted or unsubstituted ethyl acrylate containing a acid such as acetic acid, hydrochloric acid in a solvent such as toluene, N-methyl pyrrolidinone, alcohols at a temperature in the range of 0° C. to 160° C., for a period of 1 to 6 hours. Alternatively, by reacting the compound of formula (VI), with substituted or unsubstituted ethyl 3-bromopropionate in the presence of base such as potassium carbonate, sodium carbonate, or sodium hydride, in a solvent such as toluene, tetrahydrofuran, dimethylformamide, dichloromethane at a temperature in the range of 0° C. to 110° C., for a period of 1 to 12 hours, to obtain the compound of formula (VII).
iii) The compound of formula (VII) is treated in an appropriate solvent such as ethanol, methanol, butanol, toluene, tetrahydrofuran with base like sodium methoxide, sodium ethoxide, potassium tertiary-butoxide, sodium hydride, lithium hexamethyldisilazane, lithium diisopropylamide, n-butyl lithium at a temperature from −78° C. to 110° C., for a period of 3 to 12 hours to obtain the compound of formula (VIII),
iv) Further, by refluxing the compound of formula (VIII) with a mixture of dimethylsulfoxide (DMSO): water (1:1) at a temperature from 60° C. to 150° C., for 6 to 12 hours, provides the compound of formula (IX).
v) The compound of formula (X) is prepared from compound of formula (IX) by the methods as described in Scheme-I.
vi) (a) The compound of formula (I) is prepared by reacting R₆ carboxylic acid with 1-hydroxybenzotriazole and 1-(3-dimethylaminopropyl)-3-ethylcarbodiimide hydrochloride (EDCI) or benzotriazol-1-yl-oxytris(dimethylamino)phosphonium hexafluorophosphate (BOP) in a solvent such as tetrahydrofuran or dimethylformamide at a temperature from 0° C. to 25° C. for about 1 hour, followed by addition of N-ethyldiisopropylamine, the compound of formula (X) and is stirred at a room temperature for a period of 6 to 20 hours.
References: (i) (Sheehan, J. C.; Ledis, S. L.; Journal of American Chemical Society, (1973), 95, 875). (ii)(Keller-Schirlein, W; Muller, A; Hagmann, L; Schneisler, U; Zahner, H; Helv. Chim. Acta, (1985), 68, 559.; Le Nguyen, D; Castro, B; Peptide Chemistry (1987); Protein Research Foundation, Osaka, (1988), 231.; Kiso, Y; Kimura, T; Chemical Abstract, (1991), 114, 164722K).

In an alternate method, the R₆ carboxylic acid is treated with oxalyl chloride or thionyl chloride in a solvent like dichloromethane or toluene at a temperature in the range of 0° C. to 110° C., for a period of 3 to 4 hours to obtain the intermediate compound R₆ carbonyl chloride., which on further treatment with the compound of formula (X) in the presence of base, triethylamine or potassium carbonate in a solvent such as tetrahydrofuran, toluene, dimethylformamide at a temperature in the range of 0° C. to 25° C. for a period of 1 to 4 hours gives the compound of formula (I). Alternatively, when the ester of R₆ carboxylic acid is treated with the compound of formula (X), in a solvent such as toluene or xylene at a temperature in the range of 100° C. to 140° C., for a period of 1 to 12 hours provides the compound of formula (I).

(b) The compound of formula (I) is prepared by refluxing R₆ isocyanate or R₆ isothiocyanate with the compound of formula (X) in a solvent such as toluene, xylene or chloroform for a period of 6 to 12 hours.

The R₆ isocyanate is prepared by treating R₆ carboxylic acid with ethyl chloroformate, triethylamine or N-ethyl diisopropylamine in a solvent such as dichloromethane, dichloroethane, tetrahydrofuran, toluene at a temperature in the range of 0° C. to 60° C., for a period of 30 minutes to 3 hours gives mixed anhydride of R₆, which on treatment with solution of sodium azide (in water), at a temperature in the range of 25° C. to 110° C., for a period of 1 to 12 hours gives R₆ azide. Further, the R₆ azide is refluxed in toluene or xylene for a period of 1 to 4 hours to obtain R₆ isocyanate. Reference: (Carl Kaiser and Joseph Weinstock, Org. Syn. Coll. (1988), Vol. 6, 95, 910).

c) Alternatively, the compound of formula (I) is prepared by reacting R₆ NH₂ with triphosgene or thiophosgene in the presence of a base such as triethylamine, N-ethyldiisopropylamine, sodium bicarbonate, potassium or sodium carbonate in a solvent such as dichloromethane, chloroform or dichloroethane at a temperature in the range of 0° C. to 30° C., for a period of 30 minutes to 2 hours, followed by addition of the compound of formula (X) and is stirred at a temperature in the range 0° C. to 60° C. for a period 1 to 6 hours, Reference: (Iwakura, Y., Uno, K., Kang, S., J. Org. Chem., (1966), 31, 142; Kurita, K., Iwakura, Y., Org. Syn. Coll. Vol. 6, (1988), 715).
d) The compound of formula (I) is prepared by treating the compound of formula (X) with ethyl chloroformate or phenyl chloroformate in the presence of a base such as triethylamine, N-ethyldiisopropylamine, potassium or sodium carbonate in a solvent such as tetrahydrofuran, acetonitrile, toluene at a temperature in the range of 0° C. to 60° C., for a period of 10 minutes to 8 hours.

Alternatively, by treating the R₆ alcohol with phosgene or triphosgene in the presence of a base such as N-ethyl-diisopropylamine, triethylamine, potassium or sodium carbonate, in a solvent such as dichloromethane, chloroform or dichloroethane at the temperature in the range of 0° C. to 20° C. for a period of 1 hour, followed by addition of the compound of formula (X) and is stirred at a temperature in the range 0° C. to 60° C. for a period 1 to 6 hours, to obtain the compound of formula (I). Reference: (Cotarca, L., Detogan, P., Norddli, A., Sunji, V., Synthesis, (1996) 553)

e) The compound of formula (I) is prepared by treating the solution of compound of formula (X) with ethyl oxalyl chloride in the presence of base such as triethylamine or potassium carbonate in a solvent such as tetrahydrofuran, dichloromethane, toluene at a temperature in the range of 0° C. to 110° C., for a period of 3 to 6 hours, followed by treatment with R₆ amine in a solvent such as xylene, dimethylacetamide, N-methyl-2-pyrrolidinone at a temperature in the range of 100° C. to 160° C., for a period of 2 to 16 hours
(f) The compound of formula (I) is prepared by treating the compound of formula (X) with R₆-halogen or R₆ sulphonyl chloride in the presence of base such as triethylamine or potassium carbonate in a solvent such as tetrahydrofuran, dichloromethane, acetonitrile, toluene at a temperature in the range of 0° C. to 110° C., for a period of 1 to 6 hours.

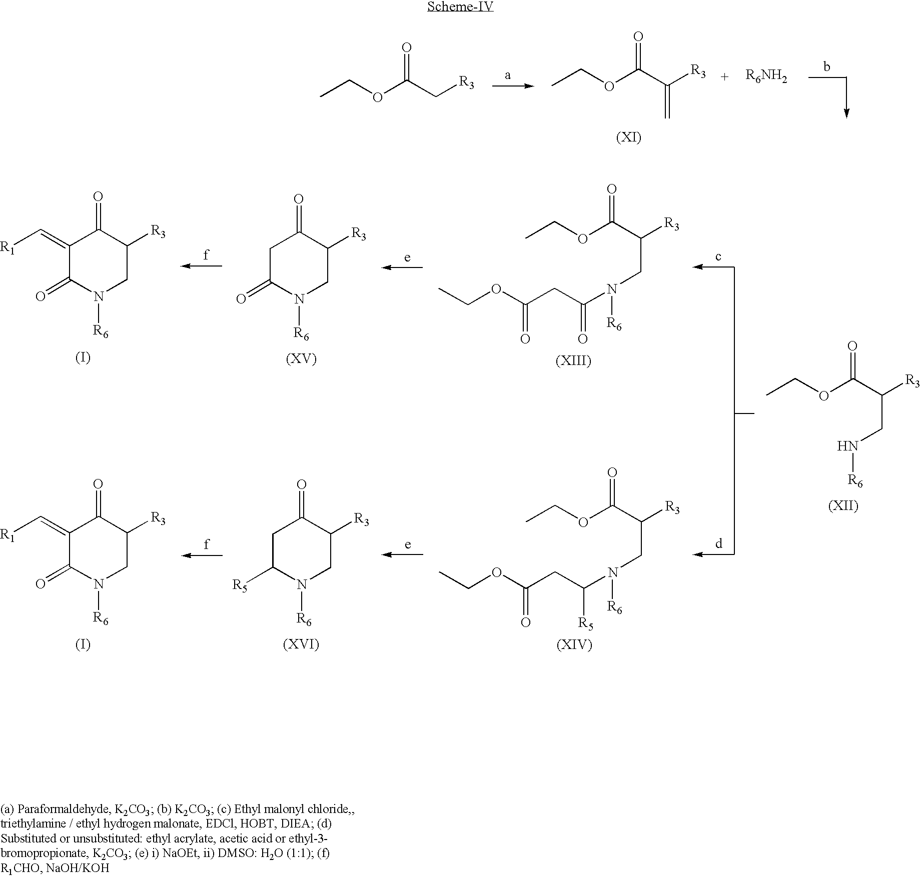

In still another embodiment, as shown in scheme-IV, the compounds of formula (I) is prepared by following procedure:

i) The ester of R₃ carboxylic acid is treated with paraformaldehyde in the presence of a base such as potassium carbonate, sodium carbonate, sodium hydride, sodium ethoxide, potassium tertiary-butoxide or sodium methoxide, in a solvents such as N-methylpyrrolidinone, toluene, dimethylformamide, dimethyl acetamide at a temperature in the range of 0° C. to 110° C., for a period of 2 to 12 hours to obtain the compound of the formula (XI),
ii) By treating the compound of the formula (XI) with R₆ amine in a solvent such as toluene, xylene, N-methylpyrrolidinone, dimethylformamide or dimethyl acetamide, in the presence of base like potassium carbonate, sodium carbonate, or sodium hydride at a temperature from 0° C. to 110° C. for 2 to 12 hours to obtain the compound of the formula (XII).
iii) By reacting the compound of formula (XII) with ethyl malonyl chloride in a solvent such as tetrahydrofuran, acetonitrile, dimethylformamide, toluene, dichloromethane containing a base such as potassium carbonate, sodium carbonate, sodium hydride, triethylamine or N-ethyldiisopropylamine at a temperature ranging from 0° C. to 110° C., for a period of 1 to 8 hours to obtain the compound of formula (XIII). Alternatively, by treating the ethyl hydrogen malonate with 1-hydroxybenzotriazole, and 1-(3-dimethylaminopropyl)-3-ethylcarbodiimide hydrochloride (EDCI) in a solvent such as tetrahydrofuran or dimethylformamide at a temperature from 0° C. to 25° C. for about 1 hour, followed by addition of N-ethyldiisopropylamine, the compound of formula (XII) and is stirred at a room temperature for a period of 6 to 20 hours to obtain the compound of formula (XIII).
iv) The compound of formula (XII) is reacted with substituted or unsubstituted ethyl acrylate containing a acid such as acetic acid, hydrochloric acid or ethyl-3-bromopropionate containing a base such as potassium carbonate, sodium carbonate, triethylamine or N-ethyldiisopropylamine in a solvent such as ethanol, methanol, butanol, acetonitrile, dimethylformamide or toluene at a temperature in the range of 0° C. to 110° C., for a period of 1 to 12 hours, provides the compound of formula (XIV).
v) Further, the compound of formula (XIII) or (XIV) is treated with an appropriate base such as sodium methoxide, sodium ethoxide, potassium tertiary-butoxide, sodium hydride, lithium hexamethyldisilazane, lithium diisopropylamide or n-butyl lithium in an appropriate solvent such as ethanol, methanol, butanol, toluene or tetrahydrofuran at a temperature in the range of −78° C. to 110° C., for a period of 3 to 12 hours to obtain the cyclized intermediate, which on treatment with a mixture of dimethylsulfoxide:water (1:1) at a temperature from 60° C. to 150° C., for a period of 6 to 12 hours, provides the compound of formula (XV) or (XVI) respectively,
vi) By following the procedure as described in Scheme-I the compound of formula (XV) or (XVI) is converted to the compound of formula (I).

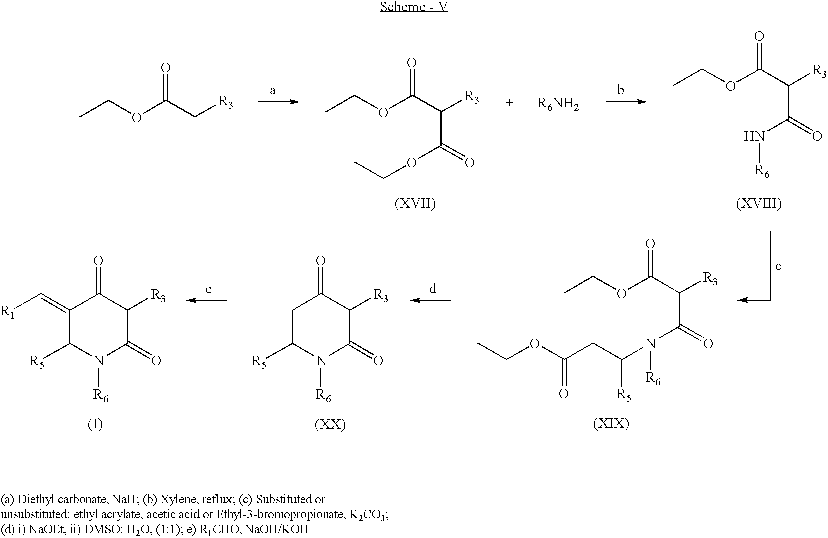

In a further embodiment of the present invention, as shown in scheme-V, the compounds of formula (I) is obtained

i) By treating the ester of R₃ carboxylic acid with diethyl carbonate in the presence of sodium hydride, potassium or sodium carbonate, in a solvent such as toluene, xylene, acetonitrile, dimethylformamide, N-methyl pyrrolidinone, dimethyl acetamide, at a temperature in the range of 60° C. to 150° C., for a period of 6 to 12 hours to obtain the compound of formula (XVII),
ii) Further, the compound of formula (XVII) is treated with R₆ amine in a solvent such as toluene or xylene at a temperature in the range of 100° C. to 140° C., for a period of 1 to 12 hours to obtain the compound of formula (XVIII),
iii) By reacting the compound of formula (XVIII) with substituted or unsubstituted ethyl acrylate containing a acid such as acetic acid, hydrochloric acid or ethyl-3-bromopropionate in an appropriate base such as potassium or sodium carbonate, triethylamine, N-ethyldiisopropylamine or sodium hydride in an appropriate solvent such as ethanol, methanol, butanol, dichloromethane, tetrahydrofuran, acetonitrile, toluene or dimethylformamide at a temperature in the range of 0° C. to 110° C., for a period of 1 to 8 hours to obtain the compound of formula (XIX),
iv) Further, the compound of formula (XIX) is treated with an appropriate base such as sodium methoxide, sodium ethoxide, potassium tertiary-butoxide, sodium hydride, lithium hexamethyldisilazane, lithium diisopropylamide or n-butyl lithium in an appropriate solvent such as ethanol, methanol, butanol, toluene or tetrahydrofuran at a temperature in the range of −78° C. to 110° C., for a period of 3 to 12 hours to obtain the cyclized intermediate, which on treatment with a mixture of dimethylsulfoxide:water (1:1) at a temperature from 60° C. to 150° C., for a period of 6 to 12 hours, provides the compound of formula (XX)
v) The compound of formula (I) is obtained from the compound of formula (XX) by the procedures as described in Scheme-I.

The compounds of formula (II) of the present invention is prepared as shown in the schemes below and further described herein after.

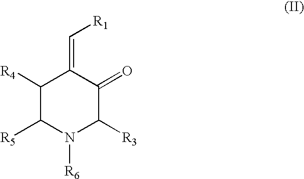

wherein, the R₁, R₃, R₄, R₅ and R₆ are as defined above.

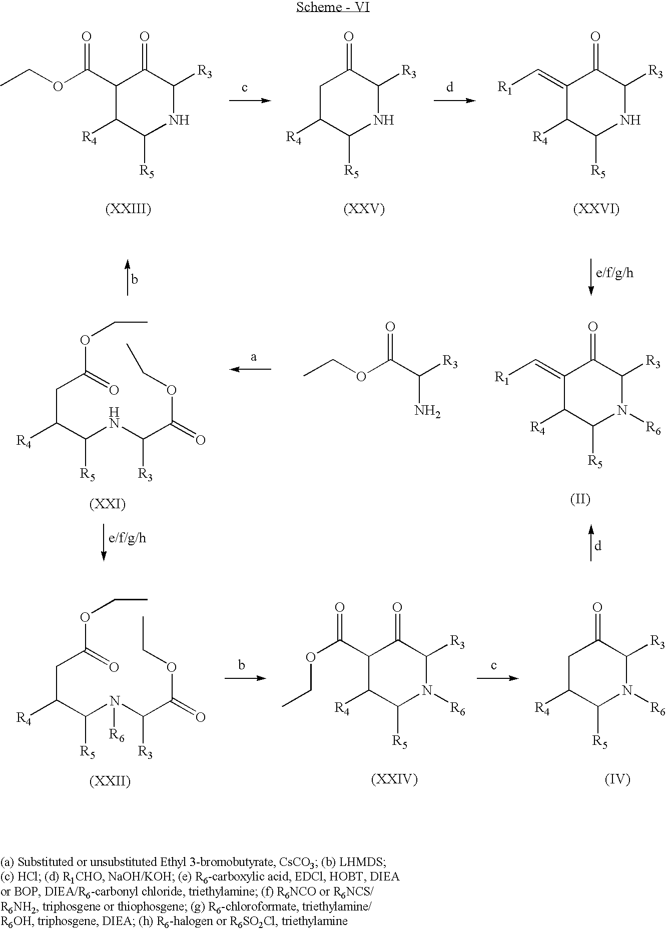

In another specific embodiment, as shown in scheme-VI, the compounds of formula (II) is prepared in following manner:

The R₃-amino acetic acid ethyl ester is treated with substituted or unsubstituted ethyl 3-bromobutyrate or ethyl 3-chlorobutyrate in the presence of a base such as triethylamine, N-ethyldiisopropylamine, cesium carbonate (CsCO₃), potassium or sodium carbonate in a solvent such as tetrahydrofuran, acetonitrile, toluene, dimethylformamide at a temperature in the range of 0° C. to 110° C., for a period of 30 minutes to 12 hours gives the compound of formula (XXI) which or further treatment with R₆ derivative by the methods as described in Scheme-III (VI) to give an intermediate (XXII)

The compound (XXI) & the intermediate (XXII) is treated with an appropriate base such as sodium methoxide, sodium ethoxide, potassium tertiary-butoxide, sodium hydride, lithium bis(trimethylsilyl)amide (LHMDS), lithium diisopropylamide or n-butyl lithium in an appropriate solvent such as ethanol, methanol, butanol, toluene or tetrahydrofuran at a temperature in the range of −78° C. to 110° C., for a period of 3 to 12 hours to obtain the cyclized intermediate (XXIII) & (XXIV) respectively, which on treatment with a hydrochloric acid solution at a temperature from 60° C. to 100° C., for a period of 6 to 12 hours, provides the compound of formula (XXV) & (IV) respectively.

The compound of formula (XXV) & (IV) gives the compound of formula (XXVI) & (II) by the methods as described in Scheme-I. Further the compound of formula (XXVI)_is treated by the methods as described in Scheme-III (IV) to give the compound of formula (II)

One of the ordinary skill will recognize to substitute appropriately modified starting material containing the various substituents. One of the ordinary skill will readily synthesize the disclosed compounds according to the present invention using conventional synthetic organic techniques and microwave techniques from starting material which are either purchased or may be readily prepared using prior art methods.

The compounds of the present invention may have chiral centers and occur as racemates, as individual diastereoisomers or enantiomers and as conformational isomers, with all isomeric forms being included in the present invention. Therefore, when a compound is chiral, the separate enantiomers, substantially free of the other, are included within the scope of the invention; further included are all mixtures of the two enantiomers.

The novel compounds of the present invention are not, however, to be construed as forming the only genus that is considered as the invention, and any combination of the compounds or their moieties may itself form a genus.

The novel compounds of the present invention were prepared according to the procedure of the schemes as described hereinabove, using appropriate materials and are further exemplified by the following specific examples. The examples are not to be considered nor construed as limiting the scope of the invention set forth in the claims appended thereto.

### Example 1

### 1-Benzyl-3,3-dimethyl-5-[1-(6-morpholin-4-yl-pyridin-2-yl)-methylidene]-piperidin-4-one

### Step A: Preparation of 6-morpholin-4-yl-pyridine-2-carboxaldehyde

The suspension of 6-bromo-pyridine-2-carboxaldehyde (1.9 g, 10 mmol), morpholine (1.75 g, 20 mmol) and potassium carbonate (3 g, 22 mmol) in acetonitrile (20 ml) was refluxed for 20 hours. The reaction mixture was then cooled to room temperature, diluted with water (20 ml) and the pH was adjusted to 7 by the aqueous solution of hydrochloric acid. The mixture was poured into water (50 ml) and extracted with ethylacetate (20 ml×3). The combined organic layers were washed with water (10 ml×2), brine (10 ml×2), dried over anhydrous sodium sulphate and evaporated under vacuo. The residue was purified by column chromatography over silica gel using 40% ethyl acetate in hexane as the eluent to afford the titled compound (1.5 g) was obtained as yellow solid.

¹HNMR (DMSOd₆): δ 3.55-3.58 (4H, t), 3.91-3.94 (4H, t), 7.15-7.18 (1H, d), 7.56-7.61 (1H, d), 7.65-7.6 (1H, t), 9.98 (1H, s). m/e: 193 (M⁺¹)

### Step B: Preparation of 1-Benzyl-3,3-dimethyl-piperidin-4-one.

The solution of benzylamine (12 g, 112 mmol) and paraformaldehyde (2 g, 66.6 mmol) in ethanol (30 ml) was stirred for 30 minutes at room temperature and then the mixture was added dropwise to the refluxing solution of 3-methyl-2-butanone (2.8 g, 32.5 mmol) in ethanol containing 10% HCl. The reaction mixture was refluxed for 8 hours. After completion of reaction, the mixture was cooled to room temperature and poured into water (100 ml), pH was adjusted to 7 using aqueous sodium bicarbonate solution and extracted with ethylacetate (50 ml×3). The combined organic layers were washed with water (50 ml×2), brine (50 ml×2), dried over anhydrous sodium sulphate and evaporated under vacuo. The residue was purified by column chromatography on silica gel using the 2% ethyl acetate in hexane as the eluent to provide the titled compound (2 g) as brown liquid.

¹HNMR (DMSOd₆): δ 1.16 (6H, s), 2.70-2.71 (2H, t), 2.72-2.77 (2H, t), 3.40-3.42 (2H, s), 3.50-3.52 (1H, d), 3.56-3.66 (1H, d), 7.20-7.22 (2H, m), 7.26-7.28 (3H, m) m/z: 218 (M⁺¹)

### Step C: Preparation of 1-Benzyl-3,3-dimethyl-5-[1-(6-morpholin-4-yl-pyridin-2-yl)-methylidene]-piperidin-4-one

The solution of 0.3 g (1.38 mmol) of the product of example 1, Step B in methanol (20 ml) was cooled to 0° C. The aqueous solution of sodium hydroxide (0.16 g, 4 mmol) and the 0.22 g (1.14 mmol) of the product of example 1, Step A was added to the reaction mixture, stirred at room temperature for 8 hours. After completion of reaction, the mixture was cooled to 0° C., diluted with water (20 ml). The solid precipitate obtained was washed with water (10 ml×2) and dried under vacuo to afford 1-Benzyl-3,3-dimethyl-5-[1-(6-morpholin-4-yl-pyridin-2-yl)-methylidene]-piperidin-4-one (0.2 g) as yellow solid.

¹HNMR (DMSOd₆): δ1.12 (6H, s), 2.60 (2H, s), 3.27-3.29 (4H, s), 3.64-3.66 (6H, m), 4.02 (2H, s), 6.80-6.82 (1H, d), 6.95-6.97 (1H, d), 7.13 (1H, s), 7.25-7.27 (1H, m), 7.31-7.37 (4H, m), 7.56-7.60 (1H, m) m/z: 392 (M⁺¹)

### Example 2

### 2-(2-Fluoro-phenyl)-5,5-dimethyl-3-[1-pyridin-3-yl-methylidene]-1-thiophen-2-ylmethyl-piperidin-4-one

### Step A: Preparation of 3,3-Dimethyl-4-[(thiophen-2-ylmethyl)-amino]-butan-2-one

The solution of thiophene-2-methylamine (2 g, 17.7 mmol) and paraformaldehyde (0.531 g, 17.7 mmol) in ethanol (20 ml) was stirred for 30 minutes at 60° C. and then the mixture was added dropwise to the refluxing solution of 3-Methyl-2-butanone (1.67 g, 19.4 mmol) in ethanol containing 10% HCl. The reaction mixture was refluxed for 8 hours. After completion of reaction, the mixture was cooled to room temperature and poured into water (100 ml), pH was adjusted to 7 using aqueous sodium bicarbonate solution and poured into water (100 ml) and extracted with ethylacetate (50 ml×3). The combined organic layers were washed with water (10 ml×2), brine (10 ml×2), dried over anhydrous sodium sulphate and evaporated under vacuo. The residue was purified by column chromatography on silica gel using the 2% ethyl acetate in hexane as the eluent to provide the titled compound (0.8 g) as brown liquid.

¹HNMR (DMSOd₆): δ 1.02 (6H, s), 2.05 (2H, s), 2.12 (1H, bs), 2.59 (3H, s), 3.85 (2H, s), 6.94-6.95 (2H, d), 7.36-7.37 (1H, m) m/z: 212 (M⁺¹)

### Step B: Preparation of 2-(2-Fluoro-phenyl)-5,5-dimethyl-1-thiophen-2-ylmethyl-piperidin-4-one

The solution of 0.5 g (2.4 mmol) of the product of example 2, Step A in methanol (10 ml) was cooled to 0° C. The aqueous solution of sodium hydroxide (0.114 g, 2.8 mmol) and the 2-fluoro benzaldehyde (0.294 g, 2.4 mmol) was added to the reaction mixture, stirred at room temperature for 10 hours. After completion of reaction, the mixture was cooled to 0° C., diluted with water (20 ml) and pH was adjusted to 7 using aqueous hydrochloric acid and poured into water (10 ml) and extracted with ethylacetate (20 ml×3). The combined organic layers were washed with water (10 ml×2), brine (5 ml×2), dried over anhydrous sodium sulphate and evaporated under vacuo. The residue was purified by column chromatography on silica gel using the 2% ethyl acetate in hexane as the eluent to provide the titled compound (0.45 g) as brown liquid.

¹HNMR (DMSOd₆): δ 0.92 (3H, s), 1.30 (3H, s), 2.29-2.34 (2H, dd), 2.75-2.82 (2H, m), 3.37-3.43 (1H, m), 3.64-3.67 (2H, d), 6.89-6.90 (1H, d), 6.93-6.95 (1H, m), 7.24-7.28 (2H, d), 7.41-7.46 (3H, d) m/z: 318 (M⁺¹)

### Step C: Preparation of 2-(2-Fluoro-phenyl)-5,5-dimethyl-3-[1-pyridin-2-yl-methylidene]-1-thiophen-2-ylmethyl-piperidin-4-one

The solution of 0.1 g (0.3 mmol) of the product of example 2, Step B in methanol (10 ml) was cooled to 0° C. The aqueous solution of sodium hydroxide (0.02 g, 0.5 mmol) and the pyridine-3-carboxaldehyde (0.034 g, 0.3 mmol) was added to the reaction mixture, stirred at room temperature for 8 hours. After completion of reaction, the mixture was cooled to 0° C., diluted with water (20 ml) and pH was adjusted to 7 using aqueous hydrochloric acid and poured into water (10 ml) and extracted with ethylacetate (5 ml×3). The combined organic layers were washed with water (5 ml×2), brine (50 ml×2), dried over anhydrous sodium sulphate and evaporated under vacuo. The residue was purified by column chromatography on silica gel using the 2% ethyl acetate in hexane as the eluent to provide the titled compound (0.02 g) as white solid.

¹HNMR (DMSOd₆): δ 1.14 (6H, s), 3.22 (2H, s), 3.39-3.46 (1H, m), 4.01-4.04 (1H, d), 5.45 (1H, d), 6.90-6.93 (2H, m), 7.21-7.26 (2H, m), 7.30 (1H, s), 7.33-7.35 (1H, m), 7.36-7.41 (1H, m), 7.42-7.44 (2H, m), 7.60-7.62 (1H, m), 8.41 (1H, s), 8.47-8.49 (1H, dd) m/z; 407 (M⁺¹)

### Example 3

### 2-(4-Methoxy-benzyl)-3,3-dimethyl-4-oxo-5-[1-pyridin-2-yl-methylidene]-piperidine-1-carboxylic acid (2,6-dimethyl-phenyl)-amide

### Step A: Preparation of 3-(2-Ethoxycarbonyl-ethylamino)-4-(4-methoxy-phenyl)-2,2-dimethyl-butyric acid ethyl ester

The solution of ethyl bromoisobutyrate (12.9 g 114.8 mmol) in tetrahydrofuran (30 ml) was added to the suspension of zinc in dichloromethane (30 ml) containing iodosotrimethylsilane (10.4 g, 67.5 mmol) under nitrogen atmosphere and stirred for 1 hour. The 4-methoxyphenylacetonitrile was then added dropwise and refluxed for 12 hours. The reaction mixture was cooled, filtered over celite and evaporated under vacuo. The crude product was dissolved in ethanol and cooled to 0° C. and then the sodium cyanoborohydride (2.47 g, 38 mmol) was added portionwise, stirred at room temperature for 8 hours. After completion of reaction, the mixture was cooled to 0° C., the pH was adjusted to 7 using ammonia solution (15 ml) and filtered over celite, evaporated under vacuo. The residue in toluene was washed with 10% hydrochloric acid (50 ml×2) and the aqueous phase was neutralized using ammonia and poured into water (100 ml) and extracted with ethylacetate (50 ml×3). The combined organic layers were washed with water (50 ml×2), brine (50 ml×2), dried over anhydrous sodium sulphate and evaporated under vacuo to provide the 3-amino-4-(4-methoxy-phenyl)-2,2-dimethyl-butyric acid ethyl ester (3 g) as brown liquid. Then this compound (3 g, 11.3 mmol) and ethyl acrylate (1.5 g, 11.3 mmol) was refluxed for 4 hours. The crude product was purified by column chromatography on silica gel using the 25% ethyl acetate in hexane as the eluent to provide the titled compound (3.48 g) as brown liquid.

¹HNMR (DMSOd₆): δ 1.1 (3H, s), 1.23 (3H, s), 1.25-1.27 (3H, t), 1.29-1.37 (3H, t), 2.09-2.16 (2H, m), 2.18-2.20 (1H, dd), 2.27-2.33 (2H, m), 2.56-2.78 (1H, d), 2.93-2.96 (1H, d), 3.67 (1H, bs), 3.81 (3H, s), 4.05-4.11 (2H, q), 4.12-4.16 (2H, q), 6.83-6.85 (2H, d), 7.15-7.17 (2H, d) m/z: 366 (M⁺¹)

### Step B: Preparation of 2-(4-Methoxy-benzyl)-3,3-dimethyl-piperidin-4-one

The solution of 3.4 g (9.4 mmol) of the product of example 3, Step A in toluene (50 ml) was added dropwise to the solution of sodium (0.43 g, 18.6 mmol) in ethanol (5 ml) and refluxed for 4 hours. After completion of reaction, the mixture was cooled to room temperature and poured into water (50 ml), pH was adjusted to 7 using aqueous hydrochloric acid and extracted with ethylacetate (50 ml×3). The combined organic layers were washed with water (50 ml×2), brine (50 ml×2), dried over anhydrous sodium sulphate and evaporated under vacuo. The residue was purified by column chromatography on silica gel using the 40% ethyl acetate in hexane as the eluent to provide 6-Benzyl-5,5-dimethyl-4-oxo-piperidine-3-carboxylic acid ethyl ester (2 g). Then this compound (2 g, 3.1 mmol) was refluxed with aqueous sodium hydroxide (1 g, 25 mmol) in ethanol (10 ml) for 3 hours. The reaction mixture was cooled to room temperature and poured into water (50 ml), pH was adjusted to 7 using aqueous hydrochloric acid and extracted with ethylacetate (50 ml×3). The combined organic layers were washed with water (50 ml×2), brine (50 ml×2), dried over anhydrous sodium sulphate and evaporated under vacuo. The residue was purified by column chromatography on silica gel using the 50% ethyl acetate in hexane as the eluent to provide the titled compound (0.88 g) as brown liquid.

¹HNMR (DMSOd₆): δ 1.20 (3H, s), 1.25 (3H, s), 2.07-2.10 (2H, d), 2.65-2.72 (1′-f, m), 2.98-3.05 (1H, m), 3.51-3.71 (2H, m), 3.74 (3H, s), 4.12-4.15 (2H, d), 6.75-6.77 (2H, d), 7.05-7.15 (2H, d) m/z: 248 (M⁺¹)

### Step C: Preparation of 2-(4-Methoxy-benzyl)-3,3-dimethyl-5-[1-pyridin-2-yl-methylidene]-piperidin-4-one

The solution of 0.2 g (0.8 mmol) of the product of example 3, Step B and potassium tert-butoxide (0.181 g, 1.6 mmol) in tetrahydrofuran (10 ml) was cooled to −20° C. and pyridine-2-carboxaldehyde (0.087 g, 0.8 mmol) was added after 15 minutes. The reaction mixture was stirred at room temperature for 2 hours. After completion of reaction, the mixture was cooled to 0° C., diluted with water (20 ml) and pH was adjusted to 7 using aqueous hydrochloric acid. The mixture was poured into water (10 ml) and extracted with ethylacetate (5 ml×3). The combined organic layers were washed with water (50 ml×2), brine (50 ml×2), dried over anhydrous sodium sulphate and evaporated under vacuo. The residue was purified by column chromatography on silica gel using the 60% ethyl acetate in hexane as the eluent to provide the titled compound (0.092 g) as yellow solid.

¹HNMR (DMSOd₆): δ 1.27 (3H, s), 1.31 (3H, s), 2.41-2.47 (1H, q), 2.87-2.91 (2H, dd), 2.96-3.0 (1H, dd), 3.83 (3H, s), 3.95-4.0 (1H, dd), 4.67-4.71 (1H, dd), 6.88-6.90 (2H, d), 7.15-7.17 (1H, m), 7.18-7.20 (2H, m), 7.35-7.37 (1H, m), 7.39 (1H, s), 7.66-7.70 (1H, m), 8.61-8.62 (1H, d) m/z: 337 (M⁺¹)

### Step D: Preparation of 2-(4-Methoxy-benzyl)-3,3-dimethyl-4-oxo-5-[1-pyridin-2-yl-methylidene]-piperidine-1-carboxylic acid (2,6-dimethyl-phenyl)-amide

The suspension of 0.11 g (3.3 mmol) of the product of example 3, Step C and 2,6-dimethylphenyl isocyanate (0.048 g, 3.3 mmol) in toluene (30 ml) was refluxed for 12 hours. The precipitate was filtered, washed with water (10 ml×2) and dried under vacuo. The residue was purified by column chromatography on silica gel using the 2% methanol in dichloromethane as the eluent to provide the titled compound (0.062 g) as yellow solid.

¹HNMR (DMSOd₆): δ 1.27 (6H, s), 1.91 (6H, s), 2.84-2.86 (1H, m), 2.88-2.92 (1H, m), 3.63-3.71 (1H, d), 3.78 (3H, s), 3.83-3.86 (1H, d), 4.29-4.33 (1H, t), 6.02 (1H, bs), 6.82-6.84 (2H, d), 6.98-7.03 (2H, m), 7.04-7.05 (1H, m), 7.06-7.15 (3H, m), 7.37-7.39 (1H, d), 7.62-7.66 (1H, m), 7.78 (1H, s), 8.51-8.52 (1H, dd) m/z: 484 (M⁺¹)

### Example 4

### 1-Benzyl-3-[1-(6-morpholin-4-yl-pyridin-2-yl)-methylidene]-5-phenyl-piperidine-2,4-dione

### Step A: Preparation of 3-benzylamino-2-phenyl-propionic acid ethyl ester

The solution of ethyl phenylacetate (5 g, 30 mmol), potassium carbonate (6.31 g, 45 mmol) and paraformaldehyde (1.37 g, 45 mmol) in 1-methyl-2-pyrrolidinone (30 ml) was heated at 90° C. for 7 hours. The mixture was poured into water (100 ml) and extracted with ethylacetate (50 ml×3). The combined organic layers were washed with water (50 ml×2), brine (50 ml×2), dried over anhydrous sodium sulphate and evaporated under vacuo. The residue was purified by column chromatography on silica gel using the 1% ethyl acetate in hexane as the eluent to provide 2-phenyl-acrylic acid ethyl ester (3 g) as colourless liquid. Then this compound (3 g, 17 mmol) and benzylamine (1.82 g, 17 mmol) in toluene was refluxed for 4 hours. The mixture was poured into water (50 ml) and extracted with ethylacetate (20 ml×3). The combined organic layers were washed with water (10 ml×2), brine (10 ml×2), dried over anhydrous sodium sulphate and evaporated under vacuo. The residue was purified by column chromatography on silica gel using the 5% ethyl acetate in hexane as the eluent to provide the titled compound (3.5 g) as brown liquid.

¹HNMR (DMSOd₆): δ 1.17-1.19 (3H, t), 2.23 (1H, bs), 2.68-2.72 (1H, m), 3.06-3.11 (1H, t), 3.69-3.70 (2H, d), 3.77-3.81 (1H, m), 4.02-4.09 (2H, q), 7.20-7.22 (2H, m), 7.26-7.32 (8H, m) m/z: 284 (M⁺¹)

### Step B: Preparation of 3-[Benzyl-(2-ethoxycarbonyl-acetyl)-amino]-2-phenyl-propionic acid ethyl ester

The solution of 3-benzylamino-2-phenyl-propionic acid ethyl ester (3.5 g) in tetrahydrofuran was cooled to 0° C. and sodium hydride (1.2 g, 24 mmol) was added portionwise. After 15 minutes the ethyl malonyl chloride (3.72 g, 24.7 mmol) was added and heated at 60° C. for 4 hours. Thd mixture was poured into water (50 ml) and extracted with ethylacetate (20 ml×3). The combined organic layers were washed with water (20 ml×2), brine (50 ml×2), dried over anhydrous sodium sulphate and evaporated under vacuo. The residue was purified by column chromatography on silica gel using the 5% ethyl acetate in hexane as the eluent to provide the titled compound (3.5 g) as brown liquid.

¹HNMR (DMSOd₆): δ 1.12-1.19 (6H, t), 3.18 (2H, s), 3.58-3.61 (2H, m), 3.70-3.72 (2H, s), 3.82-3.85 (1H, t), 4.03-4.11 (4H, q), 7.20-7.22 (2H, m), 7.26-7.34 (8H, m) m/z: 398 (M⁺¹)

### Step C: Preparation of 1-Benzyl-5-phenyl-piperidine-2,4-dione

The solution of 3.5 g (8.8 mmol) of the product of example 4, Step B in ethanol (10 ml) was cooled to at 0° C. and potassium tert-butoxide (0.56 g, 5 mmol) was added. The reaction mixture was stirred at room temperature for 4 hours. After completion of reaction, the mixture was cooled to 0° C., diluted with water (20 ml) and pH was adjusted to 7 using aqueous hydrochloric acid. The mixture was poured into water (50 ml) and extracted with ethylacetate (20 ml×3). The combined organic layers were washed with water (50 ml×2), brine (50 ml×2), dried over anhydrous sodium sulphate and evaporated under vacuo to obtained 1-Benzyl-2,4-dioxo-5-phenyl-piperidine-3-carboxylic acid ethyl ester (1 g) as colourless liquid. Then this crude compound (1 g) was dissolved in dimethylsulphoxide:water (1:1, 10 ml) and heated at 140° C. for 8 hours. The mixture was poured into water (20 ml) and extracted with ethylacetate (10 ml×3). The combined organic layers were washed with water (100 ml×2), brine (50 ml×2), dried over anhydrous sodium sulphate and evaporated under vacuo. The residue was purified by column chromatography on silica gel using the 40% ethyl acetate in hexane as the eluent to provide the titled compound (0.6 g) as brown liquid.

¹HNMR (DMSOd₆): δ 3.19-3.27 (1H, d), 3.41-3.43 (1H, d), 3.74-3.79 (1H, m), 3.86-3.87 (1H, d), 4.21-4.26 (1H, d), 4.34-4.38 (1H, d), 4.45-4.49 (1H, d), 7.15-7.16 (3H, m), 7.20-7.26 (2H, m), 7.28-7.36 (5H, m) m/z: 280 (M⁺¹)

### Step D: Preparation of 1-Benzyl-3-[1-(6-morpholin-4-yl-pyridin-2-yl)-methylidene]-5-phenyl-piperidine-2,4-dione

The solution of 0.25 g (0.89 mmol) of the product of example 4, Step C in methanol (20 ml) was cooled to 0° C., sodium hydroxide (0.07 g, 1.7 mmol) and 6-Morpholin-4-yl-pyridine-2-carboxaldehyde (0.15 g, 0.8 mmol) was added and stirred at room temperature for 8 hours. After completion of reaction, the mixture was cooled to 0° C., diluted with water (20 ml) and pH was adjusted to 7 using aqueous hydrochloric acid and extracted with ethylacetate (5 ml×3). The combined organic layers were washed with water (50 ml×2), brine (50 ml×2), dried over anhydrous sodium sulphate and evaporated under vacuo. The residue was purified by column chromatography on silica gel using the 5% ethyl acetate in hexane as the eluent to afford the titled compound (0.052 g) as yellow solid.

¹HNMR (DMSOd₆): δ 3.48-3.50 (4H, t), 3.71-3.73 (4H, t), 4.37 (2H, s), 4.80 (2H, s), 7.01-7.03 (1H, d), 7.13-7.15 (1H, d), 7.19-7.22 (1H, m), 7.26-7.28 (1H, m), 7.30-7.35 (6H, m), 7.64-7.67 (3H, m), 7.74-7.78 (1H, m), 14.65 (1H, s) m/z: 454 (M⁺¹)

### Example 5

### 1-Methanesulfonyl-2-phenyl-4-[1-pyridin-2-yl-methylidene]-piperidin-3-one

### Step A: Preparation of 4-[(Ethoxycarbonyl-phenyl-methyl)-amino]-butyric acid ethyl ester

The solution of 10 g of amino-phenyl-acetic acid ethyl ester (55.8 mmol) in dimethylformamide (30 ml) containing cesium carbonate (21.7 g, 67 mmol) was treated with ethyl bromobutyrate (9.2 ml, 61.38 mmol) at 80° C. for 12 hours. After completion of reaction, the mixture was cooled to room temperature, diluted with water (50 ml) and pH was adjusted to 7 using aqueous hydrochloric acid. The mixture was poured into water (100 ml) and extracted with ethylacetate (50 ml×3). The combined organic layers were washed with water (50 ml×2), brine (50 ml×2), dried over anhydrous sodium sulphate and evaporated under vacuo. The residue was purified by column chromatography on silica gel using the 5% ethyl acetate in hexane as the eluent to provide the titled compound (11.4 g) as yellow liquid.

¹HNMR (DMSOd₆): δ 1.10-1.17 (6H, t), 1.64-1.68 (2H, t), 2.29-2.32 (2H, t), 2.38-2.46 (2H, t), 4.00-4.09 (4H, q), 4.10-4.12 (1H, m), 4.32 (1H, s), 7.26-7.40 (5H, m) m/z: 295 (M⁺¹)

### Step B: Preparation of 3-Oxo-2-phenyl-piperidine-4-carboxylic acid ethyl ester

The solution of 11 g (37.5 mmol) of the product of example 5, Step A in tetrahydrofuran was cooled to −20° C. and lithium bis(trimethylsilyl)amide (71 ml, 75 mmol; 1.06M, LHMDS) was added dropwise and stirred for 6 h. Then the reaction was quenched with ammonium chloride solution and the mixture was poured into water (100 ml) and extracted with ethylacetate (50 ml×3). The combined organic layers were washed with water (50 ml×2), brine (50 ml×2), dried over anhydrous sodium sulphate and evaporated under vacuo. The residue was purified by column chromatography on silica gel using the 15% ethyl acetate in hexane as the eluent to provide the titled compound (7.4 g) as brown liquid.

¹HNMR (DMSOd₆): δ 1.15-1.18 (3H, t), 1.81-1.96 (2H, m), 2.26-2.32 (2H, t), 2.82-2.88 (1H, m), 3.44-3.50 (1H, m), 4.16-4.21 (2H, q), 7.25-7.27 (2H, m), 7.36-7.44 (3H, m) m/z: 248 (M⁺¹)

### Step C: Preparation of 2-Phenyl-4-[1-pyridin-2-yl-methylidene]-piperidin-3-one

(i) The solution of 7 g (28.3 mmol) of the product of example 5, Step B in ethanol:HCl (3:7) mixture (30 ml) was refluxed for 12 h. Then the mixture was cooled to room temperature, diluted with water (20 ml) and pH was adjusted to 7 using aqueous sodium hydroxide. The mixture was poured into water (100 ml) and extracted with ethylacetate (50 ml×3). The combined organic layers were washed with water (50 ml×2), brine (50 ml×2), dried over anhydrous sodium sulphate and evaporated under vacuo to obtained the 2-phenyl-piperidin-3-one (1.48 g) as brown liquid.

¹HNMR (DMSOd₆): δ 1.82-1.93 (2H, m), 2.16-2.30 (2H, t), 2.45-2.46 (2H, t), 4.05 (1H, d), 7.35-7.59 (5H, m), 5.32 (1H, bs) m/z: 176 (M⁺¹)

(ii) The solution of 2-phenyl-piperidin-3-one (1.48 g, 8.45 mmol) in methanol (10 ml) containing aqueous sodium hydroxide (0.7 g, 17 mmol) was treated with pyridine-2-carboxaldehyde (0.9 g, 9.2 mmol) at room temperature for 12 h. The mixture was poured into water (20 ml) and extracted with ethylacetate (10 ml×3). The combined organic layers were washed with water (100 ml×2), brine (50 ml×2), dried over anhydrous sodium sulphate and evaporated under vacuo. The residue was crystallized with ethanol to obtained the titled product (0.89 g) as yellow liquid.

¹HNMR (DMSOd₆): δ 2.34-2.41 (2H, t), 4.46-(2H, t), 5.33 (1H, bs), 7.11-7.16 (2H, m), 7.27-7.29 (4H, m), 7.67-7.70 (1H, m), 7.79-7.87 (1H, m), 7.91-8.025 (1H, m), 8.37-8.39 (1H, d) m/z: 265 (M⁺¹)

### Step D: Preparation of 1-Methanesulfonyl-2-phenyl-4-[1-pyridin-2-yl-methylidene]-piperidin-3-one

The solution of 0.89 g (3.37 mmol) of the product of example 5, Step C in dichloromethane (10 ml) containing triethylamine (0.95 ml, 6.74 mmol) was cooled to 0° C., methane sulphonylchloride (0.77 g, 6.74 mmol) was dropwise added and stirred at room temperature for 4 h. The mixture was poured into water (20 ml) and extracted with ethylacetate (10 ml×2). The combined organic layers were washed with water (5 ml×2), brine (5 ml×2), dried over anhydrous sodium sulphate and evaporated under vacuo. The residue was crystallized with ethanol to obtained the titled compound (0.6 g) as brown liquid.

¹HNMR (DMSOd₆): δ 2.31-2.41 (2H, t), 3.29 (3H, s), 4.10-4.15 (1H, m), 5.31-5.32 (2H, t), 7.32-7.42 (1H, m), 7.43-7.44 (1H, d), 7.67-7.73 (2H, m), 7.88-7.94 (1H, m), 8.26-8.29 (1H, m), 8.29-8.37 (1H, d), 8.44-8.60 (1H, d), 8.68-8.80 (1H, m), 9.25-9.26 (1H, d) m/z: 343 (M⁺¹)

### Example 6

### 1-(2,4-Dihydroxy-benzenesulfonyl)-3-phenyl-5-[1-pyridin-2-yl-methylidene]-piperidin-4-one

### Step A: Preparation of 2-Phenyl-acrylic acid ethyl ester

The solution of 10 g of ethyl phenylacetate (60.97 mmol) in 1-methyl-2-pyrrolidinone (50 ml) containing potassium carbonate (10.9 g, 79.3 mmol) was treated with paraformaldehyde (2.37 g, 79.3 mmol) at 90° C. for 6 h. After completion of reaction, the mixture was cooled to room temperature, diluted with water (50 ml) and pH was adjusted to 7 using aqueous hydrochloric acid. The mixture was poured into water (100 ml) and extracted with ethylacetate (50 ml×3). The combined organic layers were washed with water (50 ml×2), dried over anhydrous sodium sulphate and evaporated under vacuo. The residue was purified by column chromatography on silica gel using the 10% ethyl acetate in hexane as the eluent to provide the titled compound (3.5 g) as yellow liquid.

¹HNMR (DMSOd₆): δ 1.34-1.37 (3H, t), 4.29-4.34 (2H, m), 5.9 (1H, d), 6.3 (1H, d), 7.36-7.39 (3H, m), 7.43-7.44 (2H, m). m/z: 177 (M⁺¹)

### Step B: Preparation of 3-Benzylamino-2-phenyl-propionic acid ethyl ester

The solution of 3.5 g of the product (19.8 mmol) of example 6 Step A in toluene (10 ml) was refluxed with benzyl amine (2.76 g, 25.8 mmol) for 6 h. The mixture was poured into water (50 ml) and extracted with ethylacetate (10 ml×2). The combined organic layers were washed with water (5 ml×2), dried over anhydrous sodium sulphate and evaporated under vacuo. The residue was crystallized with ethanol to provide the titled compound (4.2 g) as colourless liquid.

¹HNMR (DMSOd₆): δ 1.12-1.15 (3H, t), 2.2 (1H, bs), 2.65-2.73 (1H, m), 3.06-3.12 (1H, t), 3.69 (2H, d), 3.77-3.81 (1H, m), 4.05-4.08 (2H, m), 7.26-7.34 (10H, m). m/z: 284 (M⁺¹)

### Step C: Preparation of 3-[Benzyl-(2-ethoxycarbonyl-ethyl)-amino]-2-phenyl-propionic acid ethy ester

The solution of 4.2 g of the product (14.8 mmol) of example 6 Step B was refluxed with ethyl acrylate (2 ml, 19.3 mmol) in the presence of acetic acid (0.15 ml, 2.9 mmol) for 12 h. After completion of reaction, the mixture was poured into water (50 ml) and extracted with ethylacetate (10 ml×2). The combined organic layers were washed with water (5 ml×2), dried over anhydrous sodium sulphate and evaporated under vacuo. The residue was purified by column chromatography on silica gel using the 6% ethyl acetate in hexane as the eluent to provide the titled compound (2.4 g) as yellow liquid.

¹HNMR (DMSOd₆): δ 1.09-1.15 (6H, m), 2.36-2.41 (1H, m), 2.43 (1H, d), 2.55-2.56 (1H, m), 2.58-2.60 (1H, t), 2.62-2.70 (1H, m), 3.11-3.16 (1H, m), 3.70-3.73 (1H, d), 3.34-3.52 (1H, d), 3.86-3.90 (1H, m), 3.95-3.99 (4H, t), 7.18-7.22 (4H, m), 7.24-7.29 (6H, m). m/z: 384 (M⁺¹)

### Step D: Preparation of 3-Phenyl-piperidin-4-one

(i) The solution of 2.4 g of the product (6.26 mmol) of example 6 Step C in methanol (20 ml) containing 0.22 g of Pd/C (10%) was stirred under hydrogen atmosphere (200 Psi) at room temperature for 10 h. Then the mixture was filtered over celite, dried over anhydrous sodium sulphate and evaporated under vacuo. The residue was crystallized with ethanol to obtained the compound 3-(2-Ethoxycarbonyl-ethylamino)-2-phenyl-propionic acid ethyl ester (1.65 g) as yellow liquid.

¹HNMR (DMSOd₆): δ 1.11-1.17 (6H, t), 1.80 (1H, bs), 2.36-2.41 (2H, t), 2.71-2.77 (2H, m), 3.07-3.12 (1H, m), 3.56-3.58 (1H, d), 3.71-3.75 (1H, m), 3.99-4.09 (4H, q), 7.24-7.34 (5H, m) m/z: 294 (M⁺¹)

(ii) To the 3-(2-Ethoxycarbonyl-ethylamino)-2-phenyl-propionic acid ethyl ester in tetrahydrofuran (10 ml) was cooled to 0° C. and Lithium bis(trimethylsilyl) amide (1.88 g, 11.26 mmol) was dropwise added. The reaction was stirred at 0° C. to 10° C. for 3 h. After completion of reaction, the pH was adjusted to 7 using aqueous hydrochloric acid. The mixture was poured into water (50 ml) and extracted with ethylacetate (10 ml×3). The combined organic layers were washed with water (10 ml×2), dried over anhydrous sodium sulphate and evaporated under vacuo. The residue was recrystallised with ethanol to obtained the compound 4-Oxo-5-phenyl-piperidine-3-carboxylic acid ethyl ester (1.1 g) as orange liquid. ¹HNMR (DMSOd₆): δ 1.11-1.17 (3H, t), 1.80 (1H, bs), 2.36-2.41 (2H, t), 3.07-3.12 (1H, m), 3.56-3.58 (1H, d), 3.71-3.75 (1H, m), 3.99-4.09 (2H, q), 4.21-4.23 (1H, t), 7.24-7.34 (5H, m); m/z: 248 (M^{+l}). The compound thus obtained was hydrolysed & decarboxylated by refluxing in mixture (10 ml) of concentrated hydrochloric acid:water (1:1) for 4 h. The pH of reaction mixture was neutralized using aqueous sodium bicarbonate and poured into water (50 ml) and extracted with ethylacetate (10 ml×3). The combined organic layers were washed with water (10 ml×2), dried over anhydrous sodium sulphate and evaporated under vacuo. The residue was recrystallized with ethanol to obtained the titled compound (0.7 g) as red liquid.

¹HNMR (DMSOd₆): δ 2.23-2.27 (1H, d), 2.52-2.55 (1H, t), 2.84-2.97 (2H, m), 3.25-3.28 (2H, t), 3.68-3.72 (1H, q), 7.12-7.14 (2H, d), 7.18-7.24 (1H, t), 7.27-7.33 (2H, m) m/z: 176 (M⁺¹)

### Step E: Preparation of 3-Phenyl-5-[1-pyridin-2-yl-methylidene]-piperidin-4-one

The solution of 0.7 g of the product (4 mmol) of example 6 Step D (ii) in methanol (5 ml) containing aqueous sodium hydroxide (0.32 g, 8 mmol) was treated with pyridine-2-carboxaldehyde (0.42 g, 4 mmol) at room temperature for 4 h. Then the mixture was poured into water (20 ml) and extracted with ethylacetate (10 ml×2). The combined organic layers were washed with water (5 ml×2), dried over anhydrous sodium sulphate and evaporated under vacuo. The residue was purified by column chromatography on silica gel using the 2% methanol in dichloromethane as the eluent to obtained the titled compound (0.4 g) as yellow solid.

¹HNMR (DMSOd₆): δ 2.15-2.19 (2H, t), 2.69 (2H, s), 3.43-3.47 (1H, t), 7.12-7.22 (6H, m), 7.32-7.37 (1H, d), 7.58 (1H, bs), 7.60-7.64 (2H, m), 8.42-8.43 (1H, d) m/z: 265 (M⁺¹)

### Step F: 1-(2,4-Dihydroxy-benzenesulfonyl)-3-phenyl-5-[1-pyridin-2-ymethylidene]-piperidin-4-one

The solution of 0.4 g of the product (1.5 mmol) of example 6 Step E in tetrahydrofuran (5 ml) was cooled to 0° C., sodium hydride (0.1 g, 4.5 mmol) was added and the mixture was stirred for 15 min, followed by addition of 2,4-dihydroxybenzene sulphonyl chloride. Then the reaction mixture was refluxed for 5 h. After completion of reaction, the mixture was poured into water (20 ml) and extracted with ethylacetate (10 ml×2). The combined organic layers were washed with water (5 ml×2), dried over anhydrous sodium sulphate and evaporated under vacuo. The residue was purified by column chromatography on silica gel using the 50% ethyl acetate in hexane as the eluent to obtained the titled compound (0.5 g) as yellow liquid.

¹ HNMR (DMSOd₆): δ 3.66-3.69 (2H, d), 3.80 (3H, s), 6.34-6.36 (1H, d), 6.43 (1H, s), 7.14 (2H, d), 7.24 (5H, s), 7.52-7.54 (1H, d), 7.75 (1H, s), 7.88 (1H, s), 8.50 (1H, s), 10.55 (1H, s), 11.09 (1H, s) m/z: 437 (M⁺¹)

The following representative compounds of the present invention were prepared following the synthetic routes as described above:

TABLE 1

| | | | Mass, |
|---|---|---|---|
| | | ¹HNMR, (400 MHz, | m/z |
| Compd. NO | Nomenclature | DMSOd₆ or CDCl₃) δ | (M⁺¹) |
| 1 | 1-Benzyl-3,3-dimethyl-5-[1- | 1.17 (6H, s), 2.5m (2H, s), 3.69 (2H, | 307 |
| | pyridin-2-yl-methylidene]- | s), 4.191-4.196 (2H, d), | |
| | piperidin-4-one | 7.18-7.21 (1H, m), 7.25-7.26 (1H, m), | |
| | | 7.27-7.29 (1H, m), 7.34-7.35 (2H, m), | |
| | | 7.39-7.41 (4H, m), 7.68-7.72 (1H, | |
| | | m) | |
| 2 | 3,3-Dimethyl-4-oxo-5-[1- | 1.20 (6H, s), 3.64 (2H, s), 5.20 (2H, | 351 |
| | pyridin-2-yl-methylidene]- | s), 5.27 (2H, s), 7.23-7.28 (1H, m), | |
| | piperidine-1-carboxylic acid | 7.30-7.31 (1H, m), 7.36-7.38 (4H, | |
| | benzyl ester | m), 7.45-7.54 (2H, m), | |
| | | 7.71-7.72 (1H, m), 8.73 (1H, s) | |
| 3 | 3,3-Dimethyl-4-oxo-5-[1- | 1.21 (6H, s), 1.30 (3H, t), 3.61 (2H, | 289 |
| | pyridin-2-yl-methylidene]- | s), 4.21 (2H, m), 5.18 (2H, d), | |
| | piperidine-1-carboxylic acid | 7.24-7.28 (1H, m), 7.44-7.48 (2H, m), | |
| | ethyl ester | 7.73 (1H, m), 8.74-8.75 (1H, d) | |
| 4 | 3,3-Dimethyl-4-oxo-5-[1- | 1.31 (6H, s), 3.72-3.81 (2H, d), | 337 |
| | pyridin-2-yl-methylidene]- | 5.29 (1H, s), 5.40 (1H, s), 7.12-7.15 (2H, | |
| | piperidine-1-carboxylic acid | m), 7.17-7.24 (2H, m), | |
| | phenyl ester | 7.36-7.40 (2H, m), 7.45-7.48 (1H, m), | |
| | | 7.50-7.54 (1H, d), 7.70-7.75 (1H, m), | |
| | | 8.73 (1H, s) | |
| 5 | 1-Acetyl-3,3-dimethyl-5-[1- | 1.06 (6H, s), 2.10 (3H, s), | 259 |
| | pyridin-2-yl-methylidene]- | 4.54-4.56 (2H, d), 5.40-5.43 (2H, m), | |
| | piperidin-4-one | 7.22-7.25 (1H, m), 7.45-7.47 (1H, d), | |
| | | 7.76-7.81 (2H, m), 8.46-8.47 (1H, | |
| | | d) | |
| 6 | 1-Benzyl-3-methyl-5-[1- | 1.11-113 (3H, d), 2.46-2.53 (1H, | 293 |
| | pyridin-2-yl-methylidene]- | m), 3.16-3.19 (2H, d), 3.6 (2H, s), | |
| | piperidin-4-one | 3.75-3.76 (2H, d), 7.14-7.16 (1H, | |
| | | m), 7.28-7.32 (2H, m), | |
| | | 7.33-7.41 (4H, m), 7.44-7.46 (1H, m), | |
| | | 7.60-7.63 (1H, m), 8.53-8.54 (1H, dd) | |
| 7 | 1-Benzyl-3,3-dimethyl-5-[1- | 1.07 (6H, s), 3.57-3.65 (12H, m), | 419 |
| | [4-(morpholine-4-carbonyl)- | 3.75 (2H, s), 7.23-7.26 (1H, m), | |
| | phenyl]-methylidene]- | 7.30-7.35 (4H, m), 7.36-7.38 (1H, | |
| | piperidin-4-one | m), 7.44-7.50 (4H, m) | |
| 8 | 1-Benzyl-3,3-dimethyl-5-[1- | 1.06 (6H, s), 2.42-2.45 (2H, m), | 352 |
| | (4-methylsulfanyl-phenyl)- | 2.56 (3H, s), 3.65 (2H, s), 3.73 (2H, | |
| | methylidene]-piperidin-4- | s), 7.27-7.28 (2H, m), | |
| | one | 7.31-7.35 (3H, m), 7.36-7.39 (3H, m), | |
| | | 7.43-7.46 (1H, m), 7.81-7.83 (1H, m) | |
| 9 | 1-Benzyl-3,3-dimethyl-5-[1- | 1.08 (6H, s), 2.51 (2H, s), 3.65 (2H, | 351 |
| | (4-nitro-phenyl)- | s), 3.75 (2H, s), 7.33-7.36 (5H, m), | |
| | methylidene]-piperidin-4- | 7.44 (1H, s), 7.68-7.70 (2H, d), | |
| | one | 8.24-8.26 (2H, d) | |
| 10 | 1-Benzyl-3,3-dimethyl-5-[1- | 1.069 (6H, s), 2.51 (2H, s), 3.64 (2H, | 306 |
| | phenyl-methylidene]- | s), 3.73 (2H, s), 7.22-7.25 (1H, m), | |
| | piperidin-4-one | 7.30-7.37 (6H, m), 7.40-7.45 (4H, | |
| | | m) | |
| 11 | 1-Benzyl-3,3-dimethyl-5-[1- | 1.08 (6H, s), 2.33 (3H, s), 2.56 (2H, | 326 |
| | (3-methyl-thiophen-2-yl)- | s), 3.63 (2H, s), 3.70 (2H, s), | |
| | methylidene]-piperidin-4- | 7.09-7.11 (1H, d), 7.25-7.29 (1H, m), | |
| | one | 7.33-7.40 (4H, m), 7.66 (1H, s), | |
| | | 7.81-7.83 (1H, d) | |
| 12 | 1-Benzyl-5-[1-(4- | 1.0 (6H, s), 2.92 (5H, s), | 392 |
| | methanesulfonyl-piperazin- | 3.06-3.09 (2H, t), 3.13-3.16 (1H, t), 3.22 (3H, | |
| | 1-yl)-methylidene]-3,3- | s), 3.44-3.47 (2H, m), 3.65 (2H, s), | |
| | dimethyl-piperidin-4-one | 4.09-4.14 (2H, m), 7.21-7.23 (1H, | |
| | | m), 7.29-7.30 (3H, m), | |
| | | 7.64-7.73 (2H, m) | |
| 13 | 2-(4-Methoxy-benzyl)-3,3- | 0.94-1.05 (3H, t), 1.35 (6H, s), | 409 |
| | dimethyl-4-oxo-5-[1-pyridin- | 2.50-2.57 (1H, m), 2.97-3.09 (1H, m), | |
| | 2-yl-methylidene]- | 3.80 (3H, s), 3.83-3.89 (2H, q), | |
| | piperidine-1-carboxylic acid | 3.92-4.06 (1H, m), 4.62-4.81 (1H, m), | |
| | ethyl ester | 5.52-5.68 (1H, m), 6.79-6.82 (2H, | |
| | | m), 7.07-7.09 (1H, d), | |
| | | 7.10-7.13 (1H, d), 7.22-7.28 (1H, m), | |
| | | 7.47-7.49 (1H, d), 7.52-7.53 (1H, m) | |
| | | 7.57-7.58 (1H, m), 7.71-7.71 (1H, m) | |
| 14 | 2-(4-Methoxy-benzyl)-3,3- | 1.35 (3H, s), 1.37 (3H, s), | 457 |
| | dimethyl-4-oxo-5-[1-pyridin- | 2.56-2.66 (1H, m), 3.07-3.17 (1H, m), | |
| | 2-yl-methylidene]- | 3.81 (3H, s), 4.59-4.63 (1H, m), | |
| | piperidine-1-carboxylic acid | 4.86-4.93 (1H, m), 5.71-5.77 (1H, d), | |
| | phenyl ester | 6.64-6.66 (1H, d), 6.84-6.89 (2H, | |
| | | m), 7.13-7.16 (3H, m), | |
| | | 7.17-7.18 (1H, m), 7.19-7.24 (1H, m), | |
| | | 7.25-7.33 (2H, m), 7.49-7.52 (1H, m), | |
| | | 7.60-7.63 (1H, m), 7.72-7.76 (1H, | |
| | | m), 8.73-8.77 (1H, dd) | |
| 15 | 2-(4-Methoxy-benzyl)-3,3- | 0.826 (3H, s), 0.84 (3H, s), | 437 |
| | dimethyl-4-oxo-5-[1-pyridin- | 1.21-1.27 (3H, d), 1.28-1.30 (3H, d), | |
| | 2-yl-methylidene]- | 1.59-1.83 (1H, m), 2.25-2.29 (1H, m), | |
| | piperidine-1-carboxylic acid | 2.45-2.59 (1H, m), 2.89-2.90 (1H, | |
| | isobutyl ester | m), 2.92-2.97 (1H, m), | |
| | | 3.0-3.09 (1H, m), 3.71-3.73 (2H, d), | |
| | | 3.78 (3H, s), 6.77-6.80 (2H, m), | |
| | | 7.01-7.06 (1H, m), 7.08-7.09 (1H, m), | |
| | | 7.11-7.13 (1H, m), 7.21-7.30 (1H, | |
| | | m), 7.46-7.50 (1H, m), | |
| | | 7.71-7.76 (1H, m), 8.71-8.79 (1H, dd) | |
| 16 | 1-(2,2-Dimethyl-propionyl)- | 1.03 (3H, s), 1.05 (3H, s), 1.21 (3H, | 421 |
| | 2-(4-methoxy-benzyl)-3,3- | s), 1.23 (3H, s), 1.32 (3H, s), | |
| | dimethyl-5-[1-pyridin-2-yl- | 2.51-2.58 (2H, m), 3.14-3.19 (1H, dd), | |
| | methylidene]-piperidin-4- | 3.79 (3H, s), 4.29-4.34 (1H, dd), | |
| | one | 5.33-5.37 (1H, dd), 6.29-6.24 (1H, | |
| | | d), 6.77-6.82 (2H, m), | |
| | | 7.15-7.18 (1H, d), 7.23-7.30 (1H, m), | |
| | | 7.47-7.49 (1H, m), 7.50-7.51 (1H, m), | |
| | | 7.74-7.78 (1H, m), 8.68-8.69 (1H, | |
| | | dd) | |
| 17 | 2-(4-Methoxy-benzyl)-3,3- | 1.27 (6H, s), 1.91 (6H, s), | 484 |
| | dimethyl-4-oxo-5-[1-pyridin- | 2.84-2.86 (1H, m), 2.88-2.92 (1H, m), | |
| | 2-yl-methylidene]- | 3.63-3.71 (1H, d), 3.78 (3H, s), | |
| | piperidine-1-carboxylic acid | 3.83-3.86 (1H, d), 4.29-4.33 (1H, t), 6.02 (1H, | |
| | (2,6-dimethyl-phenyl)- | bs), 6.82-6.84 (2H, d), | |
| | amde | 6.98-7.03 (2H, m), 7.04-7.05 (1H, m), | |
| | | 7.06-7.15 (3H, m), 7.37-7.39 (1H, d), | |
| | | 7.62-7.66 (1H, m), 7.78 (1H, s), | |
| | | 8.51-8.52 (1H, dd) | |
| 18 | 1-Benzyl-3,3-dimethyl-5-[1- | 2.50 (6H, s), 3.02 (2H, s), 3.68 (2H, | 357 |
| | quinolin-2-yl-methylidene]- | s), 4.47 (2H, s), 7.11-7.35 (6H, m), | |
| | piperidin-4-one | 7.54-7.72 (3H, m), 7.91-8.03 (2H, | |
| | | m), 8.21 (1H, s) | |
| 19 | 1-Benzyl-3,3-dimethyl-5-[1- | 1.06 (6H, s), 2.51 (2H, s), 3.54 (2H, | 295 |
| | (1H-pyrrol-2-yl)- | s), 3.69 (2H, s), 6.26-6.29 (2H, dd), | |
| | methylidene]-piperidin-4- | 7.10 (1H, s), 7.27-7.28 (1H, m), | |
| | one | 7.35-7.39 (5H, m), 11.53 (1H, bs) | |
| 20 | 1-Benzyl-3,3-dimethyl-5-[1- | 1.12 (6H, s), 2.60 (2H, s), | 392 |
| | (6-morpholin-4-yl-pyridin-2- | 3.27-3.29 (4H, s), 3.64-3.66 (6H, m), 4.02 (2H, | |
| | yl)-methylidene]-piperidin-4- | s), 6.80-6.82 (1H, d), | |
| | one | 6.95-6.97 (1H, d), 7.13 (1H, s), 7.25-7.27 (1H, | |
| | | m), 7.31-7.37 (4H, m), | |
| | | 7.56-7.60 (1H, m) | |
| 21 | 1-Benzyl-3,3-dimethyl-5-[1- | 0.85 (6H, s), 3.04 (2H, s), 3.73 (2H, | 358 |
| | quinoxalin-2-yl- | s), 4.49 (2H, s), 7.23-7.38 (4H, m), | |
| | methylidene]-piperidin-4- | 7.46-7.49 (1H, s), 7.73-7.80 (1H, | |
| | one | m), 7.781-7.82 (2H, m), | |
| | | 7.89-7.90 (1H, m), 7.99-8.05 (1H, m), | |
| | | 8.79-8.84 (1H, m) | |
| 22 | 1-Benzyl-3,3-dimethyl-5-[1- | 1.046 (6H, s), 2.36 (2H, s), 3.56 (2H, | 312 |
| | thiophen-2-yl-methylidene]- | s), 3.72 (2H, s), 6.99-7.03 (1H, m), | |
| | piperidin-4-one | 7.19-7.24 (1H, m), 7.31-7.378 (4H, | |
| | | m), 7.61-7.64 (1H, m), 7.78 (1H, s), | |
| | | 7.90-7.94 (1H, m) | |
| 23 | 1-Benzyl-3,3-dimethyl-5-[1- | 1.12 (6H, m), 1.50-1.59 (6H, m), | 390 |
| | (3,4,5,6-tetrahydro-2H- | 2.51-2.57 (6H, m), 3.66 (2H, s), | |
| | [1,2′]bipyridinyl-6′-yl)- | 4.07 (2H, s), 6.81-6.86 (2H, m), | |
| | methylidene]-piperidin-4- | 7.12 (1H, s), 7.27-7.29 (1H, m), | |
| | one | 7.34-7.38 (4H, m), 7.53 (1H, s), | |
| 24 | 1-Benzyl-5-[1-(3-hydroxy- | 0.89 (6H, s), 3.05 (2H, s), 3.29 (2H, | 374 |
| | quinoxalin-2-yl)- | s), 4.47 (2H, s), 7.24-7.30 (3H, m), | |
| | methylidene]-3,3-dimethyl- | 7.32-7.36 (2H, m), 7.39-7.42 (2H, | |
| | piperidin-4-one | m), 7.44-7.50 (1H, m), | |
| | | 7.52-7.58 (1H, d), 7.58-7.65 (1H, dd), | |
| | | 12.26 (1H, bs) | |
| 25 | 1-Benzyl-5,5-dimethyl-2- | 1.21 (6H, s), 2.4-2.51 (1H, d), | 383 |
| | phenyl-3-[1-pyridin-2-yl- | 2.67-2.70 (1H, d), 3.45-3.56 (1H, m), | |
| | methylidene]-piperidin-4- | 3.58-3.69 (1H, m), 6.08 (1H, s), | |
| | one | 7.17-7.14 (1H, m), 7.16-7.19 (2H, | |
| | | m), 7.20-7.22 (2H, m), | |
| | | 7.23-7.24 (3H, m), 7.24-7.34 (5H, m), | |
| | | 7.55-7.59 (1H, dd), 8.63-8.64 (1H, d) | |
| 26 | 1-Benzyl-5,5-dimethyl-2- | 2.46 (6H, s), 3.43-3.45 (1H, q), | 434 |
| | phenyl-3-[1-quinoxalin-2-yl- | 3.45-3.53 (1H, m), 3.55-3.62 (1H, | |
| | methylidene]-piperidin-4- | m), 3.63-3.65 (1H, q), | |
| | one | 4.00-4.02 (1H, d), 7.38-7.43 (2H, d), | |
| | | 7.51-7.52 (1H, m), 7.53-7.54 (1H, m), | |
| | | 7.62-7.64 (1H, m), 7.82-7.90 (2H, | |
| | | d), 7.91-7.96 (4H, m), | |
| | | 8.12-8.15 (3H, m), 9.37 (2H, s) | |
| 27 | 1-Benzyl-5,5-dimethyl-2- | 1.05 (3H, s), 1.13 (3H, s), | 371 |
| | phenyl-3-[1-(1H-pyrrol-2-yl)- | 3.24-3.28 (2H, d), 3.31 (1H, s), 3.86-3.89 (1H, | |
| | methylidene]-piperidin-4- | d), 5.24 (1H, s), 7.15-7.17 (2H, d), | |
| | one | 7.19-7.21 (1H, bs), 7.22-7.24 (3H, | |
| | | d), 7.25-7.27 (3H, m), | |
| | | 7.28-7.31 (2H, s), 7.32-7.34 (2H, d), | |
| | | 7.40-7.44 (2H, m) | |
| 28 | 1-Benzyl-5,5-dimethyl-3-[1- | 1.06 (3H, s), 1.22 (3H, s), | 469 |
| | (6-morpholin-4-yl-pyridin-2- | 2.36-2.39 (1H, d), 2.56-2.59 (2H, d), | |
| | yl)-methylidene]-2,3,5,6- | 3.04-3.09 (2H, m), 3.19-3.25 (2H, m), | |
| | tetrahydro-1H-[2,2′]bipyridinyl- | 3.48-3.51 (1H, d), 3.57-3.59 (4H, | |
| | 4-one | t), 3.88-3.92 (1H, d), 6.49 (1H, s), | |
| | | 6.72-6.75 (1H, d), 6.83-6.85 (1H, | |
| | | d), 7.00 (1H, s), 7.12-7.14 (1H, d), | |
| | | 7.23-7.27 (2H, m), 7.28-7.31 (1H, | |
| | | m), 7.36-7.38 (2H, d), | |
| | | 7.50-7.54 (1H, m), 7.80-7.85 (1H, dd), | |
| | | 8.51-8.52 (1H, d) | |
| 29 | 1-Benzyl-5,5-dimethyl-3-[1- | 1.10 (3H, s), 1.18 (3H, s), | 384 |
| | pyridin-2-yl-methylidene]- | 2.29-2.32 (1H, d), 2.51 (1H, d), 2.79-2.82 (1H, | |
| | 2,3,5,6-tetrahydro-1H- | d), 3.21-3.25 (1H, d), | |
| | [2,2′]bipyridinyl-4-one | 3.90-3.94 (1H, d), 6.40 (1H, s), 7.12 (1H, s), | |
| | | 7.21-7.26 (2H, m), 7.27-7.29 (2H, | |
| | | m), 7.31-7.35 (2H, m), | |
| | | 7.42-7.44 (1H, d), 7.49-7.51 (1H, d), | |
| | | 7.75-7.83 (2H, m), 8.49-8.52 (2H, m) | |
| 30 | 1-Benzyl-5,5-dimethyl-3-[1- | 0.89 (3H, s), 1.29 (3H, s), | 428 |
| | (4-methylsulfanyl-phenyl)- | 2.33-2.38 (1H, dd), 2.56 (3H, s), | |
| | methylidene]-2-phenyl- | 2.83-2.95 (2H, m), 3.59-3.67 (2H, m), | |
| | piperidin-4-one | 7.23-7.24 (2H, m), 7.28-7.33 (6H, m), | |
| | | 7.39-7.46 (3H, m), 7.52-7.54 (3H, | |
| | | d), 7.81-7.84 (1H, d), | |
| 31 | 1-Benzyl-5,5-dimethyl-3-[1- | 1.02 (3H, s), 1.23 (3H, s), | 468 |
| | (6-morpholin-4-yl-pyridin-2- | 2.54-2.57 (1H, d), 2.67-2.70 (1H, d), | |
| | yl)-methylidene]-2-phenyl- | 2.96-301 (2H, m), 3.07-3.12 (2H, m), | |
| | piperidin-4-one | 3.13-3.34 (1H, d), 3.37-3.49 (4H, | |
| | | t), 3.62-3.65 (1H, d), | |
| | | 3.85-3.89 (1H, d), 6.38 (1H, s), 6.73-6.75 (1H, | |
| | | d), 6.86-6.88 (1H, d), | |
| | | 7.082-7.10 (1H, d), 7.14 (1H, s), 7.21-7.26 (2H, | |
| | | m), 7.28-7.34 (6H, m), | |
| | | 7.51-7.55 (1H, m) | |
| 32 | 1-Benzyl-5,5-dimethyl-3-[1- | 1.30 (3H, s), 1.49 (3H, s), | 389 |
| | pyridin-2-yl-methylidene]-2- | 2.69-2.72 (1H, d), 3.76-3.77 (1H, m), | |
| | thiophen-2-yl-piperidin-4- | 4.27-4.29 (1H, d), 4.37-4.378 (1H, d), | |
| | one | 4.55-4.57 (1H, d), 6.74 (1H, s), | |
| | | 6.94-6.95 (1H, m), 6.96-6.97 (1H, m), | |
| | | 7.05 (1H, s), 7.23-7.27 (1H, m), | |
| | | 7.30-7.32 (2H, m), 7.34-7.37 (2H, | |
| | | m), 7.41-7.42 (1H, d), | |
| | | 7.54-7.56 (1H, d), 7.78-7.83 (1H, dd), | |
| | | 8.61-8.62 (1H, d) | |
| 33 | 1-Benzyl-5,5-dimethyl-3-[1- | 1.03 (3H, s), 1.15 (3H, s), | 474 |
| | (6-morpholin-4-yl-pyridin-2- | 2.51-2.53 (2H, d), 2.75-2.78 (1H, d), | |
| | yl)-methylidene]-2- | 2.94-2.98 (2H, m), 2.99-3.00 (2H, m), | |
| | thiophen-2-yl-piperidin-4- | 3.50-3.52 (4H, t), 3.74-3.78 (1H, | |
| | one | d), 3.90-3.93 (1H, d), | |
| | | 6.71-6.72 (1H, d), 6.76-6.78 (1H, m), | |
| | | 6.91-6.93 (1H, d), 6.98-7.00 (1H, m), | |
| | | 7.07 (1H, s), 7.24-7.28 (1H, m), | |
| | | 7.31-7.34 (2H, m), 7.37-7.41 (2H, | |
| | | m), 7.43-7.44 (1H, dd), | |
| | | 7.53-7.57 (1H, m), | |
| 34 | 1-Benzyl-5,5-dimethyl-3-[1- | 1.37 (3H, s), 1.44 (3H, s), | 467 |
| | (3,4,5,6-tetrahydro-2H- | 2.07-2.37 (3H, m), 2.62-2.65 (1H, m), | |
| | [1,2′]bipyridinyl-6′-yl)- | 3.29-3.33 (1H, m), 3.37-3.43 (1H, m), | |
| | methylidene]-2,3,5,6- | 3.50-3.54 (1H, d), 3.81-3.84 (1H, | |
| | tetrahydro-1H- | d), 3.93-3.96 (1H, dd), | |
| | [2,2′]bipyridinyl-4-one | 4.02-4.05 (1H, d), 4.27-4.29 (1H, d), | |
| | | 4.35-4.36 (1H, d), 4.44-4.47 (1H, d), | |
| | | 4.54-4.56 (1H, d), 4.65-4.67 (1H, dd), | |
| | | 6.52-6.54 (1H, d), 6.66-6.67 (1H, | |
| | | d), 7.02-7.18 (2H, m), | |
| | | 7.23-7.31 (4H, m), 7.33-7.54 (3H, m), | |
| | | 7.54-7.65 (1H, m), 8.58-8.59 (1H, m) | |
| 35 | 3,3-Dimethyl-4-oxo-5-[1- | 1.14 (6H, s), 1.40-1.45 (4H, m), | 420 |
| | (3,4,5,6-tetrahydro-2H- | 1.53-1.59 (4H, m), 3.37-3.46 (2H, | |
| | [1,2′]bipyridinyl-6′-yl)- | m), 3.52-3.57 (2H, s), 3.65 (1H, s), | |
| | methylidene]-piperidine-1- | 3.81 (1H, s), 6.85-6.88 (1H, d), | |
| | carboxylic acid phenyl ester | 6.94-6.98 (1H, m), 7.13-7.14 (2H, d), | |
| | | 7.24-7.26 (1H, m), 7.27-7.30 (1H, | |
| | | m), 7.38-7.42 (2H, m), | |
| | | 7.56-7.60 (1H, m) | |
| 36 | 3,3-Dimethyl-5-[1-(6- | 1.15 (6H, s), 3.46-3.49 (6H, t), | 422 |
| | morpholin-4-yl-pyridin-2-yl)- | 3.64 (1H, s), 3.69 (2H, s), 3.80 (1H, s), | |
| | methylidene]-4-oxo- | 5.22 (1H, s), 5.46 (1H, s), | |
| | piperidine-1-carboxylic acid | 6.87-6.92 (1H, m), 7.05-7.09 (1H, m), | |
| | phenyl ester | 7.15-7.16 (2H, d), 7.24-7.27 (1H, m), | |
| | | 7.32 (1H, s), 7.40-7.44 (2H, m), | |
| | | 7.63-7.67 (1H, m) | |
| 37 | 2-[1-Benzyl-5,5-dimethyl-4- | 1.11 (6H, s), 2.59 (2H, s), 3.69 (2H, | 374 |
| | oxo-piperidin-3- | s), 4.17 (2H, s), 7.01-7.02 (1H, m), | |
| | ylidenemethyl]-3H- | 7.26-7.29 (1H, m), 7.35-7.39 (2H, | |
| | quinazolin-4-one | m), 7.40-7.42 (2H, m), | |
| | | 7.52-7.59 (2H, m), 7.82-7.86 (1H, m), | |
| | | 8.09-8.11 (1H, dd), 12.47 (1H, bs), | |
| 38 | 1-Benzyl-3,3-dimethyl-5-[1- | 1.20 (6H, s), 2.59 (2H, s), 3.71 (4H, | 307 |
| | pyridin-3-yl-methylidene]- | s), 7.28 (1H, s), 7.31-7.37 (4H, m), | |
| | piperidin-4-one | 7.49 (1H, s), 7.65-7.67 (2H, m), | |
| | | 8.57-8.58 (1H, dd), 8.59-8.63 (1H, | |
| | | d) | |
| 39 | 5′-[1-Benzyl-5,5-dimethyl-4- | 1.16 (6H, s), 1.77-1.82 (2H, m), | 434 |
| | oxo-piperidin-3- | 2.04-2.12 (2H, m), 2.51 (2H, s), | |
| | ylidenemethyl]-3,4,5,6- | 2.53-2.68 (1H, m), 3.07-3.14 (2H, | |
| | tetrahydro-2H- | m), 3.68 (2H, s), 3.77 (2H, s), | |
| | [1,2′]bipyridinyl-4-carboxylic | 4.28-4.30 (1H, t), 4.31-4.33 (1H, m), | |
| | acid | 6.65-6.67 (1H, d), 7.26 (1H, s), | |
| | | 7.28-7.30 (1H, m), 7.35-7.39 (3H, m), | |
| | | 7.43 (1H, s), 7.48-7.51 (1H, dd), | |
| | | 8.26-8.268 (1H, d), 11.3 (1H, bs) | |
| 40 | 1-Benzyl-2-(4- | 1.09 (6H, s), 2.38-2.41 (1H, s), | 426 |
| | dimethylamino-phenyl)-5,5- | 2.51-2.59 (1H, s), 2.84 (6H, s), | |
| | dimethyl-3-[1-pyridin-2-yl- | 3.40-3.42 (1H, d), 3.61-3.64 (1H, d), | |
| | methylidene]-piperidin-4- | 4.13-4.15 (1H, dd), 6.07 (1H, s), | |
| | one | 6.64-6.66 (2H, d), 7.01 (1H, s), | |
| | | 7.05-7.07 (2H, d), 7.24-7.31 (4H, m), | |
| | | 7.45-7.46 (1H, d), 7.68-7.77 (2H, m), | |
| | | 8.61-8.62 (1H, d) | |
| 41 | 1-Benzyl-5-[1-[6-(3,5- | 1.17 (6H, s), 1.27-1.28 (3H, d), | 420 |
| | dimethyl-morpholin-4-yl)- | 1.30-1.31 (3H, d), 2.51 (2H, s), | |
| | pyridin-3-yl]-methylidene]- | 2.57-2.62 (2H, m), 3.62 (2H, s), | |
| | 3,3-dimethyl-piperidin-4- | 3.69-3.73 (2H, m), 3.75-3.78 (2H, d), | |
| | one | 4.13-4.16 (2H, m), 6.6-6.69 (1H, | |
| | | d), 7.28-7.30 (1H, m), | |
| | | 7.33-7.35 (2H, m), 7.36-7.40 (2H, m), | |
| | | 7.42-7.44 (1H, m), 7.50-7.52 (1H, dd), | |
| | | 8.26-8.27 (1H, d) | |
| 42 | 1-Benzyl-5,5-dimethyl-2-(4- | 1.02 (3H, s), 1.23 (3H, s), 2.39 (3H, | 514 |
| | methylsulfanyl-phenyl)-3-[1- | s), 2.65-2.71 (2H, m), | |
| | (6-morpholin-4-yl-pyridin-2- | 2.97-3.01 (2H, m), 3.10-3.14 (2H, m), | |
| | yl)-meth-(E)-ylidene]- | 3.38-3.50 (4H, t), 3.60-3.64 (1H, d), | |
| | piperidin-4-one | 3.82-3.86 (1H, d), 6.33 (1H, s), | |
| | | 6.73-6.75 (1H, d), 6.86-6.88 (1H, d), | |
| | | 7.00-7.02 (1H, d), 7.12 (1H, s), | |
| | | 7.20-7.22 (2H, m), 7.25-7.26 (2H, m), | |
| | | 7.28-7.31 (4H, m), 7.51-7.55 (1H, | |
| | | m) | |
| 43 | 1-Benzyl-5,5-dimethyl-3-[1- | 1.26 (6H, s), 2.92-2.93 (2H, m), | 536 |
| | (6-morpholin-4-yl-pyridin-2- | 2.99-3.00 (2H, m), 3.42-3.44 (2H, | |
| | yl)-methylidene]-2-(4- | t), 3.54-3.62 (2H, m), | |
| | trifluoromethyl-phenyl)- | 3.70-3.73 (2H, m), 4.15-4.51 (2H, m), | |
| | piperidin-4-one | 6.39 (1H, s), 6.74-6.76 (1H, d), | |
| | | 6.91-6.93 (1H, d), 7.14-7.19 (1H, m), | |
| | | 7.24-7.37 (7H, m), 7.53-7.57 (1H, | |
| | | m), 7.70-7.72 (1H, m), | |
| | | 7.76-7.80 (1H, m) | |
| 44 | 1-Benzyl-5,5-dimethyl-3-[1- | 1.03 (6H, s), 2.50 (1H, m), | 451 |
| | pyridin-2-yl-methylidene]-2- | 2.59-2.62 (1H, d), 3.39-3.45 (1H, d), | |
| | (4-trifluoromethyl-phenyl)- | 3.63-3.67 (1H, d), 6.28 (1H, s), 7.02 (1H, | |
| | piperidin-4-one | s), 7.13-7.31 (6H, m), | |
| | | 7.47-7.58 (3H, m), 7.66-7.73 (2H, m), | |
| | | 7.73-7.80 (1H, m), 8.61-8.62 (1H, d), | |
| 45 | 1-Benzyl-2-(3,4-dichloro- | 1.09 (6H, s), 2.27-2.58 (2H, m), | 451 |
| | phenyl)-5,5-dimethyl-3-[1- | 3.27-3.41 (1H, d), 3.49-3.64 (1H, | |
| | pyridin-2-yl-methylidene]- | d), 6.17 (1H, s), 7.12 (1H, s), | |
| | piperidin-4-one | 7.16-7.23 (2H, m), 7.25-7.28 (4H, m), | |
| | | 7.34-7.41 (1H, m), 7.52-7.58 (3H, | |
| | | m), 7.78-7.82 (1H, m), | |
| | | 8.62-8.63 (1H, d) | |
| 46 | 1-Benzyl-5,5-dimethyl-2-(4- | 0.59 (3H, s), 1.26 (3H, s), | 429 |
| | methylsulfanyl-phenyl)-3-[1- | 2.45-2.51 (1H, d), 2.53 (3H, s), 2.68-2.74 (1H, | |
| | pyridin-2-yl-methylidene]- | d), 3.17-3.25 (1H, d), | |
| | piperidin-4-one | 3.71-3.84 (1H, d), 4.43 (1H, s), 6.31 (1H, s), | |
| | | 7.22-7.26 (2H, m), 7.30-7.34 (2H, | |
| | | m), 7.42-7.44 (2H, d), | |
| | | 7.53-7.54 (2H, m), 7.90-7.94 (1H, m), | |
| | | 8.09-8.11 (1H, d), 8.60-8.64 (1H, m), | |
| | | 8.70 (1H, s), 9.21-9.22 (1H, d) | |
| 47 | 1-(4-Methoxy-benzyl)-5,5- | 1.06 (6H, s), 2.43-2.46 (1H, d), | 413 |
| | dimethyl-2-phenyl-3-[1- | 2.51-2.60 (1H, d), 3.54-3.57 (1H, | |
| | pyridin-2-yl-methylidene]- | d), 3.70 (3H, s), 3.73-3.74 (1H, d), | |
| | piperidin-4-one | 6.18 (1H, s), 6.83-6.87 (2H, d), | |
| | | 7.05 (1H, s), 7.14-7.18 (2H, m), | |
| | | 7.21-7.22 (1H, m), 7.25-7.29 (5H, m), | |
| | | 7.47-7.49 (1H, d), 7.74-7.78 (1H, | |
| | | dd), 8.62-8.63 (1H, d) | |
| 48 | 1-(4-Methoxy-benzyl)-5,5- | 1.08 (6H, s), 2.34-2.38 (1H, d), | 419 |
| | dimethyl-3-[1-pyridin-2-yl- | 3.21-3.26 (1H, d), 3.51-3.52 (1H, | |
| | methylidene]-2-thiophen-2- | m), 3.62-3.64 (1H, m), 3.74 (3H, s), | |
| | yl-piperidin-4-one | 6.70 (1H, s), 6.83-6.86 (2H, d), | |
| | | 6.89-6.96 (2H, m), 7.04 (1H, s), | |
| | | 7.25-7.31 (2H, d), 7.31-7.33 (1H, m), | |
| | | 7.34-7.43 (1H, m), 7.44-7.53 (1H, | |
| | | d), 7.78-7.82 (1H, m), | |
| | | 8.62-8.63 (1H, dd) | |
| 49 | 1-Cyclopropyl-3,3-dimethyl- | 0.51 (2H, m), 0.52 (2H, m), 1.07 (6H, | 257 |
| | 5-[1-pyridin-2-yl- | s), 2.21-2.43 (1H, m), 2.82 (2H, s), | |
| | methylidene]-piperidin-4- | 4.17 (2H, s), 7.25 (1H, s), | |
| | one | 7.36-7.38 (1H, m), 7.65-7.88 (1H, d), | |
| | | 7.85-7.88 (1H, m), 8.75 (1H, s) | |
| 50 | 3,3-Dimethyl-5-[1-(6- | 1.22 (6H, s), 2.62 (2H, s), | 398 |
| | morpholin-4-yl-pyridin-2-yl)- | 3.44-3.46 (4H, t), 3.81-3.84 (4H, t), 3.90 (2H, | |
| | methylidene]-1-thiophen-2- | s), 4.25 (2H, s), 6.58-6.60 (1H, d), | |
| | ylmethyl-piperidin-4-one | 6.85-6.86 (1H, d), 6.94-6.95 (1H, | |
| | | m), 6.96-6.98 (1H, m), | |
| | | 7.24-7.25 (1H, dd), 7.29 (1H, s), | |
| | | 7.50-7.54 (1H, m) | |
| 51 | 1-Cyclopropyl-3,3-dimethyl- | 0.33-0.34 (2H, m), 0.48-0.49 (2H, | 342 |
| | 5-[1-(6-morpholin-4-yl- | m), 1.06 (6H, s), 1.79-1.81 (1H, m), | |
| | pyridin-2-yl)-methylidene]- | 2.70 (2H, s), 3.51-3.53 (4H, t), | |
| | piperidin-4-one | 3.72-3.74 (4H, t), 4.27 (2H, s), | |
| | | 6.85-6.88 (1H, d), 6.96-6.98 (1H, d), | |
| | | 7.11 (1H, s), 7.60-7.64 (1H, m) | |
| 52 | 2-(4-Methoxy-benzyl)-3,3- | 1.16 (3H, s), 1.25 (3H, s), | 395 |
| | dimethyl-4-oxo-5-[1-pyridin- | 2.42-2.45 (1H, m), 2.96-2.99 (1H, m), | |
| | 2-yl-methylidene]- | 3.31 (3H, s), 3.69 (3H, s), 4.40-4.60 (2H, | |
| | piperidine-1-carboxylic acid | m), 5.47-5.52 (1H, m), | |
| | methyl ester | 6.78-6.88 (2H, m), 7.09-7.14 (2H, d), | |
| | | 7.39-7.41 (1H, m), 7.42-7.48 (1H, m), | |
| | | 7.75-7.77 (1H, d), 7.89-7.94 (1H, | |
| | | m), 8.76-8.79 (1H, m) | |
| 53 | 2-(4-Methoxy-benzyl)-3,3- | 1.14 (6H, s), 2.44 (3H, s), | 502 |
| | dimethyl-4-oxo-5-[1-pyridin- | 2.66-2.72 (1H, m), 2.77-2.81 (1H, m), | |
| | 2-yl-methylidene]- | 3.59 (3H, s), 3.64-3.69 (1H, d), | |
| | piperidine-1-carboxylic acid | 3.70-3.76 (1H, m), 4.68-4.72 (1H, m), | |
| | (4-methylsulfanyl-phenyl)- | 6.68-6.76 (2H, d), 7.03-7.05 (3H, | |
| | amide | d), 7.12-7.14 (2H, d), | |
| | | 7.16-7.21 (2H, d), 7.27-7.29 (1H, d), | |
| | | 7.43-7.51 (2H, d), 7.70-7.74 (1H, m), | |
| | | 9.10 (1H, bs) | |
| 54 | 2-(4-Methoxy-benzyl)-3,3- | 1.25 (6H, s), 2.66-2.82 (2H, m), | 516 |
| | dimethyl-4-oxo-5-[1-pyridin- | 3.63 (6H, s), 3.65-3.67 (2H, m), | |
| | 2-yl-methylidene]- | 3.68 (3H, s), 4.57-4.58 (1H, m), | |
| | piperidine-1-carboxylic acid | 6.58-6.63 (1H, m), 6.75-6.77 (2H, | |
| | (2,6-dimethoxy-phenyl)- | d), 6.85-6.93 (2H, m), | |
| | amde | 7.07-7.09 (2H, d), 7.15-7.20 (1H, m), | |
| | | 7.22-7.24 (1H, d), 7.47-7.77 (1H, m), | |
| | | 7.92 (1H, s), 8.02 (1H, d), 8.97 (1H, | |
| | | bs) | |
| 55 | 3,3-Dimethyl-1-(5-methyl- | 1.08 (6H, s), 2.29 (3H, s), 3.50 (2H, | 383 |
| | isoxazol-3-yl)-5-[1-(6- | s), 3.52-3.57 (4H, t), | |
| | morpholin-4-yl-pyridin-2-yl)- | 3.60-3.76 (4H, t), 4.95 (2H, s), 6.10 (1H, s), | |
| | methylidene]-piperidin-4- | 6.88-6.92 (1H, d), 7.02-7.06 (1H, | |
| | one | d), 7.28 (1H, s), 7.57-7.67 (1H, m) | |
| 56 | 2-(2-Hydroxy-phenyl)-5,5- | 1.18 (6H, s), 2.20 (3H, s), | 475 |
| | dimethyl-1-(5-methyl- | 3.17-3.33 (2H, m), 3.49-3.52 (4H, t), | |
| | isoxazol-3-yl)-3-[1-(6- | 3.68-3.73 (4H, t), 5.62 (1H, s), | |
| | morpholin-4-yl-pyridin-2-yl)- | 5.87-5.90 (1H, m), 6.90-6.92 (2H, d), | |
| | methylidene]-piperidin-4- | 7.02-7.04 (2H, d), 7.36-7.39 (1H, d), | |
| | one | 7.55-7.63 (3H, m), 12.02 (1H, bs) | |
| 57 | 2-(2-Fluoro-phenyl)-5,5- | 1.15 (3H, s), 1.28 (3H, s), | 492 |
| | dimethyl-3-[1-(6-morpholin- | 2.55-2.64 (3H, m), 3.25 (2H, s), 3.37-3.80 (2H, | |
| | 4-yl-pyridin-2-yl)- | t), 3.68-3.69 (5H, m), | |
| | methylidene]-1-thiophen-2- | 4.05-4.09 (1H, dd), 6.64 (1H, s), | |
| | ylmethyl-piperidin-4-one | 6.78-6.79 (1H, m), 6.84 (1H, s), 7.02 (1H, s), | |
| | | 7.09 (1H, s), 7.18 (1H, s), 7.23- (2H, | |
| | | m), 7.37-7.38 (1H, m), | |
| | | 7.48-7.50 (1H, m), 7.52-7.54 (1H, m) | |
| 58 | (2-Fluoro-phenyl)-5,5- | 1.21 (6H, s), 2.39-2.45 (2H, m), | 407 |
| | dimethyl-3-[1-pyridin-2-yl- | 2.68-2.74 (1H, d), 3.47-3.53 (1H, | |
| | methylidene]-1-thiophen-2- | d), 4.00-4.06 (1H, d), 6.53 (1H, s), | |
| | ylmethyl-piperidin-4-one | 6.98-7.02 (2H, m), 7.15-7.21 (2H, | |
| | | m), 7.27-7.33 (3H, m), | |
| | | 7.47-7.48 (1H, dd), 7.60-7.62 (1H, d), | |
| | | 7.80-7.84 (1H, t), 8.62-8.63 (1H, m), | |
| 59 | 2-(4-Methoxy-benzyl)-3,3- | 0.86-1.13 (8H, m), 1.22-1.24 (3H, | 462 |
| | dimethyl-4-oxo-5-[1-pyridin- | m), 1.53-1.73 (6H, m), | |
| | 2-yl-methylidene]- | 2.64-2.67 (1H, m), 2.69-2.70 (1H, m), | |
| | piperidine-1-carboxylic acid | 3.10-3.25 (1H, m), 3.51 (3H, s), | |
| | cyclohexylamide | 4.27-4.32 (1H, m), 4.35-4.50 (1H, m), | |
| | | 5.58-5.60 (1H, m), 6.73-6.84 (2H, | |
| | | m), 6.96-6.98 (1H, m), | |
| | | 7.06-7.24 (2H, m), 7.32-7.40 (1H, m), | |
| | | 7.67-7.71 (1H, m), 7.88-7.91 (1H, m), | |
| | | 8.74 (1H, bs) | |
| 60 | 2-(4-Methoxy-benzyl)-3,3- | 1.16 (6H, s), 2.55-2.66 (2H, m), | 472 |
| | dimethyl-4-oxo-5-[1-pyridin- | 3.08-3.12 (2H, m), 3.72 (3H, s), | |
| | 2-yl-methylidene]- | 4.85-4.90 (1H, d), 6.80-6.84 (4H, | |
| | piperidine-1-carbothioic | m), 7.08-7.13 (1H, m), | |
| | acid phenylamide | 7.15-7.25 (4H, m), 7.32-7.38 (1H, m), | |
| | | 7.44-7.49 (1H, m), 7.71-7.56 (1H, m), | |
| | | 7.88-7.92 (1H, m), 9.46 (2H, s) | |
| 61 | 5,5-Dimethyl-2-(4- | 1.12 (3H, s), 1.17 (3H, s), 2.31 (1H, | 435 |
| | methylsulfanyl-phenyl)-3-[1- | m), 2.41 (3H, s), 2.65-2.68 (1H, d), | |
| | pyridin-2-yl-methylidene]-1- | 3.48-3.62 (1H, d), 4.10-4.15 (1H, | |
| | thiophen-2-ylmethyl- | d), 6.25 (1H, s), 6.85-6.98 (2H, m), | |
| | piperidin-4-one | 7.11 (1H, s), 7.17-7.22 (4H, m), | |
| | | 7.28 (1H, m), 7.42-7.43 (1H, m), | |
| | | 7.50-7.52 (1H, d), 7.70-7.80 (1H, | |
| | | m), 8.61-8.63 (1H, d) | |
| 62 | 1-(4-Methoxy-benzyl)-5,5- | 0.90 (3H, s), 1.00 (3H, s), | 498 |
| | dimethyl-3-[1-(6-morpholin- | 2.68-2.69 (1H, m), 3.01-3.08 (2H, m), | |
| | 4-yl-pyridin-2-yl)- | 3.46-3.49 (2H, t), 3.57-3.61 (2H, m), | |
| | methylidene]-2-phenyl- | 3.63-3.64 (3H, m), 3.73 (3H, s), | |
| | piperidin-4-one | 3.75-3.78 (1H, m), 4.14-4.15 (1H, m), | |
| | | 6.22 (1H, s), 6.35 (1H, s), | |
| | | 6.66-6.68 (1H, d), 6.87-6.91 (2H, m), | |
| | | 7.08-7.14 (2H, m), 7.17-7.21 (2H, m), | |
| | | 7.30-7.33 (1H, m), 7.40-7.46 (2H, | |
| | | m), 7.48-7.56 (1H, m), | |
| | | 7.70-7.71 (1H, m) | |
| 63 | 1-(4-Methoxy-benzyl)-5,5- | 0.99 (3H, s), 1.24 (3H, s), 2.68 (2H, | 566 |
| | dimethyl-3-[1-(6-morpholin- | s), 2.91-2.98 (4H, m), | |
| | 4-yl-pyridin-2-yl)- | 3.36-3.44 (4H, t), 3.65-3.70 (1H, d), 3.73 (3H, | |
| | methylidene]-2-(4- | s), 3.83-3.86 (1H, d), 6.40 (1H, s), | |
| | trifluoromethyl-phenyl)- | 6.75-6.83 (3H, m), 6.92-6.94 (1H, | |
| | piperidin-4-one | d), 7.18-7.20 (2H, d), 7.25 (1H, s), | |
| | | 7.32-7.34 (2H, d), 7.54-7.58 (1H, | |
| | | dd), 7.69-7.71 (2H, d), | |
| 64 | 3,3-Dimethyl-1-(5-methyl- | 1.12 (6H, s), 2.19 (2H, d), 2.38 (3H, | 298 |
| | isoxazol-3-yl)-5-[1-pyridin- | s), 3.95 (1H, s), 4.89 (1H, s), | |
| | 2-yl-methylidene]-piperidin- | 6.72 (1H, s), 7.45 (1H, s), 7.65-7.74 (1H, | |
| | 4-one | m), 7.89-7.96 (1H, m), 8.44 (1H, s), | |
| | | 8.78-8.79 (1H, d) | |
| 65 | 5,5-Dimethyl-1-(5-methyl- | 1.13 (6H, s), 2.18 (3H, s), | 459 |
| | isoxazol-3-yl)-3-[1-(6- | 3.31-3.36 (4H, t), 3.41 (2H, s), 3.67-3.72 (4H, | |
| | morpholin-4-yl-pyridin-2-yl)- | t), 5.62 (1H, s), 5.87-5.90 (1H, m), | |
| | methylidene]-2-phenyl- | 6.89-6.92 (1H, d), 7.02-7.04 (1H, | |
| | piperidin-4-one | d), 7.35-7.39 (1H, d), | |
| | | 7.51-7.74 (6H, m) | |
| 66 | 2-(4-Methoxy-benzyl)-3,3- | 1.18 (3H, s), 1.29 (3H, s), | 470 |
| | dimethyl-4-oxo-5-[1-pyridin- | 2.13-2.19 (2H, m), 2.97-3.01 (1H, m), | |
| | 2-yl-methylidene]- | 3.73 (3H, s), 4.10-4.15 (2H, m), 4.18 (2H, | |
| | piperidine-1-carboxylic acid | s), 6.78-6.89 (2H, d), | |
| | benzylamide | 6.93-6.95 (2H, d), 7.13-7.20 (2H, m), | |
| | | 7.23 (5H, s), 7.37-7.44 (1H, m), 7.69 (1H, | |
| | | m), 7.87-7.90 (1H, m), | |
| | | 8.73-8.74 (1H, d) | |
| 67 | 2-(4-Methoxy-benzyl)-3,3- | 1.07 (3H, s), 1.12 (3H, s), | 474 |
| | dimethyl-4-oxo-5-[1-pyridin- | 3.00-3.05 (1H, m), 3.60-3.62 (1H, m), | |
| | 2-yl-methylidene]- | 3.66 (3H, s), 3.68-3.73 (1H, m), | |
| | piperidine-1-carboxylic acid | 4.40-4.43 (1H, m), 4.55-4.68 (1H, m), | |
| | (4-fluoro-phenyl)-amide | 6.19-6.22 (1H, m), 6.53-6.69 (1H, | |
| | | m), 6.74-6.82 (2H, m), | |
| | | 6.90-6.99 (2H, m), 7.01-7.14 (1H, m), | |
| | | 7.39-7.45 (1H, m), 7.47-7.53 (1H, m), | |
| | | 7.74-7.76 (1H, m), 7.90-7.93 (1H, | |
| | | m), 8.067-8.22 (1H, m), 8.48 (1H, | |
| | | bs), 8.77-8.78 (1H, m) | |
| 68 | 2-(4-Methoxy-benzyl)-3,3- | 0.94-0.97 (6H, m), 1.01-1.07 (6H, | 540 |
| | dimethyl-4-oxo-5-[1-pyridin- | m), 1.24 (6H, s), 2.51-2.59 (1H, d), | |
| | 2-yl-methylidene]- | 3.00-3.03 (2H, m), 3.14-3.17 (1H, | |
| | piperidine-1-carboxylic acid | m), 3.73 (3H, s), 4.04-4.63 (2H, m), | |
| | (2,6-diisopropyl-phenyl)- | 5.77-5.86 (1H, m), 6.79-6.85 (2H, | |
| | amide | m), 7.01-7.08 (3H, m), 7.21 (1H, s), | |
| | | 7.38-7.41 (1H, m), 7.48 (1H, s), | |
| | | 7.59 (1H, s), 7.68 (1H, m), | |
| | | 7.68-7.77 (1H, m), 7.89-7.92 (1H, m), | |
| | | 8.75-8.76 (1H, d) | |
| 69 | 3,3-Dimethyl-5-[1-(6- | 1.17 (6H, s), 2.64-2.72 (4H, m), | 412 |
| | morpholin-4-yl-pyridin-2-yl)- | 3.49-3.53 (4H, t), 3.54 (2H, s), | |
| | methylidene]-1-(2-thiophen- | 3.57 (2H, s), 3.70-3.74 (4H, t), | |
| | 2-yl-ethyl)-piperidin-4-one | 6.71-6.73 (1H, d), 6.90-6.92 (1H, d), | |
| | | 7.03-7.055 (1H, d), 7.23-7.26 (1H, d), | |
| | | 7.35-7.39 (1H, d), 7.56-7.66 (2H, | |
| | | m) | |
| 70 | 2-(2-Fluoro-phenyl)-5,5- | 1.14 (6H, s), 3.22 (2H, s), | 407 |
| | dimethyl-3-[1-pyridin-3-yl- | 3.39-3.46 (1H, m), 4.01-4.04 (1H, d), | |
| | methylidene]-1-thiophen-2- | 5.45 (1H, d), 6.90-6.93 (2H, m), | |
| | ylmethyl-piperidin-4-one | 7.21-7.26 (2H, m), 7.30 (1H, s), | |
| | | 7.33-7.35 (1H, m), 7.36-7.41 (1H, m), | |
| | | 7.42-7.44 (2H, m), 7.60-7.62 (1H, | |
| | | m), 8.41 (1H, s), 8.47-8.49 (1H, dd) | |
| 71 | 1-Benzyl-5,5-dimethyl-3-[1- | 1.02 (3H, s), 1.10 (3H, s), 2.06 (1H, | 473 |
| | pyridin-2-yl-methylidene]-2- | s), 2.13-2.19 (1H, s), | |
| | (3,4,5-trimethoxy-phenyl)- | 2.55-2.67 (1H, d), 3.59 (3H, s), 3.68 (6H, s), | |
| | piperidin-4-one | 4.13-4.15 (1H, m), 6.18 (1H, s), | |
| | | 6.57 (2H, s), 6.98 (1H, s), | |
| | | 7.24-7.30 (1H, m), 7.32-7.35 (4H, m), | |
| | | 7.51-7.53 (1H, m), 7.69-7.73 (1H, m), | |
| | | 7.78-7.82 (1H, m), 8.69-8.70 (1H, | |
| | | d) | |
| 72 | 1-(4-Fluoro-benzyl)-3,3- | 1.07 (6H, s), 3.21-3.29 (1H, m), | 325 |
| | dimethyl-5-[1-pyridin-2-yl- | 3.34-3.36 (1H, m), 3.63 (2H, s), | |
| | methylidene]-piperidin-4- | 4.08 (2H, s), 7.30-7.38 (2H, m), | |
| | one | 7.65-7.71 (1H, d), 7.93-7.96 (3H, | |
| | | m), 8.66-8.67 (1H, dd), | |
| | | 8.83-8.85 (2H, dd) | |
| 73 | 1-(4-Fluoro-benzyl)-3,3- | 1.12 (6H, s), 2.59 (2H, s), | 410 |
| | dimethyl-5-[1-(6-morpholin- | 3.29-3.34 (4H, t), 3.65-3.67 (6H, m), 4.03 (2H, | |
| | 4-yl-pyridin-2-yl)- | s), 6.81-6.83 (1H, d), | |
| | methylidene]-piperidin-4- | 6.96-6.97 (1H, d), 7.14-7.19 (3H, m), | |
| | one | 7.38-7.42 (2H, m), 7.57-7.61 (1H, m) | |
| 74 | 3,3-Dimethyl-5-[1-(6- | 1.14 (6H, s), 2.64 (2H, s), | 460 |
| | morpholin-4-yl-pyridin-2-yl)- | 3.24-3.30 (4H, t), 3.60-3.62 (4H, t), 3.77 (2H, | |
| | methylidene]-1-(4- | s), 4.06 (2H, s), 6.97-6.99 (1H, m), | |
| | trifluoromethyl-benzyl)- | 7.05-7.09 (1H, m), 7.21 (1H, s), | |
| | piperidin-4-one | 7.60-7.62 (3H, m), 7.70-7.72 (2H, | |
| | | m) | |
| 75 | 4-({2-(4-Methoxy-benzyl)- | 0.88 (3H, s), 1.04 (3H, s), | 528 |
| | 3,3-dimethyl-4-oxo-5-[1- | 1.26-1.29 (3H, t), 2.52 (1H, s), 2.79-2.82 (1H, | |
| | pyridin-2-yl-methylidene]- | d), 3.27-3.30 (1H, d), | |
| | piperidine-1-carbonyl}- | 3.37-3.46 (2H, m), 3.69 (3H, s), 4.23-4.26 (2H, | |
| | amino)-benzoic acid ethyl | q), 6.78-6.80 (1H, d), | |
| | ester | 6.87-6.89 (2H, d), 7.08-7.10 (1H, m), | |
| | | 7.11-7.12 (1H, m), 7.14-7.18 (2H, m), | |
| | | 7.62-7.64 (3H, d), 7.89-7.91 (2H, | |
| | | d), 8.40-8.41 (1H, dd), 9.74 (1H, bs) | |
| 76 | 1-(4-Fluoro-benzyl)-5,5- | 1.12 (6H, s), 2.34-2.36 (1H, m), | 401 |
| | dimethyl-2-phenyl-3-[1- | 2.56-2.62 (2H, m), 3.35-3.40 (2H, | |
| | pyridin-2-yl-methylidene]- | m), 6.17-6.21 (1H, d), | |
| | piperidin-4-one | 7.07-7.09 (1H, m), 7.11-7.14 (1H, m), | |
| | | 7.19-7.20 (1H, m), 7.21-7.32 (7H, m), | |
| | | 7.49-7.51 (1H, d), 7.74-7.79 (1H, | |
| | | m), 8.62-8.63 (1H, dd) | |
| 77 | 1-(4-Methoxy-benzyl)-5,5- | 1.08 (3H, s), 1.12 (3H, s), | 481 |
| | dimethyl-3-[1-pyridin-2-yl- | 2.54-2.56 (2H, d), 3.30-3.31 (2H, d), | |
| | methylidene]-2-(4- | 3.71 (3H, s), 6.27 (1H, s), 6.84-6.86 (2H, | |
| | trifluoromethyl-phenyl)- | d), 7.12-7.17 (2H, m), | |
| | piperidin-4-one | 7.30-7.33 (1H, m), 7.51-7.56 (4H, m), | |
| | | 7.66-7.68 (2H, d), 7.73-7.81 (1H, m), | |
| | | 8.63-8.64 (1H, dd) | |
| 78 | 2-(2-Fluoro-phenyl)-1-(4- | 1.08 (6H, s), 2.34-2.38 (1H, m), | 431 |
| | methoxy-benzyl)-5,5- | 2.53-2.56 (1H, m), 3.20-3.23 (1H, | |
| | dimethyl-3-[1-pyridin-2-yl- | d), 3.65-3.68 (1H, d), 3.72 (3H, s), | |
| | methylidene]-piperidin-4- | 6.39 (1H, s), 6.84-6.86 (2H, d), | |
| | one | 7.12-7.18 (4H, m), 7.21-7.28 (4H, m), | |
| | | 7.53-7.55 (1H, d), 7.74-7.78 (1H, | |
| | | m), 8.56-8.57 (1H, dd) | |
| 79 | 3,3-Dimethyl-5-[1-pyridin-2- | 1.18 (6H, s), 2.26-2.64 (4H, t), | 327 |
| | yl-methylidene]-1-(2- | 2.84 (2H, s), 3.53 (2H, s), 6.71-6.73 (1H, | |
| | thiophen-2-yl-ethyl)- | d), 7.22-7.23 (1H, d), | |
| | piperidin-4-one | 7.40-7.43 (1H, m), 7.51-7.55 (1H, d), | |
| | | 7.70-7.72 (1H, m), 7.78-7.80 (1H, m), | |
| | | 7.85-7.89 (1H, m), 8.65-8.66 (1H, | |
| | | d) | |
| 80 | 5,5-Dimethyl-3-[1-(6- | 0.93 (3H, s), 1.20 (3H, s), 2.68 (2H, | 488 |
| | morpholin-4-yl-pyridin-2-yl)- | s), 2.97-3.00 (4H, t), | |
| | methylidene]-2-phenyl-1-(2- | 3.47-3.49 (2H, t), 3.56-3.60 (4H, t), | |
| | thiophen-2-yl-ethyl)- | 3.59-3.65 (2H, t), 6.21 (1H, s), 6.59-6.68 (1H, | |
| | piperidin-4-one | m), 6.70-6.76 (1H, m), | |
| | | 6.86-6.92 (1H, m), 7.07-7.12 (3H, m), | |
| | | 7.16-7.22 (4H, m), 7.24-7.28 (1H, m), | |
| | | 7.37-7.46 (1H, m) | |
| 81 | 1-(4-Fluoro-benzyl)-5,5- | 1.01 (3H, s), 1.22 (3H, s), | 486 |
| | dimethyl-3-[1-(6-morpholin- | 2.98-3.01 (2H, m), 3.08-3.12 (2H, t), | |
| | 4-yl-pyridin-2-yl)- | 3.24-3.29 (1H, m), 3.38-3.47 (4H, t), | |
| | methylidene]-2-phenyl- | 3.62-3.67 (2H, t), 3.85-3.89 (2H, m), | |
| | piperidin-4-one | 6.39 (1H, s), 6.68-6.70 (1H, d), | |
| | | 6.74-6.76 (1H, d), 7.07-7.13 (3H, m), | |
| | | 7.15-7.22 (1H, m), 7.24-7.25 (1H, | |
| | | m), 7.30-7.34 (4H, m), | |
| | | 7.42-7.52 (1H, dd) | |
| 82 | 1-Furan-2-ylmethyl-5,5- | 1.09 (3H, s), 1.24 (3H, s), | 373 |
| | dimethyl-2-phenyl-3-[1- | 2.51-2.54 (1H, d), 2.67-2.70 (1H, d), | |
| | pyridin-2-yl-methylidene]- | 3.39-3.44 (1H, d), 3.67-3.71 (1H, d), | |
| | piperidin-4-one | 6.17 (1H, s), 6.28-6.29 (1H, dd), | |
| | | 6.39-36.41 (1H, m), 7.03-7.04 (1H, m), | |
| | | 7.19-7.20 (1H, m), 7.21-7.27 (4H, | |
| | | m), 7.47-7.49 (1H, d), | |
| | | 7.60-7.61 (1H, m), 7.74-7.78 (2H, m), | |
| | | 8.63-8.64 (1H, m) | |
| 83 | 1-(3,4-Difluoro-benzyl)-5,5- | 1.07 (6H, s), 2.50-2.51 (2H, d), | 419 |
| | dimethyl-2-phenyl-3-[1- | 2.56-2.61 (1H, d), 3.37-3.39 (1H, | |
| | pyridin-2-yl-methylidene]- | m), 3.66-3.67 (1H, d), 6.24 (1H, s), | |
| | piperidin-4-one | 7.09-7.13 (2H, m), 7.21-7.24 (3H, | |
| | | m), 7.25-7.28 (3H, m), | |
| | | 7.29-7.34 (1H, m), 7.50-7.52 (1H, d), | |
| | | 7.75-7.79 (1H, m), 8.61-8.62 (1H, m) | |
| 84 | 5,5-Dimethyl-2-phenyl-3-[1- | 1.00 (3H, s), 1.13 (3H, s), | 403 |
| | pyridin-2-yl-methylidene]-1- | 2.36-2.39 (1H, d), 2.62-2.70 (1H, m), | |
| | (2-thiophen-2-yl-ethyl)- | 2.76-2.87 (2H, m), 2.90-2.92 (1H, d), | |
| | piperidin-4-one | 3.60-3.63 (1H, m), 4.14-4.15 (1H, | |
| | | m), 6.70-6.71 (1H, d), | |
| | | 6.79-6.80 (1H, d), 6.86-6.90 (1H, m), | |
| | | 7.14-7.19 (1H, m), 7.24-7.28 (3H, m), | |
| | | 7.29-7.32 (3H, m), 7.33-7.34 (2H, | |
| | | m), 7.52-7.53 (1H, m) | |
| 85 | 1,5,5-Trimethyl-2-phenyl-3- | 1.18 (6H, s), 2.19 (3H, s), 3.73 (2H, | 307 |
| | [1-pyridin-2-yl-methylidene]- | s), 5.85 (1H, s), 6.48-6.53 (1H, m), | |
| | piperidin-4-one | 6.88 (1H, s), 7.02-7.25 (3H, m), | |
| | | 7.26-7.27 (1H, m), 7.28-7.29 (1H, | |
| | | m), 7.46-7.48 (1H, d), | |
| | | 7.69-7.76 (1H, m), 8.65-8.66 (1H, d) | |
| 86 | 2-(2-Fluoro-phenyl)-1-(4- | 1.04 (3H, s), 1.19 (3H, s), | 516 |
| | methoxy-benzyl)-5,5- | 2.41-2.46 (1H, m), 3.14-3.21 (2H, d), | |
| | dimethyl-3-[1-(6-morpholin- | 3.27-3.29 (1H, d), 3.30 (3H, s), | |
| | 4-yl-pyridin-2-yl)- | 3.58-3.61 (4H, t), 3.72-3.73 (4H, t), 6.48 (1H, | |
| | methylidene]-piperidin-4- | s), 6.60-6.61 (1H, d), | |
| | one | 6.79-6.81 (1H, d), 6.86-6.88 (2H, d), | |
| | | 6.98-7.01 (1H, m), 7.14-7.16 (1H, m), | |
| | | 7.17-7.18 (2H, m), 7.20-7.25 (2H, | |
| | | d), 7.28-7.30 (1H, m), | |
| | | 7.42-7.52 (1H, m) | |
| 87 | 1-(4-Fluoro-benzyl)-3,3- | 0.96 (6H, s), 2.47 (2H, s), 2.56 (3H, | 370 |
| | dimethyl-5-[1-(4- | s), 3.64 (2H, s), 3.73 (2H, s), | |
| | methylsulfanyl-phenyl)- | 7.13-7.19 (2H, m), 7.29-7.34 (3H, m), | |
| | methylidene]-piperidin-4- | 7.37-7.42 (4H, m) | |
| | one | | |
| 88 | 5,5-Dimethyl-1-(5-methyl- | 1.00 (3H, s), 1.12 (3H, s), 2.29 (3H, | 505 |
| | isoxazol-3-yl)-2-(4- | s), 2.43 (3H, s), 3.00-3.04 (1H, d), | |
| | methylsulfanyl-phenyl)-3-[1- | 3.13-3.14 (2H, m), 3.28-3.49 (2H, | |
| | (6-morpholin-4-yl-pyridin-2- | t), 3.46-3.59 (5H, m), 6.16 (1H, s), | |
| | yl)-methylidene]-piperidin-4- | 6.83-6.85 (1H, d), 7.00-7.02 (2H, | |
| | one | d), 7.07-7.09 (1H, d), | |
| | | 7.21-7.23 (2H, d), 7.29 (1H, s), 7.59-7.63 (1H, | |
| | | m), 7.75 (1H, s) | |
| 89 | 3,3-Dimethyl-1-(5-methyl- | 1.09 (6H, s), 2.34 (3H, s), 2.46 (3H, | 343 |
| | isoxazol-3-yl)-5-[1-(4- | s), 3.16 (2H, s), 3.89 (2H, s), | |
| | methylsulfanyl-phenyl)- | 6.77 (1H, s), 7.22-7.26 (2H, d), | |
| | methylidene]-piperidin-4- | 7.54-7.57 (2H, d), 8.22 (1H, s) | |
| | one | | |
| 90 | 1-Furan-2-ylmethyl-5,5- | 1.01 (3H, s), 1.15 (3H, s), | 463 |
| | dimethyl-3-[1-pyridin-2-yl- | 2.57-2.60 (1H, d), 2.67-2.71 (1H, d), | |
| | methylidene]-2-(3,4,5- | 3.42-3.56 (1H, d), 3.60 (3H, s), 3.67 (6H, | |
| | trimethoxy-phenyl)- | s), 3.71-3.76 (1H, d), 6.19 (1H, s), | |
| | piperidin-4-one | 6.30-6.31 (1H, d), 6.40-6.41 (1H, | |
| | | d), 6.57 (2H, s), 6.94 (1H, s), | |
| | | 7.32-7.35 (1H, m), 7.50-7.52 (1H, s), | |
| | | 7.69-7.72 (1H, m), 7.73-7.82 (1H, | |
| | | m), 8.70-8.71 (1H, dd) | |
| 91 | 1-Benzyl-2-(2-fluoro-4- | 0.88 (6H, s), 3.27-3.30 (1H, d), | 431 |
| | methoxy-phenyl)-5,5- | 3.55-3.56 (1H, d), 3.60-3.68 (2H, | |
| | dimethyl-3-[1-pyridin-2-yl- | m), 3.72 (3H, s), 6.16 (1H, s), | |
| | methylidene]-piperidin-4- | 6.65-6.76 (1H, m), 7.10-7.15 (3H, m), | |
| | one | 7.21-7.29 (5H, m), 7.31-7.34 (1H, | |
| | | m), 7.40-7.41 (1H, m), | |
| | | 7.68-7.69 (1H, m), 8.51-8.52 (1H, d) | |
| 92 | 1-Benzyl-2-(2-fluoro-4- | 1.06 (3H, s), 1.19 (3H, s), | 516 |
| | methoxy-phenyl)-5,5- | 2.50-2.51 (1H, d), 2.55-2.58 (1H, d), | |
| | dimethyl-3-[1-(6-morpholin- | 3.36-3.37 (4H, t), 3.58-3.60 (4H, t), | |
| | 4-yl-pyridin-2-yl)- | 3.74 (3H, s), 3.75-3.76 (2H, m), 6.14 (1H, | |
| | methylidene]-piperidin-4- | s), 6.46 (1H, s), 6.74-6.75 (1H, d), | |
| | one | 6.76-6.79 (2H, m), 6.87-6.91 (1H, | |
| | | m), 7.10 (1H, s), 7.20-7.21 (2H, m), | |
| | | 7.27-7.35 (3H, m), 7.74-7.52 (1H, | |
| | | m) | |
| 93 | 5,5-Dimethyl-3-[1-(6- | 1.02 (3H, s), 1.20 (3H, s), | 558 |
| | morpholin-4-yl-pyridin-2-yl)- | 2.99-3.06 (2H, m), 3.44-3.46 (4H, t), 3.63 (9H, | |
| | methylidene]-2-phenyl-1- | s), 3.72-3.75 (2H, m), | |
| | (3,4,5-trimethoxy-benzyl)- | 4.13-4.15 (4H, t), 6.45 (1H, s), 6.59-6.62 (2H, | |
| | piperidin-4-one | s), 6.73-6.75 (1H, d), | |
| | | 6.89-6.90 (1H, d), 7.09-7.11 (2H, d), 7.16 (1H, | |
| | | d), 7.22-7.24 (1H, m), | |
| | | 7.32-7.35 (2H, m), 7.54-7.56 (1H, m) | |
| 94 | 5,5-Dimethyl-1-phenethyl-2- | 1.14 (3H, s), 1.18 (3H, s), | 397 |
| | phenyl-3-[1-pyridin-2-yl- | 2.66-2.67 (2H, m), 2.68-2.69 (1H, m), | |
| | methylidene]-piperidin-4- | 2.72-2.73 (2H, m), 2.74-2.80 (1H, m), | |
| | one | 6.26 (1H, s), 7.04 (1H, s), | |
| | | 7.09-7.11 (2H, m), 7.15-7.17 (4H, m), | |
| | | 7.18-7.21 (3H, m), 7.22-7.24 (1H, m), | |
| | | 7.26-7.31 (1H, m), 7.48-7.50 (1H, | |
| | | d), 7.74-7.78 (1H, m), | |
| | | 8.66-8.67 (1H, dd) | |
| 95 | 5,5-Dimethyl-3-[1-(6- | 1.06 (3H, s), 1.16 (3H, s), | 482 |
| | morpholin-4-yl-pyridin-2-yl)- | 2.66-2.69 (4H, t), 2.79 (2H, s), 2.94-2.99 (2H, | |
| | methylidene]-1-phenethyl- | t), 3.50-3.62 (6H, m), 6.43 (1H, s), | |
| | 2-phenyl-piperidin-4-one | 6.79-6.86 (2H, dd), 6.96-7.03 (1H, | |
| | | m), 7.05-7.09 (2H, m), | |
| | | 7.14-7.16 (3H, m), 7.22-7.30 (5H, m), | |
| | | 7.47-7.58 (1H, m), | |
| 96 | 5,5-Dimethyl-1-(5-methyl- | 0.99 (3H, s), 1.10 (3H, s), 2.30 (3H, | 527 |
| | isoxazol-3-yl)-3-[1-(6- | s), 2.98-3.02 (4H, m), | |
| | morpholin-4-yl-pyridin-2-yl)- | 3.19-3.20 (2H, m), 3.36-3.38 (2H, m), | |
| | methylidene]-2-(4- | 3.39-3.41 (1H, m), 3.45-3.61 (1H, d), | |
| | trifluoromethyl-phenyl)- | 6.22 (1H, s), 6.83-6.86 (1H, d), | |
| | piperidin-4-one | 7.05-7.06 (1H, d), 7.36-7.38 (3H, d), | |
| | | 7.60-7.64 (1H, m), 7.71-7.73 (2H, | |
| | | d), 7.85 (1H, s) | |
| 97 | 5,5-Dimethyl-1-(5-methyl- | 0.99 (3H, s), 1.12 (3H, s), | 540 |
| | isoxazol-3-yl)-3-[1-[6-(4- | 2.04-2.06 (2H, m), 2.09-2.12 (5H, m), | |
| | methyl-piperazin-1-yl)- | 2.30 (3H, s), 2.98-3.01 (1H, d), | |
| | pyridin-2-yl]-methylidene]-2- | 3.06-3.10 (2H, m), 3.21-3.23 (2H, m), | |
| | (4-trifluoromethyl-phenyl)- | 3.61-3.64 (1H, d), 6.17 (1H, s), | |
| | piperidin-4-one | 6.83-6.85 (1H, d), 7.00-7.02 (1H, d), | |
| | | 7.35-7.38 (3H, m), 7.56-7.60 (1H, | |
| | | m), 7.71-7.73 (2H, d), 7.87 (1H, s) | |
| 98 | 5,5-Dimethyl-1-(5-methyl- | 1.08 (6H, s), 2.18 (3H, s), 3.46 (2H, | 442 |
| | isoxazol-3-yl)-3-[1-pyridin- | s), 5.61 (1H, s), 7.07-7.10 (1H, d), | |
| | 2-yl-methylidene]-2-(4- | 7.11-7.13 (1H, m), 7.54-7.56 (2H, | |
| | trifluoromethyl-phenyl)- | d), 7.59-7.63 (1H, m), | |
| | piperidin-4-one | 7.68-7.71 (3H, d), 8.36-8.37 (1H, dd), | |
| | | 11.99 (1H, s) | |
| 99 | {5,5-Dimethyl-3-[1-(6- | 1.04 (3H, s), 1.14 (3H, s), | 436 |
| | morpholin-4-yl-pyridin-2-yl)- | 2.65-2.68 (1H, d), 2.84-2.89 (1H, t), | |
| | methylidene]-4-oxo-2- | 3.28-3.29 (2H, m), 3.35-3.47 (4H, t), | |
| | phenyl-piperidin-1-yl}-acetic | 3.60-3.67 (4H, m), 6.55 (1H, s), | |
| | acid | 6.77-6.79 (1H, d), 6.83-6.84 (1H, d), | |
| | | 7.00 (1H, s), 7.18-7.22 (3H, m), | |
| | | 7.27-7.30 (2H, m), 7.51-7.55 (1H, m), | |
| | | 12.36-12.39 (1H, bs) | |
| 100 | {5,5-Dimethyl-4-oxo-2- | 0.96 (3H, s), 1.36 (3H, s), 2.75 (2H, | 351 |
| | phenyl-3-[1-pyridin-2-yl- | s), 2.87 (2H, s), 4.10 (1H, d), | |
| | methylidene]-piperidin-1-yl}- | 7.12-7.17 (2H, m), 7.23-7.31 (2H, m), | |
| | acetic acid | 7.32-7.33 (2H, m), 7.38-7.41 (4H, | |
| | | m), 12.20-12.22 (1H, bs) | |
| 101 | {2-(4-Fluoro-phenyl)-5,5- | 0.98 (3H, s), 1.08 (3H, s), 3.13 (2H, | 369 |
| | dimethyl-4-oxo-3-[1-pyridin- | s), 3.75 (2H, s), 6.89-6.94 (2H, m), | |
| | 2-yl-methylidene]-piperidin- | 7.18-7.22 (2H, m), 7.23-7.24 (2H, | |
| | 1-yl}-acetic acid | m) 7.25-7.31 (3H, m), | |
| | | 7.36-7.42 (2H, m) | |
| 102 | {5,5-Dimethyl-3-[1-[6-(4- | 1.10 (3H, s), 1.20 (3H, s), 2.21 (3H, | 449 |
| | methyl-piperazin-1-yl)- | s), 2.33-2.36 (4H, m), | |
| | pyridin-2-yl]-methylidene]-4- | 2.65-2.68 (2H, m), 2.84 (2H, m), | |
| | oxo-2-phenyl-piperidin-1- | 3.51-3.65 (4H, t), 6.57 (1H, s), 6.67-6.80 (1H, | |
| | yl}-acetic acid | m), 6.98 (1H, s), 7.19-7.21 (2H, m), | |
| | | 7.26-7.28 (3H, m), 7.36-7.37 (1H, | |
| | | m), 7.48-7.52 (1H, m), | |
| | | 12.30-12.36 (1H, bs), | |
| 103 | 1-Benzyl-3-[1-(6-morpholin- | 3.48-3.50 (4H, t), 3.71-3.73 (4H, t), | 454 |
| | 4-yl-pyridin-2-yl)- | 4.37 (2H, s), 4.80 (2H, s), | |
| | methylidene]-5-phenyl- | 7.01-7.03 (1H, d), 7.13-7.15 (1H, d), | |
| | piperidine-2,4-dione | 7.19-7.22 (1H, m), 7.26-7.28 (1H, m), | |
| | | 7.30-7.35 (6H, m), 7.64-7.67 (3H, | |
| | | m), 7.74-7.78 (1H, m), 14.65 (1H, s) | |
| 104 | 2-(4-Methanesulfonyl- | 1.04 (3H, s), 1.44 (3H, s), 3.21 (3H, | 591 |
| | phenyl)-3,3-dimethyl-5-[1- | s), 3.23-3.27 (4H, m), | |
| | (6-morpholin-4-yl-pyridin-2- | 3.37-3.45 (4H, m), 3.64-3.69 (2H, m), | |
| | yl)-methylidene]-4-oxo- | 5.96-5.98 (1H, d), 6.92-6.95 (1H, d), | |
| | piperidine-1-carbothioic | 7.11-7.15 (1H, m), 7.17-7.19 (1H, m), | |
| | acid phenylamide | 7.24-7.32 (3H, m), 7.44-7.48 (3H, | |
| | | m), 7.62-7.71 (2H, m), | |
| | | 7.86-7.88 (2H, d), 9.28 (1H, s) | |
| 105 | 2-(4-Methanesulfonyl- | 0.98 (3H, s), 1.36 (3H, s), | 506 |
| | phenyl)-3,3-dimethyl-4-oxo- | 3.16-3.18 (1H, d), 3.21 (3H, s), 3.23-3.27 (1H, | |
| | 5-[1-pyridin-2-yl- | s), 3.39-3.43 (1H, m), | |
| | methylidene]-piperidine-1- | 7.13-7.15 (1H, m), 7.23-7.32 (4H, m), | |
| | carbothioic acid | 7.41-7.43 (1H, m), 7.48-7.50 (2H, d), | |
| | phenylamide | 7.66 (1H, s), 7.83-7.91 (2H, m), | |
| | | 7.92-8.01 (2H, m), 8.72-8.73 (1H, | |
| | | d), 9.46 (1H, s) | |
| 106 | 2-(4-Methanesulfonyl- | 1.00 (3H, s), 1.39 (3H, s), 3.21 (3H, | 504 |
| | phenyl)-3,3-dimethyl-4-oxo- | s), 4.03-4.12 (2H, m), | |
| | 5-[1-pyridin-2-yl- | 4.92-4.97 (1H, m), 5.46 (2H, s), 7.18-7.20 (2H, | |
| | methylidene]-piperidine-1- | m), 7.21-7.24 (3H, m), | |
| | carboxylic acid | 7.32-7.42 (5H, m), 7.80-7.93 (4H, m), | |
| | benzylamide | 8.74-8.74 (1H, d) | |
| 107 | 1-Benzyl-5-phenyl-3-[1- | 4.11-4.17 (4H, m), 5.11 (1H, s), | 369 |
| | pyridin-2-yl-methylidene]- | 7.21-7.33 (8H, m), 7.36-7.41 (4H, | |
| | piperidine-2,4-dione | m), 7.45-7.46 (1H, d), 7.77 (1H, s), | |
| | | 8.50-8.51 (1H, d) | |
| 108 | 1-Benzyl-3-[1-[6-(4-methyl- | 2.17-2.24 (2H, m), 2.75-2.82 (4H, | 467 |
| | piperazin-1-yl)-pyridin-2-yl]- | m), 3.16-3.17 (2H, d), 3.26 (3H, s), | |
| | methylidene]-5-phenyl- | 3.40-3.72 (4H, m), 4.10 (1H, m), | |
| | piperidine-2,4-dione | 6.77-6.80 (2H, m), 6.91-6.94 (4H, | |
| | | m), 7.15-7.34 (7H, m), 8.32 (1H, s) | |
| 109 | 1-(3,4-Dimethoxy-benzyl)- | 1.01 (3H, s), 1.12 (3H, s), | 443 |
| | 5,5-dimethyl-2-phenyl-3-[1- | 2.34-2.37 (2H, m), 2.55-2.57 (2H, m), | |
| | pyridin-2-yl-methylidene]- | 3.78 (3H, s), 3.82 (3H, s), 6.22 (1H, s), | |
| | piperidin-4-one | 6.83-6.84 (1H, m), 7.18 (1H, s), | |
| | | 7.19-7.21 (1H, m), 7.25-7.31 (4H, | |
| | | m), 7.32-7.37 (1H, m), | |
| | | 7.39-7.41 (1H, m), 7.43-7.44 (1H, d), | |
| | | 7.49-7.54 (1H, d), 8.62-8.63 (1H, d) | |
| 110 | 5,5-Dimethyl-1-(4-methyl- | 1.45 (6H, s), 2.29 (3H, s), | 495 |
| | benzyl)-3-[1-[6-(4-methyl- | 2.31-2.36 (6H, t), 2.48-2.51 (2H, m), | |
| | piperazin-1-yl)-pyridin-2-yl]- | 3.21-3.63 (2H, m), 3.36-3.48 (3H, t), | |
| | methylidene]-2-phenyl- | 3.63-3.66 (2H, t), 6.34 (1H, s), | |
| | piperidin-4-one | 6.47-6.53 (1H, d), 6.67-6.69 (1H, d), | |
| | | 7.00 (1H, s), 7.06-7.09 (2H, d), | |
| | | 7.13-7.16 (4H, m), 7.21-7.25 (1H, m), | |
| | | 7.28-7.32 (1H, m), 7.35-7.43 (1H, | |
| | | m), 7.48-7.50 (1H, d), | |
| 111 | 2-(4-Methanesulfonyl- | 0.89 (3H, s), 1.43 (3H, s), 3.20 (3H, | 508 |
| | phenyl)-3,3-di | s), 3.21-3.29 (2H, m), | |
| | methyl-4-oxo-5-[1-pyridin-2- | 5.61-5.62 (1H, d), 7.03-7.06 (2H, m), | |
| | yl-methylidene]-piperidine- | 7.31-7.33 (2H, m), 7.52 (1H, s), | |
| | 1-carboxylic acid (4-fluoro- | 7.83-7.90 (4H, m), 7.94-7.96 (2H, m), | |
| | phenyl)-amide | 8.79-8.80 (2H, d) | |
| 112 | 5,5-Dimethyl-1-(2- | 0.90 (3H, s), 1.01 (3H, s), | 406 |
| | morpholin-4-yl-ethyl)-2- | 1.62-1.64 (2H, t), 2.37-2.43 (2H, t), | |
| | phenyl-3-[1-pyridin-2-yl- | 3.19-3.22 (2H, t), 3.43-3.48 (4H, t), | |
| | methylidene]-piperidin-4- | 4.13-4.15 (4H, t), 4.49-4.50 (1H, d), 7.12 (1H, | |
| | one | s), 7.19-7.25 (2H, m), | |
| | | 7.28-7.33 (5H, m), 7.37-7.38 (1H, m), | |
| | | 7.47-7.49 (1H, d) | |
| 113 | 5,5-Dimethyl-1-(2- | 0.97 (3H, s), 1.10 (3H, s), | 491 |
| | morpholin-4-yl-ethyl)-3-[1- | 2.19-2.22 (2H, t), 2.30-2.34 (2H, t), 2.64 (2H, | |
| | (6-morpholin-4-yl-pyridin-2- | s), 3.48-3.52 (8H, t), | |
| | yl)-methylidene]-2-phenyl- | 3.54-3.55 (2H, t), 3.59-3.61 (4H, t), | |
| | piperidin-4-one | 3.62-3.65 (2H, t), 6.34 (1H, s), 6.79-6.81 (1H, | |
| | | d), 6.85-6.87 (1H, d), 7.05 (1H, s), | |
| | | 7.17-7.20 (2H, m), 7.28-7.30 (2H, | |
| | | m), 7.34-7.38 (2H, m) | |
| 114 | 1-Benzyl-3-(3,4-dimethoxy- | 3.47 (2H, s), 3.56 (2H, s), 3.72 (3H, | 514 |
| | phenyl)-4-hydroxy-5-[1-(6- | s), 3.79 (3H, s), 6.77 (1H, s), | |
| | morpholin-4-yl-pyridin-2-yl)- | 6.88-6.90 (2H, m), 7.06-7.10 (1H, m), | |
| | methylidene]-5,6-dihydro- | 7.25-7.35 (6H, m), 7.69-7.71 (1H, | |
| | 1H-pyridin-2-one | d) | |
| 115 | 5,5-Dimethyl-1-(2- | 0.98 (3H, s), 1.11 (3H, s), | 505 |
| | morpholin-4-yl-ethyl)-3-[1- | 2.11-2.18 (2H, t), 2.23 (3H, s), 2.53-2.55 (2H, | |
| | (6-morpholin-4-yl-pyridin-2- | t), 2.99 (2H, s), 3.37-3.42 (8H, t), | |
| | yl)-methylidene]-2-p-tolyl- | 3.55-3.57 (8H, t), 6.22 (1H, s), | |
| | piperidin-4-one | 6.71-6.76 (1H, d), 6.77-6.78 (1H, d), | |
| | | 6.96-6.98 (2H, m), 7.03-7.12 (2H, | |
| | | m), 7.16-7.18 (1H, m), | |
| | | 7.46-7.50 (1H, m) | |
| 116 | 4-Hydroxy-1-(4-methyl- | 2.26 (3H, s), 3.44-3.48 (4H, t), | 468 |
| | benzyl)-3-[1-(6-morpholin- | 3.70-3.72 (4H, t), 4.35 (2H, s), 4.74 (2H, | |
| | 4-yl-pyridin-2-yl)- | s), 6.98-7.02 (2H, m), | |
| | methylidene]-5-phenyl-3,6- | 7.10-7.13 (4H, m), 7.18-7.23 (3H, m), | |
| | dihydro-1H-pyridin-2-one | 7.28-7.33 (3H, m), 7.63-7.66 (2H, m) | |
| 117 | 2-(4-Dimethylamino- | 0.91 (6H, s), 1.67 (3H, s), 2.84 (6H, | 525 |
| | phenyl)-5,5-dimethyl-1-(4- | s), 3.22-3.26 (2H, m), 3.56 (2H, s), | |
| | methyl-benzyl)-3-[1-(6- | 3.61-3.63 (4H, t), 4.15-4.25 (4H, t), | |
| | morpholin-4-yl-pyridin-2-yl)- | 5.81 (1H, s), 6.69-6.79 (2H, d), | |
| | methylidene]-piperidin-4- | 6.87-6.92 (2H m), 7.07-7.19 (4H, m), | |
| | one | 7.21-7.23 (2H, m), 7.28-7.30 (1H, | |
| | | d), 7.58-7.73 (1H, m) | |
| 118 | 2-(4-Dimethylamino- | 1.14 (6H, s), 2.33 (3H, s), | 440 |
| | phenyl)-5,5-dimethyl-1-(4- | 2.35-2.45 (1H, d), 2.62-2.65 (1H, d), 2.91 (6H, | |
| | methyl-benzyl)-3-[1-pyridin- | s), 3.30-3.36 (1H, d), | |
| | 2-yl-meth-ylidene]-piperidin- | 3.62-3.66 (1H, d), 6.12 (1H, s), 6.69-6.72 (2H, | |
| | 4-one | d), 7.06 (1H, s), 7.11-7.22 (6H, m), | |
| | | 7.33-7.36 (1H, m), 7.51-7.53 (1H, | |
| | | d), 7.74-7.82 (2H, m), | |
| | | 8.69-8.70 (1H, d) | |
| 119 | 5,5-Dimethyl-2-(4- | 1.04 (3H, s), 1.23 (3H, s), 2.44 (3H, | 520 |
| | methylsulfanyl-phenyl)-3-[1- | s), 2.62-2.72 (2H, m), | |
| | (6-morpholin-4-yl-pyridin-2- | 3.05-3.08 (4H, t), 3.47-3.56 (4H, t), | |
| | yl)-methylidene]-1- | 3.64-3.70 (1H, d), 4.06-4.14 (1H, d), 6.46 (1H, | |
| | thiophen-2-ylmethyl- | s), 6.73-6.76 (1H, d), | |
| | piperidin-4-one | 6.86-6.87 (1H, d), 6.91-6.95 (2H, m), | |
| | | 7.04-7.06 (2H, d), 7.12 (1H, s), | |
| | | 7.20-7.22 (2H, d), 7.43-7.44 (1H, d), | |
| | | 7.50-7.54 (1H, m) | |
| 120 | 2-(2,5-Dimethoxy-phenyl)- | 1.07 (6H, s), 2.25 (3H, s), 2.55 (2H, | 534 |
| | 3-[1-(4-methanesulfonyl- | s), 3.09-3.12 (1H, d), 3.20 (3H, s), | |
| | phenyl)-methylidene]-5,5- | 3.58 (3H, s), 3.61 (3H, s), | |
| | dimethyl-1-(4-methyl- | 3.71-3.78 (1H, d), 5.31 (1H, s), 6.65-6.69 (1H, | |
| | benzyl)-piperidin-4-one | m), 6.89-6.95 (1H, m), | |
| | | 6.95-7.15 (5H, m), 7.26 (1H, s), 7.44-7.49 (2H, | |
| | | d), 7.80-7.85 (2H, d) | |
| 121 | 2-(2,5-Dimethoxy-phenyl)- | 1.04 (6H, s), 2.25 (3H, s), 2.51 (3H, | 502 |
| | 5,5-dimethyl-1-(4-methyl- | s), 2.55 (2H, s), 3.09-3.12 (1H, d), | |
| | benzyl)-3-[1-(4- | 3.69 (6H, s), 3.71-3.78 (1H, d), | |
| | methylsulfanyl-phenyl)- | 5.31 (1H, s), 6.65-6.69 (1H, m), | |
| | methylidene]-piperidin-4- | 6.89-6.95 (1H, m), 6.95-7.15 (5H, | |
| | one | m), 7.26 (1H, s), 7.44-7.49 (2H, d), | |
| | | 7.80-7.85 (2H, d) | |
| 122 | N-(4-{1-Benzyl-4-hydroxy- | 3.00 (3H, s), 3.50-3.52 (4H, t), | 547 |
| | 5-[1-(6-morpholin-4-yl- | 3.72-3.74 (4H, t), 4.35 (2H, s), 4.80 (2H, | |
| | pyridin-2-yl)-methylidene]- | s), 7.01-7.03 (1H, d), | |
| | 6-oxo-1,2,5,6-tetrahydro- | 7.12-7.18 (3H, m), 7.28-7.38 (5H, m), | |
| | pyridin-3-yl}-phenyl)- | 7.63-7.78 (4H, m), 9.78 (1H, bs), | |
| | methanesulfonamide | 14.69 (1H, bs) | |
| 123 | 1-Benzyl-5-(3,5-dimethyl- | 2.25 (6H, s), 3.90 (1H, s), 4.05 (2H, | 397 |
| | phenyl)-3-[1-pyridin-2-yl- | s), 5.11 (2H, s), 6.88 (1H, s), | |
| | methylidene]-piperidine-2,4- | 7.05 (2H, s), 7.25-7.34 (4H, m), | |
| | dione | 7.67-7.71 (4H, m), 7.73-7.86 (1H, m), | |
| | | 8.50-8.51 (1H, m) | |
| 124 | 1-Methanesulfonyl-3-[1-(6- | 2.86 (3H, s), 3.68-3.69 (4H, t), | 442 |
| | morpholin-4-yl-pyridin-2-yl)- | 3.71 (2H, s), 4.18-4.20 (4H, t), 4.23 (1H, | |
| | methylidene]-5-phenyl- | s), 6.63-6.65 (1H, d), | |
| | piperidine-2,4-dione | 6.71-6.73 (1H, d), 7.54-7.63 (6H, m), | |
| | | 8.49 (1H, s) | |
| 125 | 2-(4-Dimethylamino- | 0.97 (6H, s), 2.26 (3H, s), 2.83 (6H, | 490 |
| | phenyl)-5,5-dimethyl-1-(4- | s), 3.27-3.28 (1H, d), | |
| | methyl-benzyl)-3-[1- | 3.39-3.40 (1H, d), 4.25 (1H, s), 6.59-6.61 (1H, | |
| | quinolin-2-yl-methylidene]- | d), 7.06-7.10 (3H, m), | |
| | piperidin-4-one | 7.14-7.22 (4H, m), 7.25-7.30 (2H, d), | |
| | | 7.37-7.41 (1H, m), 7.47-7.51 (1H, m), | |
| | | 7.79-7.81 (1H, d), 7.85-7.87 (1H, | |
| | | d), 8.14-8.16 (1H, d) | |
| 126 | 1-Benzoyl-4-hydroxy-5- | 4.07 (2H, s), 7.20 (1H, s), | 383 |
| | phenyl-3-[1-pyridin-2-yl- | 7.27-7.29 (1H, m), 7.33-7.51 (7H, m), | |
| | methylidene]-3,6-dihydro- | 7.52-7.53 (2H, t), 7.61-7.63 (1H, m), | |
| | 1H-pyridin-2-one | 7.84-7.86 (1H, m), 7.88-7.96 (1H, m), | |
| | | 8.50-8.51 (1H, m), 11.37 (1H, bs) | |
| 127 | 2-(4-Fluoro-phenyl)-5,5- | 1.23 (3H, s), 1.24 (3H, s), 2.33 (3H, | 500 |
| | dimethyl-1-(4-methyl- | s), 2.54-2.68 (2H, m), | |
| | benzyl)-3-[1-(6-morpholin- | 3.03-3.07 (4H, t), 3.49-3.56 (4H, t), | |
| | 4-yl-pyridin-2-yl)- | 3.64-3.83 (2H, m), 6.33 (1H, s), 6.68-6.70 (1H, | |
| | methylidene]-piperidin-4- | d), 6.88-6.90 (1H, d), | |
| | one | 7.07-7.17 (9H, m), 7.48-7.56 (1H, m) | |
| 128 | 4-Hydroxy-1-(4-methyl- | 2.21 (2H, s), 2.23 (3H, s), 3.49 (2H, | 383 |
| | benzyl)-5-phenyl-3-[1- | s), 7.13-7.36 (9H, m), | |
| | pyridin-2-yl-methylidene]- | 7.61-7.68 (5H, m), 14.84 (1H, s), | |
| | 3,6-dihydro-1H-pyridin-2- | | |
| | one | | |
| 129 | 1-(4-Methyl-benzyl)-3-[1-(4- | 2.28 (3H, s), 3.26 (3H, s), | 428 |
| | methylsulfanyl-phenyl)- | 3.58-3.63 (1H, m), 3.80-3.92 (1H, m), | |
| | methylidene]-5-phenyl- | 4.01-4.05 (1H, m), 4.10-4.18 (2H, m), | |
| | piperidine-2,4-dione | 7.10-7.18 (4H, m), 7.20-7.37 (9H, | |
| | | m), 7.81-7.84 (1H, m), | |
| | | 7.96-7.98 (1H, d) | |
| 130 | 1-(3-Methoxy-benzyl)-5- | 3.52-3.56 (2H, t), 3.57-3.62 (2H, t), | 399 |
| | phenyl-3-[1-pyridin-2-yl- | 3.80 (3H, s), 3.90-3.94 (1H, m), | |
| | methylidene]-piperidine-2,4- | 6.82-7.09 (6H, m), 7.13-7.27 (5H, | |
| | dione | m), 7.37-7.77 (2H, m), | |
| | | 7.86-7.90 (1H, m) | |
| 131 | 5,5-Dimethyl-3-[1-(6- | 1.03 (3H, s), 1.18 (3H, s), | 489 |
| | morpholin-4-yl-pyridin-2-yl)- | 1.32-1.43 (6H, m), 2.25-2.50 (4H, m), | |
| | methylidene]-2-phenyl-1-(2- | 2.63-2.67 (2H, m), 3.44-3.54 (4H, t), | |
| | piperidin-1-yl-ethyl)- | 3.61-3.72 (6H, m), 4.13-4.15 (3H, m), | |
| | piperidin-4-one | 6.33-6.34 (1H, m), 6.46-6.52 (1H, | |
| | | m), 6.54-6.58 (1H, m), | |
| | | 6.78-6.87 (2H, m), 7.12-7.57 (5H, m) | |
| 132 | 2-(4-Fluoro-phenyl)-5,5- | 1.03 (3H, s), 1.17 (3H, s), | 507 |
| | dimethyl-3-[1-(6-morpholin- | 1.24-1.40 (6H, m), 2.16-2.31 (2H, m), | |
| | 4-yl-pyridin-2-yl)- | 2.35-2.38 (4H, m), 2.63 (2H, s), | |
| | methylidene]-1-(2-piperidin- | 2.80-2.88 (2H, m), 3.06-3.36 (4H, t), | |
| | 1-yl-ethyl)-piperidin-4-one | 3.62-3.70 (4H, t), 6.39 (1H, s), | |
| | | 6.79-6.81 (1H, d), 6.83-6.88 (1H, m), | |
| | | 7.11 (1H, s), 7.12-7.19 (4H, m), | |
| | | 7.54-7.56 (1H, m) | |
| 133 | 5,5-Dimethyl-2-phenyl-1-(2- | 1.13 (3H, s), 1.16 (3H, s), | 404 |
| | piperidin-1-yl-ethyl)-3-[1- | 1.29-1.69 (6H, m), 2.14-2.34 (5H, m), | |
| | pyridin-2-yl-methylidene]- | 2.39-2.56 (2H, m), 3.81-3.82 (2H, m), | |
| | piperidin-4-one | 6.20 (1H, s), 7.03 (1H, s), | |
| | | 7.28-7.39 (6H, m), 7.45-7.52 (1H, m), | |
| | | 7.54-7.74 (2H, m) | |
| 134 | 2-(4-Fluoro-phenyl)-5,5- | 0.89 (3H, s), 1.12 (3H, s), | 422 |
| | dimethyl-1-(2-piperidin-1-yl | 1.22-1.45 (6H, m), 2.08-2.29 (4H, m), | |
| | ethyl)-3-[1-pyridin-2-yl- | 2.50-2.63 (4H, m), 3.21-3.22 (2H, t), | |
| | methylidene]-piperidin-4- | 6.11 (1H, s), 6.90 (1H, s), 6.95-7.06 (2H, | |
| | one | t), 7.17-7.27 (4H, m), | |
| | | 7.44-7.48 (1H, d), 8.65-8.70 (1H, m) | |
| 135 | 5,5-Dimethyl-3-[1-(6- | 1.04 (3H, s), 1.19 (3H, s), | 503 |
| | morpholin-4-yl-pyridin-2-yl)- | 1.25-1.51 (10H, m), 2.28 (3H, s), | |
| | methylidene]-1-(2-piperidin- | 2.31-2.38 (4H, t), 2.44-2.51 (2H, m), | |
| | 1-yl-ethyl)-2-p-tolyl- | 3.25-3.29 (5H, t), 3.61-3.71 (4H, t), | |
| | piperidin-4-one | 6.21 (1H, s), 6.66-6.68 (1H, d), | |
| | | 6.78-6.80 (1H, d), 6.97-7.11 (5H, m), | |
| | | 7.52-7.64 (1H, m) | |
| 136 | 2-(4-Dimethylamino- | 1.08 (3H, s), 1.19 (3H, s), | 447 |
| | phenyl)-5,5-dimethyl-1-(2- | 1.23-1.34 (6H, m), 2.51-2.56 (2H, m), | |
| | piperidin-1-yl-ethyl)-3-[1- | 2.85 (6H, s), 2.88-2.95 (6H, m), | |
| | pyridin-2-yl-methylidene]- | 3.42-3.72 (2H, m), 6.01 (1H, s), | |
| | piperidin-4-one | 6.55-6.76 (4H, m), 6.92-7.01 (2H, m), | |
| | | 7.21-7.46 (3H, m) | |
| 137 | 5,5-Dimethyl-3-[1-[6-(4- | 1.03 (3H, s), 1.18 (3H, s), | 516 |
| | methyl-piperazin-1-yl)- | 1.24-1.41 (6H, m), 2.19 (3H, s), 2.89-2.33 (8H, | |
| | pyridin-2-yl]-methylidene]-1- | m), 2.43-2.44 (2H, m), | |
| | (2-piperidin-1-yl-ethyl)-2-p- | 2.63-2.81 (2H, m), 3.34 (3H, s), 4.10-4.15 (6H, | |
| | tolyl-piperidin-4-one | m), 6.41 (1H, s), 6.78-6.83 (2H, m), | |
| | | 7.02 (1H, s), 7.15-7.31 (5H, m) | |
| 138 | 5,5-Dimethyl-1-(2- | 1.07 (3H, s), 1.32 (3H, s), | 505 |
| | morpholin-4-yl-2-oxo-ethyl)- | 2.16-2.20 (2H, t), 2.67-2.75 (2H, d), | |
| | 3-[1-(6-morpholin-4-yl- | 3.08-3.15 (2H, t), 3.44-3.56 (8H, t), | |
| | pyridin-2-yl)-methylidene]- | 3.57-3.75 (8H, t), 6.5 (1H, s), | |
| | 2-phenyl-piperidin-4-one | 6.84-6.92 (1H, m), 7.33-7.40 (2H, m), | |
| | | 7.48-7.54 (1H, m), 7.65-7.73 (3H, m), | |
| | | 7.96-7.98 (1H, d) | |
| 139 | 5,5-Dimethyl-1-(2-piperidin- | 1.04 (3H, s), 1.18 (3H, s), | 418 |
| | 1-yl-ethyl)-3-[1-pyridin-2-yl- | 1.24-1.42 (9H, m), 2.24 (3H, s), 2.28-2.29 (2H, | |
| | methylidene]-2-p-tolyl- | t), 2.64-2.81 (2H, t), 3.60-3.64 (3H, | |
| | piperidin-4-one | t), 6.17 (1H, s), 6.83-6.88 (2H, d), | |
| | | 7.01-7.03 (3H, d), 7.11-7.16 (3H, | |
| | | d), 7.45-7.54 (1H, t), | |
| 140 | 2-(4-Fluoro-phenyl)-5,5- | 1.03 (3H, s), 1.18 (3H, s), | 520 |
| | dimethyl-3-[1-[6-(4-methyl- | 1.19-1.39 (6H, m), 2.2 (3H, s), 2.20-2.33 (8H, | |
| | piperazin-1-yl)-pyridin-2-yl]- | m), 2.53-2.61 (4H, m), 6.37 (1H, s), | |
| | methylidene]-1-(2-piperidin- | 6.74-6.84 (2H, m), 7.05 (1H, s), | |
| | 1-yl-ethyl)-piperidin-4-one | 7.13-7.30 (4H, m), 7.51-7.55 (1H, | |
| | | m) | |
| 141 | 3,3-Dimethyl-5-[1-quinolin- | 0.78 (6H, s), 2.95 (2H, s), 3.57 (2H, | 363 |
| | 2-yl-methylidene]-1- | s), 4.67 (2H, s), 6.97 (1H, s), | |
| | thiophen-2-ylmethyl- | 7.18-7.29 (2H, s), 7.37-7.47 (3H, m), | |
| | piperidin-4-one | 7.52-7.66 (2H, m), 7.75-7.85 (2H, | |
| | | m), 8.07-8.14 (1H, m) | |
| 142 | 3,3-Dimethyl-5-[1-[6-(4- | 1.07 (6H, s), 2.22 (3H, s), | 411 |
| | methyl-piperazin-1-yl)- | 2.30-2.39 (4H, t), 2.61 (2H, s), 3.36-3.41 (4H, | |
| | pyridin-2-yl]-methylidene]-1- | t), 3.86 (2H, s), 4.11 (2H, s), | |
| | thiophen-2-ylmethyl- | 6.81-6.83 (1H, d), 6.90-6.93 (1H, d), | |
| | piperidin-4-one | 6.97-7.00 (2H, m), 7.13 (1H, s), | |
| | | 7.43-7.44 (1H, m), 7.54-7.58 (1H, m) | |
| 143 | 3,3-Dimethyl-5-[1-pyridin-2- | 1.09 (6H, s), 2.25 (2H, s), 3.86 (2H, | 313 |
| | yl-methylidene]-1-thiophen- | s), 4.12 (2H, s), 6.87-7.02 (3H, m), | |
| | 2-ylmethyl-piperidin-4-one | 7.33 (1H, s), 7.36-7.42 (1H, m), | |
| | | 7.64-7.66 (1H, d), 7.82-7.87 (1H, | |
| | | m), 8.67-8.68 (1H, d) | |
| 144 | 5,5-Dimethyl-3-[1-pyridin-2- | 1.07 (3H, s), 1.17 (3H, s), 2.28 (3H, | 403 |
| | yl-methylidene]-1-thiophen- | s), 2.31-2.39 (2H, m), | |
| | 2-ylmethyl-2-p-tolyl- | 3.32-3.46 (2H, m), 5.29 (1H, s), 6.89-6.96 (2H, | |
| | piperidin-4-one | m), 7.069-7.089 (2H, m), | |
| | | 7.13-7.15 (2H, m), 7.17-7.22 (2H, m), | |
| | | 7.24-7.26 (2H, m), 7.29 (1H, s), | |
| | | 7.40-7.41 (1H, d) | |
| 145 | 5,5-Dimethyl-3-[1-(6- | 1.04 (3H, s), 1.13 (3H, s), 2.25 (3H, s), | 488 |
| | morpholin-4-yl-pyridin-2-yl)- | 2.39-2.42 (1H, m), 3.26-3.29 (4H, | |
| | methylidene]-1-thiophen-2- | t), 3.36-3.42 (4H, t), 3.45-3.46 (2H, | |
| | ylmethyl-2-p-tolyl-piperidin- | d), 3.80-4.14 (1H, d), 5.29 (1H, s), | |
| | 4-one | 6.89-6.93 (2H, m), 7.06-7.09 (2H, | |
| | | d), 7.15-7.19 (2H, m), | |
| | | 7.22-7.44 (5H, m) | |
| 146 | 5,5-Dimethyl-3-[1-(6- | 1.04 (3H, s), 1.13 (3H, s), | 474 |
| | morpholin-4-yl-pyridin-2-yl)- | 2.39-2.42 (1H, m), 3.26-3.29 (4H, t), | |
| | methylidene]-2-phenyl-1- | 3.36-3.42 (4H, t), 3.45-3.46 (2H, d), | |
| | thiophen-2-ylmethyl- | 3.80-4.14 (1H, d), 5.29 (1H, s), | |
| | piperidin-4-one | 6.89-6.93 (3H, m), 7.06-7.09 (2H, d), | |
| | | 7.15-7.19 (2H, m), 7.22-7.44 (5H, | |
| | | m) | |
| 147 | 5,5-Dimethyl-2-phenyl-3-[1- | 1.04 (3H, s), 1.13 (3H, s), | 389 |
| | pyridin-2-yl-methylidene]-1- | 2.39-2.42 (1H, m), 3.45-3.46 (2H, d), | |
| | thiophen-2-ylmethyl- | 3.80-4.14 (1H, d), 5.29 (1H, s), | |
| | piperidin-4-one | 6.89-6.93 (2H, m), 7.06-7.09 (3H, d), | |
| | | 7.15-7.19 (2H, m), 7.22-7.44 (5H, m) | |
| 148 | 2-(2,5-Dimethoxy-phenyl)- | 1.14 (3H, s), 1.18 (3H, s), 2.27 (3H, | 507 |
| | 5,5-dimethyl-1-(4-methyl- | s), 2.30-2.36 (1H, d), | |
| | benzyl)-3-[1-quinolin-2-yl- | 2.75-2.78 (1H, d), 3.34-3.40 (1H, d), 3.58 (3H, | |
| | methylidene]-piperidin-4- | s), 3.63 (3H, s), 3.72-3.79 (1H, d), | |
| | one | 6.45 (1H, d), 6.70-6.86 (3H, m), | |
| | | 7.00-7.02 (2H, d), 7.20-7.29 (4H, | |
| | | m), 7.41 (1H, s), 7.46-7.54 (1H, m), | |
| | | 7.65-7.73 (2H, m), 7.93-8.00 (1H, | |
| | | m) | |
| 149 | 2-(4-Dimethylamino- | 1.16 (6H, s), 2.57-2.60 (1H, d), | 446 |
| | phenyl)-5,5-dimethyl-3-[1- | 2.73-2.74 (2H, t), 2.82 (6H, s), | |
| | pyridin-2-yl-methylidene]-1- | 2.86-2.87 (1H, d), 2.92-2.98 (2H, t), | |
| | (2-thiophen-2-yl-ethyl)- | 6.15 (1H, s), 6.59-6.61 (2H, d), | |
| | piperidin-4-one | 6.80-6.86 (2H, m), 6.88-7.03 (3H, m), | |
| | | 7.24-7.28 (2H, m), 7.45-7.47 (1H, | |
| | | d), 7.66-7.76 (2H, m), | |
| | | 8.63-8.64 (1H, d) | |
| 150 | 2-(4-Dimethylamino- | 1.16 (6H, s), 2.57-2.60 (1H, d), | 531 |
| | phenyl)-5,5-dimethyl-3-[1- | 2.73-2.74 (2H, t), 2.82 (6H, s), | |
| | (6-morpholin-4-yl-pyridin-2- | 2.86-2.87 (1H, d), 2.92-2.98 (2H, t), | |
| | yl)-methylidene]-1-(2- | 3.38-3.43 (4H, t), 3.63-3.68 (4H, t), | |
| | thiophen-2-yl-ethyl)- | 6.15 (1H, s), 6.59-6.61 (2H, d), | |
| | piperidin-4-one | 6.80-6.86 (2H, m), 6.88-7.03 (3H, m), | |
| | | 7.24-7.28 (2H, m), 7.45-7.47 (1H, | |
| | | d), 7.66-7.76 (2H, m) | |
| 151 | 1-Benzyl-3-(3,4-dimethoxy- | 3.29 (2H, s), 3.51-3.59 (1H, m), | 429 |
| | phenyl)-5-[1-pyridin-2-yl- | 3.72 (6H, s), 3.82-3.89 (2H, m), | |
| | methylidene]-piperidine-2,4- | 4.25-4.27 (2H, d), 6.79-6.77 (1H, | |
| | dione | d), 6.86-6.91 (3H, m), | |
| | | 7.22-7.39 (8H, m), 8.48-8.49 (1H, m) | |
| 152 | 3,3-Dimethyl-5-[1-[6-(4- | 1.23 (6H, s), 2.21 (2H, s), 2.39 (4H, | 425 |
| | methyl-piperazin-1-yl)- | t), 2.70 (3H, t), 2.76 (3H, s), 3.44 (3H, | |
| | pyridin-2-yl]-methylidene]-1- | t), 3.49-3.518 (4H, t), | |
| | (2-thiophen-2-yl-ethyl)- | 6.69-6.71 (1H, d), 6.88-6.90 (1H, d), | |
| | piperidin-4-one | 6.98-6.99 (1H, d), 7.21-7.22 (1H, d), | |
| | | 7.32-7.36 (1H, d), 7.54-7.66 (2H, m) | |
| 153 | 5,5-Dimethyl-2-(4- | 1.24 (3H, s), 1.25 (3H, s), 2.48 (3H, | 534 |
| | methylsulfanyl-phenyl)-3-[1- | s), 2.57-2.71 (2H, d), | |
| | (6-morpholin-4-yl-pyridin-2- | 2.80-2.85 (2H, t), 3.60-3.62 (2H, t), | |
| | yl)-methylidene]-1-(2- | 3.67-3.70 (4H, t), 3.73-3.78 (4H, t), 6.50 (1H, | |
| | thiophen-2-yl-ethyl)- | s), 6.83-6.93 (1H, m), | |
| | piperidin-4-one | 6.91-6.93 (1H, m), 6.96-6.99 (1H, m), | |
| | | 7.08 (1H, s), 7.10-7.11 (1H, m), | |
| | | 7.22-7.24 (1H, m), 7.25-7.26 (1H, m), | |
| | | 7.31-7.35 (2H, m), 7.59-7.63 (1H, | |
| | | t), 7.82-7.86 (1H, t) | |
| 154 | 2-(4-Dimethylamino- | 1.15 (6H, s), 2.46-2.47 (1H, d), | 432 |
| | phenyl)-5,5-dimethyl-3-[1- | 2.64-2.67 (1H, d), 2.85 (6H, s), | |
| | pyridin-2-yl-methylidene]-1- | 3.53-3.57 (1H, d), 3.90-3.94 (1H, d), | |
| | thiophen-2-ylmethyl- | 6.16 (1H, s), 6.65-6.67 (2H, d), | |
| | piperidin-4-one | 6.93-6.94 (2H, m), 7.04-7.06 (3H, m), | |
| | | 7.28-7.31 (1H, m), 7.41-7.48 (2H, | |
| | | m), 7.75-7.78 (1H, m), | |
| | | 8.62-8.63 (1H, d) | |
| 155 | 2-(4-Dimethylamino- | 1.15 (6H, s), 2.46-2.47 (1H, d), | 517 |
| | phenyl)-5,5-dimethyl-3-[1- | 2.64-2.67 (1H, d), 2.85 (6H, s), | |
| | (6-morpholin-4-yl-pyridin-2- | 3.22-3.26 (4H, t), 3.51-3.52 (4H, t), | |
| | yl)-methylidene]-1- | 3.53-3.57 (1H, d), 3.90-3.94 (1H, d), | |
| | thiophen-2-ylmethyl- | 6.16 (1H, s), 6.65-6.67 (2H, d), | |
| | piperidin-4-one | 6.93-6.94 (2H, m), 7.04-7.06 (1H, m), | |
| | | 7.28-7.31 (1H, m), 7.41-7.48 (2H, | |
| | | m), 7.75-7.78 (1H, m), | |
| | | 8.62-8.63 (1H, d) | |
| 156 | 1-Benzyl-3-(3,4-dimethoxy- | 2.17 (3H, s), 2.28-2.36 (4H, t), | 527 |
| | phenyl)-5-[1-[6-(4-methyl- | 3.08-3.14 (4H, t), 3.45 (2H, s), 3.60 (1H, | |
| | piperazin-1-yl)-pyridin-2-yl]- | s), 3.67 (6H, s), 4.13 (2H, s), | |
| | methylidene]-piperidine-2,4- | 6.76-6.92 (3H, m), 7.15-7.39 (8H, m), | |
| | dione | 7.99 (1H, s) | |
| 157 | 5,5-Dimethyl-2-(4- | 1.20 (6H, s), 2.42 (3H, s), | 449 |
| | methylsulfanyl-phenyl)-3-[1- | 2.56-2.84 (4H, m), 2.96-3.02 (2H, m), | |
| | pyridin-2-yl-methylidene]-1- | 6.25 (1H, s), 6.81-6.88 (1H, m), | |
| | (2-thiophen-2-yl-ethyl)- | 6.89-6.95 (1H, m), 6.97-6.99 (1H, m), | |
| | piperidin-4-one | 7.08 (1H, s), 7.11-7.18 (3H, m), | |
| | | 7.26-7.31 (2H, m), 7.51-7.53 (1H, | |
| | | m), 7.72-7.78 (1H, m), | |
| | | 8.65-8.66 (1H, d) | |
| 158 | 5,5-Dimethyl-3-[1-quinolin- | 1.07 (3H, s), 1.17 (3H, s), | 453 |
| | 2-yl-methylidene]-1- | 2.25-2.26 (2H, d), 2.28 (3H, s), 2.36-2.39 (1H, | |
| | thiophen-2-ylmethyl-2-p- | d), 3.34-3.37 (1H, d), 6.48 (1H, s), | |
| | tolyl-piperidin-4-one | 6.87-6.96 (3H, m), 7.07-7.09 (2H, | |
| | | d), 7.13-7.15 (2H, d), | |
| | | 7.17-7.29 (5H, m), 7.36-7.41 (2H, m) | |
| 159 | 5,5-Dimethyl-2-(4- | 1.07 (3H, s), 1.17 (3H, s), | 485 |
| | methylsulfanyl-phenyl)-3-[1- | 2.25-2.26 (2H, d), 2.27 (3H, s), 2.36-2.39 (1H, | |
| | quinolin-2-yl-methylidene]- | d), 3.34-3.37 (1H, d), 6.48 (1H, s), | |
| | 1-thiophen-2-ylmethyl- | 6.87-6.96 (3H, m), 7.07-7.09 (2H, | |
| | piperidin-4-one | d), 7.13-7.15 (2H, d), | |
| | | 7.17-7.29 (5H, m), 7.36-7.41 (2H, m) | |
| 160 | 2-(4-Dimethylamino- | 1.05 (6H, s), 2.13-2.17 (2H, t), | 496 |
| | phenyl)-5,5-dimethyl-3-[1- | 2.81 (6H, s), 2.98-3.01 (2H, t), | |
| | quinolin-2-yl-methylidene]- | 3.47-3.49 (2H, d), 5.29 (1H, s), | |
| | 1-(2-thiophen-2-yl-ethyl)- | 6.53-6.56 (2H, d), 6.69-6.83 (1H, d), | |
| | piperidin-4-one | 6.89-7.10 (3H, m), 7.12-7.27 (4H, m), | |
| | | 7.45-7.47 (1H, m), 7.66-7.67 (1H, | |
| | | d), 7.68-7.70 (1H, d), | |
| | | 8.09-8.12 (1H, d) | |
| 161 | 5,5-Dimethyl-2-(4- | 1.05 (6H, s), 2.13-2.17 (2H, t), | 499 |
| | methylsulfanyl-phenyl)-3-[1- | 2.48 (3H, s), 2.98-3.01 (2H, t), | |
| | quinolin-2-yl-methylidene]- | 3.47-3.49 (2H, d), 5.29 (1H, s), | |
| | 1-(2-thiophen-2-yl-ethyl)- | 6.53-6.56 (2H, d), 6.69-6.83 (1H, d), | |
| | piperidin-4-one | 6.89-7.10 (3H, m), 7.12-7.27 (4H, m), | |
| | | 7.45-7.47 (1H, m), 7.66-7.67 (1H, | |
| | | d), 7.68-7.70 (1H, d), | |
| | | 7.96-7.812 (1H, d) | |
| 162 | 5,5-Dimethyl-3-[1-(6- | 1.03 (3H, s), 1.17 (3H, s), 2.21 (3H, | 502 |
| | morpholin-4-yl-pyridin-2-yl)- | s), 2.25-2.29 (2H, t), 2.60 (2H, s), | |
| | methylidene]-1-(2-thiophen- | 2.96-3.15 (4H, t), 3.38-3.39 (2H, t), | |
| | 2-yl-ethyl)-2-p-tolyl- | 3.56-3.61 (4H, t), 6.35 (1H, s), | |
| | piperidin-4-one | 6.76-6.94 (4H, m), 6.96-7.01 (2H, t), | |
| | | 7.07-7.15 (3H, m), 7.22-7.25 (1H, | |
| | | d), 7.43-7.54 (1H, t) | |
| 163 | 2-(2,5-Dimethoxy-phenyl)- | 1.07 (6H, s), 2.28 (3H, s), | 458 |
| | 5,5-dimethyl-1-(4-methyl- | 2.61-2.64 (1H, d), 3.18-3.21 (1H, s), | |
| | benzyl)-3-[1-pyrazin-2-yl- | 3.48-3.50 (1H, s), 3.53 (3H, s), 3.73 (3H, | |
| | methylidene]-piperidin-4- | s), 3.75-3.76 (1H, d), 6.10 (1H, s), | |
| | one | 6.78-6.80 (2H, m), 6.81-6.93 (1H, | |
| | | d), 7.07-7.21 (5H, m), | |
| | | 8.45-8.46 (1H, d), 8.59-8.60 (1H, d), | |
| | | 8.70-8.71 (1H, d) | |
| 164 | 5,5-Dimethyl-3-[1-pyridin-2- | 1.10 (6H, s), 2.14 (3H, s), | 417 |
| | yl-methylidene]-1-(2- | 2.55-2.58 (1H, s), 2.67-2.70 (2H, t), | |
| | thiophen-2-yl-ethyl)-2-p- | 2.87-2.90 (3H, m), 6.10 (1H, s), | |
| | tolyl-piperidin-4-one | 6.72-6.73 (1H, d), 6.80-6.82 (1H, m), | |
| | | 6.96-7.01 (5H, m), 7.17-7.22 (2H, | |
| | | m), 7.41-7.43 (1H, d), | |
| | | 7.66-7.71 (1H, m), 8.57-8.58 (1H, d) | |
| 165 | 1-Benzyl-3-(3,4-dimethyl- | 2.14 (3H, s), 2.16 (3H, s), 2.26 (2H, | 482 |
| | phenyl)-5-[1-(6-morpholin- | s), 3.18-3.22 (4H, t), | |
| | 4-yl-pyridin-2-yl)- | 3.53-3.59 (4H, t), 3.90 (2H, s), 5.08 (1H, s), | |
| | methylidene]-piperidine-2,4- | 6.65-6.67 (1H, d), 6.74-6.77 (2H, | |
| | dione | d), 6.80-6.87 (2H, m), | |
| | | 6.93-6.95 (1H, d), 7.03-7.09 (2H, m), | |
| | | 7.24-7.32 (4H, m) | |
| 166 | 1-Benzyl-5,5-dimethyl-3-[1- | 1.11 (3H, s), 1.15 (3H, s), | 429 |
| | (4-methylsulfanyl-phenyl)- | 2.45-2.48 (2H, d), 2.55 (3H, s), 3.27-3.30 (1H, | |
| | methylidene]-2,3,5,6- | d), 3.93-3.96 (1H, d), 5.45 (1H, s), | |
| | tetrahydro-1H- | 7.19-7.36 (6H, m), 7.37-7.38 (2H, | |
| | [2,3′]bipyridinyl-4-one | d), 7.39-7.40 (2H, d), | |
| | | 7.48-7.51 (1H, m), 7.62-7.64 (1H, m), | |
| | | 8.56-8.58 (2H, m) | |
| 167 | 1-Benzyl-5,5-dimethyl-3-[1- | 1.09 (3H, s), 1.11 (3H, s), | 451 |
| | (4-trifluoromethyl-phenyl)- | 3.26-3.27 (2H, d), 3.29-3.36 (1H, d), 4.59 (1H, | |
| | methylidene]-2,3,5,6- | s), 5.27 (1H, s), 7.20-7.28 (3H, m), | |
| | tetrahydro-1H- | 7.37-7.44 (2H, m), 7.51-7.55 (1H, | |
| | [2,3′]bipyridinyl-4-one | t), 7.67-7.70 (2H, m), | |
| | | 7.87-7.89 (2H, d), 8.13-8.15 (2H, d), | |
| | | 8.33-8.34 (1H, d), 8.47-8.48 (1H, d), | |
| 168 | 1-(2-Fluoro-benzyl)-5,5- | 1.06 (6H, s), 2.42 (3H, s), | 447 |
| | dimethyl-2-(4- | 2.43-2.44 (1H, d), 2.61-2.64 (1H, d), | |
| | methylsulfanyl-phenyl)-3-[1- | 3.37-3.42 (1H, d), 3.46-3.72 (1H, d), | |
| | pyridin-2-yl-methylidene]- | 6.22 (1H, s), 7.06 (1H, d), 7.11-7.21 (6H, | |
| | piperidin-4-one | m), 7.27-7.31 (2H, m), | |
| | | 7.35-7.38 (1H, m), 7.51-7.59 (1H, d), | |
| | | 7.71-7.79 (1H, m), 8.61-8.62 (1H, d) | |
| 169 | 1-(2-Fluoro-benzyl)-5,5- | 1.06 (6H, s), 2.43-2.44 (1H, d), | 401 |
| | dimethyl-2-phenyl-3-[1- | 2.61-2.64 (1H, d), 3.37-3.42 (1H, d), | |
| | pyridin-2-yl-methylidene]- | 3.46-3.72 (1H, d), 6.22 (1H, s), | |
| | piperidin-4-one | 7.06 (1H, d), 7.11-7.21 (6H, m), | |
| | | 7.27-7.31 (3H, m), 7.35-7.38 (1H, m), | |
| | | 7.51-7.59 (1H, d), 7.71-7.79 (1H, | |
| | | m), 8.61-8.62 (1H, d) | |
| 170 | 1-(2-Fluoro-benzyl)-5,5- | 0.99 (3H, s), 1.19 (3H, s), | 486 |
| | dimethyl-3-[1-(6-morpholin- | 2.43-2.44 (1H, d), 2.61-2.64 (1H, d), | |
| | 4-yl-pyridin-2-yl)- | 3.00-3.09 (4H, t), 3.37-3.42 (1H, d), | |
| | methylidene]-2-phenyl- | 3.46-3.72 (1H, d), 3.92-3.54 (4H, t), | |
| | piperidin-4-one | 6.22 (1H, s), 7.06 (1H, d), | |
| | | 7.11-7.21 (6H, m), 7.27-7.31 (2H, m), | |
| | | 7.35-7.38 (1H, m), 7.51-7.59 (1H, d), | |
| | | 7.71-7.79 (1H, m), 8.61-8.62 (1H, | |
| | | d) | |
| 171 | {5,5-Dimethyl-3-[1-(6- | 1.15 (3H, s), 1.22 (3H, s), 2.23 (3H, | 450 |
| | morpholin-4-yl-pyridin-2-yl)- | s), 2.68-2.71 (1H, d), | |
| | methylidene]-4-oxo-2-p- | 2.84-2.87 (1H, d), 3.28 (2H, s), 3.39-3.49 (4H, | |
| | tolyl-piperidin-1-yl}-acetic | t), 3.66-3.75 (4H, t), 6.51 (1H, s), | |
| | acid | 6.72-6.91 (2H, m), 7.02 (1H, s), | |
| | | 7.08-7.19 (3H, m), | |
| | | 7.50-7.21-7.27 (1H, m), 7.60 (1H, m) | |
| 172 | 1-Benzyl-3-[1-(6-morpholin- | 3.05-3.08 (1H, d), 3.25-3.28 (1H, | 440 |
| | 4-yl-pyridin-2-yl)- | d), 3.33-3.39 (4H, t), 3.70 (2H, s), | |
| | methylidene]-5-phenyl- | 3.74-3.79 (4H, t), 4.14 (1H, s), | |
| | piperidin-4-one | 4.51-4.55 (1H, d), 6.60-6.63 (1H, d), | |
| | | 6.89-6.90 (1H, d), 7.18-7.20 (2H, | |
| | | m), 7.23-7.30 (4H, m), | |
| | | 7.32-7.40 (3H, m), 7.43-7.56 (3H, m) | |
| 173 | 1-Benzyl-3-phenyl-5-[1- | 2.87-2.92 (1H, m), 3.09-3.18 (1H, | 355 |
| | pyridin-2-yl-methylidene]- | m), 3.65-3.76 (2H, m), | |
| | piperidin-4-one | 3.88-4.02 (2H, m), 4.43-4.47 (1H, d), | |
| | | 7.17-7.41 (12H, m), 7.83-7.88 (2H, m), | |
| | | 8.66-8.67 (1H, d) | |
| 174 | 4-Oxo-3-phenyl-5-[1- | 3.84-3.90 (1H, m), 4.07-4.11 (1H, | 418 |
| | pyridin-2-yl-methylidene]- | m), 4.28-4.33 (1H, m), | |
| | piperidine-1-carboxylic acid | 5.04-5.09 (1H, m), 5.43-5.47 (1H, d), | |
| | (4-chloro-phenyl)-amide | 7.22 (1H, s), 7.25-7.30 (4H, m), | |
| | | 7.34-7.38 (2H, d), 7.40-7.46 (3H, m), | |
| | | 7.74-7.76 (1H, d), 7.89-7.93 (1H, | |
| | | m), 8.78-8.78 (1H, d), 8.84 (1H, s) | |
| 175 | 4-Oxo-3-phenyl-5-[1- | 2.35 (3H, s), 3.83-3.97 (1H, m), | 430 |
| | pyridin-2-yl-methylidene]- | 4.06-4.13 (1H, m), 4.25-4.36 (1H, | |
| | piperidine-1-carboxylic acid | m), 4.97-5.10 (1H, m), | |
| | (4-methylsulfanyl-phenyl)- | 5.43-5.50 (1H, m), 7.12-7.16 (2H, d) | |
| | amide | 7.20-7.30 (6H, m), 7.34-7.52 (4H, m), | |
| | | 7.54-7.60 (2H, m), | |
| 176 | 3,3-Dimethyl-5-[1-(6- | 0.98 (3H, s), 1.40 (3H, s), | 497 |
| | morpholin-4-yl-pyridin-2-yl)- | 3.32-3.49 (4H, t), 3.51-3.65 (4H, t), | |
| | methylidene]-4-oxo-2- | 5.40-5.71 (2H, d), 6.90 (1H, s), 6.91-6.97 (2H, | |
| | phenyl-piperidine-1- | m), 7.17-7.21 (3H, m), | |
| | carboxylic acid | 7.23-7.26 (3H, m), 7.29-7.37 (4H, m), | |
| | phenylamide | 7.44-7.46 (1H, m), 7.65-7.69 (1H, m), | |
| | | 8.4 (1H, bs) | |
| 177 | 2-Benzyl-3,3-dimethyl-5-[1- | 1.22 (3H, s), 1.21 (3H, s), | 541 |
| | (6-morpholin-4-yl-pyridin-2- | 2.69-2.70 (1H, t), 3.28-3.30 (2H, t), | |
| | yl)-methylidene]-4-oxo- | 3.34-3.40 (4H, t), 3.42-3.51 (4H, t), 3.53 (3H, | |
| | piperidine-1-carboxylic acid | s), 3.67-3.68 (1H, d), | |
| | (4-methoxy-phenyl)-amide | 4.64-4.68 (1H, m), 5.51-5.55 (1H, d), | |
| | | 6.73-6.75 (2H, d), 6.84-6.91 (2H, m), | |
| | | 6.98-7.14 (3H, m), 7.17-7.23 (5H, | |
| | | m), 7.25-7.36 (2H, m), 8.41 (1H, bs) | |
| 178 | 2-Benzyl-3,3-dimethyl-5-[1- | 1.25 (6H, s), 2.68 (2H, d), | 527 |
| | (6-morpholin-4-yl-pyridin-2- | 3.07-3.11 (2H, d), 3.55-3.61 (8H, t), | |
| | yl)-methylidene]-4-oxo- | 5.27-5.28 (1H, m), 6.83-6.88 (2H, d), | |
| | piperidine-1-carbothioic | 7.06-7.09 (2H, d), 7.23-7.35 (8H, | |
| | acid phenylamide | m), 7.42-7.43 (1H, m), | |
| | | 7.65-7.68 (1H, t), 8.9 (1H, s) | |
| 179 | 2-Benzyl-3,3-dimethyl-5-[1- | 1.05 (3H, s), 1.19 (3H, s), | 529 |
| | (6-morpholin-4-yl-pyridin-2- | 3.03-3.08 (1H, m), 3.35-3.40 (2H, t), | |
| | yl)-methylidene]-4-oxo- | 3.42-3.45 (4H, t), 3.51-3.55 (4H, t) | |
| | piperidine-1-carboxylic acid | 3.66-3.69 (2H, m), 6.71-6.89 (3H, t), | |
| | (4-fluoro-phenyl)-amide | 6.98-7.00 (4H, m), 7.03-7.13 (4H, m), | |
| | | 7.37-7.45 (2H, m), 7.64-7.66 (1H, | |
| | | t), 8.15 (1H, bs) | |
| 180 | 2-Benzyl-3,3-dimethyl-5-[1- | 0.82 (6H, s), 1.21-1.23 (6H, d), | 477 |
| | (6-morpholin-4-yl-pyridin-2- | 1.29-1.3 (3H, t), 1.36-1.37 (2H, t), | |
| | yl)-methylidene]-4-oxo- | 2.45-2.58 (1H, m), 3.35-3.52 (4H, t), | |
| | piperidine-1-carboxylic acid | 3.67-3.68 (4H, t), 4.52-4.53 (1H, m), | |
| | isopropylamide | 6.90-7.03 (1H, d), 7.19-7.20 (1H, | |
| | | d), 7.31-7.41 (5H, m), 7.67 (1H, s), | |
| | | 7.68-7.70 (1H, m), 10.51 (1H, bs) | |
| 181 | 2-Benzyl-3,3-dimethyl-5-[1- | 1.13 (3H, s), 1.17 (3H, s), 2.17 (3H, | 525 |
| | (6-morpholin-4-yl-pyridin-2- | s), 2.55-2.58 (1H, m), | |
| | yl)-methylidene]-4-oxo- | 3.02-3.06 (1H, m), 3.39-3.40 (2H, | |
| | piperidine-1-carboxylic acid | 3.41-3.44 (3H, m), 3.50-3.51 (4H, t), | |
| | p-tolylamide | 3.51-3.62 (4H, t), 3.65-3.68 (2H, t), | |
| | | 6.69-6.76 (1H, m), 6.89-6.91 (1H, d), | |
| | | 6.96-7.01 (3H, m), 7.06-7.13 (4H, | |
| | | m), 7.16-7.18 (2H, t), | |
| | | 7.28-7.36 (3H, m), 9.73 (1H, bs) | |
| 182 | 2-Benzyl-3,3-dimethyl-5-[1- | 1.16 (3H, s), 1.19 (3H, s), | 511 |
| | (6-morpholin-4-yl-pyridin-2- | 2.53-2.55 (1H, d), 3.03-3.08 (1H, d), | |
| | yl)-methylidene]-4-oxo- | 3.39-3.41 (4H, t), 3.43-3.50 (4H, t), | |
| | piperidine-1-carboxylic acid | 3.66-3.69 (1H m), 4.68-4.72 (1H, m), | |
| | phenylamide | 5.52-5.57 (1H, d), 6.87-6.91 (2H, | |
| | | m), 7.07-7.18 (8H, m), | |
| | | 7.20-7.23 (2H, m), 7.43-7.46 (1H, m), | |
| | | 7.64-7.68 (1H, m), 8.09 (1H, bs) | |
| 183 | 3,3-Dimethyl-5-[1-(6- | 0.93 (3H, s), 1.39 (3H, s), 2.22 (3H, | 511 |
| | morpholin-4-yl-pyridin-2-yl)- | s), 3.43-3.48 (4H, t), | |
| | methylidene]-4-oxo-2- | 3.49-3.55 (4H, t), 5.07-5.12 (1H, d), 5.39 (1H, | |
| | phenyl-piperidine-1- | s), 5.69-5.74 (1H, d), | |
| | carboxylic acid p-tolylamide | 6.90-6.92 (1H, d), 7.05-7.12 (4H, m), | |
| | | 7.14-7.17 (2H, d), 7.18-7.29 (4H, m), | |
| | | 7.46 (1H, s), 7.65-7.69 (1H, m), | |
| | | 8.40 (1H, bs) | |
| 184 | 3,3-Dimethyl-5-[1-(6- | 0.93 (3H, s), 1.39 (3H, s), | 527 |
| | morpholin-4-yl-pyridin-2-yl)- | 3.43-3.48 (4H, t), 3.49-3.55 (4H, t), 3.64 (3H, | |
| | methylidene]-4-oxo-2- | s), 5.07-5.12 (1H, d), 5.39 (1H, s), | |
| | phenyl-piperidine-1- | 5.69-5.74 (1H, d), 6.90-6.92 (1H, | |
| | carboxylic acid (4-methoxy- | d), 7.05-7.12 (4H, m), | |
| | phenyl)-amide | 7.14-7.17 (2H, d), 7.18-7.29 (4H, m), | |
| | | 7.46 (1H, s), 7.65-7.69 (1H, m), 8.40 (1H, | |
| | | bs) | |
| 185 | 4-Oxo-3-phenyl-5-[1- | 3.69 (3H, s), 3.76 (3H, s), | 444 |
| | pyridin-2-yl-methylidene]- | 3.80-3.83 (1H, m), 4.04-4.08 (1H, m), | |
| | piperidine-1-carboxylic acid | 4.20-4.28 (1H, m), 4.96-5.01 (1H, m), | |
| | (2,4-dimethoxy-phenyl)- | 5.45-5.49 (1H, d), 6.42-6.45 (1H, | |
| | amide | m), 6.52-6.55 (1H, m), | |
| | | 7.27-7.43 (8H, m), 7.74-7.75 (1H, d), | |
| | | 7.76-7.80 (1H, m), 7.89-7.91 (1H, m), | |
| | | 8.75 (1H, d) | |
| 186 | 4-Oxo-3-phenyl-5-[1- | 3.83-3.93 (1H, m), 4.04-4.08 (1H, | 384 |
| | pyridin-2-yl-methylidene]- | m), 4.20-4.28 (1H, m), | |
| | piperidine-1-carboxylic acid | 4.96-5.01 (1H, m), 5.45-5.49 (1H, d), | |
| | phenylamide | 6.89-6.98 (1H, m), 7.15-7.29 (5H, m), | |
| | | 7.32-7.48 (6H, m), 7.61-7.74 (1H, | |
| | | d), 7.89-7.93 (1H, m), 8.72 (1H, bs), | |
| | | 8.78-8.79 (1H, d) | |
| 187 | 4-Oxo-3-phenyl-5-[1- | 2.22 (3H, s), 3.83-3.93 (1H, m), | 398 |
| | pyridin-2-yl-methylidene]- | 4.04-4.08 (1H, m), 4.20-4.28 (1H, | |
| | piperidine-1-carboxylic acid | m), 4.96-5.01 (1H, m), | |
| | p-tolylamide | 5.45-5.49 (1H, d), 6.89-6.98 (1H, m), | |
| | | 7.15-7.29 (4H, m), 7.32-7.48 (6H, m), | |
| | | 7.61-7.74 (1H, d), 7.89-7.93 (1H, | |
| | | m), 8.72 (1H, bs), 8.78-8.79 (1H, d) | |
| 188 | 3,3-Dimethyl-5-[1-(6- | 0.93 (3H, s), 1.39 (3H, s), | 515 |
| | morpholin-4-yl-pyridin-2-yl)- | 3.43-3.48 (4H, t), 3.49-3.55 (4H, t), | |
| | methylidene]-4-oxo-2- | 5.07-5.12 (1H, d), 5.39 (1H, s), | |
| | phenyl-piperidine-1- | 5.69-5.74 (1H, d), 6.90-6.92 (1H, d), | |
| | carboxylic acid (4-fluoro- | 7.05-7.12 (4H, m), 7.14-7.17 (2H, d), | |
| | phenyl)-amide | 7.18-7.29 (4H, m), 7.46 (1H, s), | |
| | | 7.65-7.69 (1H, m), 8.40 (1H, bs) | |
| 189 | 3-[1-(4-Methylsulfanyl- | 2.53 (3H, s), 3.83-3.93 (1H, m), | 429 |
| | phenyl)-methylidene]-4- | 4.04-4.08 (1H, m), 4.20-4.28 (1H, | |
| | oxo-5-phenyl-piperidine-1- | m), 4.96-5.01 (1H, m), | |
| | carboxylic acid | 5.45-5.49 (1H, d), 6.89-6.98 (1H, m), | |
| | phenylamide | 7.15-7.29 (5H, m), 7.32-7.48 (6H, m), | |
| | | 7.61-7.74 (1H, d), 7.89-7.93 (1H, | |
| | | m), 8.69 (1H, bs), | |
| 190 | 3-[1-(4-Methylsulfanyl- | 2.50 (3H, s), 3.83-3.93 (1H, m), | 464 |
| | phenyl)-methylidene]-4- | 4.04-4.08 (1H, m), 4.20-4.28 (1H, | |
| | oxo-5-phenyl-piperidine-1- | m), 4.96-5.01 (1H, m), | |
| | carboxylic acid (4-chloro- | 5.45-5.49 (1H, d), 6.89-6.98 (1H, m), | |
| | phenyl)-amide | 7.15-7.29 (4H, m), 7.32-7.48 (6H, m), | |
| | | 7.61-7.74 (1H, d), 7.89-7.93 (1H, | |
| | | m), 8.69 (1H, bs), | |
| 191 | 3-[1-(4-Methanesulfonyl- | 3.20 (3H, s), 3.83-3.93 (1H, m), | 461 |
| | phenyl)-methylidene]-4- | 4.04-4.08 (1H, m), 4.20-4.28 (1H, | |
| | oxo-5-phenyl-piperidine-1- | m), 4.96-5.01 (1H, m), | |
| | carboxylic acid | 5.45-5.49 (1H, d), 6.89-6.98 (1H, m), | |
| | phenylamide | 7.15-7.29 (5H, m), 7.32-7.48 (6H, m), | |
| | | 7.61-7.74 (1H, d), 7.89-7.93 (1H, | |
| | | m), 8.69 (1H, bs), | |
| 192 | 1,5,5-Trimethyl-3-[1-(6- | 1.12 (3H, s), 1.23 (2H, s), 2.31 (3H, | 392 |
| | morpholin-4-yl-pyridin-2-yl)- | s), 2.46-2.49 (2H, t), | |
| | methylidene]-2-phenyl- | 3.37-3.41 (4H, t), 3.64-3.72 (4H, t), 6.07 (1H, | |
| | piperidin-4-one | s), 6.77-6.81 (2H, t), 6.97 (1H, s), | |
| | | 7.04-7.06 (2H, d), 7.20-7.23 (3H, | |
| | | m), 7.50-7.54 (1H, m) | |
| 193 | 3,3-Dimethyl-2-morpholin- | 0.85 (3H, s), 1.24 (3H, s), | 481 |
| | 4-ylmethyl-4-oxo-5-[1- | 2.27-2.37 (4H, t), 2.46 (3H, s), 2.46-2.59 (2H, | |
| | pyridin-2-yl-methylidene]- | t), 3.38-3.42 (4H, t), 3.52-3.63 (2H, | |
| | piperidine-1-carboxylic acid | t), 4.49-4.55 (1H, d), | |
| | (4-methylsulfanyl-phenyl)- | 7.16-7.27 (4H, m), 7.7.50-7.52 (2H, d), | |
| | amide | 7.67-7.74 (2H, m), 8.41 (1H, d), 9.53 (1H, | |
| | | bs) | |
| 194 | 3,3-Dimethyl-2-morpholin- | 0.85 (3H, s), 1.24 (3H, s), | 465 |
| | 4-ylmethyl-4-oxo-5-[1- | 2.27-2.37 (4H, t), 2.46-2.59 (2H, t), | |
| | pyridin-2-yl-methylidene]- | 3.38-3.42 (4H, t), 3.52-3.63 (2H, t), 3.74 (3H, | |
| | piperidine-1-carboxylic acid | s), 4.49-4.55 (1H, d), | |
| | (4-methoxy-phenyl)-amide | 7.16-7.27 (4H, m), 7.7.50-7.52 (2H, d), | |
| | | 7.67-7.74 (2H, m), 8.41 (1H, d), 9.53 (1H, | |
| | | bs) | |
| 195 | 4-({3,3-Dimethyl-2- | 0.85 (3H, s), 1.24 (3H, s), | 507 |
| | morpholin-4-ylmethyl-4- | 1.23-1.30 (3H, t), 2.27-2.37 (4H, t), | |
| | oxo-5-[1-pyridin-2-yl- | 2.46-2.59 (2H, t), 3.38-3.42 (4H, t), | |
| | methylidene]-piperidine-1- | 3.52-3.63 (2H, t), 4.24-4.30 (2H, q), | |
| | carbonyl}-amino)-benzoic | 4.49-4.55 (1H, d), 7.16-7.27 (4H, m), | |
| | acid ethyl ester | 7.7.50-7.52 (2H, d), 7.67-7.74 (2H, | |
| | | m), 8.41 (1H, d), 9.53 (1H, bs) | |
| 196 | N-{3,3-Dimethyl-5-[1-(6- | 0.83 (3H, s), 1.09 (3H, s), | 561 |
| | morpholin-4-yl-pyridin-2-yl)- | 2.30-2.31 (2H, m), 3.49-3.52 (4H, t), | |
| | methylidene]-4-oxo-2- | 3.65-3.73 (4H, t), 6.94 (1H, s), | |
| | phenyl-piperidine-1- | 7.08-7.10 (1H, d), 7.30-7.35 (3H, m), | |
| | carbonyl}- | 7.46-7.56 (6H, m), 7.60-7.64 (2H, m), | |
| | benzenesulfonamide | 7.87-7.81 (2H, m), 7.88 (1H, bs) | |
| 197 | 1-Methanesulfonyl-3,3- | 1.23 (3H, s), 1.28 (3H, s), | |
| | dimethyl-2-morpholin-4- | 2.30-2.38 (4H, t), 2.53-2.67 (2H, t), 3.21 (3H, | |
| | ylmethyl-5-[1-pyridin-2-yl- | s), 3.47-3.59 (4H, t), | |
| | methylidene]-piperidin-4- | 4.03-4.04 (1H, d), 4.54-4.59 (1H, d), | |
| | one | 5.37-5.41 (1H, d), 7.39-7.45 (2H, m), | |
| | | 7.74-7.76 (1H, d), 7.88-7.92 (1H, | |
| | | t), 8.76-8.77 (1H, d) | |
| 198 | 3,3-Dimethyl-2-morpholin- | 1.23 (3H, s), 1.28 (3H, s), 2.29 (3H, | 471 |
| | 4-ylmethyl-5-[1-pyridin-2-yl- | s), 2.30-2.38 (4H, t), | |
| | methylidene]-1-(toluene-4- | 2.53-2.67 (2H, t), 3.47-3.59 (4H, t), | |
| | sulfonyl)-piperidin-4-one | 4.03-4.04 (1H, d), 4.54-4.59 (1H, d), | |
| | | 5.37-5.41 (1H, d), 7.36-7.44 (4H, m), | |
| | | 7.74-7.76 (1H, d), 7.2-7.86 (2H, d), | |
| | | 7.88-7.92 (1H, t), 8.76-8.77 (1H, d) | |
| 199 | 1-Methanesulfonyl-3,3- | 1.01 (3H, s), 1.33 (3H, s), 2.67 (3H, | 371 |
| | dimethyl-2-phenyl-5-[1- | s), 4.62-4.67 (1H, dd), | |
| | pyridin-2-yl-methylidene]- | 4.95-4.99 (1H, d), 5.47-5.52 (1H, d), | |
| | piperidin-4-one | 7.13-7.14 (2H, d), 7.23-7.43 (5H, m), | |
| | | 7.65 (1H, s), 7.83-7.84 (1H, d), | |
| | | 7.91-7.96 (1H, m) | |
| 200 | 1-Methanesulfonyl-3,3- | 1.01 (3H, s), 1.33 (3H, s), 2.67 (3H, | 456 |
| | dimethyl-5-[1-(6-morpholin- | s), 3.41-3.48 (4H, t), | |
| | 4-yl-pyridin-2-yl)- | 3.63-3.68 (4H, t), 4.62-4.67 (1H, dd), | |
| | methylidene]-2-phenyl- | 4.95-4.99 (1H, d), 5.47-5.52 (1H, d), | |
| | piperidin-4-one | 7.13-7.14 (2H, d), 7.23-7.43 (4H, m), | |
| | | 7.65 (1H, s), 7.83-7.84 (1H, d), | |
| | | 7.91-7.96 (1H, m) | |
| 201 | 3-[1-(6-Morpholin-4-yl- | 2.59 (3H, s), 3.56-3.64 (4H, t), | 504 |
| | pyridin-2-yl)-methylidene]- | 3.87-3.96 (4H, t), 3.97-3.98 (1H, m), | |
| | 5-phenyl-1-(toluene-4- | 4.08-4.12 (1H, m), 4.69-4.73 (1H, | |
| | sulfonyl)-piperidin-4-one | d), 5.26-5.30 (1H, d), 6.86 (1H, d), | |
| | | 6.98-7.05 (1H, d), 7.15-7.18 (1H, | |
| | | d), 7.35-7.41 (5H, m), | |
| | | 7.46-7.53 (2H, m), 7.73-7.78 (3H, m) | |
| 202 | 3-Phenyl-5-[1-pyridin-2-yl- | 2.59 (3H, s), 3.97-3.98 (1H, m), | 419 |
| | methylidene]-1-(toluene-4- | 4.08-4.12 (1H, m), 4.69-4.73 (1H, | |
| | sulfonyl)-piperidin-4-one | d), 5.26-5.30 (1H, d), 6.86 (1H, d), | |
| | | 6.98-7.05 (1H, d), 7.15-7.18 (1H, | |
| | | d), 7.35-7.41 (5H, m), | |
| | | 7.46-7.53 (3H, m), 7.73-7.78 (3H, m) | |
| 203 | 1-Acetyl-3-[1-(6-morpholin- | 2.24 (3H, s), 3.38-3.41 (4H, t), | 392 |
| | 4-yl-pyridin-3-yl)- | 3.48 (2H, s), 3.66-3.69 (4H, t), | |
| | methylidene]-5-phenyl- | 4.16-4.17 (2H, d), 6.51-6.53 (1H, d), | |
| | piperidin-4-one | 6.60-6063 (1H, d) 7.16-7.18 (2H, d), | |
| | | 7.25-7.45 (4H, m), 8.05 (1H, s) | |
| 204 | 1-Acetyl-3-methyl-5-[1-(6- | 1.21 (3H, s), 2.24 (3H, s), | 406 |
| | morpholin-4-yl-pyridin-2-yl)- | 3.38-3.41 (4H, t), 3.48 (2H, s), 3.66-3.69 (4H, | |
| | methylidene]-3-phenyl- | t), 4.16-4.17 (1H, d), | |
| | piperidin-4-one | 6.51-6.53 (1H, d), 6.60-6063 (1H, d), | |
| | | 7.16-7.18 (2H, d), 7.25-7.45 (4H, m), | |
| | | 8.05 (1H, s) | |
| 205 | 3-[1-(6-Morpholin-4-yl- | 3.41-3.53 (4H, t), 3.56 (2H, s), | 469 |
| | pyridin-2-yl)-methylidene]- | 3.65-3.66 (4H, t), 3.89-3.93 (1H, m), | |
| | 4-oxo-5-phenyl-piperidine- | 4.13-4.19 (1H, m), 4.27-4.32 (1H, | |
| | 1-carboxylic acid | m), 6.55-6.57 (1H, d), | |
| | phenylamide | 6.61-6.63 (1H, d), 7.04-7.07 (1H, t), | |
| | | 7.23-7.25 (3H, m), 7.28-7.36 (4H, m), | |
| | | 7.43-7.51 (3H, m), 8.16 (1H, s), | |
| | | 9.46 (1H, bs) | |
| 206 | 1-Methanesulfonyl-3-[1-(6- | 3.24 (3H, s), 3.39-3.42 (4H, t), | 428 |
| | morpholin-4-yl-pyridin-2-yl)- | 3.51 (2H, s), 3.67-3.69 (4H, t), | |
| | methylidene]-5-phenyl- | 3.90-3.94 (1H, t), 4.08-4.10 (2H, d), | |
| | piperidin-4-one | 6.52-6.54 (1H, d), 6.61-6.63 (1H, d), | |
| | | 7.20-7.21 (2H, m), 7.27-7.33 (3H, | |
| | | m), 7.42-7.46 (1H, m), 7.79 (1H, s) | |
| 207 | 3-[1-(6-Morpholin-4-yl- | 2.30 (3H, s), 3.41-3.53 (4H, t), | 483 |
| | pyridin-2-yl)-methylidene]- | 3.56 (2H, s), 3.65-3.66 (4H, t), | |
| | 4-oxo-5-phenyl-piperidine- | 3.89-3.93 (1H, m), 4.13-4.19 (1H, m), | |
| | 1-carboxylic acid p- | 4.27-4.32 (1H, m), 6.55-6.57 (1H, | |
| | tolylamide | d), 6.61-6.63 (1H, d), | |
| | | 7.04-7.07 (1H, t), 7.23-7.25 (2H, m), | |
| | | 7.28-7.36 (4H, m), 7.43-7.51 (3H, m), | |
| | | 8.16 (1H, s), 9.46 (1H, bs) | |
| 208 | 3-[1-(6-Morpholin-4-yl- | 3.41-3.53 (4H, t), 3.56 (2H, s), | 529 |
| | pyridin-2-yl)-methylidene]- | 3.65-3.66 (4H, t), 3.75 (6H, s), | |
| | 4-oxo-5-phenyl-piperidine- | 3.89-3.93 (1H, m), 4.13-4.19 (1H, m), | |
| | 1-carboxylic (2,4- | 4.27-4.32 (1H, m), 6.55-6.57 (1H, | |
| | dimethoxy-phenyl)-amide | d), 6.61-6.63 (1H, d), | |
| | | 7.04-7.07 (1H, t), 7.23-7.25 (2H, m), | |
| | | 7.28-7.36 (3H, m), 7.43-7.51 (3H, m), | |
| | | 8.16 (1H, s), 9.46 (1H, bs) | |
| 209 | 4-Oxo-3-phenyl-5-[1- | 2.53 (3H, s), 3.56 (2H, s), | 426 |
| | pyridin-2-yl-methylidene]- | 3.89-3.93 (1H, m), 4.13-4.19 (1H, m), | |
| | piperidine-1-carboxylic acid | 4.27-4.32 (1H, m), 7.18-7.36 (7H, m), | |
| | (4-acetyl-phenyl)-amide | 7.66-7.70 (3H, m), 7.92-7.94 (2H, | |
| | | d), 8.12 (1H, s), 8.46-8.47 (1H, d), | |
| | | 9.80 (1H, bs) | |
| 210 | 1-Methanesulfonyl-3- | 3.24 (3H, s), 3.51 (2H, s) | 343 |
| | phenyl-5-[1-pyridin-2-yl- | 3.90-3.94 (1H, t), 4.08-4.10 (2H, d), | |
| | methylidene]-piperidin-4- | 6.52-6.54 (1H, d), 6.61-6.63 (1H, d), | |
| | one | 7.20-7.21 (2H, m), 7.27-7.33 (4H, m), | |
| | | 7.42-7.46 (1H, m), 7.79 (1H, s) | |
| 211 | 4-Oxo-3-phenyl-5-[1- | 3.64 (2H, s), 3.84-3.88 (1H, m), | 416 |
| | pyridin-2-yl-methylidene]- | 4.11-4.17 (1H, m), 4.23-4.27 (1H, | |
| | piperidine-1-carboxylic acid | m), 6.16-6.19 (1H, q), | |
| | (2,4-dihydroxy-phenyl)- | 6.31-6.318 (1H, d), 6.92-6.94 (1H, d), | |
| | amide | 7.17-7.35 (7H, m), 7.64-7.69 (1H, m), | |
| | | 8.10 (1H, s), 8.46-8.47 (1H, d), | |
| | | 8.64 (1H, s), 9.21 (1H, s), 9.30 (1H, s) | |
| 212 | 4-Oxo-3-phenyl-5-[1- | 3.64 (2H, s), 3.84-3.88 (1H, m), | 400 |
| | pyridin-2-yl-methylidene]- | 4.11-4.17 (1H, m), 4.23-4.27 (1H, | |
| | piperidine-1-carboxylic acid | m), 6.16-6.19 (1H, q), | |
| | (4-hydroxy-phenyl)-amide | 6.31-6.318 (1H, d), 6.92-6.94 (1H, d), | |
| | | 7.17-7.35 (8H, m), 7.64-7.69 (1H, m), | |
| | | 8.10 (1H, s), 8.46-8.47 (1H, d), | |
| | | 9.21 (1H, s), 9.30 (1H, s) | |
| 213 | 4-Oxo-3-phenyl-5-[1- | 3.17 (3H, s), 3.65 (2H, s), | 462 |
| | pyridin-2-yl-methylidene]- | 3.92-3.96 (1H, m), 4.16-4.22 (1H, m), | |
| | piperidine-1-carboxylic (4- | 4.29-4.34 (1H, m), 7.18 (1H, m), | |
| | methanesulfonyl-phenyl)- | 7.20-7.36 (6H, m), 7.66-7.70 (1H, m), | |
| | amide | 7.76-7.78 (2H, d), 7.85-7.87 (2H, | |
| | | d), 8.122 (1H, s), 8.47-8.48 (1H, d), | |
| | | 9.89 (1H, s) | |
| 214 | 1-(2,4-Dihydroxy- | 3.66-3.69 (2H, d), 3.80 (3H, s), | 437 |
| | benzenesulfonyl)-3-phenyl- | 6.34-6.36 (1H, d), 6.43 (1H, s), 7.14 (2H, | |
| | 5-[1-pyridin-2-yl- | d), 7.24 (5H, s), 7.52-7.54 (1H, d), | |
| | methylidene]-piperidin-4- | 7.75 (1H, s), 7.88 (1H, s), 8.50 (1H, | |
| | one | s), 10.55 (1H, s) 11.09 (1H, s) | |
| 215 | 4-{4-Oxo-3-phenyl-5-[1- | 3.55 (2H, s), 3.94-4.13 (3H, m), | 448 |
| | pyridin-2-yl-methylidene]- | 7.09-7.23 (8H, m), 7.44 (2H, s), | |
| | piperidine-1-carbonyl}- | 7.58-7.60 (3H, m), 7.80-7.82 (2H, | |
| | benzenesulfonamide | m), 8.34 (1H, m) | |
| 216 | 3-(4-Hydroxy-phenyl)-4- | 3.66 (2H, s), 3.75 (1H, s), | 400 |
| | oxo-5-[1-pyridin-2-yl- | 4.12-4.21 (2H, m), 6.70-6.72 (2H, m), | |
| | methylidene]-piperidine-1- | 6.88-7.06 (3H, m), 7.19-7.67 (5H, m), | |
| | carboxylic acid | 8.09 (1H, s), 8.47 (1H, s), | |
| | phenylamide | 9.34-9.46 (2H, d) | |
| 217 | 3-(4-Hydroxy-phenyl)-4- | 3.64 (2H, s), 3.72-3.73 (1H, m), | 416 |
| | oxo-5-[1-pyridin-2-yl- | 4.09-4.18 (2H, m), 6.71-6.72 (4H, | |
| | methylidene]-piperidine-1- | m), 6.91-7.02 (2H, m), | |
| | carboxylic acid (4-hydroxy- | 7.23-7.25 (4H, m), 7.67 (1H, s), 8.07 (1H, s), | |
| | phenyl)-amide | 8.45-8.46 (1H, d), 9.21-9.23 (2H, | |
| | | s), 9.34 (1H, s) | |
| 218 | 1-(4-Acetyl-benzoyl)-3- | 2.62 (3H, s), 3.64 (2H, s); | 411 |
| | phenyl-5-[1-pyridin-2-yl- | 3.96-40.5 (2H, m), 4.09-4.23 (2H, m), | |
| | methylidene]-piperidin-4- | 7.19-7.33 (8H, m), 7.62-7.72 (3H, m), | |
| | one | 8.03-8.05 (2H, d), 8.43-8.44 (1H, | |
| | | d) | |
| 219 | 3-(4-Hydroxy-phenyl)-5-[1- | 2.29 (3H, s), 3.52-3.63 (3H, m), | 435 |
| | pyridin-2-yl-methylidene]-1- | 3.67-3.77 (2H, m), 6.1 (2H, m), | |
| | (toluene-4-sulfonyl)- | 6.86-6.87 (2H, m), 7.17-7.20 (2H, m), | |
| | piperidin-4-one | 7.46-7.47 (2H, m), 7.66-7.73 (3H, | |
| | | m), 7.89 (1H, s), 8.49 (1H, s), | |
| | | 9.34 (1H, s) | |
| 220 | 3-(4-Hydroxy-phenyl)-1-(4- | 3.69 (2H, s), 3.92-3.95 (1H, t), | 464 |
| | methyl-benzoyl)-5-[1- | 4.08-4.21 (2H, m), 6.75-6.77 (2H, d), | |
| | pyridin-2-yl-methylidene]- | 7.02-7.03 (2H, m), 7.22-7.30 (2H, | |
| | piperidin-4-one | m), 7.60 (2H, s), 7.70-7.75 (3H, m), | |
| | | 7.96-7.98 (2H, d), 8.49-8.50 (1H, | |
| | | m), 9.44 (1H, s) | |
| 221 | 1-Benzenesulfonyl-3- | 3.71 (2H, s), 3.89-3.93 (3H, m), | 405 |
| | phenyl-5-[1-pyridin-2-yl- | 7.18-7.21 (2H, m), 7.23-7.29 (5H, | |
| | methylidene]-piperidin-4- | m), 7.71-7.74 (3H, m), | |
| | one | 7.83-7.86 (1H, m), 7.93-7.95 (2H, m), | |
| | | 8.01 (1H, s), 8.54-8.55 (1H, d) | |
| 222 | 1-Benzoyl-3-(4-hydroxy- | 3.68 (2H, s), 3.89-3.93 (1H, m), | 385 |
| | phenyl)-5-[1-pyridin-2-yl- | 4.21-4.29 (2H, m), 6.75-6.77 (2H, | |
| | methylidene]-piperidin-4- | d), 7.03-7.05 (2H, d), | |
| | one | 7.22-7.24 (1H, t), 7.25-7.28 (1H, d), | |
| | | 7.54-7.55 (4H, m), 7.62-7.63 (1H, m), | |
| | | 7.70-7.74 (1H, t), 8.49-8.50 (1H, | |
| | | d), 9.43 (1H, bs) | |
| 223 | 1-(4-Hydroxy-benzyl)-2-(4- | 1.06 (3H, s), 1.08 (3H, s), | 415 |
| | hydroxy-phenyl)-5,5- | 2.33-2.39 (2H, d), 3.26-3.29 (1H, d), | |
| | dimethyl-3-[1-pyridin-2-yl- | 3.43-3.47 (1H, d), 6.01 (1H, s), | |
| | methylidene]-piperidin-4- | 6.65-6.67 (4H, d), 7.00-7.04 (5H, m), | |
| | one | 7.27-7.30 (1H, m), | |
| | | 7.44-7.46 (1H, d), 7.73-7.77 (1H, t), | |
| | | 8.62-8.63 (1H, d), 9.21-9.29 (2H, d) | |
| 224 | 1-(4-Hydroxy-benzyl)-2-(5- | 1.06 (6H, s), 2.22-2.25 (1H, d), | 445 |
| | hydroxy-2-methoxy- | 2.56-2.59 (1H, d), 3.06-3.09 (1H, | |
| | phenyl)-5,5-dimethyl-3-[1- | d), 3.58 (3H, s), 3.68-3.72 (1H, d), | |
| | pyridin-2-yl-methylidene]- | 6.14 (1H, s), 6.58-6.66 (4H, m), | |
| | piperidin-4-one | 6.80-6.82 (1H, d), 6.92-6.93 (2H, | |
| | | d), 7.11 (1H, s), 7.24-7.27 (1H, t), | |
| | | 7.40-7.42 (1H, d), 7.72-7.75 (1H, | |
| | | t), 8.56-8.57 (1H, d), 8.93 (1H, bs), | |
| | | 9.23 (1H, bs) | |
| 225 | 1-Methanesulfonyl-2- | 2.31-2.41 (2H, t), 3.29 (3H, s), | 343 |
| | phenyl-4-[1-pyridin-2-yl- | 4.10-4.15 (1H, m), 5.31-5.32 (2H, t), | |
| | methylidene]-piperidin-3- | 7.32-7.42 (1H, m), 7.43-7.44 (1H, | |
| | one | d), 7.67-7.73 (2H, m), | |
| | | 7.88-7.94 (1H, m), 8.26-8.29 (1H, m), | |
| | | 8.29-8.37 (1H, d), 8.44-8.60 (1H, d), | |
| | | 8.68-8.80 (1H, m), 9.25-9.26 (1H, | |
| | | d) | |
| 226 | 1-Benzenesulfonyl-3-(4- | 3.63 (2H, s), 3.67-3.71 (1H, m), | 421 |
| | hydroxy-phenyl)- | 3.79-3.81 (2H, m), 6.60-6.62 (2H, | |
| | 5-[1-pyridin-2-yl- | d), 6.88-6.90 (2H, d), | |
| | methylidene]-piperidin-4- | 7.14-7.21 (2H, m), 7.63-7.69 (4H, m), | |
| | one | 7.77-7.91 (4H, m), 8.47-8.48 (1H, d), | |
| | | 9.35 (1H, s) | |
| 227 | 1-Benzyl-2-(4- | 0.88 (3H, s), 1.02 (3H, s), 2.92 (3H, | 546 |
| | methanesulfonyl-phenyl)- | s), 2.94-3.00 (2H, m), | |
| | 5,5-dimethyl-3-[1-(6- | 3.29-3.30 (4H, t), 3.31-3.34 (4H, t), | |
| | morpholin-4-yl-pyridin-2-yl)- | 3.40-3.45 (1H, d), 3.46-3.47 (1H, d), 6.38 (1H, | |
| | methylidene]-piperidin-4- | s), 6.75-6.77 (1H, d), | |
| | one | 6.92-6.94 (1H, d), 7.23-7.34 (5H, m), | |
| | | 7.36-7.38 (2H, d), 7.53-7.57 (1H, m), | |
| | | 7.68-7.70 (2H, m), 7.90-7.96 (1H, | |
| | | d) | |
| 228 | 1-Benzyl-5-[1-(4- | 1.19 (6H, s), 2.56 (2H, s), 3.08 (3H, | 384 |
| | methanesulfonyl-phenyl)- | s), 3.65 (2H, s), 3.71 (2H, s), | |
| | methylidene]-3,3-dimethyl- | 7.28 (2H, s), 7.34-7.35 (3H, m), | |
| | piperidin-4-one | 7.48-7.50 (3H, m), 7.94-7.96 (2H, d) | |
| 229 | 1-Benzyl-2-(4- | 1.14 (3H, s), 1.24 (3H, s), | 461 |
| | methanesulfonyl-phenyl)- | 2.57-2.60 (1H, d), 2.65-2.68 (1H, d), 3.02 (3H, | |
| | 5,5-dimethyl-3-[1-pyridin-2- | s), 3.50-3.57 (2H, d), 6.30 (1H, s), | |
| | yl-methylidene]-piperidin-4- | 7.02-7.20 (2H, m), 7.24-7.28 (4H, | |
| | one | m), 7.31-7.35 (2H, m), | |
| | | 7.54-7.57 (2H, d), 7.59-7.66 (1H, m), | |
| | | 7.81-7.83 (2H, d), 8.62-8.63 (1H, d) | |
| 230 | 2-(2,5-Dimethoxy-phenyl)- | 1.15 (3H, s), 1.27 (3H, s), 2.31 (3H, | 542 |
| | 5,5-dimethyl-1-(4-methyl- | s), 2.71-2.74 (1H, d), | |
| | benzyl)-3-[1-(6-morpholin- | 3.28-3.31 (2H, m), 3.32-3.42 (3H, m), | |
| | 4-yl-pyridin-2-yl)- | 3.60 (3H, s), 3.75 (6H, s), 3.79-3.82 (2H, | |
| | methylidene]-piperidin-4- | m), 4.22-4.25 (1H, m), 6.15 (1H, s), | |
| | one | 6.47-6.50 (1H, d), 6.61-6.65 (2H, | |
| | | m), 6.75-6.78 (1H, dd), | |
| | | 6.83-6.85 (1H, d), 7.10-7.12 (2H, d), | |
| | | 7.26-7.30 (3H, m), 7.37-7.41 (1H, m) | |
| 231 | 5,5-Dimethyl-1-(4-methyl- | 1.18 (3H, s), 1.27 (3H, s), 2.31 (3H, | 397 |
| | benzyl)-2-phenyl-3-[1- | s), 2.46-2.53 (1H, d), | |
| | pyridin-2-yl-methylidene]- | 2.65-2.71 (1H, d), 3.45-3.50 (1H, m), | |
| | piperidin-4-one | 3.58-3.61 (1H, d), 6.06 (1H, s), | |
| | | 7.07-7.11 (2H, d), 7.12-7.14 (1H, m), | |
| | | 7.15-7.18 (3H, m), 7.19-7.22 (2H, m), | |
| | | 7.23-7.28 (4H, m), 7.54-7.59 (1H, | |
| | | m), 8.63-8.64 (1H, d) | |
| 232 | 5,5-Dimethyl-1-(4-methyl- | 1.10 (3H, s), 1.33 (3H, s), 2.35 (3H, | 482 |
| | benzyl)-3-[1-(6-morpholin- | s), 2.52-2.56 (1H, d), | |
| | 4-yl-pyridin-2-yl)- | 2.79-2.83 (1H, d), 3.03-3.09 (2H, m), | |
| | methylidene]-2-phenyl- | 3.13-3.19 (2H, m), 3.58-3.61 (4H, t), | |
| | piperidin-4-one | 3.69-3.72 (1H, d), 3.80-3.87 (1H, d), | |
| | | 6.30 (1H, s), 6.49-6.51 (1H, d), | |
| | | 6.73-6.75 (1H, d), 7.07-7.09 (2H, d), | |
| | | 7.18-7.23 (5H, m), 7.25-7.27 (2H, | |
| | | m), 7.28 (1H, s), 7.42-7.46 (1H, m) | |
| 233 | 2-(2,5-Dimethoxy-phenyl)- | 1.19 (6H, s), 2.33 (5H, s), | 457 |
| | 5,5-dimethyl-1-(4-methyl- | 2.68-2.71 (1H, d), 3.28-3.31 (1H, t), 3.65 (3H, | |
| | benzyl)-3-[1-pyridin-2-yl- | s), 3.78 (3H, s), 6.14 (1H, s), | |
| | methylidene]-piperidin-4- | 6.75-6.77 (1H, m), 6.78-7.84 (2H, m), | |
| | one | 7.07-7.15 (3H, m), 7.20-7.27 (3H, | |
| | | d), 7.28 (1H, s), 7.54-7.58 (1H, m), | |
| | | 8.59-8.60 (1H, d) | |
| 234 | 2-(2,5-Dimethoxy-phenyl)- | 1.15 (3H, s), 1.23 (3H, s), | 555 |
| | 5,5-dimethyl-1-(4-methyl- | 2.30-2.38 (6H, m), 2.50 (3H, s), 2.68-2.75 (1H, | |
| | benzyl)-3-[1-[6-(4-methyl- | t), 3.22-3.24 (1H, m), | |
| | piperazin-1-yl)-pyridin-2-yl]- | 3.31-3.34 (2H, t), 3.39-3.44 (2H, m), | |
| | methylidene]-piperidin-4- | 3.49-3.50 (2H, d), 3.66 (3H, s), 3.84 (3H, | |
| | one | s), 3.88-3.94 (1H, m), 6.01 (1H, s), | |
| | | 6.16 (1H, s), 6.49-6.53 (1H, m), | |
| | | 6.56-6.63 (1H, m), 6.64-6.68 (1H, | |
| | | m), 6.69-6.71 (1H, m), | |
| | | 6.78-6.82 (1H, m), 6.83-6.85 (1H, m), | |
| | | 7.10-7.11 (1H, m), 7.20-7.28 (2H, m), | |
| | | 7.34-7.42 (1H, m) | |
| 235 | 1-(3,4-Dimethoxy-benzyl)- | 1.02 (6H, s), 2.23-2.24 (2H, m), | 528 |
| | 5,5-dimethyl-3-[1-(6- | 2.50-2.55 (2H, m), 3.04-3.06 (4H, | |
| | morpholin-4-yl-pyridin-2-yl)- | t), 3.62-3.64 (4H, t), 3.75 (3H, s), | |
| | methylidene]-2-phenyl- | 3.82 (3H, s), 6.74-6.76 (2H, m), | |
| | piperidin-4-one | 6.92-6.97 (2H, d), 7.28-7.30 (2H, | |
| | | d), 7.31-7.37 (2H, m), | |
| | | 7.45-7.57 (1H, d), 7.53-7.54 (1H, m), | |
| | | 7.78-8.04 (1H, m), 8.15-8.16 (1H, d), | |
| | | 9.07-9.09 (1H, d) | |

### Method of Protecting Cells Against Stress

The present invention relates to a method of inducing the expression of Heat Shock Protein 70 (HSP-70) in cells, by administering an effective amount of one or more compound of present invention, represented by the formula (I) or (II), their pharmaceutically acceptable salts and their hydrates, solvates, stereoisomers, conformers, tautomers, polymorphs and prodrugs, thereof and their pharmaceutically acceptable composition.

In the present context, “HSP-70” refers to proteins of the HSP family having an approximate molecular mass of 70 kDa, which are induced in response to a pathological stress. “Pathological stress” refers to factors which disturb the homeostasis of the cells thus leading to the increased expression of stress proteins like HSP-70. Such factors are, for example, metabolic, oxidative, stresses caused by hypoxia, ischemia, infections, stresses induced by metals and exogenous substances, immunogenic stresses, cell malignancy, neurodegeneration, trauma, or aging. Other forms of pathological stresses include those causing the formation of free radicals or increase in the quantity of inflammatory cytokines.

In one embodiment of the present invention, diseases accompanying pathological stress are selected from cerebrovascular diseases, cardiovascular diseases, neurodegenerative diseases and immune disorders, such as ischemic stroke, myocardial infarction, inflammatory disorders, hepatotoxicity, sepsis, diseases of viral origin, allograft rejection, tumourous diseases, gastric mucosal damage, brain hemorrhage, endothelial dysfunctions, diabetic complications, neuro-degenerative diseases, epilepsy, post-traumatic neuronal damage, acute renal failure, glaucoma and aging related skin degeneration. The compounds of the present invention possess the ability to induce HSP-70 and thereby protect cells against stress-induced damage in the above disease conditions.

The present invention also relates to a method of inhibiting TNF-α in cells, by administering an effective amount of one or more compound, represented by the formula (I) or (II), their pharmaceutically acceptable salts and their hydrates, solvates, stereoisomers, tautomers, polymorphs and prodrugs, thereof and their pharmaceutically acceptable composition. Cytokines such as TNF-α produced by activated monocytes and macrophages play an important role in the regulation of the immune response. Studies have shown that TNF-α is involved in the pathogenesis of diabetes, myocardial infarction, liver failure, infectious diseases like sepsis syndrome, auto immune diseases like rheumatic arthritis, graft rejection, organ transplant rejection, chronic inflammatory disorders such as rheumatoid diseases, arthritic disorders and connective tissue disorders. Reference may be made to [Han, H. S, and Yenari, M. A., Current Opinion in Investigational Drugs, 2003, Vol. 4(5), pp. 522-529]. Treatment with compound of the instant invention which shows TNF-α inhibitory activity exerts a cytoprotective effect in the above disease conditions.

In a specific embodiment of the invention, a method of increasing HSP-70 expression in cells is provided.

In still another embodiment of the invention, a method of inhibition of TNF-αexpression is provided.

### BIOLOGICAL ACTIVITY

### In Vitro Activity

### (i) Effect of Compounds of the Instant Invention on Cellular Expression of HSP

Experiments set forth in this section were conducted to determine whether the compounds of the present invention are able to elevate the expression of HSP-70 gene in cells.

Hela cell-line or primary mixed neurons derived from neonatal rat cerebellum were employed. Induction was carried out for the indicated dose(s) for 4 hours duration and total RNA was isolated. Expression of HSP70b mRNA along-with expression of 18S rRNA was monitored by real-time PCR. HSP70b mRNA expression was normalized relative to the expression of 18S rRNA.

The results for test compounds were expressed as fold induction of HSP-70 mRNA relative to vehicle treated control and are as shown in Table 2 & 3.

TABLE 2

| Compound | HSP Induction in |
|---|---|
| No. | Hela cells |
| 13 | (+++) |
| 14 | (+++) |
| 15 | (++++) |
| 16 | (++++) |
| 17 | (++) |
| 20 | (+++) |
| 23 | (+++) |
| 25 | (++++) |
| 28 | (++++) |
| 29 | (+++) |
| 30 | (+) |
| 31 | (++) |
| 33 | (+++) |
| 34 | (++) |
| 35 | (++++) |
| 36 | (++++) |
| 37 | (++++) |

0 indicates <4 fold;
+, ++, +++ and ++++ indicate 4-24 fold, 25-192 fold, 193-1536 fold, and >1536 fold induction of HSP-70b mRNA, respectively, relative to the vehicle treated control.

TABLE 3

| Compound | HSP Induction in |
|---|---|
| no. | mixed neuron |
| 42 | (+++) |
| 43 | (+++) |
| 45 | (++) |
| 46 | (+++) |
| 47 | (++++) |
| 52 | (++) |
| 57 | (+) |
| 58 | (+) |
| 60 | (++++) |
| 61 | (++++) |
| 62 | (+) |
| 63 | (++) |
| 64 | (++++) |
| 65 | (+++) |
| 66 | (++) |
| 68 | (++++) |
| 69 | (++++) |
| 73 | (+) |
| 76 | (+) |
| 79 | (+++) |
| 81 | (+) |
| 82 | (+++) |
| 85 | (++) |
| 86 | (+) |
| 90 | (+) |
| 92 | (++) |
| 94 | (+) |
| 95 | (++) |
| 103 | (+) |
| 105 | (++) |
| 227 | (++++) |
| 229 | (++) |
| 232 | (++) |
| 234 | (+++) |

0 indicates <2 fold while
+, ++, +++, ++++ indicate 2-4 fold, 5-8 fold, 9-16 fold, and >16 fold induction of HSP70b mRNA, respectively, relative to vehicle treated control.

### Discussion

As seen in Table 2 & 3, HSP-70 mRNA levels were increased over control after treatment with compounds of the invention. Thus, the compounds of the instant invention have the ability to induce HSP-70.

### (ii) Effect of Compounds of the Present Invention for TNF-α Expression

The purpose of the present study was to determine the inhibition of lipopolysaccharide(LPS)-induced TNF-α expression in phorbol merstyl ester (PMA) differentiated THP-1 cells.

Human monocytic leukaemia cell line (THP-1), differentiated into macrophage-like cells by PMA treatment was employed. Differentiated cells were treated with either LPS (1 ug/ml) alone or with LPS (1 ug/ml) and compound for 4 hours. Total RNA was isolated and expression of TNF-αmRNA along-with expression of 18S rRNA was monitored by real-time PCR. TNF-α mRNA expression was normalized relative to the expression of 18S rRNA Considering TNF-α expression for cells treated with LPS alone as 100%; the results for test compounds were expressed as % inhibition of TNF-α expression and are as shown in Table 4

TABLE 4

| Compound | TNF-alpha |
|---|---|
| No. | Inhibition |
| 1 | (++++) |
| 2 | (+++) |
| 7 | (+++) |
| 8 | (++++) |
| 16 | (++++) |
| 20 | (++) |
| 42 | (++) |
| 43 | (++) |
| 46 | (++) |
| 47 | (+++) |
| 52 | (++++) |
| 60 | (+++) |
| 61 | (+++) |
| 63 | (++) |
| 65 | (+++) |
| 68 | (++++) |
| 69 | (+++) |
| 79 | (++++) |
| 82 | (++++) |
| 85 | (+) |
| 90 | (++) |
| 103 | (+) |
| 105 | (++) |
| 227 | (++++) |
| 229 | (++++) |
| 234 | (+++) |

0 indicates <20% while
+, ++, +++, ++++ indicate 21-40%, 41-60%, 61-80% and >80% inhibition of TNF-α expression, respectively.

### Discussion

As seen in Table 4, LPS-induced TNF-α expression was inhibited by the treatment with compounds of the present invention.

### In Vivo Activity

### Assessment of Neuroprotective Activity

Transient cerebral ischemia (for 2 hit) was induced in male Sprague Dawley rats of 240-270 g body weight under halothane anaesthesia by the intraluminal suture occlusion technique—inserting a 3-0 polyamide suture from proximal external carotid artery into the lumen of internal carotid artery (Longa E Z. et al. Stroke 20: 84-91; 1989). During the entire surgical procedure for the induction of stroke, the body temperature of the animal was maintained at 37° C., using a homoeothermic blanket. At the end of 2 hrs the suture was removed for reperfusion. The test compound was administered to animals at 8^{th} hour post initiation of occlusion and subsequently at specified interval. At the end of 7 days all the animals were sacrificed and infarct was characterized after staining with triphenyl tetrazolium chloride (TTC). The images of the stained slices were captured using a scanner and was analyzed for infarct size and edema using Scion image software. Neurological scores were obtained at different time points after surgical recovery and improvement was assessed after reperfusion by calculating the percentage change from baseline scores (scores during ischemia).

### Neurological Score

| Score | Parameters |
|---|---|
| 0 | No Deficit |
| 1 | Failure to extend Right forepaw fully (Mild focal |
| | neurological deficit) |
| 2 | Circling to the contra lateral side (Moderate focal |
| | neurological deficit) |
| 3 | Falling to the contra lateral side |
| | (Moderate to severe focal neurological deficit) |
| 4 | Depressed level of Consciousness (severe focal |
| | neurological deficit) |

RESULTS

| | | | | % improvement |
|---|---|---|---|---|
| | i.p. Dose | % reduction | | Mean Neurological |
| Compound | (mg/kg) | Infarct | Edema | Score |
| Compound No. 68 | 15.2 | 30.0 | 51.0 | 61.1 |
| (Multiple dose) | | | | |

### Discussion

The ability of neuronal population to survive an ischemic insult (like stroke) is correlated with increased expression of HSP70. This test compound has shown the ability to induce HSP70 in-vitro, and inhibit TNF-α in cultured cells. HSP70 mRNA was induced in neurons at the periphery of ischemia (Penumbra). It is proposed and demonstrated that the penumbra can be rescued from getting infracted by pharmacological agents. (Dienel G. A. et. al., J. Cereb Blood Flow Metab., 1986, 6: pp 505-510; Kinouchi H. et. al., Brain Research., 1993, 619: pp 334-338). The in vivo efficacy carried out with the representative test compound No. 68 to assess the neuroprotective activity in an animal model of cerebral ischemia has demonstrated neuroprotection i.e. reduced infarct size and brain edema with improvement in neurological deficit following cerebral ischemia. These results very well correlate with our in vitro data and hence can be concluded that compounds of present invention would be useful as neuroprotective agents by virtue of their ability to induce HSP70 protein.

1. A compound of formula (I) or (II), 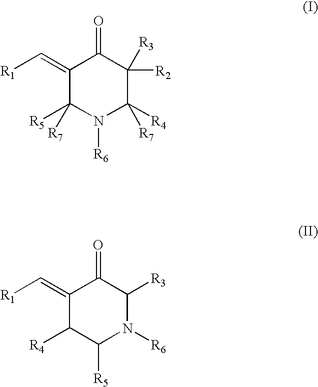
or its pharmaceutically acceptable salts and their hydrates, solvates, stereoisomers, conformers, tautomers and polymorphs;
wherein, R₁ is selected from unsubstituted or substituted:
a. Five to twelve membered monocyclic or bicyclic aryl,
b. Five to twelve membered monocyclic or bicyclic heteroaryl
wherein, it contains one or more heteroatoms selected from nitrogen, oxygen and sulphur, or
c. Four to twelve membered monocyclic or bicyclic heterocyclyl wherein, it contains one or more heteroatoms selected from nitrogen, oxygen and sulphur;
Said aryl, heteroaryl, heterocyclyl when substituted, it is substituted by one to four substituents of R₈, preferably one to three substituents of R₈, more preferably one to two substituents of R₈, wherein R₈ is independently selected from the group consisting of:
halogen, —OH, —SH, —C₁₋₈alkyl, nitro, amino, cyano, —N(R₉)C(O)(C₁₋₈alkyl), —N(R₉)C(O) (aryl), —N(R₉)C(O) (heteroaryl), —N(R₉)C(O) (heterocyclyl), —N(R₉) SO₂(C₁₋₈alkyl), —N(R₉) SO₂ (aryl), —N(R₉) SO₂ (heteroaryl), —N(R₉) SO₂ (heterocyclyl), (R₉) SO₂CF₃, —COOH, —C(O)N(R₉)(R₉), —C(O)N(R₉(aryl), —C(O)N(R₉)(heteroaryl), —C(O)N(R₉)(heterocyclyl), —SO₂N(R₉), (R₉), —SO₂N(R₉) (aryl), —SO₂N(R₉) (heteroaryl), —SO₂N(R₉) (heterocyclyl), —C(O)O—(C₁₋₈alkyl), —C(O)O-aryl, —C(O)O-heteroaryl, —C(O)O-heterocyclyl, —N(R₉)C(O)O—(C₁₋₈alkyl), —N(R₉)C(O)O-aryl, —N(R₉)C(O)O-heteroaryl, —N(R₉)C(O)O-heterocyclyl, —CF₃, —C(O)CF₃, —SO₂CF₃, —(C₁₋₈alkyl)ₘ-O(C₁₋₈alkyl), —(C₁₋₈alkyl)ₘ-O(aryl), —(C₁₋₈alkyl), —O(heteroaryl), —(C₁₋₈alkyl)ₘ O(heterocyclyl), —(C₁₋₈alkyl)ₘ-N(R₉)(C₁₋₈alkyl), —(C₁₋₈alkyl)ₘ-N (R₉) (aryl), —(C₁₋₈alkyl)ₘ-N(R₉)(heteroaryl), —(C₁₋₈alkyl)ₘ-N (R₉) (heterocyclyl), —(C₁₋₈alkyl)ₘ-C(O)(C₁₋₈alkyl), —(C₁₋₈alkyl)ₘ-C(O)(aryl), —(C₁₋₈alkyl)ₘ-C(O)(heteroaryl), —(C₁₋₈alkyl)ₘ-C(O)(heterocyclyl), —C(O) (C₁₋₈alkyl)-aryl, —C(O)(C₁₋₈alkyl)-heteroaryl, —C(O)(C₁₋₈alkyl)-heterocyclyl, —(C₁₋₈alkyl)ₘ-S(O)(C₁₋₈alkyl), —(C₁₋₈alkyl)ₘ-S(O) (aryl), —(C₁₋₈alkyl)ₘ-S(O) (heteroaryl)-(C₁₋₈alkyl)ₘ-S(O)(heterocyclyl), —(C₁₋₈alkyl)ₘ-S(O)₂ (C₁₋₈alkyl), —(C₁₋₈alkyl)ₘ-S(O)₂O—(C₁₋₈alkyl), —(C₁₋₈alkyl)ₘ-SO₂ (aryl)-(C₁₋₈alkyl)ₘ-SO₂(heteroaryl), —(C₁₋₈alkyl)ₘ-SO₂(heterocyclyl), N(R₉)(SO₂-aryl), —N(R₉)(SO₂-heteroaryl), —N(R₉)(SO₂-heterocyclyl), —N(R₉)C(O)N(R₉)(R₉), —N(R₉)C(O)N(R₉) (aryl), —N(R₉)C(O)N(R₉) (heteroaryl), —N(R₉)C(O)N(R₉) (heterocyclyl), —N(R₉)C(O)C(O)N(R₉)(R₉), —N(R₉)C(O)C(O)N(R₉) (aryl), NR₉C(O)C(O)N(R₉) (heteroaryl), —N(R₉)C(O)C(O)N(R₉) (heterocyclyl), —N(R₉)C(S)N(R₉)(R₉), —N(R₉)C(S)N(R₉) (aryl), —N(R₉)C(S)N(R₉) (heteroaryl), —N(R₉)C(S)N(R₉) (heterocyclyl), —N(R₉) SO₂N(R₉)(R₉)—N(R₉) SO₂N(R₉) (aryl), —N(R₉) SO₂N(R₉) (heteroaryl), —N(R₉) SO₂N(R₉)(heterocyclyl), —S(C₁₋₈alkyl), —SO₂OH, —NHC(NH)NH₂, —N(R₉)(aryl), —N(R₉)(heteroaryl), —N(R₉) (heterocyclyl), —(C₁₋₈alkyl)ₘ-aryl, —(C₁₋₈alkyl)ₘ-heteroaryl, —(C₁₋₈alkyl)ₘ-heterocyclyl-oxo, and -thioxo;
R₉ is selected from hydrogen or (C₁₋₈alkyl);
wherein, aryl present as a substituent in R₈ is five to seven membered monocyclic ring and heteroaryl and heterocyclyl present as a substituent in R₈ is three to seven membered monocyclic ring system which contains one or more heteroatoms selected from nitrogen, oxygen and sulphur; wherein the aryl, heteroaryl and heterocyclyl are unsubstituted or substituted with one to three substituents independently selected from the group consisting of:
oxo, thioxo, halogen, —OH, —SH, —O(C₁₋₈alkyl), nitro, amino, mono(C₁₋₈alkyl)amino, di(C₁₋₈alkyl)amino, —COOH, —CONH₂, —CF₃, —C(O)CF₃, —SO₂CF₃, —S(C₁₋₈alkyl), —SO₂(C₁₋₈alkyl), and —SO₂NH₂;
wherein, the above said C₁₋₈alkyl is straight, branched or cyclic and may contain one double bond and is substituted with one to two substituents independently selected from the group consisting of:
—OH, —SH, oxo, thioxo, amino, mono(C₁₋₃alkyl)amino, di(C₁₋₃alkyl)amino, —S(C₁₋₃alkyl), and —C₁₋₃ alkoxy;
wherein C₁₋₃alkoxy is straight or branched, may contain one or two double or triple bonds; C₁₋₃alkyl is straight or branched;
R₉ is selected from hydrogen or (C₁-C₈)alkyl;
m is zero or one;
with the proviso that when R₁ is selected from unsubstituted or substituted
a) cyclohexane,
b) cyclohexene or
c) six membered monocyclic heteroaryl or heterocyclyl having one to two heteroatoms selected from nitrogen, oxygen or sulphur, then R₈ as substituent on R₁ is not selected from hydroxyl and oxo group.
R₂ is selected from the group consisting of:
hydrogen, halogen, —C₁₋₃alkyl, —OH, —SH, —O(C₁₋₃alkyl), amino, mono(C₁₋₃alkyl)amino, di(C₁₋₃alkyl)amino, —C(O)CF₃, —C(O)CH₃, —SO₂CF₃—CF₃, —S(C₁₋₈alkyl), —SO₂(C₁₋₈alkyl), and —SO₂NH₂;
wherein, the above said C₁₋₈alkyl is straight, branched or cyclic and may contain one or two double or triple bonds and is substituted with one to two substituents independently selected from the group consisting of:
—OH, —SH, oxo, thioxo, amino, mono(C₁₋₃alkyl)amino, di(C₁₋₃alkyl)amino, —S(C₁₋₃alkyl), and —C₁₋₃ alkoxy;
Wherein, C₁₋₃alkoxy is straight or branched, may contain one double bond; C₁₋₃alkyl is straight or branched.
R₃ is selected from the group consisting of:
halogen, nitro, amino, —OH, —SH, —N(R₉)C(O)(C₁₋₈alkyl), —N(R₉)C(O)(aryl), —N(R₉)C(O)(heteroaryl), —N(R₉)C(O)(heterocyclyl), —N(R₉) SO₂ (O₁₋₈alkyl), —N(R₉) SO₂ (aryl), —N(R₉) SO₂ (heteroaryl), —N(R₉) SO₂ (heterocyclyl), —(C₁₋₃alkyl), —(C₁₋₃alkyl)ₘ-aryl, —(C₁₋₃alkyl)ₘ-heteroaryl, —(C₁₋₃alkyl)ₘ-heterocyclyl, —C(O)N(R₉)(R₉), —C(O)N(R₉)(aryl), —C(O)N(R₉)(heteroaryl), —C(O)N(R₉)(heterocyclyl), —SO₂N((R₉)(R₉), —SO₂N(R₉) (aryl), —SO₂N(R₉) (heteroaryl), —SO₂N(R₉) (heterocyclyl), —N(R₉) SO₂CF₃, —C(O)O—(C₁₋₈alkyl), —C(O)O-aryl, —C(O)O-heteroaryl, —C(O)O-heterocyclyl, —N(R₉)C(O)O—(C₁₋₈alkyl), —N(R₉)C(O)O-aryl, —N(R₉)C(O)O-heteroaryl, —N(R₉)C(O)O-heterocyclyl, —CF₃, —C(O)CF₃, —SO₂CF₃, COOH, —(C₁₋₃alkyl)ₘ-O(C₁₋₈alkyl), —(C₁₋₃alkyl)ₘ-N((R₉)(C₁₋₈alkyl), —(C₁₋₃alkyl)ₘ-C(O)(C₁₋₈alkyl), —(C₁₋₃alkyl)ₘ-C(O)(aryl), —(C₁₋₃alkyl)ₘ-C(O)(heteroaryl), —(C₁₋₃alkyl)ₘ-C(O)(heterocyclyl), —C(O)(C₁₋₃alkyl)-aryl, —C(O)(C₁₋₃alkyl)-heteroaryl, —C(O)(C₁₋₃alkyl)-heterocyclyl, —(C₁₋₃alkyl)-C(O)(C₁₋₃alkyl)-aryl, —(C₁₋₃alkyl)-C(O)(C₁₋₃alkyl)-heteroaryl, —(C₁₋₃alkyl)-C(O)(C₁₋₃alkyl)-heterocyclyl, —(C₁₋₃alkyl)ₘ-S(O)(C₁₋₈alkyl), —(C₁₋₃alkyl)ₘ-S(O)(aryl), —(C₁₋₃alkyl)ₘ-S(O)(heteroaryl)-(C₁₋₃alkyl)ₘ-S(O)(heterocyclyl), —(C₁₋₃ alkyl)ₘ-S(O)₂(C₁₋₈alkyl), —(C₁₋₃alkyl)ₘ-S(O)₂O—(C₁₋₈alkyl), —(C₁₋₃alkyl)ₘ-SO₂ (aryl), —(C₁₋₃alkyl)ₘ-SO₂ (heteroaryl), —(C₁₋₃alkyl)ₘ-SO₂ (heterocyclyl), —S(O)₂—(C₁₋₃alkyl)-aryl, —S(O)₂—(C₁₋₃alkyl)-heteroaryl, —S(O)₂—(C₁₋₃alkyl)-heterocyclyl, —(C₁₋₃alkyl) SO₂—(C₁₋₃alkyl)-aryl, —(C₁₋₃alkyl) SO₂—(C₁₋₃alkyl)-heteroaryl, —(C₁₋₃alkyl)SO₂—(C₁₋₃alkyl)-(C₁₋₃alkyl) SO₂—(C₁₋₃alkyl)-hetrocyclyl, —N(R₉) SO₂ (aryl), —N(R₉) SO₂ (heteroaryl)-N(R₉) SO₂ (heterocyclyl), —N(R₉)C(O)N((R₉)(R₉), —N(R₉)C(O)N(R₉)(aryl), —N(R₉)C(O)N(R₉)(heteroaryl), —N(R₉)C(O)N(R₉)(heterocyclyl), —N(R₉)C(O)C(O)N((R₉)(R₉), —N(R₉)C(O)C(O)N(R₉)(aryl), —N(R₉)C(O)C(O)N(R₉)(heteroaryl), —N(R₉)C(O)C(O)N(R₉)(heterocyclyl), —N(R₉)C(S)N(R₉)(R₉), —N(R₉)C(S)N(R₉)(aryl), —N(R₉)C(S)N(R₉)(heteroaryl), —N(R₉)C(S)N(R₉)(heterocyclyl), —N(R₉) SO₂N(R₉)(R₉), —N(R₉)SO₂N(R₉)(aryl), —N(R₉)SO₂N(R₉)(heteroaryl), —N(R₉)SO₂N(R₉)(heterocyclyl), —S(C₁₋₈alkyl), —SO₂OH, —NHC(═NH)NH₂, —(C₁₋₃alkyl)ₘ-O(aryl), —(C₁₋₃alkyl)ₘ-O(heteroaryl), —(C₁₋₃alkyl)ₘ-O(heterocyclyl), —(C₁₋₃alkyl)ₘ-N(R₉)(aryl), —(C₁₋₃alkyl)ₘ-N(R₉)(heteroaryl), —(C₁₋₃alkyl)ₘ-N(R₉) (heterocyclyl), —C(O)C(O)(aryl), —C(O)C(O)(heteroaryl), and —C(O)C(O)(heterocyclyl);
wherein, said aryl present as a substituent in R₃ is five to seven membered monocyclic ring and heteroaryl and heterocyclyl present as a substituent in R₃ are three to seven membered monocyclic ring containing one or more heteroatoms selected from nitrogen, oxygen and sulphur, wherein the said aryl, heteroaryl and heterocyclyl are unsubstituted or substituted with one to three substituents independently selected from the group consisting of:
oxo, thioxo, —OH, —SH, halogen, —C₁₋₈alkyl, —O(C₁₋₈alkyl), nitro, amino, mono(C₁₋₈alkyl)amino, di(C₁₋₈alkyl)amino, —COOH, —CONH₂, —CF₃, —C(O)CF₃, —SO₂CF₃, —S(C₁₋₈alkyl), —N(R₉) SO₂ (C₁₋₈alkyl), —SO₂ (C₁₋₈alkyl), and —SO₂NH₂;
Wherein, the above said C₁₋₈alkyl is straight, branched or cyclic, may contain one or two double or triple bonds and is with one to two substituents independently selected from the group consisting of:
—OH, —SH, Oxo, thioxo, amino, mono(C₁₋₃alkyl)amino, di(C₁₋₃alkyl)amino, —S(C₁₋₃alkyl), and —C₁₋₃ alkoxy;
wherein C₁₋₃alkoxy is straight or branched, may contain one double bond; C₁₋₃alkyl is straight or branched;
m is zero or one.
R₄ and R₅ is independently selected at each occurrence from hydrogen or R₈ or either R₄ or R₅ together with R₇ is oxo;
with the proviso that when R₄ is oxo, R₃ is not selected from —C(O)(C₁₋₈alkyl), —C(O)O(C₁₋₈alkyl), —C(O)(C₁₋₈alkyl)-aryl, —C(O)aryl, —C(O)thienyl, and —C(O)furyl;
R₆ is selected from the group consisting of:
(C₁₋₈alkyl), —C(O)N(R₉)(R₉), —C(O)N(R₉)(aryl), —C(O)N(R₉)((C₁₋₈alkyl)-aryl), —C(O)N(R₉) (heteroaryl), —C(O)N(R₉) SO₂ (aryl), —C(O)N(R₉)(heterocyclyl), —C(S)N(R₉)(R₉), —C(S)N(R₉)(aryl), —C(S)N(R₉) (heteroaryl), —C(S)N(R₉)(heterocyclyl), —SO₂N(R₉)(R₉), —SO₂N(R₉)(aryl)-SO₂N(R₉)(heteroaryl), —SO₂N(R₉)(heterocyclyl), —C(O)C(O)N(R₉)(R₉), —C(O)C(O)N(R₉)(aryl), —C(O)C(O)N(R₉)(heteroaryl), —C(O)C(O)N(R₉) (heterocyclyl), —C(O)O—(C₁₋₈alkyl), —C(O)O—(C₁₋₈alkyl)ₘ-aryl, —C(O)O—(C₁₋₈alkyl)ₘ-heteroaryl, —C(O)O—(C₁₋₈alkyl)ₘ-heterocyclyl, —CF₃, —C(O)CF₃, —SO₂CF₃, —(C₁₋₈alkyl)O(C₁₋₈alkyl), —(C₁₋₈alkyl)-O(aryl), —(C₁₋₈alkyl)-O(heteroaryl), —(C₁₋₈alkyl)-O(heterocyclyl), —(C₁₋₈alkyl)-N(R₉)(C₁₋₈alkyl), —(C₁₋₈alkyl)-N(R₉)(aryl), —(C₁₋₈alkyl)-N(R₉)(heteroaryl), —(C₁₋₈alkyl)-N(R₉)(heterocyclyl), —(C₁₋₈alkyl)ₘC(O)(C₁₋₈alkyl), —(C₁₋₈alkyl)ₘ-C(O)(aryl), —(C₁₋₈alkyl)ₘ-C(O)(heteroaryl), —(C₁₋₈alkyl)ₘ-C(O)(heterocyclyl), —C(O)—(C₁₋₃alkyl)-aryl, —C(O)—(C₁₋₃alkyl)-heteroaryl, —C(O)—(C₁₋₃alkyl)-heterocyclyl, —(C₁₋₈alkyl)-C(O)(C₁₋₈alkyl)-aryl, —(C₁₋₈alkyl)-C(O)(C₁₋₈alkyl)-heteroaryl, —(C₁₋₈alkyl)-C(O)(C₁₋₈alkyl)-heterocyclyl, —(C₁₋₈alkyl)ₘ-SO₂ (C₁₋₈alkyl), —(C₁₋₈alkyl)ₘ-SO₂ (aryl), —(C₁₋₈alkyl)ₘ-SO₂(heteroaryl), —(C₁₋₈alkyl)ₘ-SO₂(heterocyclyl), —(C₁₋₈alkyl)-S(O)(C₁₋₈alkyl), —(C₁₋₈alkyl)-S(O)(aryl), —(C₁₋₈alkyl)-S(O)(heteroaryl), —(C₁₋₈alkyl)-S(O)(heterocyclyl), —S(O)₂(C₁₋₈alkyl)-aryl, —S(O)₂(C₁₋₈alkyl)-heteroaryl, —S(O)₂(C₁₋₈alkyl)-heterocyclyl, —(C₁₋₈alkyl)SO₂—(C₁₋₈alkyl)-aryl, —(C₁₋₈alkyl)SO₂—(C₁₋₈alkyl)-heteroaryl, —(C₁₋₈alkyl)SO₂—(C₁₋₈alkyl)-heterocyclyl, —(C₁₋₈alkyl)ₘ-S(C₁₋₈alkyl), —(C₁₋₈alkyl)-S(C₁₋₈alkyl)-aryl, —(C₁₋₈alkyl)-S(C₁₋₈alkyl)-heteroaryl, —(C₁₋₈alkyl)-S(C₁₋₈alkyl)-hetrocyclyl, —(C₁₋₈alkyl)-S(aryl), —(C₁₋₈alkyl)-S(heteroaryl), —(C₁₋₈alkyl)-S(heterocyclyl), —(C₁₋₈alkyl)ₘ-aryl, —(C₁₋₈alkyl)ₘ-heteroaryl, —(C₁₋₈alkyl)ₘ-heterocyclyl, —C(O)C(O)(heteroaryl), —C(O)C(O)(heterocyclyl) and —C(O)C(O)(aryl);
wherein aryl present as a substituent in R₆ is five to seven membered monocyclic ring and heteroaryl and heterocyclyl present as a substituent in R₆ are three to seven membered monocyclic ring containing one or more heteroatoms selected from nitrogen, oxygen and sulphur; wherein said aryl, heteroaryl and heterocyclyl are unsubstituted or substituted with one to three groups independently selected from:
oxo, thioxo, halogen, —OH, —SH, —O(C₁₋₈alkyl), nitro, amino, mono(C₁₋₈alkyl)amino, di(C₁₋₈alkyl)amino, COOH, —COO(C₁₋₈alkyl), —CONH₂, —CF₃, —C(O)CF₃, —S(C₁₋₈alkyl), —SO₂ (C₁₋₈alkyl), —SO₂CF₃, and —SO₂NH₂;
wherein, the above said C₁₋₈alkyl is straight, branched or cyclic, may contain one or two double or triple bonds and may be substituted with one to two substituents independently selected from:
—OH, —SH, oxo, thioxo, amino, mono(C₁₋₃alkyl)amino, di(C₁₋₃alkyl)amino, —S(C₁₋₃alkyl), —COOH, CONH₂, and —C₁₋₃ alkoxy;
wherein, C₁₋₃alkoxy is straight or branched, may contain one double bond; C₁₋₃alkyl is straight or branched; m is independently selected at each occurrence, from zero to one.
with the proviso that:
i) when R₆ is selected from methyl, —CH₂—CH═CH₂ or —CH₂phenyl and R₂=H or methyl, then R₁ is not selected from:
a. trimethoxyphenyl,
b. benzdioxole or chlorosubstituted benzdioxole or
c. furyl;
ii) when R₆ is selected from methyl and R₂=H, R₃=Phenyl then R₁ is not selected from unsubstituted phenyl;
iii) when R₄, R₅ and R₇ are hydrogen and R₆ is selected from the group consisting of
—(C₁₋₈alkyl), —(C₁₋₈alkyl)-O(C₁₋₈alkyl), —(C₁₋₈alkyl)-O(aryl), —(C₁₋₈alkyl)-O(heteroaryl), —(C₁₋₈alkyl)-O(heterocyclyl), —(C₁₋₈alkyl)-N(R₉)(C₁₋₈alkyl), —(C₁₋₈alkyl)-N(R₉)(aryl), —(C₁₋₈alkyl)-N(R₉)(heteroaryl), —(C₁₋₈alkyl)-N(R₉)(heterocyclyl), —(C₁₋₈alkyl)-C(O)(C₁₋₈alkyl), —(C₁₋₈alkyl)-C(O)(aryl), —(C₁₋₈alkyl)-C(O)(heteroaryl), —(C₁₋₈alkyl)-C(O)(heterocyclyl), —(C₁₋₈alkyl)-C(O)(C₁₋₈alkyl)-aryl, —(C₁₋₈alkyl)-C(O)(C₁₋₈alkyl)-heteroaryl, —(C₁₋₈alkyl)-C(O)(C₁₋₈alkyl)-heterocyclyl, —(C₁₋₈alkyl)ₘ-aryl, —(C₁₋₈alkyl)ₘ-heteroaryl, —(C₁₋₈alkyl)ₘ-heterocyclyl, —C(O)N(R₉)(R₉), —(C₁₋₈alkyl)-SO₂(C₁₋₈alkyl), —(C₁₋₈alkyl)-S(O)(C₁₋₈alkyl), —(C₁₋₈alkyl)-S(O)(aryl), —(C₁₋₈alkyl)-S(O)(heteroaryl), —(C₁₋₈alkyl)-S(O)(heterocyclyl), —(C₁₋₈alkyl)-SO₂(C₁₋₈alkyl)-aryl, —(C₁₋₈alkyl)-SO₂(C₁₋₈alkyl)-heteroaryl, —(C₁₋₈alkyl)-SO₂(C₁₋₈alkyl)-aryl, hetrocyclyl, —(C₁₋₈alkyl)-S(C₁₋₈alkyl), —(C₁₋₈alkyl)-S(C₁₋₈alkyl)-aryl, —(C₁₋₈alkyl)-S(C₁₋₈alkyl)-heteroaryl, —(C₁₋₈alkyl)-S(C₁₋₈alkyl)-hetrocyclyl, —(C₁₋₈alkyl)-S(aryl), —(C₁₋₈alkyl)-S(heteroaryl), —(C₁₋₈alkyl)-S(heterocyclyl), —(C₁₋₈alkyl)-SO₂(aryl), —(C₁₋₈alkyl)-SO₂ (heteroaryl) —(C₁₋₈alkyl)-SO₂(heterocyclyl), acyl, and —C(O)O—(C₁₋₈alkyl),
then R₃ is not
—CH₂-phenyl, —CH₂-substituted phenyl, —CH₂-pyridyl, —CH₂-substituted pyridyl, —CH₂-pyrimidinyl, —CH₂-substituted pyrimidinyl wherein the substitution on aryl, pyridyl and pyrimidinyl is selected from hydroxyl, alkoxy, halogen and CF₃;
R₇ is selected from the group consisting of:
hydrogen, halogen, —OH, —SH, —O(C₁₋₈alkyl), nitro, amino, mono(C₁₋₈alkyl)amino, di(C₁₋₈alkyl)amino, —COOH, —CONH₂, —CF₃, —C(O)CF₃, —SO₂CF₃, —S(C₁₋₈alkyl), —SO₂(C₁₋₈alkyl), and —SO₂NH₂;
Wherein, the above said C₁₋₈alkyl is straight, branched or cyclic, may containing one or two double or triple bonds and substituted with one to two substituents selected from the group consisting of:
—OH, —SH, oxo, thioxo, amino, mono(C₁₋₃alkyl)amino, di(C₁₋₃alkyl)amino, —S(C₁₋₃alkyl), and alkoxy;
wherein, C₁₋₃alkoxy is straight or branched, may contain one double bond and C₁₋₃alkyl is straight or branched.

2. The compound of claim 1, wherein said compound is selected from the group consisting of:

| Compd. | |
|---|---|
| No | Nomenclature |
| 1 | 1-Benzyl-3,3-dimethyl-5-[1-pyridin-2-yl- |
| | methylidene]-piperidin-4- |
| | one |
| 2 | 3,3-Dimethyl-4-oxo-5-[1-pyridin-2-yl- |
| | methylidene]-piperidine-1-carboxylic acid |
| | benzyl ester |
| 3 | 3,3-Dimethyl-4-oxo-5-[1-pyridin-2-yl- |
| | methylidene]-piperidine-1-carboxylic acid |
| | ethyl ester |
| 4 | 3,3-Dimethyl-4-oxo-5-[1-pyridin-2-yl- |
| | methylidene]-piperidine-1-carboxylic acid |
| | phenyl ester |
| 5 | 1-Acetyl-3,3-dimethyl-5-[1-pyridin-2-yl- |
| | methylidene]-piperidin-4-one |
| 6 | 1-Benzyl-3-methyl-5-[1-pyridin-2-yl- |
| | methylidene]-piperidin-4-one |
| 7 | 1-Benzyl-3,3-dimethyl-5-[1-[4-(morpholine-4- |
| | carbonyl)-phenyl]-methylidene]-piperidin-4-one |
| 8 | 1-Benzyl-3,3-dimethyl-5-[1-(4-methylsulfanyl- |
| | phenyl)-methylidene]-piperidin-4-one |
| 9 | 1-Benzyl-3,3-dimethyl-5-[1-(4-nitro-phenyl)- |
| | methylidene]-piperidin-4-one |
| 10 | 1-Benzyl-3,3-dimethyl-5-[1-phenyl- |
| | methylidene]-piperidin-4-one |
| 11 | 1-Benzyl-3,3-dimethyl-5-[1-(3-methyl-thiophen- |
| | 2-yl)-methylidene]-piperidin-4-one |
| 12 | 1-Benzyl-5-[1-(4-methanesulfonyl-piperazin-1- |
| | yl)-methylidene]-3,3-dimethyl-piperidin-4-one |
| 13 | 2-(4-Methoxy-benzyl)-3,3-dimethyl-4-oxo-5-[1- |
| | pyridin-2-yl-methylidene]-piperidine-1- |
| | carboxylic acid ethyl ester |
| 14 | 2-(4-Methoxy-benzyl)-3,3-dimethyl-4-oxo-5-[1- |
| | pyridin-2-yl-methylidene]-piperidine-1- |
| | carboxylic acid phenyl ester |
| 15 | 2-(4-Methoxy-benzyl)-3,3-dimethyl-4-oxo-5-[1- |
| | pyridin-2-yl-methylidene]-piperidine-1- |
| | carboxylic acid isobutyl ester |
| 16 | 1-(2,2-Dimethyl-propionyl)-2-(4-methoxy- |
| | benzyl)-3,3-dimethyl-5-[1-pyridin-2-yl- |
| | methylidene]-piperidin-4-one |
| 17 | 2-(4-Methoxy-benzyl)-3,3-dimethyl-4-oxo-5-[1- |
| | pyridin-2-yl-methylidene]-piperidine-1- |
| | carboxylic acid (2,6-dimethyl-phenyl)-amde |
| 18 | 1-Benzyl-3,3-dimethyl-5-[1-quinolin-2-yl- |
| | methylidene]-piperidin-4-one |
| 19 | 1-Benzyl-3,3-dimethyl-5-[1-(1H-pyrrol-2-yl)- |
| | methylidene]-piperidin-4-one |
| 20 | 1-Benzyl-3,3-dimethyl-5-[1-(6-morpholin-4-yl- |
| | pyridin-2-yl)-methylidene]-piperidin-4-one |
| 21 | 1-Benzyl-3,3-dimethyl-5-[1-quinoxalin-2-yl- |
| | methylidene]-piperidin-4-one |
| 22 | 1-Benzyl-3,3-dimethyl-5-[1-thiophen-2-yl- |
| | methylidene]-piperidin-4-one |
| 23 | 1-Benzyl-3,3-dimethyl-5-[1-(3,4,5,6- |
| | tetrahydro-2H-[1,2′]bipyridinyl-6′-yl)- |
| | methylidene]-piperidin-4-one |
| 24 | 1-Benzyl-5-[1-(3-hydroxy-quinoxalin-2-yl)- |
| | methylidene]-3,3-dimethyl-piperidin-4-one |
| 25 | 1-Benzyl-5,5-dimethyl-2-phenyl-3-[1-pyridin-2- |
| | yl-methylidene]-piperidin-4-one |
| 26 | 1-Benzyl-5,5-dimethyl-2-phenyl-3-[1- |
| | quinoxalin-2-yl-methylidene]-piperidin-4-one |
| 27 | 1-Benzyl-5,5-dimethyl-2-phenyl-3-[1-(1H- |
| | pyrrol-2-yl)-methylidene]-piperidin-4-one |
| 28 | 1-Benzyl-5,5-dimethyl-3-[1-(6-morpholin-4-yl- |
| | pyridin-2-yl)-methylidene]-2,3,5,6-tetrahydro- |
| | 1H-[2,2′]bipyridinyl-4-one |
| 29 | 1-Benzyl-5,5-dimethyl-3-[1-pyridin-2-yl- |
| | methylidene]-2,3,5,6-tetrahydro-1H- |
| | [2,2′]bipyridinyl-4-one |
| 30 | 1-Benzyl-5,5-dimethyl-3-[1-(4-methylsulfanyl- |
| | phenyl)-methylidene]-2-phenyl-piperidin-4-one |
| 31 | 1-Benzyl-5,5-dimethyl-3-[1-(6-morpholin-4-yl- |
| | pyridin-2-yl)-methylidene]-2-phenyl-piperidin- |
| | 4-one |
| 32 | 1-Benzyl-5,5-dimethyl-3-[1-pyridin-2-yl- |
| | methylidene]-2-thiophen-2-yl-piperidin-4-one |
| 33 | 1-Benzyl-5,5-dimethyl-3-[1-(6-morpholin-4-yl- |
| | pyridin-2-yl)-methylidene]-2-thiophen-2-yl- |
| | piperidin-4-one |
| 34 | 1-Benzyl-5,5-dimethyl-3-[1-(3,4,5,6- |
| | tetrahydro-2H-[1,2′]bipyridinyl-6′-yl)- |
| | methylidene]-2,3,5,6-tetrahydro-1H- |
| | [2,2′]bipyridinyl-4-one |
| 35 | 3,3-Dimethyl-4-oxo-5-[1-(3,4,5,6-tetrahydro- |
| | 2H-[1,2′]bipyridinyl-6′-yl)-methylidene]- |
| | piperidine-1-carboxylic acid phenyl ester |
| 36 | 3,3-Dimethyl-5-[1-(6-morpholin-4-yl-pyridin-2- |
| | yl)-methylidene]-4-oxo-piperidine-1-carboxylic |
| | acid phenyl ester |
| 37 | 2-[1-Benzyl-5,5-dimethyl-4-oxo-piperidin-3- |
| | ylidenemethyl]-3H-quinazolin-4-one |
| 38 | 1-Benzyl-3,3-dimethyl-5-[1-pyridin-3-yl- |
| | methylidene]-piperidin-4-one |
| 39 | 5′-[1-Benzyl-5,5-dimethyl-4-oxo-piperidin-3- |
| | ylidenemethyl]-3,4,5,6-tetrahydro-2H- |
| | [1,2′]bipyridinyl-4-carboxylic acid |
| 40 | 1-Benzyl-2-(4-dimethylamino-phenyl)-5,5- |
| | dimethyl-3-[1-pyridin-2-yl-methylidene]- |
| | piperidin-4-one |
| 41 | 1-Benzyl-5-[1-[6-(3,5-dimethyl-morpholin-4- |
| | yl)-pyridin-3-yl]-methylidene]-3,3-dimethyl- |
| | piperidin-4-one |
| 42 | 1-Benzyl-5,5-dimethyl-2-(4-methylsulfanyl- |
| | phenyl)-3-[1-(6-morpholin-4-yl-pyridin-2-yl)- |
| | meth-(E)-ylidene]-piperidin-4-one |
| 43 | 1-Benzyl-5,5-dimethyl-3-[1-(6-morpholin-4-yl- |
| | pyridin-2-yl)-methylidene]-2-(4- |
| | trifluoromethyl-phenyl)- |
| | piperidin-4-one |
| 44 | 1-Benzyl-5,5-dimethyl-3-[1-pyridin-2-yl- |
| | methylidene]-2-(4-trifluoromethyl-phenyl)- |
| | piperidin-4-one |
| 45 | 1-Benzyl-2-(3,4-dichloro-phenyl)-5,5-dimethyl- |
| | 3-[1-pyridin-2-yl-methylidene]-piperidin-4-one |
| 46 | 1-Benzyl-5,5-dimethyl-2-(4-methylsulfanyl- |
| | phenyl)-3-[1-pyridin-2-yl-methylidene]- |
| | piperidin-4-one |
| 47 | 1-(4-Methoxy-benzyl)-5,5-dimethyl-2-phenyl-3- |
| | [1-pyridin-2-yl-methylidene]-piperidin-4-one |
| 48 | 1-(4-Methoxy-benzyl)-5,5-dimethyl-3-[1- |
| | pyridin-2-yl-methylidene]-2-thiophen-2-yl- |
| | piperidin-4-one |
| 49 | 1-Cyclopropyl-3,3-dimethyl-5-[1-pyridin-2-yl- |
| | methylidene]-piperidin-4-one |
| 50 | 3,3-Dimethyl-5-[1-(6-morpholin-4-yl-pyridin-2- |
| | yl)-methylidene]-1-thiophen-2-ylmethyl- |
| | piperidin-4-one |
| 51 | 1-Cyclopropyl-3,3-dimethyl-5-[1-(6-morpholin- |
| | 4-yl-pyridin-2-yl)-methylidene]-piperidin-4- |
| | one |
| 52 | 2-(4-Methoxy-benzyl)-3,3-dimethyl-4-oxo-5-[1- |
| | pyridin-2-yl-methylidene]-piperidine-1- |
| | carboxylic acid methyl ester |
| 53 | 2-(4-Methoxy-benzyl)-3,3-dimethyl-4-oxo-5-[1- |
| | pyridin-2-yl-methylidene]-piperidine-1- |
| | carboxylic acid (4-methylsulfanyl-phenyl)- |
| | amide |
| 54 | 2-(4-Methoxy-benzyl)-3,3-dimethyl-4-oxo-5-[1- |
| | pyridin-2-yl-methylidene]-piperidine-1- |
| | carboxylic acid (2,6-dimethoxy-phenyl)-amde |
| 55 | 3,3-Dimethyl-1-(5-methyl-isoxazol-3-yl)-5-[1- |
| | (6-morpholin-4-yl-pyridin-2-yl)-methylidene]- |
| | piperidin-4-one |
| 56 | 2-(2-Hydroxy-phenyl)-5,5-dimethyl-1-(5-methyl- |
| | isoxazol-3-yl)-3-[1-(6-morpholin-4-yl-pyridin- |
| | 2-yl)-methylidene]-piperidin-4-one |
| 57 | 2-(2-Fluoro-phenyl)-5,5-dimethyl-3-[1-(6- |
| | morpholin-4-yl-pyridin-2-yl)-methylidene]-1- |
| | thiophen-2-ylmethyl-piperidin-4-one |
| 58 | (2-Fluoro-phenyl)-5,5-dimethyl-3-[1-pyridin-2- |
| | yl-methylidene]-1-thiophen-2-ylmethyl- |
| | piperidin-4-one |
| 59 | 2-(4-Methoxy-benzyl)-3,3-dimethyl-4-oxo-5-[1- |
| | pyridin-2-yl-methylidene]-piperidine-1- |
| | carboxylic acid cyclohexylamide |
| 60 | 2-(4-Methoxy-benzyl)-3,3-dimethyl-4-oxo-5-[1- |
| | pyridin-2-yl-methylidene]-piperidine-1- |
| | carbothioic acid phenylamide |
| 61 | 5,5-Dimethyl-2-(4-methylsulfanyl-phenyl)-3-[1- |
| | pyridin-2-yl-methylidene]-1-thiophen-2- |
| | ylmethyl-piperidin-4-one |
| 62 | 1-(4-Methoxy-benzyl)-5,5-dimethyl-3-[1-(6- |
| | morpholin-4-yl-pyridin-2-yl)-methylidene]-2- |
| | phenyl-piperidin-4-one |
| 63 | 1-(4-Methoxy-benzyl)-5,5-dimethyl-3-[1-(6- |
| | morpholin-4-yl-pyridin-2-yl)-methylidene]-2- |
| | (4-trifluoromethyl-phenyl)-piperidin-4-one |
| 64 | 3,3-Dimethyl-1-(5-methyl-isoxazol-3-yl)-5-[1- |
| | pyridin-2-yl-methylidene]-piperidin-4-one |
| 65 | 5,5-Dimethyl-1-(5-methyl-isoxazol-3-yl)-3-[1- |
| | (6-morpholin-4-yl-pyridin-2-yl)-methylidene]- |
| | 2-phenyl-piperidin-4-one |
| 66 | 2-(4-Methoxy-benzyl)-3,3-dimethyl-4-oxo-5-[1- |
| | pyridin-2-yl-methylidene]-piperidine-1- |
| | carboxylic acid benzylamide |
| 67 | 2-(4-Methoxy-benzyl)-3,3-dimethyl-4-oxo-5-[1- |
| | pyridin-2-yl-methylidene]-piperidine-1- |
| | carboxylic acid (4-fluoro-phenyl)-amide |
| 68 | 2-(4-Methoxy-benzyl)-3,3-dimethyl-4-oxo-5-[1- |
| | pyridin-2-yl-methylidene]-piperidine-1- |
| | carboxylic acid (2,6-diisopropyl-phenyl)-amide |
| 69 | 3,3-Dimethyl-5-[1-(6-morpholin-4-yl-pyridin-2- |
| | yl)-methylidene]-1-(2-thiophen-2-yl-ethyl)- |
| | piperidin-4-one |
| 70 | 2-(2-Fluoro-phenyl)-5,5-dimethyl-3-[1-pyridin- |
| | 3-yl-methylidene]-1-thiophen-2-ylmethyl- |
| | piperidin-4-one |
| 71 | 1-Benzyl-5,5-dimethyl-3-[1-pyridin-2-yl- |
| | methylidene]-2-(3,4,5-trimethoxy-phenyl)- |
| | piperidin-4-one |
| 72 | 1-(4-Fluoro-benzyl)-3,3-dimethyl-5-[1-pyridin- |
| | 2-yl-methylidene]-piperidin-4-one |
| 73 | 1-(4-Fluoro-benzyl)-3,3-dimethyl-5-[1-(6- |
| | morpholin-4-yl-pyridin-2-yl)-methylidene]- |
| | piperidin-4-one |
| 74 | 3,3-Dimethyl-5-[1-(6-morpholin-4-yl-pyridin-2- |
| | yl)-methylidene]-1-(4-trifluoromethyl-benzyl)- |
| | piperidin-4-one |
| 75 | 4-({2-(4-Methoxy-benzyl)-3,3-dimethyl-4-oxo-5- |
| | [1-pyridin-2-yl-methylidene]-piperidine-1- |
| | carbonyl}-amino)-benzoic acid ethyl ester |
| 76 | 1-(4-Fluoro-benzyl)-5,5-dimethyl-2-phenyl-3- |
| | [1-pyridin-2-yl-methylidene]-piperidin-4-one |
| 77 | 1-(4-Methoxy-benzyl)-5,5-dimethyl-3-[1- |
| | pyridin-2-yl-methylidene]-2-(4- |
| | trifluoromethyl-phenyl)-piperidin-4-one |
| 78 | 2-(2-Fluoro-phenyl)-1-(4-methoxy-benzyl)-5,5- |
| | dimethyl-3-[1-pyridin-2-yl-methylidene]- |
| | piperidin-4-one |
| 79 | 3,3-Dimethyl-5-[1-pyridin-2-yl-methylidene]-1- |
| | (2-thiophen-2-yl-ethyl)-piperidin-4-one |
| 80 | 5,5-Dimethyl-3-[1-(6-morpholin-4-yl-pyridin-2- |
| | yl)-methylidene]-2-phenyl-1-(2-thiophen-2-yl- |
| | ethyl)-piperidin-4-one |
| 81 | 1-(4-Fluoro-benzyl)-5,5-dimethyl-3-[1-(6- |
| | morpholin-4-yl-pyridin-2-yl)-methylidene]-2- |
| | phenyl-piperidin-4-one |
| 82 | 1-Furan-2-ylmethyl-5,5-dimethyl-2-phenyl-3-[1- |
| | pyridin-2-yl-methylidene]-piperidin-4-one |
| 83 | 1-(3,4-Difluoro-benzyl)-5,5-dimethyl-2-phenyl- |
| | 3-[1-pyridin-2-yl-methylidene]-piperidin-4-one |
| 84 | 5,5-Dimethyl-2-phenyl-3-[1-pyridin-2-yl- |
| | methylidene]-1-(2-thiophen-2-yl-ethyl)- |
| | piperidin-4-one |
| 85 | 1,5,5-Trimethyl-2-phenyl-3-[1-pyridin-2-yl- |
| | methylidene]-piperidin-4-one |
| 86 | 2-(2-Fluoro-phenyl)-1-(4-methoxy-benzyl)-5,5- |
| | dimethyl-3-[1-(6-morpholin-4-yl-pyridin-2-yl)- |
| | methylidene]-piperidin-4-one |
| 87 | 1-(4-Fluoro-benzyl)-3,3-dimethyl-5-[1-(4- |
| | methylsulfanyl-phenyl)-methylidene]-piperidin- |
| | 4-one |
| 88 | 5,5-Dimethyl-1-(5-methyl-isoxazol-3-yl)-2-(4- |
| | methylsulfanyl-phenyl)-3-[1-(6-morpholin-4-yl- |
| | pyridin-2-yl)-methylidene]-piperidin-4-one |
| 89 | 3,3-Dimethyl-1-(5-methyl-isoxazol-3-yl)-5-[1- |
| | (4-methylsulfanyl-phenyl)-methylidene]- |
| | piperidin-4-one |
| 90 | 1-Furan-2-ylmethyl-5,5-dimethyl-3-[1-pyridin- |
| | 2-yl-methylidene]-2-(3,4,5-trimethoxy-phenyl)- |
| | piperidin-4-one |
| 91 | 1-Benzyl-2-(2-fluoro-4-methoxy-phenyl)-5,5- |
| | dimethyl-3-[1-pyridin-2-yl-methylidene]- |
| | piperidin-4-one |
| 92 | 1-Benzyl-2-(2-fluoro-4-methoxy-phenyl)-5,5- |
| | dimethyl-3-[1-(6-morpholin-4-yl-pyridin-2-yl)- |
| | methylidene]-piperidin-4-one |
| 93 | 5,5-Dimethyl-3-[1-(6-morpholin-4-yl-pyridin-2- |
| | yl)-methylidene]-2-phenyl-1-(3,4,5-trimethoxy- |
| | benzyl)-piperidin-4-one |
| 94 | 5,5-Dimethyl-1-phenethyl-2-phenyl-3-[1- |
| | pyridin-2-yl-methylidene]-piperidin-4-one |
| 95 | 5,5-Dimethyl-3-[1-(6-morpholin-4-yl-pyridin-2- |
| | yl)-methylidene]-1-phenethyl-2-phenyl- |
| | piperidin-4-one |
| 96 | 5,5-Dimethyl-1-(5-methyl-isoxazol-3-yl)-3-[1- |
| | (6-morpholin-4-yl-pyridin-2-yl)-methylidene]- |
| | 2-(4-trifluoromethyl-phenyl)-piperidin-4-one |
| 97 | 5,5-Dimethyl-1-(5-methyl-isoxazol-3-yl)-3-[1- |
| | [6-(4-methyl-piperazin-1-yl)-pyridin-2-yl]- |
| | methylidene]-2-(4-trifluoromethyl-phenyl)- |
| | piperidin-4-one |
| 98 | 5,5-Dimethyl-1-(5-methyl-isoxazol-3-yl)-3-[1- |
| | pyridin-2-yl-methylidene]-2-(4- |
| | trifluoromethyl-phenyl)-piperidin-4-one |
| 99 | {5,5-Dimethyl-3-[1-(6-morpholin-4-yl-pyridin- |
| | 2-yl)-methylidene]-4-oxo-2-phenyl-piperidin-1- |
| | yl}-acetic acid |
| 100 | {5,5-Dimethyl-4-oxo-2-phenyl-3-[1-pyridin-2- |
| | yl-methylidene]-piperidin-1-yl}-acetic acid |
| 101 | {2-(4-Fluoro-phenyl)-5,5-dimethyl-4-oxo-3-[1- |
| | pyridin-2-yl-methylidene]-piperidin-1-yl}- |
| | acetic acid |
| 102 | {5,5-Dimethyl-3-[1-[6-(4-methyl-piperazin-1- |
| | yl)-pyridin-2-yl]-methylidene]-4-oxo-2-phenyl- |
| | piperidin-1-yl}-acetic acid |
| 103 | 1-Benzyl-3-[1-(6-morpholin-4-yl-pyridin-2-yl)- |
| | methylidene]-5-phenyl-piperidine-2,4-dione |
| 104 | 2-(4-Methanesulfonyl-phenyl)-3,3-dimethyl-5- |
| | [1-(6-morpholin-4-yl-pyridin-2-yl)- |
| | methylidene]-4-oxo-piperidine-1-carbothioic |
| | acid phenylamide |
| 105 | 2-(4-Methanesulfonyl-phenyl)-3,3-dimethyl-4- |
| | oxo-5-[1-pyridin-2-yl-methylidene]-piperidine- |
| | 1-carbothioic acid phenylamide |
| 106 | 2-(4-Methanesulfonyl-phenyl)-3,3-dimethyl-4- |
| | oxo-5-[1-pyridin-2-yl-methylidene]-piperidine- |
| | 1-carboxylic acid benzylamide |
| 107 | 1-Benzyl-5-phenyl-3-[1-pyridin-2-yl- |
| | methylidene]-piperidine-2,4-dione |
| 108 | 1-Benzyl-3-[1-[6-(4-methyl-piperazin-1-yl)- |
| | pyridin-2-yl]-methylidene]-5-phenyl- |
| | piperidine-2,4-dione |
| 109 | 1-(3,4-Dimethoxy-benzyl)-5,5-dimethyl-2- |
| | phenyl-3-[1-pyridin-2-yl-methylidene]- |
| | piperidin-4-one |
| 110 | 5,5-Dimethyl-1-(4-methyl-benzyl)-3-[1-[6-(4- |
| | methyl-piperazin-1-yl)-pyridin-2-yl]- |
| | methylidene]-2-phenyl-piperidin-4-one |
| 111 | 2-(4-Methanesulfonyl-phenyl)-3,3-dimethyl- |
| | 4-oxo-5-[1-pyridin-2-yl-methylidene]- |
| | piperidine-1-carboxylic acid (4-fluoro- |
| | phenyl)-amide |
| 112 | 5,5-Dimethyl-1-(2-morpholin-4-yl-ethyl)-2- |
| | phenyl-3-[1-pyridin-2-yl-methylidene]- |
| | piperidin-4-one |
| 113 | 5,5-Dimethyl-1-(2-morpholin-4-yl-ethyl)-3-[1- |
| | (6-morpholin-4-yl-pyridin-2-yl)-methylidene]- |
| | 2-phenyl-piperidin-4-one |
| 114 | 1-Benzyl-3-(3,4-dimethoxy-phenyl)-4-hydroxy-5- |
| | [1-(6-morpholin-4-yl-pyridin-2-yl)- |
| | methylidene]-5,6-dihydro-1H-pyridin-2-one |
| 115 | 5,5-Dimethyl-1-(2-morpholin-4-yl-ethyl)-3-[1- |
| | (6-morpholin-4-yl-pyridin-2-yl)-methylidene]- |
| | 2-p-tolyl-piperidin-4-one |
| 116 | 4-Hydroxy-1-(4-methyl-benzyl)-3-[1-(6- |
| | morpholin-4-yl-pyridin-2-yl)-methylidene]-5- |
| | phenyl-3,6-dihydro-1H-pyridin-2-one |
| 117 | 2-(4-Dimethylamino-phenyl)-5,5-dimethyl-1-(4- |
| | methyl-benzyl)-3-[1-(6-morpholin-4-yl-pyridin- |
| | 2-yl)-methylidene]-piperidin-4-one |
| 118 | 2-(4-Dimethylamino-phenyl)-5,5-dimethyl-1-(4- |
| | methyl-benzyl)-3-[1-pyridin-2-yl-methylidene]- |
| | piperidin-4-one |
| 119 | 5,5-Dimethyl-2-(4-methylsulfanyl-phenyl)-3-[1- |
| | (6-morpholin-4-yl-pyridin-2-yl)-methylidene]- |
| | 1-thiophen-2-ylmethyl-piperidin-4-one |
| 120 | 2-(2,5-Dimethoxy-phenyl)-3-[1-(4- |
| | methanesulfonyl-phenyl)-methylidene]-5,5- |
| | dimethyl-1-(4-methyl-benzyl)-piperidin-4-one |
| 121 | 2-(2,5-Dimethoxy-phenyl)-5,5-dimethyl-1-(4- |
| | methyl-benzyl)-3-[1-(4-methylsulfanyl-phenyl)- |
| | methylidene]-piperidin-4-one |
| 122 | N-(4-{1-Benzyl-4-hydroxy-5-[1-(6-morpholin-4- |
| | yl-pyridin-2-yl)-methylidene]-6-oxo-1,2,5,6- |
| | tetrahydro-pyridin-3-yl}-phenyl)- |
| | methanesulfonamide |
| 123 | 1-Benzyl-5-(3,5-dimethyl-phenyl)-3-[1-pyridin- |
| | 2-yl-methylidene]-piperidine-2,4-dione |
| 124 | 1-Methanesulfonyl-3-[1-(6-morpholin-4-yl- |
| | pyridin-2-yl)-methylidene]-5-phenyl- |
| | piperidine-2,4-dione |
| 125 | 2-(4-Dimethylamino-phenyl)-5,5-dimethyl-1-(4- |
| | methyl-benzyl)-3-[1-quinolin-2-yl- |
| | methylidene]-piperidin-4-one |
| 126 | 1-Benzoyl-4-hydroxy-5-phenyl-3-[1-pyridin-2- |
| | yl-methylidene]-3,6-dihydro-1H-pyridin-2-one |
| 127 | 2-(4-Fluoro-phenyl)-5,5-dimethyl-1-(4-methyl- |
| | benzyl)-3-[1-(6-morpholin-4-yl-pyridin-2-yl)- |
| | methylidene]-piperidin-4-one |
| 128 | 4-Hydroxy-1-(4-methyl-benzyl)-5-phenyl-3-[1- |
| | pyridin-2-yl-methylidene]-3,6-dihydro-1H- |
| | pyridin-2-one |
| 129 | 1-(4-Methyl-benzyl)-3-[1-(4-methylsulfanyl- |
| | phenyl)-methylidene]-5-phenyl-piperidine-2,4- |
| | dione |
| 130 | 1-(3-Methoxy-benzyl)-5-phenyl-3-[1-pyridin-2- |
| | yl-methylidene]-piperidine-2,4-dione |
| 131 | 5,5-Dimethyl-3-[1-(6-morpholin-4-yl-pyridin-2- |
| | yl)-methylidene]-2-phenyl-1-(2-piperidin-1-yl- |
| | ethyl)-piperidin-4-one |
| 132 | 2-(4-Fluoro-phenyl)-5,5-dimethyl-3-[1-(6- |
| | morpholin-4-yl-pyridin-2-yl)-methylidene]-1- |
| | (2-piperidin-1-yl-ethyl)-piperidin-4-one |
| 133 | 5,5-Dimethyl-2-phenyl-1-(2-piperidin-1-yl- |
| | ethyl)-3-[1-pyridin-2-yl-methylidene]- |
| | piperidin-4-one |
| 134 | 2-(4-Fluoro-phenyl)-5,5-dimethyl-1-(2- |
| | piperidin-1-yl-ethyl)-3-[1-pyridin-2-yl- |
| | methylidene]-piperidin-4-one |
| 135 | 5,5-Dimethyl-3-[1-(6-morpholin-4-yl-pyridin-2- |
| | yl)-methylidene]-1-(2-piperidin-1-yl-ethyl)-2- |
| | p-tolyl-piperidin-4-one |
| 136 | 2-(4-Dimethylamino-phenyl)-5,5-dimethyl-1-(2- |
| | piperidin-1-yl-ethyl)-3-[1-pyridin-2-yl- |
| | methylidene]-piperidin-4-one |
| 137 | 5,5-Dimethyl-3-[1-[6-(4-methyl-piperazin-1- |
| | yl)-pyridin-2-yl]-methylidene]-1-(2-piperidin- |
| | 1-yl-ethyl)-2-p-tolyl-piperidin-4-one |
| 138 | 5,5-Dimethyl-1-(2-morpholin-4-yl-2-oxo-ethyl)- |
| | 3-[1-(6-morpholin-4-yl-pyridin-2-yl)- |
| | methylidene]-2-phenyl-piperidin-4-one |
| 139 | 5,5-Dimethyl-1-(2-piperidin-1-yl-ethyl)-3-[1- |
| | pyridin-2-yl-methylidene]-2-p-tolyl-piperidin- |
| | 4-one |
| 140 | 2-(4-Fluoro-phenyl)-5,5-dimethyl-3-[1-[6-(4- |
| | methyl-piperazin-1-yl)-pyridin-2-yl]- |
| | methylidene]-1-(2-piperidin-1-yl-ethyl)- |
| | piperidin-4-one |
| 141 | 3,3-Dimethyl-5-[1-quinolin-2-yl-methylidene]- |
| | 1-thiophen-2-ylmethyl-piperidin-4-one |
| 142 | 3,3-Dimethyl-5-[1-[6-(4-methyl-piperazin-1- |
| | yl)-pyridin-2-yl]-methylidene]-1-thiophen-2- |
| | ylmethyl-piperidin-4-one |
| 143 | 3,3-Dimethyl-5-[1-pyridin-2-yl-methylidene]-1- |
| | thiophen-2-ylmethyl-piperidin-4-one |
| 144 | 5,5-Dimethyl-3-[1-pyridin-2-yl-methylidene]-1- |
| | thiophen-2-ylmethyl-2-p-tolyl-piperidin-4-one |
| 145 | 5,5-Dimethyl-3-[1-(6-morpholin-4-yl-pyridin-2- |
| | yl)-methylidene]-1-thiophen-2-ylmethyl-2-p- |
| | tolyl-piperidin-4-one |
| 146 | 5,5-Dimethyl-3-[1-(6-morpholin-4-yl-pyridin-2- |
| | yl)-methylidene]-2-phenyl-1-thiophen-2- |
| | ylmethyl-piperidin-4-one |
| 147 | 5,5-Dimethyl-2-phenyl-3-[1-pyridin-2-yl- |
| | methylidene]-1-thiophen-2-ylmethyl-piperidin- |
| | 4-one |
| 148 | 2-(2,5-Dimethoxy-phenyl)-5,5-dimethyl-1-(4- |
| | methyl-benzyl)-3-[1-quinolin-2-yl- |
| | methylidene]-piperidin-4-one |
| 149 | 2-(4-Dimethylamino-phenyl)-5,5-dimethyl-3-[1- |
| | pyridin-2-yl-methylidene]-1-(2-thiophen-2-yl- |
| | ethyl)-piperidin-4-one |
| 150 | 2-(4-Dimethylamino-phenyl)-5,5-dimethyl-3-[1- |
| | (6-morpholin-4-yl-pyridin-2-yl)-methylidene]- |
| | 1-(2-thiophen-2-yl-ethyl)-piperidin-4-one |
| 151 | 1-Benzyl-3-(3,4-dimethoxy-phenyl)-5-[1- |
| | pyridin-2-yl-methylidene]-piperidine-2,4-dione |
| 152 | 3,3-Dimethyl-5-[1-[6-(4-methyl-piperazin-1- |
| | yl)-pyridin-2-yl]-methylidene]-1-(2-thiophen- |
| | 2-yl-ethyl)-piperidin-4-one |
| 153 | 5,5-Dimethyl-2-(4-methylsulfanyl-phenyl)-3-[1- |
| | (6-morpholin-4-yl-pyridin-2-yl)-methylidene]- |
| | 1-(2-thiophen-2-yl-ethyl)-piperidin-4-one |
| 154 | 2-(4-Dimethylamino-phenyl)-5,5-dimethyl-3-[1- |
| | pyridin-2-yl-methylidene]-1-thiophen-2- |
| | ylmethyl-piperidin-4-one |
| 155 | 2-(4-Dimethylamino-phenyl)-5,5-dimethyl-3-[1- |
| | (6-morpholin-4-yl-pyridin-2-yl)-methylidene]- |
| | 1-thiophen-2-ylmethyl-piperidin-4-one |
| 156 | 1-Benzyl-3-(3,4-dimethoxy-phenyl)-5-[1-[6-(4- |
| | methyl-piperazin-1-yl)-pyridin-2-yl]- |
| | methylidene]-piperidine-2,4-dione |
| 157 | 5,5-Dimethyl-2-(4-methylsulfanyl-phenyl)-3-[1- |
| | pyridin-2-yl-methylidene]-1-(2-thiophen-2-yl- |
| | ethyl)-piperidin-4-one |
| 158 | 5,5-Dimethyl-3-[1-quinolin-2-yl-methylidene]- |
| | 1-thiophen-2-ylmethyl-2-p-tolyl-piperidin-4- |
| | one |
| 159 | 5,5-Dimethyl-2-(4-methylsulfanyl-phenyl)-3-[1- |
| | quinolin-2-yl-methylidene]-1-thiophen-2- |
| | ylmethyl-piperidin-4-one |
| 160 | 2-(4-Dimethylamino-phenyl)-5,5-dimethyl-3-[1- |
| | quinolin-2-yl-methylidene]-1-(2-thiophen-2-yl- |
| | ethyl)-piperidin-4-one |
| 161 | 5,5-Dimethyl-2-(4-methylsulfanyl-phenyl)-3-[1- |
| | quinolin-2-yl-methylidene]-1-(2-thiophen-2-yl- |
| | ethyl)-piperidin-4-one |
| 162 | 5,5-Dimethyl-3-[1-(6-morpholin-4-yl-pyridin-2- |
| | yl)-methylidene]-1-(2-thiophen-2-yl-ethyl)-2- |
| | p-tolyl-piperidin-4-one |
| 163 | 2-(2,5-Dimethoxy-phenyl)-5,5-dimethyl-1-(4- |
| | methyl-benzyl)-3-[1-pyrazin-2-yl-methylidene]- |
| | piperidin-4-one |
| 164 | 5,5-Dimethyl-3-[1-pyridin-2-yl-methylidene]-1- |
| | (2-thiophen-2-yl-ethyl)-2-p-tolyl-piperidin-4- |
| | one |
| 165 | 1-Benzyl-3-(3,4-dimethyl-phenyl)-5[1-(6- |
| | morpholin-4-yl-pyridin-2-yl)-methylidene]- |
| | piperidine-2,4-dione |
| 166 | 1-Benzyl-5,5-dimethyl-3-[1-(4-methylsulfanyl- |
| | phenyl)-methylidene]-2,3,5,6-tetrahydro-1H- |
| | [2,3′]bipyridinyl-4-one |
| 167 | 1-Benzyl-5,5-dimethyl-3-[1-(4-trifluoromethyl- |
| | phenyl)-methylidene]-2,3,5,6-tetrahydro-1H- |
| | [2,3′]bipyridinyl-4-one |
| 168 | 1-(2-Fluoro-benzyl)-5,5-dimethyl-2-(4- |
| | methylsulfanyl-phenyl)-3-[1-pyridin-2-yl- |
| | methylidene]-piperidin-4-one |
| 169 | 1-(2-Fluoro-benzyl)-5,5-dimethyl-2-phenyl-3- |
| | [1-pyridin-2-yl-methylidene]-piperidin-4-one |
| 170 | 1-(2-Fluoro-benzyl)-5,5-dimethyl-3-[1-(6- |
| | morpholin-4-yl-pyridin-2-yl)-methylidene]-2- |
| | phenyl-piperidin-4-one |
| 171 | {5,5-Dimethyl-3-[1-(6-morpholin-4-yl-pyridin- |
| | 2-yl)-methylidene]-4-oxo-2-p-tolyl-piperidin- |
| | 1-yl}-acetic acid |
| 172 | 1-Benzyl-3-[1-(6-morpholin-4-yl-pyridin-2-yl)- |
| | methylidene]-5-phenyl-piperidin-4-one |
| 173 | 1-Benzyl-3-pheny-5-[1-pyridin-2-yl- |
| | methylidene]-piperidin-4-one |
| 174 | 4-Oxo-3-phenyl-5-[1-pyridin-2-yl-methylidene]- |
| | piperidine-1-carboxylic acid (4-chloro- |
| | phenyl)-amide |
| 175 | 4-Oxo-3-phenyl-5-[1-pyridin-2-yl-methylidene]- |
| | piperidine-1-carboxylic acid (4- |
| | methylsulfanyl-phenyl)-amide |
| 176 | 3,3-Dimethyl-5-[1-(6-morpholin-4-yl-pyridin-2- |
| | yl)-methylidene]-4-oxo-2-phenyl-piperidine-1- |
| | carboxylic acid phenylamide |
| 177 | 2-Benzyl-3,3-dimethyl-5-[1-(6-morpholin-4-yl- |
| | pyridin-2-yl)-methylidene]-4-oxo-piperidine-1- |
| | carboxylic acid (4-methoxy-phenyl)-amide |
| 178 | 2-Benzyl-3,3-dimethyl-5-[1-(6-morpholin-4-yl- |
| | pyridin-2-yl)-methylidene]-4-oxo-piperidine-1- |
| | carbothioic acid phenylamide |
| 179 | 2-Benzyl-3,3-dimethyl-5-[1-(6-morpholin-4-yl- |
| | pyridin-2-yl)-methylidene]-4-oxo-piperidine-1- |
| | carboxylic acid (4-fluoro-phenyl)-amide |
| 180 | 2-Benzyl-3,3-dimethyl-5-[1-(6-morpholin-4-yl- |
| | pyridin-2-yl)-methylidene]-4-oxo-piperidine-1- |
| | carboxylic acid isopropylamide |
| 181 | 2-Benzyl-3,3-dimethyl-5-[1-(6-morpholin-4-yl- |
| | pyridin-2-yl)-methylidene]-4-oxo-piperidine-1- |
| | carboxylic acid p-tolylamide |
| 182 | 2-Benzyl-3,3-dimethyl-5-[1-(6-morpholin-4-yl- |
| | pyridin-2-yl)-methylidene]-4-oxo-piperidine-1- |
| | carboxylic acid phenylamide |
| 183 | 3,3-Dimethyl-5-[1-(6-morpholin-4-yl-pyridin-2- |
| | yl)-methylidene]-4-oxo-2-phenyl-piperidine-1- |
| | carboxylic acid p-tolylamide |
| 184 | 3,3-Dimethyl-5-[1-(6-morpholin-4-yl-pyridin-2- |
| | yl)-methylidene]-4-oxo-2-phenyl-piperidine-1- |
| | carboxylic acid (4-methoxy-phenyl)-amide |
| 185 | 4-Oxo-3-phenyl-5-[1-pyridin-2-yl-methylidene]- |
| | piperidine-1-carboxylic acid (2,4-dimethoxy- |
| | phenyl)-amide |
| 186 | 4-Oxo-3-phenyl-5-[1-pyridin-2-yl-methylidene]- |
| | piperidine-1-carboxylic acid phenylamide |
| 187 | 4-Oxo-3-phenyl-5-[1-pyridin-2-yl-methylidene]- |
| | piperidine-1-carboxylic acid p-tolylamide |
| 188 | 3,3-Dimethyl-5-[1-(6-morpholin-4-yl-pyridin-2- |
| | yl)-methylidene]-4-oxo-2-phenyl-piperidine-1- |
| | carboxylic acid (4-fluoro-phenyl)-amide |
| 189 | 3-[1-(4-Methylsulfanyl-phenyl)-methylidene]-4- |
| | oxo-5-phenyl-piperidine-1-carboxylic acid |
| | phenylamide |
| 190 | 3-[1-(4-Methylsulfanyl-phenyl)-methylidene]-4- |
| | oxo-5-phenyl-piperidine-1-carboxylic acid (4- |
| | chloro-phenyl)-amide |
| 191 | 3-[1-(4-Methanesulfonyl-phenyl)-methylidene]- |
| | 4-oxo-5-phenyl-piperidine-1-carboxylic acid |
| | phenylamide |
| 192 | 1,5,5-Trimethyl-3-[1-(6-morpholin-4-yl- |
| | pyridin-2-yl)-methylidene]-2-phenyl-piperidin- |
| | 4-one |
| 193 | 3,3-Dimethyl-2-morpholin-4-ylmethyl-4-oxo-5- |
| | [1-pyridin-2-yl-methylidene]-piperidine-1- |
| | carboxylic acid (4-methylsulfanyl-phenyl)- |
| | amide |
| 194 | 3,3-Dimethyl-2-morpholin-4-ylmethyl-4-oxo-5- |
| | [1-pyridin-2-yl-methylidene]-piperidine-1- |
| | carboxylic acid (4-methoxy-phenyl)-amide |
| 195 | 4-({3,3-Dimethyl-2-morpholin-4-ylmethyl-4-oxo- |
| | 5-[1-pyridin-2-yl-methylidene]-piperidine-1- |
| | carbonyl}-amino)-benzoic acid ethyl ester |
| 196 | N-{3,3-Dimethyl-5-[1-(6-morpholin-4-yl- |
| | pyridin-2-yl)-methylidene]-4-oxo-2-phenyl- |
| | piperidine-1-carbonyl}-benzenesulfonamide |
| 197 | 1-Methanesulfonyl-3,3-dimethyl-2-morpholin-4- |
| | ylmethyl-5-[1-pyridin-2-yl-methylidene]- |
| | piperidin-4-one |
| 198 | 3,3-Dimethyl-2-morpholin-4-ylmethyl-5-[1- |
| | pyridin-2-yl-methylidene]-1-(toluene-4- |
| | sulfonyl)-piperidin-4-one |
| 199 | 1-Methanesulfonyl-3,3-dimethyl-2-phenyl-5-[1- |
| | pyridin-2-yl-methylidene]-piperidin-4-one |
| 200 | 1-Methanesulfonyl-3,3-dimethyl-5-[1-(6- |
| | morpholin-4-yl-pyridin-2-yl)-methylidene]-2- |
| | phenyl-piperidin-4-one |
| 201 | 3-[1-(6-Morpholin-4-yl-pyridin-2-yl)- |
| | methylidene]-5-phenyl-1-(toluene-4-sulfonyl)- |
| | piperidin-4-one |
| 202 | 3-Phenyl-5-[1-pyridin-2-yl-methylidene]-1- |
| | (toluene-4-sulfonyl)-piperidin-4-one |
| 203 | 1-Acetyl-3-[1-(6-morpholin-4-yl-pyridin-3-yl)- |
| | methylidene]-5-phenyl-piperidin-4-one |
| 204 | 1-Acetyl-3-methyl-5-[1-(6-morpholin-4-yl- |
| | pyridin-2-yl)-methylidene]-3-phenyl-piperidin- |
| | 4-one |
| 205 | 3-[1-(6-Morpholin-4-yl-pyridin-2-yl)- |
| | methylidene]-4-oxo-5-phenyl-piperidine-1- |
| | carboxylic acid phenylamide |
| 206 | 1-Methanesulfonyl-3-[1-(6-morpholin-4-yl- |
| | pyridin-2-yl)-methylidene]-5-phenyl-piperidin- |
| | 4-one |
| 207 | 3-[1-(6-Morpholin-4-yl-pyridin-2-yl)- |
| | methylidene]-4-oxo-5-phenyl-piperidine-1- |
| | carboxylic acid p-tolylamide |
| 208 | 3-[1-(6-Morpholin-4-yl-pyridin-2-yl)- |
| | methylidene]-4-oxo-5-phenyl-piperidine-1- |
| | carboxylic (2,4-dimethoxy-phenyl)-amide |
| 209 | 4-Oxo-3-phenyl-5-[1-pyridin-2-yl-methylidene]- |
| | piperidine-1-carboxylic acid (4-acetyl- |
| | phenyl)-amide |
| 210 | 1-Methanesulfonyl-3-phenyl-5-[1-pyridin-2-yl- |
| | methylidene]-piperidin-4-one |
| 211 | 4-Oxo-3-phenyl-5-[1-pyridin-2-yl-methylidene]- |
| | piperidine-1-carboxylic acid (2,4-dihydroxy- |
| | phenyl)-amide |
| 212 | 4-Oxo-3-phenyl-5-[1-pyridin-2-yl-methylidene]- |
| | piperidine-1-carboxylic acid (4-hydroxy- |
| | phenyl)-amide |
| 213 | 4-Oxo-3-phenyl-5-[1-pyridin-2-yl-methylidene]- |
| | piperidine-1-carboxylic (4-methanesulfonyl- |
| | phenyl)-amide |
| 214 | 1-(2,4-Dihydroxy-benzenesulfonyl)-3-phenyl-5- |
| | [1-pyridin-2-yl-methylidene]-piperidin-4-one |
| 215 | 4-{4-Oxo-3-phenyl-5-[1-pyridin-2-yl- |
| | methylidene]-piperidine-1-carbonyl}- |
| | benzenesulfonamide |
| 216 | 3-(4-Hydroxy-phenyl)-4-oxo-5-[1-pyridin-2-yl- |
| | methylidene]-piperidine-1-carboxylic acid |
| | phenylamide |
| 217 | 3-(4-Hydroxy-phenyl)-4-oxo-5-[1-pyridin-2-yl- |
| | methylidene]-piperidine-1-carboxylic acid (4- |
| | hydroxy-phenyl)-amide |
| 218 | 1-(4-Acetyl-benzoyl)-3-phenyl-5-[1-pyridin-2- |
| | yl-methylidene]-piperidin-4-one |
| 219 | 3-(4-Hydroxy-phenyl)-5-[1-pyridin-2-yl- |
| | methylidene]-1-(toluene-4-sulfonyl)-piperidin- |
| | 4-one |
| 220 | 3-(4-Hydroxy-phenyl)-1-(4-methyl-benzoyl)-5- |
| | [1-pyridin-2-yl-methylidene]-piperidin-4-one |
| 221 | 1-Benzenesulfonyl-3-phenyl-5-[1-pyridin-2-yl- |
| | methylidene]-piperidin-4-one |
| 222 | 1-Benzoyl-3-(4-hydroxy-phenyl)-5-[1-pyridin-2- |
| | yl-methylidene]-piperidin-4-one |
| 223 | 1-(4-Hydroxy-benzyl)-2-(4-hydroxy-phenyl)-5,5- |
| | dimethyl-3-[1-pyridin-2-yl-methylidene]- |
| | piperidin-4-one |
| 224 | 1-(4-Hydroxy-benzyl)-2-(5-hydroxy-2-methoxy- |
| | phenyl)-5,5-dimethyl-3-[1-pyridin-2-yl- |
| | methylidene]-piperidin-4-one |
| 225 | 1-Methanesulfonyl-2-phenyl-4-[1-pyridin-2-yl- |
| | methylidene]-piperidin-3-one |
| 226 | 1-Benzenesulfonyl-3-(4-hydroxy-phenyl)- |
| | 5-[1-pyridin-2-yl-methylidene]-piperidin- |
| | 4-one |
| 227 | 1-Benzyl-2-(4-methanesulfonyl-phenyl)-5,5- |
| | dimethyl-3-[1-(6-morpholin-4-yl-pyridin-2-yl)- |
| | methylidene]-piperidin-4-one |
| 228 | 1-Benzyl-5-[1-(4-methanesulfonyl-phenyl)- |
| | methylidene]-3,3-dimethyl-piperidin-4-one |
| 229 | 1-Benzyl-2-(4-methanesulfonyl-phenyl)-5,5- |
| | dimethyl-3-[1-pyridin-2-yl-methylidene]- |
| | piperidin-4-one |
| 230 | 2-(2,5-Dimethoxy-phenyl)-5,5-dimethyl-1-(4- |
| | methyl-benzyl)-3-[1-(6-morpholin-4-yl-pyridin- |
| | 2-yl)-methylidene]-piperidin-4-one |
| 231 | 5,5-Dimethyl-1-(4-methyl-benzyl)-2-phenyl-3- |
| | [1-pyridin-2-yl-methylidene]-piperidin-4-one |
| 232 | 5,5-Dimethyl-1-(4-methyl-benzyl)-3-[1-(6- |
| | morpholin-4-yl-pyridin-2-yl)-methylidene]-2- |
| | phenyl-piperidin-4-one |
| 233 | 2-(2,5-Dimethoxy-phenyl)-5,5-dimethyl-1-(4- |
| | methyl-benzyl)-3-[1-pyridin-2-yl-methylidene]- |
| | piperidin-4-one |
| 234 | 2-(2,5-Dimethoxy-phenyl)-5,5-dimethyl-1-(4- |
| | methyl-benzyl)-3-[1-[6-(4-methyl-piperazin-1- |
| | yl)-pyridin-2-yl]-methylidene]-piperidin-4-one |
| 235 | 1-(3,4-Dimethoxy-benzyl)-5,5-dimethyl-3-[1-(6- |
| | morpholin-4-yl-pyridin-2 yl)-methylidene]-2- |
| | phenyl-piperidin-4-one |
| 236 | 3-(4-Hydroxy-phenyl)-1-methanesulfonyl-5-[1- |
| | pyridin-2-yl-methylidene]-piperidin-4-one |
| 237 | 1-Benzenesulfonyl-3-(4-hydroxy-phenyl)-5-[1- |
| | pyridin-2-yl-methylidene]-piperidin-4-one |
| 238 | 1-(4-Amino-benzenesulfonyl)-3-phenyl-5-[1- |
| | pyridin-2-yl-methylidene]-piperidin-4-one |
| 239 | 1-(4-Hydroxy-benzoyl)-3-(4-hydroxy-phenyl)-5- |
| | [1-pyridin-2-yl-methylidene]-piperidin-4-one |
| 240 | 1-(3,5-Dihydroxy-benzoyl)-3-phenyl-5-[1- |
| | pyridin-2-yl-methylidene]-piperidin-4-one |
| 241 | 1-(4-Amino-benzenesulfonyl)-3-phenyl-5-[1- |
| | pyridin-2-yl-methylidene]-piperidin-4-one |
| 242 | 4-{4-Oxo-3-phenyl-5-[1-pyridin-2-yl- |
| | methylidene]-piperidine-1-sulfonyl}-benzamide |
| 243 | 4-{3-(4-Hydroxy-phenyl)-4-oxo-5-[1-pyridin-2- |
| | yl-methylidene]-piperidine-1-sulfonyl}- |
| | benzamide |
| 244 | 1-(3-Amino-4-hydroxy-benzoyl)-3-phenyl-5-[1- |
| | pyridin-2yl-methylidene]-piperidin-4-one |
| 245 | 1-(3-Amino-4-hydroxy-benzoyl)-3-(4-hydroxy- |
| | phenyl)-5-[1-pyridin-2-yl-methylidene- |
| | piperidin-4-one |
| 246 | 1-(2,4-Dihydroxy-benzenesulfonyl)-3-(4- |
| | hydroxy-phenyl)-5-[1-pyridin-2-yl- |
| | methylidene]-piperidin-4-one |
| 247 | 2-{4-Oxo-3-phenyl-5-[1-pyridin-2-yl- |
| | methylidene]-piperidin-1-yl}-acetamide |
| 248 | 2-{3-(4-Hydroxy-phenyl)-4-oxo-5-[1-pyridin-2- |
| | yl-methylidene]-piperidin-1-yl}-acetamide |
| 249 | 4-Oxo-3-phenyl-5-[1-pyridin-2-yl-methylidene]- |
| | piperidine-1-sulfonic acid amide |
| 250 | 3-(4-Hydroxy-phenyl)-4-oxo-5-[1-pyridin-2-yl- |
| | methylidene]-piperidine-1-sulfonic acid amide |
| 251 | 4-Oxo-3-phenyl-5-[1-pyridin-2-yl-methylidene]- |
| | piperidine-1-carboxylic acid (4-amino-phenyl)- |
| | amide |
| 252 | 3-(4-Hydroxy-phenyl)-4-oxo-5-[1-pyridin-2-yl- |
| | methylidene]-piperidine- |
| | 1-carboxylic acid (4-amino-phenyl)-amide |
| 253 | 1-(4-Amino-benzoyl)-3-(4-hydroxy-phenyl)-5-[1- |
| | pyridin-2-yl-methylidene]-piperidin-4-one |
| 254 | 1-(4-Amino-benzoyl)-3-phenyl-5-[1-pyridin-2- |
| | yl-methylidene]-piperidin-4-one |
| 255 | 4-{4-Oxo-3-phenyl-5-[1-pyridin-2-yl- |
| | methylidene]-piperidine-1-carbonyl}-benzamide |
| 256 | 4-{3-(4-Hydroxy-phenyl)-4-oxo-5-[1-pyridin-2- |
| | yl-methylidene]-piperidine-1-carbonyl}- |
| | benzamide |
| 257 | 3-{4-Oxo-3-phenyl-5-[1-pyridin-2-yl- |
| | methylidene]-piperidine-1-sulfonyl}-benzoic |
| | acid |
| 258 | 3-{3-(4-Hydroxy-phenyl)-4-oxo-5-[1-pyridin-2- |
| | yl-methylidene]-piperidine-1-sulfonyl}-benzoic |
| | acid |
| 259 | 3-{4-Oxo-3-phenyl-5-[1-pyridin-2-yl- |
| | methylidene]-piperidine-1-carbonyl}-benzoic |
| | acid |
| 260 | 3-{3-(4-Hydroxy-phenyl)-4-oxo-5-[1-pyridin-2- |
| | yl-methylidene]-piperidine-1-carbonyl}-benzoic |
| | acid |
| 261 | 4-{4-Oxo-3-phenyl-5-[1-pyridin-2-yl- |
| | methylidene]-piperidine-1-carbonyl}- |
| | benzenesulfonamide |
| 262 | 1-(4-Methanesulfonyl-benzoyl)-3-phenyl-5-[1- |
| | pyridin-2-yl-methylidene]-piperidin-4-one |
| 263 | 4-({4-Oxo-3-phenyl-5-[1-pyridin-2-yl- |
| | methylidene]-piperidine-1-carbonyl}-amino)- |
| | benzoic acid |
| 264 | 4-({3-(4-Hydroxy-phenyl)-4-oxo-5-[1-pyridin-2- |
| | yl-methylidene]-piperidine- |
| | 1-carbonyl}-amino)-benzoic acid |
| 265 | 1-(4-Hydroxy-benzoyl)-3-(4-hydroxy-phenyl)-5- |
| | [1-(4-methanesulfonyl-phenyl)-methylidene]- |
| | piperidin-4-one |
| 266 | 3-(4-Hydroxy-phenyl)-5-[1-(4-methanesulfonyl- |
| | phenyl)-methylidene]- |
| | 4-oxo-piperidine-1-carboxylic acid (4- |
| | hydroxy-phenyl)-amide |
| 267 | 2-(4-Amino-phenyl)-5,5-dimethyl-3-[1-pyridin- |
| | 2-yl-methylidene]-1-(2-thiophen-2-yl-ethyl)- |
| | piperidin-4-one |
| 268 | 2-(2,4-Dihydroxy-phenyl)-5,5-dimethyl-3-[1- |
| | pyridin-2-yl-methylidene]-1-(2-thiophen-2-yl- |
| | ethyl)-piperidin-4-one |
| 269 | 2-(3-Amino-4-hydroxy-phenyl)-5,5-dimethyl-3- |
| | [1-pyridin-2-yl-ethylidene]-1-(2-thiophen-2- |
| | yl-ethyl)-piperidin-4-one |
| 270 | 4-[5,5-Dimethyl-4-oxo-3-[1-pyridin-2-yl- |
| | methylidene]-1-(2-thiophen-2-yl-ethyl)- |
| | piperidin-2-yl]-benzamide |
| 271 | 1-(3-Hydroxy-benzenesulfonyl)-3,3-dimethyl-2- |
| | phenyl-5-[1-pyridin-2-yl-methylidene]- |
| | piperidin-4-one |
| 272 | 1-(2,5-Dihydroxy-benzenesulfonyl)-3,3- |
| | dimethyl-2-phenyl-5-[1-pyridin-2-yl- |
| | methylidene]-piperidin-4-one |
| 273 | 4-{3,3-Dimethyl-4-oxo-2-phenyl-5-[1-pyridin-2- |
| | yl-methylidene]-piperidine-1-carbonyl}- |
| | benzenesulfonamide |
| 274 | 2-(4-Amino-phenyl)-1-(4-hydroxy-benzyl)-5,5- |
| | dimethyl-3-[1-pyridin-2-yl-methylidene]- |
| | piperidin-4-one |
| 275 | 4-{1-(4-Hydroxy-benzyl)-5,5-dimethyl-4-oxo-3- |
| | [1-pyridin-2-yl-methylidene]-piperidin-2-yl}- |
| | benzamide |
| 276 | 2-(4-Amino-phenyl)-1-(3,4-dihydroxy-benzyl)- |
| | 5,5-dimethyl-3-[1-pyridin-2-yl-methylidene]- |
| | piperidin-4-one |
| 277 | 4-{1-(3,4-Dihydroxy-benzyl)-5,5-dimethyl-4- |
| | oxo-3-[1-pyridin-2-yl-methylidene]-piperidin- |
| | 2-yl}-benzamide |
| 278 | 1-(3,4-Dihydroxy-benzyl)-2-(4-hydroxy-phenyl)- |
| | 5,5-dimethyl-3-[1-pyridin-2-yl-methylidene]- |
| | piperidin-4-one |
| 279 | 4-({4-Oxo-3-phenyl-5-[1-pyridin-2-yl- |
| | methylidene]-piperidine-1-carbonyl}-amino)- |
| | benzoic acid |
| 280 | 4-Oxo-3-phenyl-5-[1-pyridin-2-yl-methylidene]- |
| | piperidine-1-carboxylic acid (4-carbamoyl- |
| | phenyl)-amide |
| 281 | 1-(4-Hydroxy-benzoyl)-3-(4-hydroxy-phenyl)-5- |
| | [1-pyridin-2-yl-methylidene]-piperidin-4-one |
| 282 | 3-(4-Hydroxy-phenyl)-4-oxo-5-[1-pyridin-2-yl- |
| | methylidene]-piperidine- |
| | 1-carboxylic acid (4-hydroxy-phenyl)-amide |
| 283 | 3-Oxo-2-phenyl-4-[1-pyridin-2-yl-methylidene]- |
| | piperidine-1-carboxylic acid amide |
| 284 | 3-Oxo-2-phenyl-4-[1-pyridin-2-yl-methylidene]- |
| | piperidine-1-sulfonic acid amide |
| 285 | 1-(4-Hydroxy-benzenesulfonyl)-2-phenyl-4-[1- |
| | pyridin-2-yl-methylidene]-piperidin-3-one |
| 286 | 1-(4-Hydroxy-benzoyl)-2-phenyl-4-[1-pyridin-2- |
| | yl-methylidene]-piperidin-3-one |
| 287 | 3-Oxo-2-phenyl-4-[1-pyridin-2-yl-methylidene]- |
| | piperidine-1-carboxylic acid (4-hydroxy- |
| | phenyl)-amide |
| 288 | 3-Oxo-2-phenyl-4-[1-pyridin-2-yl-methylidene]- |
| | piperidine-1-carboxylic acid (4-sulfamoyl- |
| | phenyl)-amide |

or its pharmaceutically acceptable salts and their hydrates, solvates, stereoisomers, conformers, tautomers, polymorphs and prodrugs thereof.

3. The method of treating disease condition accompanying pathological stress in a living mammalian organism, including a human being, comprising administering to said living mammalian organism in need thereof a therapeutically effective amount of one or more compound as claimed in claim 1.

4. The method as claimed in claim 3, wherein said disease condition accompanying pathological stress is selected from stroke, myocardial infarction, inflammatory disorders, hepatotoxicity, sepsis, diseases of viral origin, allograft rejection, tumourous diseases, gastric mucosal damage, brain haemorrhage, endothelial dysfunctions, diabetic complications, neuro-degenerative diseases, epilepsy, post-traumatic neuronal damage, acute renal failure, glaucoma and aging related skin degeneration.

5. The method as claimed in claim 4, wherein said disease condition accompanying pathological stress is stroke.

6. The method as claimed in claim 4, wherein said disease condition accompanying pathological stress is myocardial infarction.

7. The method as claimed in claim 4, wherein said disease condition accompanying pathological stress is inflammatory disorders.

8. The method as claimed in claim 4, wherein said diabetic complications are selected from diabetic neuropathy, diabetic retinopathy and chronic wound healing.

9. The method as claimed in claim 4, wherein said neuro-degenerative diseases are selected from Alzheimer's disease, amyotrophic lateral sclerosis and Parkinson's disease.

10. The method as claimed in claim 4, wherein said disease condition accompanying pathological stress is epilepsy.

11. The pharmaceutical composition comprising a therapeutically effective amount of one or more compound as claimed in claim 1, in association with a pharmaceutically acceptable carrier, diluent or excipient.

12. The pharmaceutical composition as claimed in claim 11, in the form of an oral formulation or a parenteral formulation.

13. The use of one or more compound as claimed in claim 1, in the manufacture of a medicament for the treatment of disease condition accompanying pathological stress in a living mammalian organism, including a human being.

14. The use as claimed in claim 13, wherein said disease condition accompanying pathological stress is selected from stroke, myocardial infarction, inflammatory disorders, hepatotoxicity, sepsis, diseases of viral origin, allograft rejection, tumourous diseases, gastric mucosal damage, brain haemorrhage, endothelial dysfunctions, diabetic complications, neuro-degenerative diseases, epilepsy, post-traumatic neuronal damage, acute renal failure, glaucoma and aging related skin degeneration.

15. The use as claimed in claim 14, wherein said disease condition accompanying pathological stress is stroke.

16. The use as claimed in claim 14, wherein said disease condition accompanying pathological stress is myocardial infarction.

17. The use as claimed in claim 14, wherein said disease condition accompanying pathological stress is inflammatory disorders.

18. The use as claimed in claim 14, wherein said diabetic complications are selected from diabetic neuropathy, diabetic retinopathy and chronic wound healing.

19. The use as claimed in claim 14, wherein said neuro-degenerative diseases are selected from Alzheimer's disease, amyotrophic lateral sclerosis and Parkinson's disease.

20. The use as claimed in claim 14, wherein said disease condition accompanying pathological stress is epilepsy.
